SEQUENCE LISTING (83 sequences)

SEQ ID NO: 1 - DNA, 1464 bp, Unknown
  Collinsella aerofaciens_F
agagttcgatcctggctcaggatgaacgctggcggcgcgcctaacacatgcaagtcgaacggcacccctctccggagggaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgccggcccaaccgagaggggggaggcgccgaaggtgtggagggtgaggggggtgaagtcgtaacaaggta

SEQ ID NO: 2 - DNA, 1517 bp, Unknown
  Bacteroides xylanisolvens
agagtttgatcctggctcaggatgaacgctagctacaggcttaacacatgcaagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacct

SEQ ID NO: 3 - DNA, 1485 bp, Unknown
  Alistipes putredinis
agagtttgatcctggctcaggatgaacgctagcggcaggcttaacacatgcaagtcgaggggcagcataatggatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctgaagttcgtgaccgcaaggagcgacctagggcaaaactggtgactggggctaagtcgtaacaaggta

SEQ ID NO: 4 - DNA, 1512 bp, Unknown
  Alistipes shahii
agagtttgatcctggctcaggatgaacgctagcggcaggcctaacacatgcaagtcgaggggcagcacggtgtagcaatacactggtggcgaccggcgcacgggtgcgtaacgcgtatgcaacctacccataacagggggataacactgagaaattggtactaataccccataacatcaggaccggcatcggttctggttgaaaactccggtggttatggatgggcatgcgttgtattagctggttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagagtaaacgctcttacgtgtaagagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaaggagcgacctagggcaaaaccggtgactggggctaagtcgtaacaaggtagccgtaccggaaggtgcggctggaacacct

SEQ ID NO: 5 - DNA, 1472 bp, Unknown
  Eubacterium_E hallii
ctcaggatgaacgctagctacaggcttaacacatgcaagtcgaggggcagcattccagtttgcttgcaaactggagatggcgaccggcgcacgggtgagtaacacgtatccaacctgccgataactcggggatagcctttcgaaagaaagattaatacccgatggcataatagaaccgcatggtttgattattaaagaatttcggttatcgatggggatgcgttccattaggcagttggtggggtaacggcccaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtggtccacgtgtggatttttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggcgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 6 - DNA, 1488 bp, Unknown
  Blautia_A obeum
agagtttgatcctggctcaggatgaacgctggcggcgtgcttaacacatgcaagtcgaacgggaaaccttttattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgttccttaaccggaactttccttcgggacaggggagacaggtggtgcatggttgtcgtcagctcgtgtcgtgagatgttgggttaagtcccgcaacgagcgcaacccctatccccagtagccagcagtccggctgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggta

SEQ ID NO: 7 - DNA, 1512 bp, Unknown
  Odoribacter splanchnicus
agagtttgatcctggctcaggatgaacgctagcgacaggcttaacacatgcaagtcgaggggcatcatgaggtagcaataccttgatggcgaccggcgcacgggtgagtaacgcgtatgcaacctgcctgataccggggtatagcccatggaaacgtggattaacaccccatagtacttttatcctgcatgggatgtgagttaaatgtttaaggtatcggatgggcatgcgtcctattagttagttggcggggtaacagcccaccaagacgatgataggtaggggttctgagaggaaggtcccccacattggaactgagacacggtccaaactcctacgggaggcagcagtgaggaatattggtcaatggacgagagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacgggggcccgcacaagcggaggaacatgtggtttaattcgatgatacgcgaggaaccttacctgggtttaaatgggaaatgtcgtatttggaaacagatattctcttcggagcgtttttcaaggtgctgcatggttgtcgtcagctcgtgccgtgaggtgtcgggttaagtcccataacgagcgcaacccttaccgttagttgctagcatgtaatgatgagcactctaacgggactgccaccgtaaggtgagaggaaggcggggatgacgtcaaatcagcacggcccttacacccagggctacacacgtgttacaatggccggtacagagggccgctaccaggtgactggatgccaatctcaaaagccggtcgtagttcggattggagtctgtaacccgactccatgaagttggattcgctagtaatcgcgcatcagccatggcgcggtgaatacgttcccgggccttgtacacaccgcccgtcaagccatggaagccgggggtgcctgaagtccgtaaccgcgaggatcggcctagggcaaaactggtaactggggctaagtcgtaacaaggtagccgtaccggaaggtgcggctggaacacct

SEQ ID NO: 8 - DNA, 1473 bp, Unknown
  Bacteroides cellulosilyticus
agagtttgatcctggctcaggatgaacgctagctacaggcttaacacatgcaagtcgaggggcagcatgacctagcaataggttgatggcgaccggcgcacgggtgagtaacacgtatccaacctaccggttattccgggatagcctttcgaaagaaagattaataccggatagtataacgagaaggcatctttttgttattaaagaatttcgataaccgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgagccacgtgtggctttttgtatgtaccatacgaataaggatcggctaactccgtgccagcagccgcggtaatacggaggatccgagcgttatccggatttattgggtttaaagggagcgtaggcggactattaagtcagctgtgaaagtttgcggctcaaccgtaaaattgcagttgatactggtcgtcttgagtgcagtagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacggcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaatatagtggaaacattatagccgcaaggcatttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtccgtaaccgtaaggagcggcctagggtaaaactggtaattggggctaagtcgta

SEQ ID NO: 9 - DNA, 1474 bp, Bacteroides uniformis
ctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcgaggagcgccctagggtaaaactggtgattggggctaagtcgtaacaaggta

SEQ ID NO: 10 - DNA, 1503 bp, Unknown
  Bacteroides_B vulgatus
atgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacagcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 11 - DNA, 1525 bp, Bacteroides fragilis
atgaagagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatggcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaagcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggatcgtcctagggtaaaactggtgactggggctaagtcgtaacaaggtagccgtaccggaaggtgcggctggaacacctcctt

SEQ ID NO: 12 - DNA, 1475 bp, Bacteroides caccae
tggctcaggatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagctcactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccataggccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 13 - DNA, 1492 bp, Unknown
  Paeniclostridium sordellii
gtttgatcctggctcaggatgaacgctggcggcgtgcctaacacatgcaagtcgagcgaacccttcggggtgagcggcggacgggtgagtaacgcgtgggtaacctgccctgtacacacggataacataccgaaaggtatgctaatacgggataatatatgagagtcgcatggcttttgtatcaaagctctggcggtacaggatggacccgcgtctgattagctagttggtaaggtaacggcttaccaaggcaacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggcggtctttcaagccagaagtgaaaggctacggctcaaccgtagtaagcttttggaactgtaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgtgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttgggggcgcccgaagccggttagctaaccttttaggaagcggccgtcgaaggtgaaaccaatgactggggtgaagtcgtaacaaggtagccgtatcggaaggtgcggctggatcacctccttt

SEQ ID NO: 14 - DNA, 1490 bp, Unknown
  Blautia producta
agagtttgatcctggctcaggatgaacgctggcggcgtgcttaacacatgcaagtcgagcgaagcactaagacagatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggta

SEQ ID NO: 15 - DNA, 1475 bp, Unknown
  Absiella innocuum
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccga

SEQ ID NO: 16 - DNA, 1496 bp, Unknown
  Clostridium_M clostridioforme
agagtttgatcctggctcaggatgaacgctggcggcgtgcctaacacatgcaagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccggggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcaggtagagccgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgta

SEQ ID NO: 17 - DNA, 1490 bp, Unknown
  Paraclostridium massiliensis
agagtttgatcctggctcaggatgaacgctggcggcgtgcctaacacatgcaagtcgagcgatctcttcggagagagcggcggacgggtgagtaacgcgtgggtaacctgccctgtacacacggataacataccgaaaggtatactaatacgggataacatacgaaagtcgcatggcttttgtatcaaagctccggcggtacaggatggacccgcgtctgattagctagttggtaaggtaatggcttaccaaggcaacgatcagtagccgacctgagagggtgatcggccacactggaactgagacacggtccagactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggtggttttttaagtcagaagtgaaaggctacggctcaaccgtagtaagcttttgaaactagagaacttgagtgcaggagaggagagtagaattcctagtgtagcggtgaaatgcgtagatattaggaggaataccagtagcgaaggcggctctctggactgtaactgacactgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttacctaagcttgacatcccactgacctctccctaatcggagatttcccttcggggacagtggtgacaggtggtgcatggttgtcgtcagctcgtgtcgtgagatgttgggttaagtcccgcaacgagcgcaacccttgcctttagttgccagcattaagttgggcactctagagggactgccgaggataactcggaggaaggtggggatgacgtcaaatcatcatgccccttatgcttagggctacacacgtgctacaatgggtggtacagagggttgccaagccgcgaggtggagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatggaagttgggggcgcccgaagccggttagctaaccttttaggaagcggccgtcgaaggtgaaaccaatgactggggtgaagtcgtaacaaggtagccgtatcggaaggtgcggctggatcacct

SEQ ID NO: 18 - DNA, 1524 bp, Unknown
  Dorea longicatena B
agagtttgatcctggctcaggatgaacgctggcggcgtgcttaacacatgcaagtcgagcgaagcacttaagtctgattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctcctt

SEQ ID NO: 19 - DNA, 1474 bp, Bacteroides thetaiotaomicron
atgaagagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggcataatagaaccgcatggtttttttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggc

SEQ ID NO: 20 - DNA, 1483 bp, Parabacteroides distasonis
cgaagagtttgatcctggctcaggatgaacgctagcgacaggcttaacacatgcaagtcgaggggcagcacaggtagcaataccgggtggcgaccggcgcacgggtgagtaacgcgtatgcaacttgcctatcagagggggataacccggcgaaagtcggactaataccgcatgaagcagggatcccgcatgggaatatttgctaaagattcatcgctgatagataggcatgcgttccattaggcagttggcggggtaacagcccaccaaaccgacgatggataggggttctgagaggaaggtcccccacattggtactgagacacggaccaaactcctacgggaggcagcagtgaggaatattggtcaatggccgagaggctgaaccagccaagtcgcgtgagggatgaaggttctatggatcgtaaacctcttttataagggaataaagtgcgggacgtgtcccgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacactgtaagcggcacagcgaaagcgttaagtgatccacctggggagtacgccggcaacggtgaaactcaaaggaattgacgggggcccgcacaagcggaggaacatgtggtttaattcgatgatacgcgaggaaccttacccgggtttgaacgcattcggaccgaggtggaaacaccttttctagcaatagccgtttgcgaggtgctgcatggttgtcgtcagctcgtgccgtgaggtgtcggcttaagtgccataacgagcgcaacccttgccactagttactaacaggtaaagctgaggactctggtgggactgccagcgtaagctgcgaggaaggcggggatgacgtcaaatcagcacggcccttacatccggggcgacacacgtgttacaatggcgtggacaaagggaagccacctggcgacagggagcgaatccccaaaccacgtctcagttcggatcggagtctgcaacccgactccgtgaagctggattcgctagtaatcgcgcatcagccatggcgcggtgaatacgttcccgggccttgtacacaccgcccgtcaagccatgggagccgggggtacctgaagtccgtaaccgcgaggatcggcctagggtaaaactggtgactggggctaagtcgtaacaag

SEQ ID NO: 21 - DNA, 1538 bp, Escherichia coli
ttgaagagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt

SEQ ID NO: 22 - DNA, 1506 bp, Bifidobacterium longum
tggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacctcctt

SEQ ID NO: 23 - DNA, 1475 bp, Unknown
  Absiella innocuum
atggagagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcggccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccga

SEQ ID NO: 24 - DNA, 1500 bp, Unknown
  Fusobacterium_A sp
agagtttgatcctggctcaggatgaacgctgacagaatgcttaacacatgcaagtctacttgatccttcgggtgatggtggcggacgggtgagtaacgcgtaaagaacttgccctgcagtctgggacaacatttggaaacgaatgctaataccggatattatgtatttctcgcatgagttttacatgaaagctatatgcgctgcaggagagctttgcgtcctattagctagttggtgaggtaacggctcaccaaggccatgataggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgctaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgtcgtgagatgttgggttaagtcccgcaacgagcgcaacccctttcgtatgttgccatcattaagttgggcactcatgcgatactgcctgcgatgagcaggaggaaggtggggatgacgtcaagtcatcatgccccttatacgctgggctacacacgtgctacaatgggtagtacagagagtcgcaaacctgcgagggggagctaatctcagaaaactattctcagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtagcaggcctaaccgcaaggagggatgctccgagggtgtgattagcgattggggtgaagtcgtaacaaggtatccgtacgggaacgtgcggatggatcacctcctt

SEQ ID NO: 25 - DNA, 1489 bp, Unknown
  Parabacteroides merdae
agagtttgatcctggctcaggatgaacgctagcgacaggcttaacacatgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactctttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggtgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaaggatcggcctagggtaaaactggtgactggggctaagtcgtaacaaggta

SEQ ID NO: 26 - DNA, 1522 bp, Unknown
  Coprococcus_B comes
gagagtttgatcctggctcaggatgaacgctggcggcgtgcttaacacatgcaagtcgaacgaagcacttacctttgattcttcggatgaaggtttttgtgactgagtggcggacgggtgagtaacgcgtgggtaacctgcctcatacagggggataacagttagaaatgactgctaataccgcataagaccacagagctgcatggctcagtgggaaaaactccggtggtatgagatggacccgcgtctgattaggtagttggtggggtaacggcctaccaagccaacgatcagtagccgacctgagagggtgaccggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaagcccggggctcaaccccgggactgctttggaaactatgcagctagagtgtcggagaggtaagtggaattcccagtgtagcggtgaaatgcgtagatattgggaggaacaccagtggcgaaggcggcttactggacgatgactgacgttgaggctcgaaagcgtggggagcaaacaggattagataccctggtagtccacgccgtaaacgatgactactaggtgtcggggagcagagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccggcatgtaatgatgccttttcttcggaacaccggtgacaggtggtgcatggttgtcgtcagctcgtgtcgtgagatgttgggttaagtcccgcaacgagcgcaacccttatcttcagtagccagcaattcggatgggcactctggagagactgccagggataacctggaggaaggtggggatgacgtcaaatcatcatgccccttatgagcagggctacacacgtgctacaatgacgtaaacaaagggaagcgagcctgcgagggtaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggtaacgcccgaagtcagtgacccaaccgtaaggagggagctgccgaaggtgggaccgataactggggtgaagtcgtaacaaggtagccgtatcggaaggtgcggctggatcacctcc

SEQ ID NO: 27 - DNA, 1521 bp, Unknown
  Agathobacter rectale
agagtttgatcctggctcaggatgaacgctggcggcgtgcttaacacatgcaagtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaaggagctgtcgaaggcaggctcgataactggggtgaagtcgtaacaaggtagccgtatcggaaggtgcggctggatcacct

SEQ ID NO: 28 - DNA, 1509 bp, Unknown
  Erysipelatoclostridium ramosum
gagagtttgatcctggctcaggatgaacgctggcggcgtgcctaatacatgcaagtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgcccttatgacctgggctacacgtgctacaatggatggtgcagagagaggcgagccgcgggcgaaacaaaagcccataaaaccatttcagttccggattgtagtctgcaactcgactttattacatgagagttggaatcgctagtaatcgcgaatcagcatgtcgcggtttgaatacgttctcgggccttaccgcccgtcacaccaaaggattgataacacggcggtggctaaccgcaaggaaaggctgtctaaggtgggattgatgattggggtaaagtcgtaacgaaggtatccctacgggaacgtggggatggatcacctcc

SEQ ID NO: 29 - DNA, 1496 bp, Unknown
  Clostridium_M citroniae
agagtttgatcctggctcaggatgaacgctggcggcgtgcctaacacatgcaagtcgaacgaagcaattagaaagaagttttcggatggatttctgattgactgagtggcggacgggtgagtaacgcgtggataacctgcctcacactgggggataacagttagaaatggctgctaataccgcataagcgcacagtaccgcatggtacggtgtgaaaaactccggtggtgtgagatggatccgcgtctgattagttagttggcggggtaacggcccaccaagacagcgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaactgacggtacctgactaagaagccccggctaactacgtgccagcagccgcggtaatacgtagggggcaagcgttatccggatttactgggtgtaaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgta

SEQ ID NO: 30 - DNA, 1293 bp, Unknown
  Bacteroides faecis
atgaagagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatggcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgatagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtaggtggatgtttaagtcagttgtgaaagtttgcggctcaaccgtaaaattgcagttgatactggctgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggtgatgctgaggactctggagagactgccatcgtaagatgtgaggaaggtggggatgacgtcaaatcagcacggcccttacgtccggggctacacacgtgttacaatgggaggtacagaaggcagctacccggcgacgggatgccaatccccaaaacctctctcagt

SEQ ID NO: 31 - DNA, 1505 bp, Bacteroides ovatus
atgaagagtttgatcctggctcaggatgaacgctagctacaggcttaacacatgcaagtcgaggggcagcatggtcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagttggcggggtaacggcccaccaaaccgacgatggataggggttctgagaggaaggtcccccacattggtactgagacacggaccaaactcctacgggaggcagcagtgaggaatattggtcaatgggcgagagcctgaaccagccaagtagcgtgaaggatgactgccctatgggttgtaaacttcttttataagggaataaagtcgggtatgcatacccgtttgcatgtactttatgaataaggatcggctaactccgtgccagcagccgcggtaatacggaggatccgagcgttatccggatttattgggtttaaagggagcgtaggtggacgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagacgaattacgaggaaacttgtaagccgcaaggcgtctgtgaaggtgctgcatggttgtcgtcagctcgtgccgtgaggtgtcggcttaagtgccataacgagcgcaacccttatctttagttactaacaggttatgctgaggactctagagagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggatcgccctagggtaaaactggtgactggggctaagtcgtaacaaggtagccgtaccggaag

SEQ ID NO: 32 - DNA, 1511 bp, Bifidobacterium pseudocatenulatum
ttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcctaaccctttgtggatggagccgtctaaggtgagactcgtgattgggactaagtcgtaacaaggtagccgtaccggaaggtgcggctggatcacc

SEQ ID NO: 33 - DNA, 1452 bp, Unknown
  Anaerostipes caccae
gcttaacacatgcaagtcgaacgaagcatttaggattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatcccaatgaccgaaccttaaccggttttttctttcgagacattggagacaggtggtgcatggttgtcgtcagctcgtgtcgtgagatgttgggttaagtcccgcaacgagcgcaacccctatctttagtagccagcatttaaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaagg

SEQ ID NO: 34 - DNA, 1436 bp, Bifidobacterium adolescentis
gtgagagtggcgaacgggtgagtaatgcgtgaccgacctgccccatacaccggaatagctcctggaaacgggtggtaatgccggatgctccagttggatgcatgtccttctgggaaagattcatcggtatgggatggggtcgcgtcctatcagcttgatggcggggtaacggcccaccatggcttcgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgactgggagcaagcccttcggggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccaacctttttggggggagccgtctaaggtgagactcgtgattgggactaagtcgtaacaaggtagccgtaccggaaggtgcggctggatcacctccttt

SEQ ID NO: 35 - DNA, 1476 bp, Unknown
  Agathobaculum sp MUR_P3C8a
agtttgatcctggctcaggatgaacgctggcggcgtgcctaacacatgcaagtcgaacggggttattttggaaaatccttcgggattggaattcttaacctagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggcggggtaacggcccaccaaggcgacgatcagtagccggactgagaggttgaacggccacattgggactgagacacggcccagactcctacgggaggcagcagtggggaatattgcgcaatggggggaaccctgacgcagcaacgccgcgtgattgaagaaggccttcgggttgtaaagatctttaatcagggacgaaacatgacggtacctgaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggccttgacatcccgatgaccggtctagagatagaccttctcttcggagcatcggagacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatggcctgggctacacacgtactacaatggcagtcatacagagggaagcaaaatcgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccgtaaggagggcgcggccgaaggtaggggtggtaattagggtgaagtcgt

SEQ ID NO: 36 - DNA, 1511 bp, Unknown
  Phascolarctobacterium faecium MUR_P6E9
atcctggctcaggacgaacgctggcggcgtgcctaacacatgcaagtcgaacggagaattttatttcggtagaattcttagtggcgaacgggtgagtaacgcgtaggcaacctgccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtctgacgctgagatgcgaaagccagggtagcgaacgggattagataccccggtagtcctggccgtaaacgatgggtactaggtgtaggaggtatcgaccccttctgtgccggagttaacgcaataagtaccccgcctggggagtacggccgcaaggttgaaactcaaaggaattgacgggggcccgcacaagcggtggagtatgtggtttaattcgacgcaacgcgaagaaccttaccaaggcttgacattgattgaacgctctagagatagagctttcccttcggggacaagaaaacaggtggtgcatggctgtcgtcagctcgtgtcgtgagatgttgggttaagtcccgcaacgagcgcaacccctatcctatgttaccagcaagtaaagttggggactcatgggagactgccagggacaacctggaggaaggcggggatgacgtcaagtcatcatgccccttatgtcttgggctacacacgtactacaatggtcggaaacagagggaagcgaagccgcgaggcagagcaaaccccagaaacccgatctcagttcggatcgcaggctgcaacccgcctgcgtgaagtcggaatcgctagtaatcgcaggtcagcatactgcggtgaatacgttcccgggccttgtacacaccgcccgtcacaccacgaaagttggtaacacccgaagccggtgaggtaacctataaggagccagccgtctaaggtggggccgatgattggggtgaagtcgtaacaaggtagc

SEQ ID NO: 37 - DNA, 1505 bp, Unknown
  Akkermansia muciniphila
agagtttgattctggctcagaacgaacgctggcggcgtggataagacatgcaagtcgaacgagagaattgctagcttgctaataattctctagtggcgcacgggtgagtaacacgtgagtaacctgcccccaagagtgggatagccccgggaaactgggattaataccgcataaaatcgcaagattaaagcagcaatgcgcttggggatgggctcgcgtcctattagttagttggtgaggtaacggctcaccaaggcgatgacgggtagccggtctgagaggatgtccggccacactggaactgagacacggtccagacacctacgggtggcagcagtcgagaatcattcacaatgggggaaaccctgatggtgcgacgccgcgtgggggaatgaaggtcttcggattgtaaacccctgtcatgtgggagcaaattaaaaagatagtaccacaagaggaagagacggctaactctgtgccagcagccgcggtaatacagaggtctcaagcgttgttcggaatcactgggcgtaaagcgtgcgtaggcggtttcgtaagtcgtgtgtgaaaggcgggggctcaacccccggactgcacatgatactgcgagactagagtaatggagggggaaccggaattctcggtgtagcagtgaaatgcgtagatatcgagaggaacactcgtggcgaaggcgggttcctggacattaactgacgctgaggcacgaaggccaggggagcgaaagggattagatacccctgtagtcctggcagtaaacggtgcacgcttggtgtgcggggaatcgaccccctgcgtgccggagctaacgcgttaagcgtgccgcctggggagtacggtcgcaagattaaaactcaaagaaattgacggggacccgcacaagcggtggagtatgtggcttaattcgatgcaacgcgaagaaccttacctgggcttgacatgtaatgaacaacatgtgaaagcatgcgactcttcggaggcgttacacaggtgctgcatggccgtcgtcagctcgtgtcgtgagatgtttggttaagtccagcaacgagcgcaacccctgttgccagttaccagcacgtaaaggtggggactctggcgagactgcccagatcaactgggaggaaggtggggacgacgtcaggtcagtatggcccttatgcccagggctgcacacgtactacaatgcccagtacagagggggccgaagccgcgaggcggaggaaatcctaaaaactgggcccagttcggactgtaggctgcaacccgcctacacgaagccggaatcgctagtaatggcgcatcagctacggcgccgtgaatacgttcccgggtcttgtacacaccgcccgtcacatcatggaagccggtcgcacccgaagtatctgaagccaaccgcaaggaggcagggtcctaaggtgagactggtaactgggatgaagtcgtaacaaggtagccgtaggggaacctgcggctggatcacctccttt

SEQ ID NO: 38 - DNA, 1484 bp, Unknown
  Barnesiella intestinihomins
gatgaacgctagcgacaggcctaacacatgcaagtcgaggggcagcgaagaggtagcaatacctctgtcggcgaccggcgcacgggtgagtaacacgtatgcaatccacctgtaacagggggataacccggagaaatccggactaataccccataatatgggcgctccgcatggagagttcattaaagagagcaattttggttacagacgagcatgcgctccattagccagttggcggggtaacggcccaccaaagcgacgatggataggggttctgagaggaaggtcccccacattggaactgagacacggtccaaactcctacgggaggcagcagtgaggaatattggtcaatggtcggcagactgaaccagccaagtcgcgtgagggaagacggccctacgggttgtaaacctcttttgtcggagagtaaagtacgctacgtgtagcgtattgcaagtatccgaagaaaaagcatcggctaactccgtgccagcagccgcggtaatacggaggatgcgagcgttatccggatttattgggtttaaagggtgcgtaggcggcacgccaagtcagcggtgaaatttccgggctcaacccggagtgtgccgttgaaactggcgagctagagtgcacaagaggcaggcggaatgcgtggtgtagcggtgaaatgcatagatatcacgcagaaccccgattgcgaaggcagcctgctagggtgaaacagacgctgaggcacgaaagcgtgggtatcgaacaggattagataccctggtagtccacgcagtaaacgatgaatactaactgtttgcgatacaatgtaagcggtacagcgaaagcgttaagtattccacctggggagtacgccggcaacggtgaaactcaaaggaattgacgggggcccgcacaagcggaggaacatgtggtttaattcgatgatacgcgaggaaccttacccgggctcaaacgcagggggaatgtcggtgaaagccggcagctagtaatagtcacctgcgaggtgctgcatggttgtcgtcagctcgtgccgtgaggtgtcggcttaagtgccataacgagcgcaacccctatcgacagttactaacgggtgaagccgaggactctgtcgagactgccggcgcaagccgcgaggaaggtggggatgacgtcaaatcagcacggcccttacgtccggggcgacacacgtgttacaatggcaggtacagaaggcagccagtcagcaatgacgcgcgaatcccgaaaacctgtctcagttcggattggagtctgcaacccgactccatgaagctggattcgctagtaatcgcgcatcagccatggcgcggtgaatacgttcccgggccttgtacacaccgcccgtcaagccatggaagccgggagtacctgaagcatgcaaccgcaaggagcgtacgaaggtaataccggtaactggggctaagtcgtaacaaggtagccgtaccggaagg

SEQ ID NO: 39 - DNA, 1543 bp, Bilophila wadsworthia
agagtttgattctggctcagattgaacgctggcggcgtgcttaacacatgcaagtcgaacgtgaaagtccttcggggcgagtaaagtggcgcacgggtgagtaacgcgtggataatctacccttaagatggggataacggctggaaacggtcgctaataccgaatacgctcccgattttatcgttggggggaaagatggcctctgcttgcaagctatcgcttaaggatgagtccgcgtcccattagctagttggcggggtaacggcccaccaaggcaacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagagaagcagccgtctacggtagggccgatgattggggtgaagtcgtaacaaggtagccgtaggggaacctgcggctggatcacctcctt

SEQ ID NO: 40 - DNA, 1487 bp, Unknown
  Blautia faecis
agagtttgatcctggctcaggatgaacgctggcggcgtgcttaacacatgcaagtcgaacgggaaatactttattgaaacttcggtcgatttaatttatttctagtggcggacgggtgagtaacgcgtgggtaacctgccttataccgggggataacagccagaaatggctgctaataccgcataagcgcacagaaccgcatggttcggtgtgaaaaactccggtggtataagatggacccgcgttggattagctagttggcagggcagcggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagataatgacggtacctgactaagaagccccggctaactacgtgccagcagccgcggtaatacgtagggggcaagcgttatccggatttactgggtgtaaagggagcgtagacggcgcagcaagtctgatgtgaaaggcaggggcttaacccctggactgcattggaaactgctgtgcttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcagggagcacagctctttggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaaatcttgacatccctctgaccgggacttaaccgtccctttccttcgggacaggggagacaggtggtgcatggttgtcgtcagctcgtgtcgtgagatgttgggttaagtcccgcaacgagcgcaacccctatccttagtagccagcacgatggtgggcactctgaggagactgccagggataacctggaggaaggcggggatgacgtcaaatcatcatgccccttatgatttgggctacacacgtgctacaatggcgtaaacaaagggaagcgaacctgtgagggtgggcaaatctcaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgcaagggaggagctgccgaaggcgggaccgatgactggggtgaagtcgtaacaaggta

SEQ ID NO: 41 - DNA, 1488 bp, Unknown
  Blautia wexlerae
agagtttgatcctggctcaggatgaacgctggcggcgtgcttaacacatgcaagtcgaacgggaattactttattgaagcttcggcagatttagctggtttctagtggcggacgggtgagtaacgcgtgggtaacctgccttgtacagggggataacagtcagaaatgactgctaataccgcataagcgcacaggaccgcatggtccggtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggatggacaagtctgatgtgaaaggctggggctcaaccccgggactgcattggaaactgcccgtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgcggtaaacgatgaatgctaggtgtcgggtgacaaagtcattcggtgccgccgcaaacgcattaagcattccacctggggagtacgttcgcaagaatgaaactcaaaggaattgacggggacccgcacaagcggtggagcatgtggtttaattcgaagcaacgcgaagaaccttaccaagtcttgacatccctctgaccggaacttaaccgttccttcccttcggggcagaggagacaggtggtgcatggttgtcgtcagctcgtgtcgtgagatgttgggttaagtcccgcaacgagcgcaacccctatccccagtagccagcatttaaggtgggcactctggggagactgccagggataacctggaggaaggcggggatgacgtcaaatcatcatgccccttatgatttgggctacacacgtgctacaatggcgtaaacaaagggaagcgaacccgcgagggtgggcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaaccgtaaggagggagctgccgaaggcgggaccgatgactggggtgaagtcgtaacaaggta

SEQ ID NO: 42 - DNA, 1477 bp, Unknown
  Blautia luti
ctggctcaggatgaacgctggcggcgtgcttaacacatgcaagtcgaacgggaaatatttcattgagacttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagctggttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagtatggctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccggtccttaaccggacctttccttcgggacaggagagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagatcgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggta

SEQ ID NO: 43 - DNA, 1483 bp, Unknown
  Butyricimonas synergistica
agagtttgatcctggctcaggatgaacgctagcgacaggcttaacacatgcaagtcgaggggcagcacggtgtagcaatacactggtggcgaccggcgcacgggtgagtaacacgtgtgcaaccaaccccgtaccgggagataacccgcggaaacgtggactaacatcccataagactctagagccgcatggctctggatttaaaattccggtggtacgggacgggcacgcgcgacattaggtagttggcggggtaacggcccaccaagccgacgatgtctaggggttctgagaggaaggtcccccacactggaactgagacacggtccagactcctacgggaggcagcagtgaggaatattggtcaatgggcgcgagcctgaaccagccaagtcgcgtgagggaagaatggtctatggcctgtaaacctcttttgtcaaggaagaataaaaggtacgtgtaccttcttgccagtacttgacgaataagcatcggctaactccgtgccagcagccgcggtaatacgggggatgcgagcgttatccggatttattgggtttaaagggcgcgtaggcgggacgccaagtcagcggtaaaagactgcagctaaactgtagcacgccgttgaaactggcgacctggagacgagacgagggaggcggaacaagtgaagtagcggtgaaatgcttagatatcacttggaaccccgatagcgaaggcagcttcccaggctcgatctgacgctgatgcgcgagagcgtgggtagcgaacaggattagataccctggtagtccacgccgtaaacgatgctcactggatcttggcgatacactgccagggttcaagcgaaagtattaagtgagccacctggggagtacgtcggcaacgatgaaactcaaaggaattgacgggggcccgcacaagcggaggaacatgtggtttaattcgatgatacgcgaggaaccttacccgggtttaaatgtattttgcatttcttggaaacaggaattcccttcggggctagatacaaggtgctgcatggttgtcgtcagctcgtgccgtgaggtgtcgggttaagtcccataacgagcgcaacccctatcgccagttgccatcggttgaagccgggcactctgtcgagactgccaccgtaaggtgcgaggaaggcggggatgacgtcaaatcagcacggcccttacacccggggcgacacacgtgttacaatggccggtacagagggcagccacggggtgacccggagcgaatctctaaagccggtcgtagttcggactggagtctgcaacccgactccacgaagttggattcgctagtaatcgcgcatcagccatggcgcggtgaatacgttcccgggccttgtacacaccgcccgtcaagccatggaagccgggagtacctgaagatcgtgaccgcgaggaacgggctagggtaataccggtaactggggctaagtcgtaacaaggta

SEQ ID NO: 44 - DNA, 1496 bp, Unknown
  Clostridium bolteae
agagtttgatcctggctcaggatgaacgctggcggcgtgcctaacacatgcaagtcgaacgaagcaattaaaggaagttttcggatggaatttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttggccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgta

SEQ ID NO: 45 - DNA, 1494 bp, Unknown
  Parasutterella excrementihominis
attgaacgctggcggaacgctttacacatgcaagtcgaacggtaacgtggggaggagcttgctccaccccgacgacgagtggcgaacgggtgagtaatacatcggaacgtgtccgcttgtgggggacaaccagccgaaaggttggctaataccgcatgagttctacggaagaaagagggggacccgcaagggcctctcgcgagcggagcggccgatgactgattagccggttggtgaggtaacggctcaccaaagcaacgatcagtagctggtctgagaggacgaccagccacactgggactgagacacggcccagactcctacgggaggcagcagtggggaattttggacaatgggcgcaagcctgatccagctattccgcgtgtgggatgaaggccctcgggttgtaaaccacttttgtagagaacgaaaagacaccatcgaataaatggtgttgctgacggtactctaagaataagcaccggctaactacgtgccagcagccgcggtaatacgtagggtgcgagcgttaatcggaattactgggcgtaaagggtgcgcaggcggttgagtaagacagatgtgaaatccccgagcttaactcgggaatggcatatgtgactgctcgactagagtgtgtcagagggaggtggaattccacgtgtagcagtgaaatgcgtagatatgtggaagaacaccgatggcgaaggcagcctcctgggacataactgacgctcaggcacgaaagcgtggggagcaaacaggattagataccctggtagtccacgccctaaacgatgttaactagttgttgggatgtaacaatctcagtaacgcagccaacgcgagaagttaaccgcctgggaagtacggtcgcaagactaaaactcaaaggaattgacggggacccgcacaagcggtggatgatgtggattaattcgatgcaacgcgaaaaaccttacctacccttgacatgtcaggaagctcttgtaatgagagcgtgcccgcaagggagcctgaacacaggtgctgcatggctgtcgtcagctcgtgtcgtgagatgttgggttaagtcccgcaacgagcgcaacccttgtcactagttgctacgaaagggcactctagtgagactgccggtgacaaaccggaggaaggtggggatgacgtcaagtcctcatggcccttatgggtagggcttcacacgtcatacaatggtcggaacagagggcagcgaagccgtgaggcggagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcaaacaatgggagtggtgtttaccagaagtcgttagcctaaccgcaaggagggcggcgaccacggtgagcaccgtgactaatgttaagtcgtaacaaggtagccgtaccggaagg

SEQ ID NO: 46 - DNA, 1490 bp, Unknown
  Prevotella copri
agagtttgatcctggctcaggatgaacgctagctacaggcttaacacatgcaagtcgaggggaaacgatattggaagcttgcttccgataggcgtcgaccggcgcacgggtgagtaacgcgtatccaacctgcccaccacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatgggcgagagcctgaaccagccaagtagcgtgcaggatgacggccctatgggttgtaaactgcttttataagggaataaagtgagagtcgtgactctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaataggtcagcggccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttggagacaatgacgcccttcggggcctctgtgaaggtgctgcatggttgtcgtcagctcgtgccgtgaggtgtcggcttaagtgccataacgagcgcaacccctctccttagttgccatcaggttaagctgggcactctgggggcactgccaccgtaaggtgtgaggaaggtggggatgacgtcaaatcagcacggcccttacgtccggggctacacacgtgttacaatggcaggtacagagagatggtcccttgcaaaacggatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggta

SEQ ID NO: 47 - DNA, 1520 bp, Unknown
  Ruminococcus faecis
tttgatcctggctcaggatgaacgctggcggcgtgcttaacacatgcaagtcgaacgaagcaccttgatttgattcttcggatgaagatcctggtgactgagtggcggacgggtgagtaacgcgtgggtaacctgcctcatacagggggataacagttagaaatgactgctaataccgcataagaccacagcaccgcatggtgcaggggtaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgactactaggtgtcgggcagcaaagctgttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatctccctgaccggcaagtaatgttgcctttccttcgggacagggatgacaggtggtgcatggttgtcgtcagctcgtgtcgtgagatgttgggttaagtcccgcaacgagcgcaacccctatctttagtagccagcggtcaggccgggcactctagagagactgccagggataacctggaggaaggtggggatgacgtcaaatcatcatgccccttatgagcagggctacacacgtgctacaatggcgtaaacaaagggaggcagaaccgcgaggtcgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacctccttt

SEQ ID NO: 48 - DNA, 1474 bp, Bacteroides caccae
tggctcaggatgaacgctagctacaggcttaacacatgcaagtcgaggggcatcagtttggtttgcttgcaaaccaaagctggcgaccggcgcacgggtgagtaacacgtatccaacctgcctcatactcggggatagcctttcgaaagaaagattaatatccgatagcatatatttcccgcatgggttttatattaaagaaattcggtatgagatggggatgcgttccattagtttgttgggggggtaacggcccaccaagactacgatggataggggttctgagaggaaggtcccccacattggaactgagacacggtccaaactcctacgggaggcagcagtgaggaatattggtcaatggacgcgagtctgaaccagccaagtagcgtgaaggatgactgccctatgggttgtaaacttcttttatatgggaataaagttgtccacgtgtggatttttgtatgtaccatatgaataaggatcggctaactccgtgccagcagccgcggtaatacggaggatccgagcgttatccggatttattgggtttaaagggagcgtaggcggattgttaagtcagttgtgaaagtttgcggctcaaccgtaaaattgcagttgatactggcagtcttgagtgcagtagaggtgggcggaattcgtggtgtagcggtgaaatgcttagatatcacgaagaactccgattgcgaaggcagccactggagtgtaactgacgctgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaatgaattatggggaaacccataggccgcaaggcatttgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 49 - DNA, 1473 bp, Unknown; identical to SEQ ID NO: 8
  Bacteroides cellulosilyticus/ Bacteroides intestinalis

SEQ ID NO: 50 - DNA, 1472 bp, Unknown
  Bacteroides faecis
ctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggcataatagaaccgcatggtttttttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagtcgtaagacaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggta

SEQ ID NO: 51 - DNA, 929 bp, Bacteroides ovatus
cgatatccggatttattggagtttaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtkatgctgaggactctagagagactgccgtcgtaagatgtgaggaaggtggggatgacgtcaaatcagcacggcccttacgtccggggctacacacgtgttacaatggggggtacagaaggcsgctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctaggggtaaaactggtaattggggc

SEQ ID NO: 52 - DNA, 873 bp, Bacteroides thetaiotaomicron
tttaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccwagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcawwtgaatawwytggaaacagkwtagycgyaagrcawwtgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgca

SEQ ID NO: 53 - DNA, 1474 bp, Bacteroides uniformis; identical to SEQ ID NO: 9

SEQ ID NO: 54 - DNA, 1503 bp, Bacteroides vulgatus
atgaagagtttgatcctggctcaggatgaacgctagctacaggcttaacacatgcaagtcgaggggcagcatggtcttagcttgctaaggccgatggcgaccggcgcacgggtgagtaacacgtatccaacctgccgtctactcttggacagccttctgaaaggaagattaatacaagatggcatcatgagttcacatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatcccaaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgaggagcgccctagggtaaaactggtgactggggctaagtcgtaacaaggtagccgtaccggaag

SEQ ID NO: 55 - DNA, 887 bp, Bifidobacterium adolescentis
gggctcgtagkcggttcgtcgcgtccggtgtgaaagtccaycgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccccagagatggggcctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacactgtgaggtggagcggatcccttaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcccatcctttttgggg

SEQ ID NO: 56 - DNA, 1495 bp, Bifidobacterium longum
tggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctgg

SEQ ID NO: 57 - DNA, 689 bp, Bifidobacterium pseudocatenulatum
ggttcgtcgcgtccggtgtgaaagtccatcgtttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgwcggagctaacgcgttaagcatcccgcctggggagtacggccgcaaggctaaaacwmaaakaaattgacgggggcccgcacaagcggcggagcatgcggattaattcgatgcaacgcgaagaaccttacctgggcttgacatgttcccgacagccgtagagatatggcctcccttcggggcgggttcacaggtggtgcatggtcgtcgtcagctcgtgtcgtgagatgttgggttaagtcccgcaacgagcgcaaccctcgccctgtgttgccagcacgtcatggtgggaactcacgggggaccgccggggtcaactcggaggaaggtggggatgacgtcagatcatcatgccccttacgttcagggcttcacgcatgctacaatggccggtacaacgggatgcgacacggcgacgtg

SEQ ID NO: 58 - DNA, 1394 bp, Unknown
  Blautia coccoides/ Blautia producta
tgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggta

SEQ ID NO: 59 - DNA, 918 bp, Unknown
  Clostridium citroniae
tccggatttactggagtagtaagggagcgtagacggcgaagcaagtctggagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggtcagtaaagtgacctttccttcgggacaggggagacaggtggtgcatggttgtcgtcagctcgtgtcgtgagatgttgggttaagtcccgcaacgagcgcaacccttatccttagtagccagcaggtaaagctgggcactctagggagactgccagggataacctggaggaaggtggggatgacgtcaaatcatcatgccccttatgatttgggctacacacgtgctacaatggcgtaaacaaagggaagcgaccctgcgaaggcaagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaa

SEQ ID NO: 60 - DNA, 1307 bp, Unknown
  Clostridium clostridioforme
taataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccggggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcaggtagagccgggcactctagggagactgccagggataacctggaggaaggtggggatgacgtcaaatcatcatgccccttatgatttgggctacacacgtgctacaatggcgtaaacaaagggaagcgagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgt

SEQ ID NO: 61 - DNA, 1475 bp, Clostridium innocuum; identical to SEQ ID NO: 23

SEQ ID NO: 62 - DNA, 1475 bp, Unknown; identical to SEQ ID NO: 15
  Clostridium innocuum  / Erysipelotrichaceae bacterium 6_1_45

SEQ ID NO: 63 - DNA, 906 bp, Clostridium sordellii
acacatgcaagtcgagcgaacccttcggggtgagcggcggacgggtgagtaacgcgtgggtaacctgccctgtacacacggataacataccgaaaggtatgctaatacgggatrayatatgagagtcgcatggcttttgtatcaaagctccggcggtacaggatggacccgcgtctgattagctagttggtaaggtaacggcttaccaaggcaacgatcagtagccgacctgagagggtgatcggccacattggaactgagacacggtccaaactcmtacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggaattactgggcgtaaagggtgcgtaggcggtctttcaagccagaagtgaaaggctacggctcaaccgtagtaagcttttggaactgtaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactaggtgtcgggggttacccccctcggtgccgcagctaacgcattaagtactccgcctgggaagtacgctcgcaagagtgaaactcaaaggaattgacggggacccgcacaagtagcggagcatgtggtttaattcgaagcaacgcgaagaaccttatctaarcttgacatc

SEQ ID NO: 64 - DNA, 380 bp, Unknown
  Coprococcus comes
acrggaggcagcagtggggaatattgcacaatgggggaaaccctgatgcagcgacgccrcgtgagcgaagaagtattkcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggctgtgtaagtctgaagtgaaaggcgggggctcccccccggggactgctttggaaactatgcagctagactgtcggacaggtaagtggaattcccagtgtagcggtgaaatgcgtagatattgggaggaacagcaktgggtaaggctscttacaggacrat

SEQ ID NO: 65 - DNA, 1317 bp, Unknown
  Dorea longicatena
taacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgacccaaccgtaagg

SEQ ID NO: 66 - DNA, 808 bp, Unknown
  Erysipelatoclostridium ramosum
gagggagcaggcggcagcaagggtctgtggtgaaagcctgaagttaaacttcagtaagccatagaaaccaggcagctagagtgcaggagaggakcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactragtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaargcggggatgacgtcaaatcatcatgccccttatgacctgggctacacacgtgctacaatggatggtgcagagggaagcgaagccgcgaggtgaagcaaaacccataaaaccattctcagttcggattgtagtctgcarctcgactacatgaagttggaatcgctagtaatcgcgaatcarcatgtcgcgatgaatamgttctcgggcctt

SEQ ID NO: 67 - DNA, 1521 bp, Eubacterium rectale; identical to SEQ ID NO: 27

SEQ ID NO: 68 - DNA, 1482 bp, Unknown
  Odoribacter splanchnicus
agagtttgatcctggctcaggatgaacgctagcgacaggcttaacacatgcaagtcgaggggcatcatgaggtagcaataccttgatggcgaccggcgcacgggtgagtaacgcgtatgcaacctgcccgataccggggtatagcccatggaaacgtggattaacaccccatagtacttttatcctgcatgggatgtgagttaaatgtttaaggtatcggatgggcatgcgtcctattagttagttggcggggtaacagcccaccaagacgatgataggtaggggttctgagaggaaggtcccccacattggaactgagacacggtccaaactcctacgggaggcagcagtgaggaatattggtcaatggacgagagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacgggggcccgcacaagcggaggaacatgtggtttaattcgatgatacgcgaggaaccttacctgggtttaaatgggaaatgtcgtatttggaaacagatattctcttcggagcgtttttcaaggtgctgcatggttgtcgtcagctcgtgccgtgaggtgtcgggttaagtcccataacgagcgcaacccttaccgttagttgctagcatgtaatgatgagcactctaacgggactgccaccgtaaggtgagaggaaggcggggatgacgtcaaatcagcacggcccttacacccagggctacacacgtgttacaatggccggtacagagggccgctaccaggtgactggatgccaatctcaaaagccggtcgtagttcggattggagtctgtaacccgactccatgaagttggattcgctagtaatcgcgcatcagccatggcgcggtgaatacgttcccgggccttgtacacaccgcccgtcaagccatggaagccgggggtgcctgaagtccgtaaccgcgaggatcggcctagggcaaaactggtaactggggctaagtcgtaacaaggta

SEQ ID NO: 69 - DNA, 738 bp, Unknown
  Parabacteroides distasonis
cgaggggcagcrcaggagttagcaataccsggtggcgaccggcgcacgggtgagtaacgcgtatgcaacttrcctatcagagggggataacccggcgaaagtcggactaataccgcatgaagcagggatyccgcatgggratatttgctaaagattcatcgctgatagataggcatgcgttccattaggcagttggcggggtaacrgcccaccaaaccgacgatggataggggttctgagaggaaggtcccccacattggtactgagacacggaccaaactcctacgggaggcagcagtgaggaatattggtcaatggscgwragsctgaaccagccaagtcgcgtgagggatgaaggttctatggatcgtaaacctcttttataagggaataaagtgcgggacgtgtccyrttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcatagatatcacgcagaaccccgwttgcgaaggcagcctgccaagccgtaactgacgcggatgcacgaaagcgtggggatcaaacaggattagataccctggta

SEQ ID NO: 70 - DNA, 521 bp, Unknown
  Parabacteroides merdae
catgcaagtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttckgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgmsarcmgccgcgggaatacggaagatgcagagcgttatccggatwtattggggtta

SEQ ID NO: 71 - DNA, 1368 bp, Unknown
  Bacteroides xylanisolvens
catgcaagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggg

SEQ ID NO: 72 - DNA, 1399 bp, Unknown
  Blautia obeum
ggcgtgcttaacacatgcaagtcgaacgggaaaccttttattgaagcttcggcagatttagctggtttctagtggcggacgggtgagtaacgcgtgggtaacctgccttatacagggggataacaaccagaaatggttgctaataccgcataagcgcacaggaccgcatggtccggtgtgaaaaactccggtggtataagatggacccgcgttggattagctagttggcagggtaacggcctaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggactggcaagtctgatgtgaaaggcgggggctcaacccctggactgcattggaaactgttagtcttgagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgttggggagcaaagctcttcggtgccgccgcaaacgcattaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgttccttaaccggaactttccttcgggacaggggagacaggtggtgcatggttgtcgtcagctcgtgtcgtgagatgttgggttaagtcccgcaacgagcgcaacccctatccccagtagccagcagtccggctgggcactctgaggagactgccagggataacctggaggaaggcggggatgacgtcaaatcatcatgccccttatgatttgggctacacacgtgctacaatggcgtaaacaaagggaagcaagcctgcgaaggtaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaactgc

SEQ ID NO: 73 - DNA, 1485 bp, Unknown; identical to SEQ ID NO: 3
  Alistipes putredinis

SEQ ID NO: 74 - DNA, 1464 bp, Unknown
  Collinsella aerofaciens
agagttcgatcctggctcaggatgaacgctggcggcgcgcctaacacatgcaagtcgaacggcacctatcttcggatagaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgccggcccaaccgagaggggggaggcgccgaaggtgtggagggtgaggggggtgaagtcgtaacaaggta

SEQ ID NO: 75 - DNA, 1472 bp, Unknown; identical to SEQ ID NO: 5
  Bacteroides faecis /Eubacterium hallii

SEQ ID NO: 76 - DNA, 317 bp, Unknown
  Alistipes shahii
acatagggggwstgwkaggttwrccscccacattsrtactgagmcatgawcmaactctmtacgggargsagsagtgaggaatattggtcrrtggacgcaagtctgaaccagccatgccgsgtgcrggaagacggctckatgagtkgkaaactgcttttgtacrarrgtaaacgctcttacgtgtaagagcctgaaagtatsgtacraatgaggatcggctaactccgtgccagcagccgcggtaatacggaggatccaagcgttatccggatttattgggtttaaagggtgcgtaggcgggttgataaagttagrgg

SEQ ID NO: 77 - DNA, 811 bp, Unknown
  Anaerostipes caccae
gcttacacatgcaagtcgaacgaagcatttargattgaagttttcggatggatttcctatatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaa

SEQ ID NO: 78 - DNA, 728 bp, Phascolarctobacterium faecium
cggagaattttcatttcggtagaattcttagtggcgaacgggtgagtaacgcgtaggcaacctgccctttagacggggacaacattccgaaaggagtgctaataccggatgtgatcatcgtgccgcatggcaggatgaagaaagatggcctctacaagtaagctatcgctaaaggatgggcctgcgtctgattagctagttggtagtgtaacggactaccaaggcgatgatcagtagccggtctgagaggatgaacggccacattgggactgagacacggcccaaactcctacgggaggcagcagtggggaatcttccgcaatggacgaaagtctgacagagcaacgccgcgtgagtgatgaaggatttcggtctgtaaagctctgttgtttatgacgaacgtgcagtgtgtgaacaatgcattgcaatgacggtagtaaacgaggaagccacggctaactacgtgccagcagccgcggtaatacgtaggtggcgagcgttgtccggaattattgggcgtaaagagcatgtaggcggcttaataagtcgagcgtgaaaaatgcggggctcaaccccgtatggcgctggaaactgttaggcttgagtgcaggagaggaaaggggaattcccagtgtagcggtgaaatgcgtagatattgggaggaacaccagtggcgaaggcgcctttctggactgtgtttgacgctgagatgcgaaagccagggtagc

SEQ ID NO: 79 - DNA, 707 bp, Unknown
  Agathobaculum butyriciproducens
tagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggcggggtaacggcccaccaaggcgacgatcagtagccggactgagaggttgaacggccacattgggactgagacacggcccagactcctacgggaggcagcagtggggaatattgcgcaatgggggraaccctgacgcagcaacgccgcgtgattgaagaaggccttcgggttgtaaagatctttaatcagggacgaaamatgacggtacctgaagaataagctccggctaactacgtgccagcagccgcggtaatacgtagggagcaagcgttatccggatttactgggtgtaaagggcgcgcaggcgggccggcaagttggaagtgaaatccgggggcttaacccccgaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggata

SEQ ID NO: 80 - DNA, 1525 bp, Bacteroides fragilis; identical to SEQ ID NO: 11

SEQ ID NO: 81 - DNA, 1022 bp, Fusobacterium mortiferum
tggctcaggatgaacgctgacagaatgcttaacacatgcaagtctacttgatccttcgggtgatggtggcggacgggtgagtaacgcgtaaagaacttgccctgcagtctgggacaacatttggaaacgaatgctaataccggatattatgtatttctcgcatgagttttacatgaaagctatatgcgctgcaggagagctttgcgtcctattagctagttggtgaggtaacggctcaccaaggccatgataggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggacgaagtaagtgacggtaccaacagaagaagcgacggctaaatacgtgccagcagccgcggtaatacgtatgtcgcaagcgttatccggatttattgggcgtaaagcgcgtctaggcggtttggtaagtctgatgtgaaaatgcggggctcaactccgtattgcgttggaaactgctaaactagagtactggagaggtgggcggaactacaagtgtagaggtgaaattcgtagatatttgtaggaatgccgatggggaagccagcccactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctaagaaattagcagagatgcttttgtgccccttcgggggaacttagtgacaggtggtgcatggctgtcgtcagctcgtgt

SEQ ID NO: 82 - DNA, 1490 bp, Unknown; identical to SEQ ID NO: 17
  Paraclostridium benzoelyticum/ Clostridium bifermantans

SEQ ID NO: 83 - DNA, 1538 bp, Unknown; identical to SEQ ID NO: 21
  Escherichia fergusonii / Escherichia coli

(19) United States
(12) Patent Application Publication
(10) Pub. No.: US 2024/0316119 A1
(43) Pub. Date: Sep. 26, 2024

(54) COMPOSITIONS AND METHODS FOR SUPPRESSING PATHOGENIC ORGANISMS

(71) Applicant: VEDANTA BIOSCIENCES, INC., Cambridge, MA (US)

(72) Inventors: Bernat Olle, Cambridge, MA (US); Silvia Caballero, Cambridge, MA (US)

(73) Assignee: VEDANTA BIOSCIENCES, INC., Cambridge, MA (US)

(21) Appl. No.: 18/277,572

(22) PCT Filed: Feb. 18, 2022
(86) PCT No.: PCT/US2022/017043

§ 371 Date: Aug. 16, 2023

Related U.S. Application Data
(60) Provisional application No. 63/151,003, filed on Feb. 18, 2021.

(51) Int. Cl. A61K 35/74; A61K 9/19; A61K 31/095; A61P 31/04

(52) U.S. Cl. CPC A61K 35/74; A61K 9/19; A61K 31/095; A61P 31/04

(57) ABSTRACT

Provided herein are compositions and methods for suppressing infection by pathogenic organisms. Also provided herein are compositions and methods for reducing or preventing colonization by pathogenic organisms and methods of treating infection by a pathogenic organism. Also provided herein are compositions comprising taurine and methods of using such compositions for suppressing infection by pathogenic organisms, preventing colonization by pathogenic organisms, and methods of treating infection pathogenic organisms.

### RELATED APPLICATION

This application claims the benefit under 35 U.S.C. § 119(e) of U.S. provisional application No. 63/151,003 filed Feb. 18, 2021, the entire contents of which are incorporated by reference herein.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Feb. 17, 2022, is named P074570026WO00-SEQ-NTJ.txt, and is 157,149 bytes in size.

### BACKGROUND

Multidrug resistant organisms (MDROs; “superbugs”), microorganisms that have developed resistance to one or more classes of antimicrobial agents, such as antibiotics, are emerging as serious global health threat. It is estimated that over 2 million people in the United States contract serious bacterial infections that are resistant to one or more antibiotics each year (CDC, Antibiotic Resistance Threats in the United States, 2013. Publication No. CS239559-B). Treatment options for subjects with MDROs are extremely limited; prevention of transmission is critical. The most important factor contributing to the generation and propagation of MDROs is the use and overuse/misuse of antibiotics and it is thought that the problem will increase in severity as further pathogenic organisms with antibiotic resistance arise (CDC, 2013; WHO 2017).

### SUMMARY

In some aspects, the present disclosure provides compositions comprising two or more purified bacterial strains of species selected from the group consisting of a purified bacterial strain of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri.

In some aspects, the present disclosure provides compositions comprising two or more purified bacterial strains, wherein the two or more purified bacterial strains comprise 16S rDNA sequences having at least 97% sequence identity with nucleic acid sequences selected from the group consisting of SEQ ID NOs: 1-47.

In some embodiments of the compositions provided herein, the composition comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, or at least 47 purified bacterial strains.

In some aspects, the present disclosure provides compositions comprising purified bacterial strains of species Bacteroides faecis, Bacteroides vulgatus, Phascolartcobacterium faecium, and Clostridium citroniae. In some aspects, the present disclosure provides compositions comprising purified bacterial strains comprising 16S rDNA sequences having at least 97% sequence identity to nucleic acid sequences of SEQ ID NOs: 10, 29, 30, and 36. In some embodiments of the compositions provided herein, the composition comprises bacterial strains that originate from more than one human donor. In some embodiments, any of the compositions described herein may further comprise taurine.

In some aspects, the present disclosure provides compositions comprising one or more purified bacterial strains belonging to the phylum Firmicutes or Bacteroidetes; one or more purified bacterial strains selected from the group consisting of an Escherichia species and a Fusobacterium species; and taurine. In some aspects, the present disclosure provides compositions comprising one or more purified bacterial strains belonging to the phylum Firmicutes; one or more purified bacterial strains of Escherichia species; and taurine. In some aspects, the present disclosure provides compositions comprising one or more purified bacterial strains belonging to the phylum Firmicutes; one or more purified bacterial strains of Fusobacterium species; and taurine. In some aspects, the present disclosure provides compositions comprising one or more purified bacterial strains belonging to the phylum Bacteroidetes; one or more purified bacterial strains of Escherichia species; and taurine. In some aspects, the present disclosure provides compositions comprising one or more purified bacterial strains belonging to the phylum Bacteroidetes; one or more purified bacterial strains of Fusobacterium species; and taurine.

In some aspects, the present disclosure provides compositions comprising a purified bacterial strain belonging to Escherichia species, a purified bacterial strain belonging to Fusobacterium species, and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

In some aspects, the present disclosure provides compositions comprising a purified bacterial strain belonging to Escherichia species, a purified bacterial strain belonging to Fusobacterium species, and one or more purified bacterial strains of species selected from the group consisting of Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, and Phascolarctobacterium faecium; and taurine.

In some aspects, the present disclosure provides compositions comprising a purified bacterial strain belonging to Escherichia species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

In some aspects, the present disclosure provides compositions comprising purified bacterial strain belonging to Fusobacterium species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

In some aspects, the present disclosure provides compositions comprising a purified bacterial strain belonging to Escherichia species, a purified bacterial strain belonging to Fusobacterium species, and one or more purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

In some aspects, the present disclosure provides compositions comprising a purified bacterial strain belonging to Escherichia species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

In some aspects, the present disclosure provides compositions comprising a purified bacterial strain belonging to Fusobacterium species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

In some aspects, the present disclosure provides compositions comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species, a purified bacterial strain belonging to Fusobacterium species; and taurine.

In some aspects, the present disclosure provides compositions comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species; and taurine.

In some aspects, the present disclosure provides compositions comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Fusobacterium species; and taurine.

In some aspects, the present disclosure provides compositions comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species, a purified bacterial strain belonging to Fusobacterium species; and taurine.

In some aspects, the present disclosure provides compositions comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species; and taurine.

In some aspects, the present disclosure provides compositions comprising Anaerotruncus colihominis, Sellimonas intestinalis, Clostridium symbiosum, Dorea longicatena, Erysipelotrichaceae bacterium, Flavinofractor plautii, a purified bacterial strain belonging to Fusobacterium species; and taurine.

In some aspects, the present disclosure provides compositions comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species, a purified bacterial strain belonging to Fusobacterium species; and taurine.

In some aspects, the present disclosure provides compositions comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species; and taurine.

In some aspects, the present disclosure provides compositions comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales 3_1_57FAA_CT1), a purified bacterial strain belonging to Fusobacterium species; and taurine.

In some aspects, the present disclosure provides compositions comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species, a purified bacterial strain belonging to Fusobacterium species; and taurine.

In some aspects, the present disclosure provides compositions comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species; and taurine.

In some aspects, the present disclosure provides compositions comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Fusobacterium species; and taurine.

In some aspects, the present disclosure provides compositions comprising Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, a purified bacterial strain belonging to Fusobacterium species; and taurine.

In some aspects, the present disclosure provides compositions comprising Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, a purified bacterial strain belonging to Fusobacterium species; and taurine.

In some aspects, the present disclosure provides compositions comprising Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, a purified bacterial strain belonging to Escherichia species, a purified bacterial strain belonging to Fusobacterium species; and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, Flavonifractor plautii;

and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

In some aspects, the present disclosure provides compositions comprising Bacteroides cellulosilyticus, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Clostridium innocuum, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, Escherichia coli; and taurine.

In some aspects, the present disclosure provides compositions comprising Bacteroides caccae, Bacteroides cellulosilyticus, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, Escherichia coli; and taurine.

In some aspects, the present disclosure provides compositions comprising Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Clostridium innocuum, Paeniclostridium sordellii, Coprococcus comes, Dorea longicatena, Erysipelatoclostridium ramosum, Eubacterium rectale, Odoribacter splanchnicus, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Fusobacterium mortiferum, Paraclostridium bifermentans, Escherichia coli; and taurine.

In some aspects, the present disclosure provides compositions comprising Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, Escherichia coli; and taurine.

In some aspects, the present disclosure provides compositions comprising Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Clostridium innocuum, Paeniclostridium sordellii, Dorea longicatena, Erysipelatoclostridium ramosum, Odoribacter splanchnicus, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Fusobacterium mortiferum, Paraclostridium bifermentans, Escherichia coli; and taurine.

In some aspects, the present disclosure provides compositions comprising Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Phascolarctobacterium faecium, Agathobaculum butyriciproducens, Fusobacterium mortiferum, Escherichia coli; and taurine.

In some aspects, the present disclosure provides compositions comprising bacterial strains of species Bacteroides vulgatus, Clostridium citroniae, and Phascolarctobacterium faecium, and one or more purified bacterial strains of species selected from the group consisting of Bacteroides faecis, Bacteroides ovatus, Bifidobacterium longum, Blautia producta, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Agathobaculum butyriciproducens, Fusobacterium mortiferum, Escherichia coli; and taurine.

In some aspects, the present disclosure provides compositions comprising Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, Fusobacterium mortiferum; and taurine.

In some aspects, the present disclosure provides compositions comprising Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, Fusobacterium mortiferum; and taurine.

In some aspects, the present disclosure provides compositions comprising Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, Fusobacterium mortiferum; and taurine.

In some aspects, the present disclosure provides compositions comprising purified bacterial strains of species Bacteroides faecis, Bacteroides vulgatus, Phascolartcobacterium faecium, and Clostridium citroniae; and taurine. In some aspects, the present disclosure provides compositions comprising purified bacterial strains comprising 16S rDNA sequences having at least 97% sequence identity to the nucleic acid sequences of SEQ ID NOs: 10, 29, 30, and 36; and taurine.

In some embodiments, any of the compositions described herein may further comprise one or more bacterial strain selected from the group consisting of Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri.

In some embodiments, the composition further comprises taurine. In some embodiments, the composition comprises between about 40 mg to about 3000 mg taurine. In some embodiments, the composition comprises between about 100 mg to about 2000 mg taurine. In some embodiments, the composition comprises between about 500 mg to about 1500 mg taurine.

In some embodiments, the composition is effective in suppressing the replication, survival, and/or colonization of one or more pathogenic organisms. In some embodiments, the composition is effective in treating an infection by a pathogenic organism in a subject. In some embodiments, the pathogenic organism is susceptible to antibiotics. In some embodiments, the pathogenic organism is resistant to one or more antibiotics. In some embodiments, the pathogenic organism is a multi-drug resistant organism.

In some embodiments, the multi-drug resistant organism is Carbapenem Resistant Enterobacteriaceae (CRE) or Extended spectrum beta-lactamase (ESBL)-producing Enterobacteriaceae. In some embodiments, the Carbapenem Resistant Enterobacteriaceae (CRE) is carbapenem-resistant Klebsiella pneumoniae. In some embodiments, the carbapenem-resistant Klebsiella pneumoniae is carbapenem-resistant Klebsiella pneumoniae ATCC 700721. In some embodiments, the ESBL-producing Enterobacteriaceae is ESBL-producing K. pneumoniae or EBSL-producing E. coli. In some embodiments, the ESBL-producing Enterobacteriaceae is ESBL-producing K. pneumoniae subsp. pneumoniae (ATCC 700721), ESBL-producing E. coli ATCC BAA 2777, adherent/invasive E. coli (AIEC), Shiga toxin-producing E. coli (STEC), Verocytotoxin-producing E. coli (VTEC), enterohemorrhagic E. coli (EHEC), enteropathogenic E. coli (EPEC), enteroaggregative E. coli (EAEC), enteroinvasive E. coli (EIEC), or diffusely adherent E. coli (DAEC).

In some embodiments, the bacterial strains are lyophilized. In some embodiments, the bacterial strains are spray-dried. In some embodiments, one or more of the bacterial strains are in spore form. In some embodiments, each of the bacterial strains is in spore form. In some embodiments, one or more of the bacterial strains are in vegetative form. In some embodiments, each of the bacterial strains is in vegetative form. In some embodiments, the composition further comprises one or more enteric polymers.

In some embodiments, the pharmaceutical composition comprises between 1×10⁶ and 1×10¹⁰ colony forming units (CFUs) per bacterial strain. In some embodiments, each bacterial strain is present in the composition in the same CFU quantities (e.g., each strain is present at 1×10⁸ CFU).

In some aspects, the present disclosure provides pharmaceutical compositions comprising any of the compositions described herein and a pharmaceutically acceptable excipient.

In some embodiments, the pharmaceutical composition is formulated for oral delivery. In some embodiments, the pharmaceutical composition is formulated for delivery to the intestine. In some embodiments, the pharmaceutical composition is formulated for delivery to the colon. In some embodiments, the pharmaceutical composition is administered as one dose. In some embodiments, the pharmaceutical composition is administered as multiple doses. In some embodiments, each dose comprises the administration of multiple capsules.

In some aspects, the present disclosure provides a food product comprising any of the compositions comprising bacterial strains provided herein, and a nutrient.

In some aspects, the present disclosure provides a method of suppressing infection by a pathogenic organism in a subject, comprising administering to the subject a therapeutically effective amount of any of the compositions comprising bacterial strains, pharmaceutical compositions, or food products described herein.

In some aspects, the present disclosure provides a method of reducing or preventing colonization by a pathogenic organism in a subject, comprising administering to the subject a therapeutically effective amount of any of the compositions comprising bacterial strains, pharmaceutical compositions, or food products described herein.

In some aspects, the present disclosure provides a method of treating infection by a pathogenic organism in a subject, comprising administering to the subject a therapeutically effective amount of any of the compositions comprising bacterial strains, pharmaceutical compositions, or food products described herein.

In some embodiments of the methods provided herein, the pathogenic organism is a multi-drug resistant organism. In some embodiments, the pathogenic organism is Klebsiella pneumoniae. In some embodiments, the Klebsiella pneumoniae is multi-drug resistant. In some embodiments, the multi-drug resistant Klebsiella pneumoniae is carbapenem-resistant Klebsiella pneumoniae.

In some embodiments, the pathogenic organism is Extended spectrum beta-lactamase (ESBL)-producing Enterobacteriaceae. In some embodiments, the ESBL producing Enterobacteriaceae is Escherichia coli (E. coli). In some embodiments, the E. coli is adherent/invasive E. coli (AIEC), Shiga toxin-producing E. coli (STEC), Verocytotoxin-producing E. coli (VTEC), enterohemorrhagic E. coli (EHEC), enteropathogenic E. coli (EPEC), enteroaggregative E. coli (EAEC), enteroinvasive E. coli (EIEC), or diffusely adherent E. coli (DAEC).

In some embodiments, the subject is human. In some embodiments, the composition is administered to the subject by oral administration. In some embodiments, the composition is administered to the subject by rectal administration. In some embodiments, the administering suppresses the replication, survival, and/or colonization of the pathogenic organism. In some embodiments, the administration of the composition is not preceded by administration of vancomycin. In some embodiments, further comprising administering an antibiotic to the subject prior to administration of the composition. In some embodiments, the antibiotic is vancomycin.

In some embodiments, the method further comprises administering a composition comprising taurine to the subject. In some embodiments, the composition comprising taurine is administered to the subject prior to or after administration of any of the compositions or food products described herein containing a purified bacterial mixture. In some embodiments, the subject is administered between about 40 mg to about 3000 mg taurine per day. In some embodiments, the subject is administered between about 100 mg to about 2000 mg taurine per day. In some embodiments, the subject is administered between about 500 mg to about 1500 mg taurine per day.

### BRIEF DESCRIPTION OF THE DRAWINGS

The accompanying drawings are not intended to be drawn to scale. The figures are illustrative only and are not required for enablement of the disclosure. For purposes of clarity, not every component may be labeled in every drawing. In the drawings:

FIG. 1 presents a timeline of an exemplary mouse model of carbapenem-resistant Enterobacteriaceae (“CRE”) or extended spectrum beta-lactamase-producing Enterobacteriaceae (“ESBL”) colonization experiment.

FIG. 2 shows extended spectrum beta-lactamase (ESBL) producing Enterobacteriaceae colonization (colony forming units, “CFU”) levels in mice at 14 days post-treatment (“D14 post Tx”). From left to right, control (PBS), a composition of 36 bacterial strains (“36-mix”), or stool fraction from a human donor (Donor 1 “SFL”). Dashed line represents the limit of detection.

FIG. 3 is a table showing the phylum, family, and species names of the bacterial strains present in a composition of 47 bacterial strains, referred to herein as “47-mix.”

FIG. 4 shows carbapenem-resistant Klebsiella pneumoniae (CRE) or extended spectrum beta-lactamase (ESBL) producing Enterobacteriaceae colonization (colony forming units, “CFU”) levels in mice at 14 days post-treatment (“D14 post Tx”). Mice were treated with control (PBS) or a composition of 47 bacterial strains. For each treatment, data points in the left column represent CRE colonization, and data points in the right column represent ESBL colonization, L.o.D. (dashed line) represents the limit of detection.

FIGS. 5A and 5B show ESBL-producing Enterobacteriaceae and carbapenem-resistant Klebsiella pneumoniae (CRE) colonization in mice treated with the indicated compositions. FIG. 5A shows extended spectrum beta-lactamase (ESBL) producing Enterobacteriaceae colonization (colony forming units, “CFU”) levels in mice at 7, 10, and 14 days post-treatment (“days post Tx”). FIG. 5B shows CRE colonization (colony forming units, “CFU”) levels in mice at 7, 10, and 14 days post-treatment (“days post Tx”). For each time point, data are shown from left to right from mice treated with control (PBS), a composition of 47 strains (47-mix), a stool fraction from a second human donor (Donor 2 “SFL”), or a bacterial composition obtained by introducing fecal matter from a first human donor into a germ-free mouse and obtaining stool from the mouse to prepare a fecal matter transplant (Donor 1-inoculated mouse FMT). The dashed line represents the limit of detection.

### DETAILED DESCRIPTION

Suppressing or preventing undesired bacteria in a subject or suppressing or preventing colonization of bacteria in a particular region of the body can be challenging. Bacterial colonization may induce immune responses (local or systemic) in the subject, which may lead to serious disease. In particular, for multi-drug resistant organisms, or organisms that have acquired antibiotic resistance, elimination with many conventional therapeutics, such as antibiotics, may not be possible due to resistance or tolerance to the therapeutic. Additionally, it has been recently appreciated that intestinal colonization by bacteria of the oral microbiome may influence the immune environment of the intestine, such as induce Th1-dominated immune responses and lead to chronic inflammation and inflammatory conditions (see, e.g., Atarashi et al. Science (2017) 358 (359-365)). Normal bacterial colonization of different regions of the body, such as the oral cavity, may provide a reservoir of bacteria that can migrate and colonize other regions, such as the intestines.

Development of a live biotherapeutic product that is effective in promoting intestinal decolonization against multiple pathogens (e.g., both carbapenem-resistant Enterobacteriaceae and extended spectrum beta-lactamase-producing Enterobacteriaceae) has been challenging. Provided herein are compositions and methods for reducing/suppressing and/or preventing colonization by pathogenic organisms, such as multi-drug resistant organisms, for example carbapenem resistant Enterobacteriaceae (CRE) and extended spectrum beta-lactamase (ESBL)-producing Enterobacteriaceae. Provided herein are compositions and methods for suppressing infection by pathogenic organisms. Provided herein are compositions and methods for treating infection by a pathogenic organism. Also provided herein are compositions and methods for inducing regulatory T cell (Treg) production in response to pathogenic organisms. Provided herein are compositions and methods for inducing production of short chain fatty acids (SCFAs) in response to pathogenic organisms. Also provided herein are compositions comprising purified bacterial mixture and taurine, which may further aid in the colonization of the microbiota by beneficial bacterial strains and/or reduction in colonization (resistance) to pathogens.

In some embodiments, the one or more of the bacterial strains of the compositions provided herein colonize or recolonize the intestinal tract or parts of the intestinal tract (e.g., the colon or the cecum) of the subject. Such colonization or recolonization may also be referred to as grafting. In some embodiments, the one or more of the bacterial strains of the compositions recolonize the intestinal tract (e.g., the colon or the cecum) of the subject, for example after another organism or population of organisms has been partially or completely removed (e.g., by antibiotic treatment). In some embodiments, one or more of the bacterial strains of the compositions recolonize the intestinal tract (e.g., the colon or the cecum) after one or more pathogenic organisms or other organism (e.g., bacteria that induce an immune response) has been removed. In some embodiments, the recolonization of the intestinal tract or parts thereof by the bacterial strains of the compositions described herein prevents or suppresses colonization by undesired organisms (e.g., pathogenic organisms, multi-drug resistant organisms, oral microbiome bacteria, bacteria that induce immune responses, pathobionts, bacteria that reduce Treg production, bacteria that reduce SCFA production).

In some embodiments, the one or more of the bacterial strains of the compositions can “outgrow” a pathogen or undesired bacteria, such as a pathogenic organism, for example carbapenem resistant Enterobacteriaceae (CRE) and/or extended spectrum beta-lactamase (ESBL)-producing Enterobacteriaceae. Thus, in some embodiments, if a pathogen or undesired bacteria (e.g., pathogenic organisms, multi-drug resistant organism, for example carbapenem resistanct Enterobacteriaceae (CRE) and extended spectrum beta-lactamase (ESBL)-producing Enterobacteriaceae) and one or more bacteria of compositions provided herein are both present in the intestinal tract (e.g., the colon or the cecum), the one or more bacteria of compositions provided herein grow faster (e.g., have a shorter doubling time) than the pathogen, thereby preventing the pathogen from accumulating in the intestinal tract (e.g., the colon or the cecum). In some embodiments, the one or more bacteria of compositions provided herein grow faster than the pathogen in an otherwise intact or complete microbiome. In some embodiments, the one or more bacteria of compositions provided herein grow faster than the pathogen in a depleted microbiome (e.g., following antibiotic treatment). In some embodiments, the faster growth results because the one or more bacteria of the compositions provided herein are better at grafting in the intestinal tract (e.g., the colon or the cecum). In some embodiments, the faster growth results because the one or more bacteria of the compositions provided herein are better at metabolizing nutrients present in the intestinal tract (e.g., the colon or the cecum). In some embodiments, the compositions of bacterial strains provided herein prevent or inhibit replication of the pathogen. In some embodiments, the compositions of bacterial strains provided herein induce death of (kill) the pathogen. In some embodiments, the bacterial strains of the compositions provided herein can treat pathogenic infections, because of the synergy between the bacterial strains.

In some embodiments, the bacterial compositions described herein prevent recolonization by a pathogen or undesired bacteria (e.g., pathogenic organisms, multi-drug resistant organisms, for example carbapenem resistanct Enterobacteriaceae (CRE) and extended spectrum beta-lactamase (ESBL)-producing Enterobacteriaceae). For example, in some embodiments, the pathogen or undesired bacteria have been reduced or eliminated from the subject, for example, using a first therapeutic agent (such as an antibiotic), and the bacterial compositions described herein are administered to prevent recolonization of the subject. In some embodiments, the bacterial compositions described herein reduce or eliminate a pathogen or undesired bacteria from the subject and prevent recolonization of the subject.

In some embodiments, the combination of bacterial strains of the compositions provided herein is superior in the use of nutrients when compared to the pathogen or undesired bacteria, thereby suppressing the growth of the pathogen or undesired bacteria. In some embodiments, the combination of bacterial strains of the compositions provided herein is superior in grafting when compared to the pathogen or undesired bacteria, thereby suppressing the growth of the pathogen or undesired bacteria. In some embodiments, the combination of bacterial strains of the compositions provided herein is superior in the use of nutrients and in grafting when compared to the pathogen or undesired bacteria, thereby suppressing the growth of the pathogen or undesired bacteria. In some embodiments, the combination of bacterial strains of the compositions provided herein inhibits the growth, survival, and/or colonization of the pathogen or undesired bacteria.

In some embodiments, the combination of bacterial strains of the compositions provided herein has antagonizing or inhibitory activity towards the pathogen or undesired bacteria, thereby inhibiting the growth, survival, and/or colonization of the pathogen or undesired bacteria. In some embodiments, at least one bacterial strain of the compositions provided herein has antagonizing or inhibitory activity towards the pathogen or undesired bacteria, thereby inhibiting the growth, survival, and/or colonization of the pathogen or undesired bacteria.

In some embodiments, the bacterial strains of the compositions provide a synergistic effect in colonizing specific niches in the intestinal tract (e.g., the colon or the cecum). In some embodiments, the synergistic effect is provided by the capacity of the combination to metabolize specific nutrients. In some embodiments, the synergistic effect is provided by the capacity of the combination to provide specific metabolites to the environment.

The bacterial strains used in the compositions provided herein generally are isolated from the microbiome of healthy individuals. In some embodiments, the compositions include strains originating from a single individual. In some embodiments, the compositions include strains originating from multiple individuals. In some embodiments, the bacterial strains are obtained from multiple individuals, isolated, and grown up individually. The bacterial compositions that are grown up individually may subsequently be combined to provide the compositions of the disclosure. It should be appreciated that the origin of the bacterial strains of the compositions provided herein is not limited to the human microbiome from a healthy individual. In some embodiments, the bacterial strains originate from a human with a microbiome in dysbiosis. In some embodiments, the bacterial strains originate from non-human animals or the environment (e.g., soil or surface water). In some embodiments, the combinations of bacterial strains provided herein originate from multiple sources (e.g., human and non-human animals).

In some embodiments, the bacteria of the compositions provided herein are anaerobic bacteria. In some embodiments, the bacteria of the compositions provided herein are obligate anaerobic bacteria.

In general, bacterial strains may be classified phylogenetically with other closely related strains and species based on their 16S rRNA (or 16S rDNA) nucleic acid sequence. Methods for determining the identity of specific bacterial species based on their 16S rRNA (or 16S rDNA) nucleic acid sequence are well known in the art (See, e.g., Jumpstart Consortium Human Microbiome Project Data Generation Working, G. PLOS One (2012) 7, e39315).

It should be appreciated that any of the compositions described herein may contain multiple strains of a particular bacterial species. For example, in some embodiments, the composition may comprise two strains of the species Absiella innocuum.

The disclosure encompasses compositions comprising bacterial strains having close sequence identity or homology to and/or fall within the species of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Clostridium bifermentans, Paeniclostridium sordellii, Clostridium sordellii, Absiella innocuum, Absiella innocuum, Erysipelatoclostridium ramosum, Clostridium_M citroniae, Clostridium citroniae, Clostridium_M clostridioforme, Clostridium clostridioforme, Eubacterium_E hallii, Eubacterium hallii, Agathobacter rectale, Eubacterium rectale, Blautia producta, Blautia_A obeum, Blautia obeum Dorea longicatena, Coprococcus_B comes, Coprococcus comes, Anaerostipes caccae, Agathobaculum sp, Agathobaculum buryriciproducens, Phascolarctobacterium faecium, Escherichia coli, Escherichia sp. 3_2_53FAA, Fusobacterium_A sp, Fusobacterium sp, Fusobacterium mortiferum, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri.

In some embodiments, the compositions disclosed herein comprise two or more bacterial strains. In some embodiments, the compositions described herein comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47 or more bacterial strains (e.g., purified bacterial strains).

It should be appreciated that the terms “bacteria” and “bacterial strains” as used herein are interchangeable. The compositions described herein containing multiple purified bacterial strains may also be referred to as “live bacterial products.”

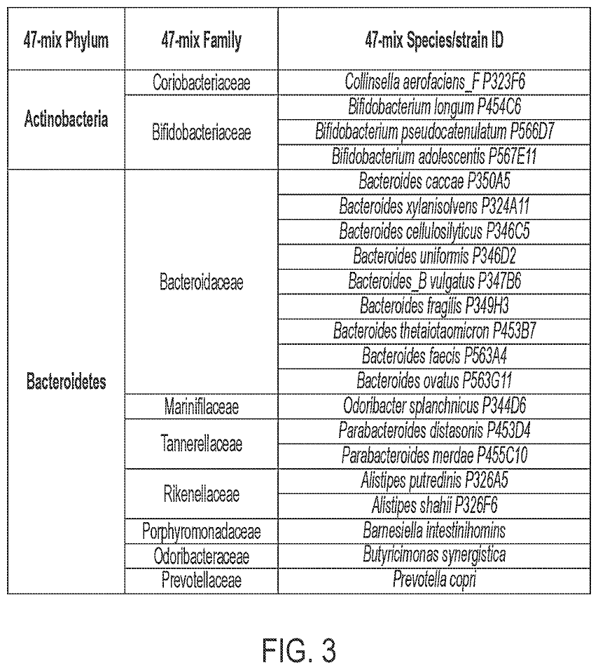

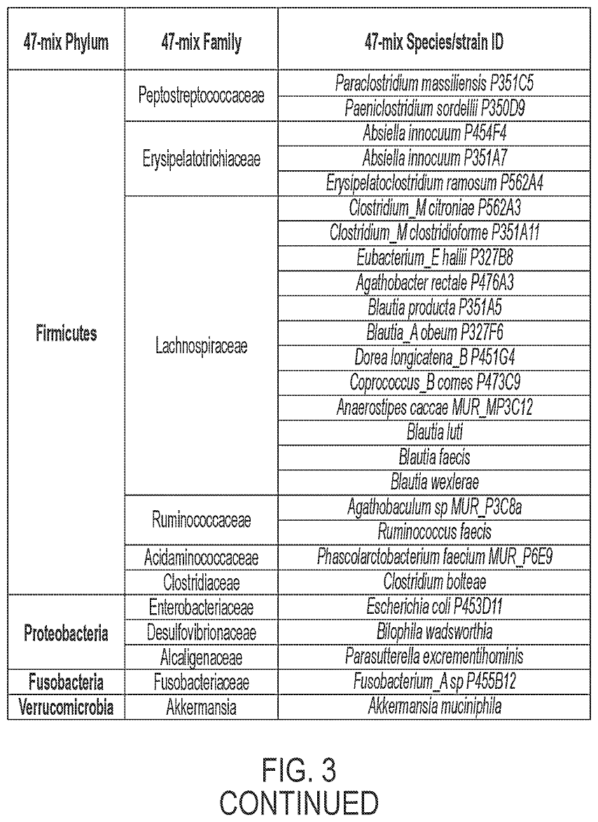

In one aspect, the disclosure provides a live bacterial product referred to as “47-mix” (see, FIGS. 3-5). As shown in FIG. 3 and Table 1, the composition referred to as the 47-mix contains 47 bacterial strains related to the following species: Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions comprise two or more (e.g., 2, 3, 4, 5, or more) purified bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions consist of two or more (e.g., 2, 3, 4, 5, or more) bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri.

In some embodiments, the compositions consist essentially of two or more (e.g., 2, 3, 4, 5, or more) bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions comprise bacterial strains of species Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions consist of bacterial strains of species Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions consist essentially of bacterial strains of species Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine. In some embodiments, consisting essentially of, as used herein, refers to compositions that do not include any additional therapeutically active bacterial strains.

In some embodiments, the compositions comprise 38 bacterial strains, which may be referred to as a “38-mix.” In some embodiments, the compositions comprise 38 bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions consist of 38 bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions consist essentially of 38 bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions comprise 33 bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions consist of 33 bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some embodiments, the compositions consist essentially of 33 bacterial strains of species selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri. In some embodiments, the composition further comprises taurine.

In some aspects, the present disclosure provides compositions comprising one or more purified bacterial strains belonging to the phylum Firmicutes or Bacteroidetes; one or more purified bacterial strains selected from the group consisting of an Escherichia species and a Fusobacterium species; and taurine. In some aspects, the present disclosure provides compositions comprising one or more purified bacterial strains belonging to the phylum Firmicutes; one or more purified bacterial strains of Escherichia species; and taurine. In some aspects, the present disclosure provides compositions comprising one or more purified bacterial strains belonging to the phylum Firmicutes; one or more purified bacterial strains of Fusobacterium species; and taurine.

The Firmicutes phylum, also referred to as the phylum Bacillota, is a phylum of Gram-positive bacteria, many of which are spore-forming. Classes within the Firmicutes phylum include the obligate anaerobe class Clostridia, and Bacilli, which may be obligate or facultative anaerobes. In some embodiments of compositions comprising a purified bacterial strain belonging to the phylum Firmicutes, the bacterial strain belongs to a class selected from the group consisting of Bacilli, Clostridia, and Negativicutes. In some embodiments, the composition comprises two or more bacterial strains belonging to the phylum Firmicutes, and each of the bacterial strains independently belongs to a class selected from the group consisting of Bacilli, Clostridia, and Negativicutes.

Families within the Firmicutes phylum include Peptostreptococcaceae, Erysipelatotrichiaceae, Lachnospiraceae, Ruminococcaceae, Acidaminococcaceae, and Clostridiaceae. In some embodiments of compositions comprising a purified bacterial strain belonging to the phylum Firmicutes, the bacterial strain belongs to a family selected from the group consisting of Peptostreptococcaceae, Erysipelatotrichiaceae, Lachnospiraceae, Ruminococcaceae, Acidaminococcaceae, and Clostridiaceae. In some embodiments, the composition comprises two or more bacterial strains belonging to the phylum Firmicutes, and the two or more bacterial strains independently belong to a family independently selected from the group consisting of Peptostreptococcaceae, Erysipelatotrichiaceae, Lachnospiraceae, Ruminococcaceae, Acidaminococcaceae, and Clostridiaceae. In some embodiments, each of the bacterial strains belonging to the phylum Firmicultes independently belongs to a family selected from the group consisting of Peptostreptococcaceae, Erysipelatotrichiaceae, Lachnospiraceae, Ruminococcaceae, Acidaminococcaceae, and Clostridiaceae.

The Bacteroidetes phylum, also referred to as the Bacteroidota phylum, is a phylum of Gram-negative, non-spore-forming bacteria, which may be aerobic or anaerobic. Classes within the Bacteroidetes phylum include Bacteroidia, Chitinophagia, Cytophagia, Flavobacteria, Saprospiria, and Sphingobacteriia. In some embodiments of compositions comprising a purified bacterial strain belonging to the phylum Bacteroidetes, the bacterial strain belongs to the class Bacteroidia. In some embodiments, the composition comprises two or more bacterial strains belonging to the phylum Bacteroidetes, and two or more of the bacterial strains belong to the class Bacteroidia. In some embodiments, each of the bacterial strains of the composition belonging to the phylum Bacteroidetes belongs to the class Bacteroidia.

Families within the Bacteroidetes phylum include Bacteroidaceae, Marinifilaceae, Tannerellaceae, Rikenellaceae, Porphyromonadaceae, Odorbacteraceae, and Prevotellaceae. In some embodments of compositions comprising a purified bacterial strain belonging to the phylum Bacteroidetes, the bacterial strain belongs to a family selected from the group consisting of Bacteroidaceae, Marinifilaceae, Tannerellaceae, Rikenellaceae, Porphyromonadaceae, Odorbacteraceae, and Prevotellaceae. In some embodiments, the composition comprises two or more bacterial strains belonging to the phylum Bacteroidetes, and the two or more bacterial strains belongs to a family independently selected from the group consisting of Bacteroidaceae, Marinifilaceae, Tannerellaceae, Rikenellaceae, Porphyromonadaceae, Odorbacteraceae, and Prevotellaceae.

In some aspects, the present disclosure provides compositions comprising one or more purified bacterial strains belonging to the phylum Bacteroidetes; one or more purified bacterial strains of Escherichia species; and taurine. In some aspects, the present disclosure provides compositions comprising one or more purified bacterial strains belonging to the phylum Bacteroidetes; one or more purified bacterial strains of Fusobacterium species; and taurine.

In some aspects, the present disclosure provides compositions comprising a purified bacterial strain belonging to Escherichia species, a purified bacterial strain belonging to Fusobacterium species, and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

In some aspects, the present disclosure provides compositions comprising a purified bacterial strain belonging to Escherichia species, a purified bacterial strain belonging to Fusobacterium species, and one or more purified bacterial strains of species selected from the group consisting of Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, and Phascolarctobacterium faecium; and taurine.

In some aspects, the present disclosure provides compositions comprising a purified bacterial strain belonging to Escherichia species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

In some aspects, the present disclosure provides compositions comprising a purified bacterial strain belonging to Fusobacterium species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

In some aspects, the present disclosure provides compositions comprising a purified bacterial strain belonging to Escherichia species, a purified bacterial strain belonging to Fusobacterium species, and one or more purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

In some aspects, the present disclosure provides compositions comprising a purified bacterial strain belonging to Escherichia species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

In some aspects, the present disclosure provides compositions comprising a purified bacterial strain belonging to Fusobacterium species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

In some aspects, the present disclosure provides compositions comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species, a purified bacterial strain belonging to Fusobacterium species; and taurine.

In some aspects, the present disclosure provides compositions comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species; and taurine.

In some aspects, the present disclosure provides compositions comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Fusobacterium species; and taurine.

In some aspects, the present disclosure provides compositions comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species, a purified bacterial strain belonging to Fusobacterium species; and taurine.

In some aspects, the present disclosure provides compositions comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species; and taurine.

In some aspects, the present disclosure provides compositions comprising Anaerotruncus colihominis, Sellimonas intestinalis, Clostridium symbiosum, Dorea longicatena, Erysipelotrichaceae bacterium, Flavinofractor plautii, a purified bacterial strain belonging to Fusobacterium species; and taurine.

In some aspects, the present disclosure provides compositions comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species, a purified bacterial strain belonging to Fusobacterium species; and taurine.

In some aspects, the present disclosure provides compositions comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species; and taurine.

In some aspects, the present disclosure provides compositions comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium boltede ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Fusobacterium species; and taurine.

In some aspects, the present disclosure provides compositions comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species, a purified bacterial strain belonging to Fusobacterium species; and taurine.

In some aspects, the present disclosure provides compositions comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species; and taurine.

In some aspects, the present disclosure provides compositions comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Fusobacterium species; and taurine.

In some aspects, the present disclosure provides compositions comprising Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, a purified bacterial strain belonging to Fusobacterium species; and taurine.

In some aspects, the present disclosure provides compositions comprising Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, a purified bacterial strain belonging to Fusobacterium species; and taurine.

In some aspects, the present disclosure provides compositions comprising Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, a purified bacterial strain belonging to Escherichia species, a purified bacterial strain belonging to Fusobacterium species; and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

In some aspects, the present disclosure provides compositions comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

In some aspects, the present disclosure provides compositions comprising Bacteroides cellulosilyticus, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Clostridium innocuum, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, Escherichia coli; and taurine.

In some aspects, the present disclosure provides compositions comprising Bacteroides caccae, Bacteroides cellulosilyticus, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, Escherichia coli; and taurine.

In some aspects, the present disclosure provides compositions comprising Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Clostridium innocuum, Paeniclostridium sordellii, Coprococcus comes, Dorea longicatena, Erysipelatoclostridium ramosum, Eubacterium rectale, Odoribacter splanchnicus, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Fusobacterium mortiferum, Paraclostridium bifermentans, Escherichia coli; and taurine.

In some aspects, the present disclosure provides compositions comprising Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, Escherichia coli; and taurine.

In some aspects, the present disclosure provides compositions comprising Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Clostridium innocuum, Paeniclostridium sordellii, Dorea longicatena, Erysipelatoclostridium ramosum, Odoribacter splanchnicus, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Fusobacterium mortiferum, Paraclostridium bifermentans, Escherichia coli; and taurine.

In some aspects, the present disclosure provides compositions comprising Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Phascolarctobacterium faecium, Agathobaculum butyriciproducens, Fusobacterium mortiferum, Escherichia coli; and taurine.

In some aspects, the present disclosure provides compositions comprising bacterial strains of species Bacteroides vulgatus, Clostridium citroniae, and Phascolarctobacterium faecium, and one or more purified bacterial strains of species selected from the group consisting of Bacteroides faecis, Bacteroides ovatus, Bifidobacterium longum, Blautia producta, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Agathobaculum butyriciproducens, Fusobacterium mortiferum, Escherichia coli; and taurine.

In some aspects, the present disclosure provides compositions comprising Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, Fusobacterium mortiferum; and taurine.

In some aspects, the present disclosure provides compositions comprising Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, Fusobacterium mortiferum; and taurine.

In some aspects, the present disclosure provides compositions comprising Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, Fusobacterium mortiferum; and taurine.

In some aspects, the present disclosure provides compositions comprising a purified bacterial mixture comprising bacterial strains of the species Bacteroides faecis, Bacteroides vulgatus, Phascolartcobacterium faecium, and Clostridium citroniae. In some aspects, the disclosure provides compositions comprising a purified bacterial mixture consisting of bacterial strains of the species Bacteroides faecis, Bacteroides vulgatus, Phascolartcobacterium faecium, and Clostridium citroniae. In some aspects, the present disclosure provides compositions comprising bacterial strains of the species Bacteroides faecis, Bacteroides vulgatus, Phascolartcobacterium faecium, and Clostridium citroniae; and taurine. In some aspects, the disclosure provides a composition comprising taurine and a purified bacterial mixture consisting of bacterial strains of the species Bacteroides faecis, Bacteroides vulgatus, Phascolartcobacterium faecium, and Clostridium citroniae. In some embodiments, a bacterial strain of Bacteroides faecis comprises a 16S rDNA sequence comprising at least 97% sequence identity to the nucleic acid sequence of SEQ ID NO: 30. In some embodiments, a bacterial strain of Bacteroides vulgatus comprises a 16S rDNA sequence comprising at least 97% sequence identity to the nucleic acid sequence of SEQ ID NO: 10. In some embodiments, a bacterial strain of Phascolartcobacterium faecium comprises a 16S rDNA sequence comprising at least 97% sequence identity to the nucleic acid sequence of SEQ ID NO: 36. In some embodiments, a bacterial strain of Clostridium citroniae comprises a 16S rDNA sequence comprising at least 97% sequence identity to the nucleic acid sequence of SEQ ID NO: 29.

In some aspects, the present disclosure provides compositions comprising purified bacterial strains comprising 16S rDNA sequences having at least 97% sequence identity to nucleic acid sequences of SEQ ID NOs: 10, 29, 30, and 36. In some aspects, the present disclosure provides compositions comprising purified bacterial strains comprising 16S rDNA sequences having at least 97% sequence identity to nucleic acid sequences of SEQ ID NOS: 10, 29, 30, and 36; and taurine. In some aspects, the present disclosure provides compositions comprising a purified bacterial mixture consisting of bacterial strains comprising 16S rDNA sequences having at least 97% sequence identity to the nucleic acid sequences of SEQ ID NOs: 10, 29, 30, and 36. In some aspects, the present disclosure provides compositions comprising taurine and a purified bacterial mixture consisting of bacterial strains comprising 16S rDNA sequences having at least 97% sequence identity to the nucleic acid sequences of SEQ ID NOs: 10, 29, 30, and 36.

In some embodiments, any of the compositions described herein further comprises one or more bacterial strain selected from the group consisting of Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri.

As will be appreciated by one of skill in the art, any one or more bacterial strain identified as having pathogen-antagonizing activity may be included in any of the compositions described herein.

Aspects of the disclosure relate to bacterial strains with 16S rDNA sequences that have sequence identity to a nucleic acid sequence of any one of the sequences of the bacterial strains or species described herein. The terms “identical,” or percent “identity,” in the context of two or more nucleic acids or amino acid sequences, refer to two or more sequences or subsequences that are the same. Two sequences are “substantially identical” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% sequence identity) over a specified region of a nucleic acid or amino acid sequence or over the entire sequence, when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length. In some embodiments, the identity exists over the length the 16S rRNA or 16S rDNA sequence.

In some embodiments, the bacterial strain has at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, at least 99.9%, or up to 100% sequence identity relative to any of the strains or bacterial species described herein over a specified region or over the entire sequence. It would be appreciated by one of skill in the art that the term “sequence identity” or “percent sequence identity,” in the context of two or more nucleic acid sequences or amino acid sequences, refers to a measure of similarity between two or more sequences or portion(s) thereof.

Additionally, or alternatively, two or more sequences may be assessed for the alignment between the sequences. The terms “alignment” or percent “alignment” in the context of two or more nucleic acids or amino acid sequences, refer to two or more sequences or subsequences that are the same. Two sequences are “substantially aligned” if two sequences have a specified percentage of amino acid residues or nucleotides that are the same (e.g., at least 80%, 85%, 90%, 95%, 96%, 97%, 98%, 99%, 99.5%, 99.6%, 99.7%, 99.8% or 99.9% identical) over a specified region or over the entire sequence, when compared and aligned for maximum correspondence over a comparison window, or designated region as measured using one of the following sequence comparison algorithms or by manual alignment and visual inspection. Optionally, the alignment exists over a region that is at least about 50 nucleotides in length, or more preferably over a region that is 100 to 500 or 1000 or more nucleotides in length. In some embodiments, the identity exists over the length the 16S rRNA or 16S rDNA sequence.

For sequence comparison, typically one sequence acts as a reference sequence, to which test sequences are compared. Methods of alignment of sequences for comparison are well known in the art. See, e.g., by the local homology algorithm of Smith and Waterman (1970) Adv. Appl. Math. 2:482c, by the homology alignment algorithm of Needleman and Wunsch, J. Mol. Biol. (1970) 48:443, by the search for similarity method of Pearson and Lipman. Proc. Natl. Acad. Sci. USA 85:2444, 1988, by computerized implementations of these algorithms (GAP, BESTFIT, FASTA, and TFASTA in the Wisconsin Genetics Software Package, Genetics Computer Group. Madison. WI), or by manual alignment and visual inspection (sec. e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively.

In one aspect, the bacterial composition comprises two or more (e.g., 2, 3, 4, 5 or more) purified bacterial strains comprising 16S rDNA sequences having at least 97% (e.g., 97%, 98%, 99.%, 99.1%, 99.2%, 99.3%, 99.4%, 99.5%, 99.6%, 99.7%, 99.8%, 99.9%, or 100%) sequence identity with the nucleic acid sequence selected from SEQ ID NOs: 1-83. In one aspect, the bacterial composition consists of two or more (e.g., 2, 3, 4, 5 or more) purified bacterial strains comprising 16S rDNA sequences having at least 97% (e.g., 97%, 98%, 99,%, 99.1%. 99.2%, 99.3%, 99.4%, 99.5%, 99.6%, 99.7%, 99.8%, 99.9%, or 100%) sequence identity with the nucleic acid sequence selected from SEQ ID NOs: 1-83. In one aspect, the bacterial composition consists essentially of two or more (e.g., 2, 3, 4, 5 or more) purified bacterial strains comprising 16S rDNA sequences having at least 97% (e.g., 97%, 98%, 99,%, 99.1%, 99.2%, 99.3%, 99.4%, 99.5%, 99.6%, 99.7%, 99.8%, 99.9%, or 100%) sequence identity with the nucleic acid sequence selected from SEQ ID NOs: 1-83.

In one aspect, the bacterial composition comprises two or more (e.g., 2, 3, 4, 5 or more) purified bacterial strains comprising 16S rDNA sequences having at least 97% (e.g., 97%, 98%, 99,%, 99.1%, 99.2%, 99.3%, 99.4%, 99.5%, 99.6%, 99.7%, 99.8%, 99.9%, or 100%) sequence identity with the nucleic acid sequence selected from SEQ ID NOs: 1-47. In one aspect, the bacterial composition consists of two or more (e.g., 2, 3, 4, 5 or more) purified bacterial strains comprising 16S rDNA sequences having at least 97% (e.g., 97%, 98%, 99,%. 99.1%, 99.2%, 99.3%, 99.4%, 99.5%, 99.6%, 99.7%, 99.8%, 99.9%, or 100%) sequence identity with the nucleic acid sequence selected from SEQ ID NOs: 1-47. In one aspect, the bacterial composition consists essentially of two or more (e.g., 2, 3, 4, 5 or more) purified bacterial strains comprising 16S rDNA sequences having at least 97% (e.g., 97%, 98%, 99,%, 99.1%, 99.2%, 99.3%, 99.4%, 99.5%, 99.6%, 99.7%, 99.8%, 99.9%, or 100%) sequence identity with the nucleic acid sequence selected from SEQ ID NOs: 1-47.

In some embodiments, the compositions disclosed herein comprise two or more bacterial strains. In some embodiments, the compositions described herein comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, or more bacterial strains (e.g., purified bacterial strains).

In some embodiments, the bacterial composition comprises purified bacterial strains comprising 16S rDNA sequences having at least 97% (e.g., 97%, 98%, 99,%, 99.1%, 99.2%. 99.3%. 99.4%, 99.5%, 99.6%, 99.7%, 99.8%, 99.9%, or 100%) sequence identity with the nucleic acid sequences provided by SEQ ID NOs: 1-47. In some embodiments, the bacterial composition consists of purified bacterial strains comprising 16S rDNA sequences having at least 97% (e.g., 97%, 98%, 99,%, 99.1%, 99.2%, 99.3%, 99.4%, 99.5%, 99.6%, 99.7%, 99.8%, 99.9%, or 100%) sequence identity with the nucleic acid sequences of SEQ ID NOS: 1-47. In some embodiments, the bacterial composition consists essentially of purified bacterial strains comprising 16S rDNA sequences having at least 97% (e.g., 97%, 98%, 99,%. 99.1%, 99.2%, 99.3%, 99.4%, 99.5%, 99.6%, 99.7%, 99.8%, 99.9%, or 100%) sequence identity with the nucleic acid sequence of SEQ ID NOs: 1-47.

In some embodiments, the bacterial composition comprises 38 purified bacterial strains comprising 16S rDNA sequences having at least 97% (e.g., 97%, 98%, 99,%, 99.1%, 99.2%, 99.3%, 99.4%, 99.5%, 99.6%, 99.7%, 99.8%, 99.9%, or 100%) sequence identity with any of the nucleic acid sequences provided by SEQ ID NOs: 1-47. In some embodiments, the bacterial composition consists of 38 purified bacterial strains comprising 16S rDNA sequences having at least 97% (e.g., 97%, 98%, 99,%, 99.1%, 99.2%, 99.3%. 99.4%, 99.5%, 99.6%, 99.7%, 99.8%, 99.9%, or 100%) sequence identity with any of the nucleic acid sequences of SEQ ID NOs: 1-47. In some embodiments, the bacterial composition consists essentially of 38 purified bacterial strains comprising 16S rDNA sequences having at least 97% (e.g., 97%, 98%, 99,%, 99.1%, 99.2%, 99.3%, 99.4%, 99.5%, 99.6%. 99.7%, 99.8%, 99.9%, or 100%) sequence identity with any of the nucleic acid sequence of SEQ ID NOs: 1-47.

In some embodiments, the bacterial composition comprises 33 purified bacterial strains comprising 16S rDNA sequences having at least 97% (e.g., 97%, 98%, 99,%, 99.1%, 99.2%, 99.3%, 99.4%, 99.5%, 99.6%, 99.7%, 99.8%, 99.9%, or 100%) sequence identity with any of the nucleic acid sequences provided by SEQ ID NOs: 1-47. In some embodiments, the bacterial composition consists of 33 purified bacterial strains comprising 16S rDNA sequences having at least 97% (e.g., 97%, 98%, 99,%, 99.1%, 99.2%, 99.3%, 99.4%, 99.5%, 99.6%, 99.7%, 99.8%, 99.9%, or 100%) sequence identity with any of the nucleic acid sequences of SEQ ID NOs: 1-47. In some embodiments, the bacterial composition consists essentially of 33 purified bacterial strains comprising 16S rDNA sequences having at least 97% (e.g., 97%, 98%, 99,%, 99.1%, 99.2%, 99.3%, 99.4%, 99.5%, 99.6%. 99.7%, 99.8%, 99.9%, or 100%) sequence identity with any of the nucleic acid sequence of SEQ ID NOs: 1-47.

Provided herein are bacterial strains and combinations of bacterial strains that are homologous or have a high percent of homology with bacterial strains comprising 16S rDNA sequences selected from SEQ ID NO: 1-83. It should be appreciated that the bacterial strains described herein have a 16S rDNA sequence selected from SEQ ID NO: 1-83 may also be homologous to other strains based on their whole genome sequence, or subset of their whole genome sequence.

In one aspect, the compositions comprise a fraction of a fecal sample. In some embodiments, the compositions comprise a non-spore forming fraction of a fecal sample. In some embodiments, the compositions comprise a spore forming fraction of a fecal sample.

In some embodiments, the compositions described herein comprise spore forming and non-spore forming bacterial strains. In some embodiments, the compositions described herein comprise spore forming bacterial strains. In some embodiments, the compositions described herein comprise only spore forming bacterial strains. In some embodiments, the compositions described herein comprise only non-spore forming bacterial strains. The spore-forming bacteria can be in spore form (i.e., as spores) or in vegetative form (i.e., as vegetative cells). In spore form, bacteria are generally more resistant to environmental conditions, such as heat, acid, radiation, oxygen, chemicals, and antibiotics. In contrast, in the vegetative state or actively growing state, bacteria are more susceptible to such environmental conditions, compared to in the spore form. In general, bacterial spores are able to germinate from the spore form into a vegetative/actively growing state, under appropriate conditions. For instance, bacteria in spore form may germinate when they are introduced in the intestine.

In some embodiments, at least one (e.g., 1, 2, 3, 4, 5, or more) of the bacterial strains in the composition is a spore former. In some embodiments, at least one (e.g., 1, 2, 3, 4, 5, or more) of the bacterial strains in the composition is in spore form. In some embodiments, at least one (e.g., 1, 2, 3, 4, 5, or more) of the bacterial strains in the composition is a non-spore former. In some embodiments, at least one (e.g., 1, 2, 3, 4, 5, or more) of the bacterial strains in the composition is in vegetative form. As discussed above, spore forming bacteria can also be in vegetative form. In some embodiments, at least one (e.g., 1, 2, 3, 4, 5, or more) of the bacterial strains in the composition is in spore form and at least one (e.g., 1, 2, 3, 4, 5, or more) of the bacterial strains in the composition is in vegetative form. In some embodiments, at least one bacterial strain that is considered able to form spores (i.e., a spore-former) is present in the composition in vegetative form. In some embodiments, at least one bacterial strain that is considered able to form spores is present in the composition both in spore form and in vegetative form.

In some embodiments, the compositions comprise bacterial strains that are spore forming bacterial strains. In some embodiments, the compositions comprise bacterial strains that are non-spore forming bacterial strains. In some embodiments, the compositions comprise bacterial strains that are spore forming bacterial strains and bacterial strains that are non-spore forming bacterial strains. In some embodiments, the compositions comprise a mixture of bacterial strains wherein at least 10% of the bacterial strains are spore forming bacterial strains, at least 20% of the bacterial strains are spore forming bacterial strains, at least 30% of the bacterial strains are spore forming bacterial strains, at least 40% of the bacterial strains are spore forming bacterial strains, at least 50% of the bacterial strains are spore forming bacterial strains, at least 60% of the bacterial strains are spore forming bacterial strains, at least 70% of the bacterial strains are spore forming bacterial strains, at least 80% of the bacterial strains are spore forming bacterial strains, at least 90% of the bacterial strains are spore forming bacterial strains bacteria or up to 100% of the bacterial strains are spore forming bacterial strains. Whether a bacterial strain is a spore forming strain can be determined for instance by evaluating the genome of the bacterial strain for the presence of sporulation genes. However, it should be appreciated that not all bacteria that are predicted to encode spore forming genes can be made to sporulate. In addition, whether a bacterial strain is a spore forming strain can be determined by exposing the bacterial strain to stress conditions, e.g., heat or exposure to chemicals (e.g., ethanol or chloroform), that are known to induce sporulation.

It should be appreciated that spore forming bacteria can be in spore form or in vegetative form. In some embodiments of the compositions provided herein, the spore forming bacteria are in spore form. In some embodiments, the spore forming bacteria are in vegetative form. In some embodiments, the spore forming bacteria are both present in spore form and in vegetative form. In some embodiments, compositions comprise spore forming bacteria and at least 10% of the spore forming bacteria are in spore format, at least 20% of the spore forming bacteria are in spore format, at least 30% of the spore forming bacteria are in spore format, at least 40% of the spore forming bacteria are in spore format, at least 50% of the spore forming bacteria are in spore format, at least 60% of the spore forming bacteria are in spore format, at least 70% of the spore forming bacteria are in spore format, at least 80% of the spore forming bacteria are in spore format, at least 90% of the spore forming bacteria are in spore format, or up to 100% of the spore forming bacteria are in spore format.

It is envisioned that the bacterial strains of the compositions provided herein are alive and will be alive when they reach the target area (e.g., the intestines). Bacterial spores are considered to be alive in this regard. In some embodiments, bacteria that are administered as spores may germinate in the target area (e.g., the intestines). It should further be appreciated that not all of the bacteria are alive, and the compositions can include a percentage (e.g., by weight) that is not alive. In addition, in some embodiments, the compositions include bacterial strains that are not alive when administered or at the time when the composition reaches the target area (e.g., the intestines). It is envisioned that non-living bacteria may still be useful by providing some nutrients and metabolites for the other bacterial strains in the composition.

Methods of inducing sporulation of spore-forming bacterial strains are well-known in the art (see, e.g., Paredes-Sabja et al., Trends Microbiol. (2011) 19(2):85-94). Generally, bacterial strains that are spore-formers can be made to go into spore form by stressing the bacterial strains. Non-limiting examples of stresses that can induce sporulation are an increase in temperature, change in the nutrients available and/or exposure to chemicals (e.g., ethanol or chloroform). It should be noted that bacteria that are non-spore formers, for instance because they are missing sporulation genes, cannot be made to sporulate by stress. To prepare compositions in which all the bacterial strains are in the spore form, the composition or bacterial cultures used to prepare the composition may be subjected to treatment to kill any bacteria not in spore form (e.g., in vegetative form), for example by exposing the composition to heat and chemically breaking down the non-spore bacteria. The bacteria in spore format can subsequently be separated from the non-spore bacteria for instance by filtration.

The amount of spores can be quantified using techniques known in the art. These techniques include phase contrast microscopy for enumerating spores using a hemocytometer. In addition, the viability of spores can be determined by plating the spores and growing the spores. For instance, spores can be plated in appropriate media and incubated in the anaerobic chamber for a period of time (e.g., 48-96 hrs.). Viability can subsequently be determined by quantifying the colony forming units which correspond to spores that germinated. For instance, spores can be plated on TCCFA plates (taurocholate, cycloserine, cefoxintin, fructose agar plates), in which taurocholate helps the spores to germinate. In addition, spores can be quantified using the dipicolinic assay (DPA assay). DPA is an agent that allows for spore selection and is a clear indicator of endospores. When complexed with terbium, bright green luminescence is observed.

In some embodiments, the compositions comprise bacterial strains that are non-spore forming bacterial strains. In some embodiments, the compositions comprise bacterial strains that are spore forming bacterial strains and bacterial strains that are non-spore forming bacterial strains. In some embodiments, the compositions comprise a mixture of bacterial strains wherein at least 10% of the bacterial strains are non-spore forming bacterial strains, at least 20% of the bacterial strains are non-spore forming bacterial strains, at least 30% of the bacterial strains are non-spore forming bacterial strains, at least 40% of the bacterial strains are non-spore forming bacterial strains, at least 50% of the bacterial strains are non-spore forming bacterial strains, at least 60% of the bacterial strains are non-spore forming bacterial strains, at least 70% of the bacterial strains are non-spore forming bacterial strains, at least 80% of the bacterial strains are non-spore forming bacterial strains, at least 90% of the bacterial strains are non-spore forming bacterial strains, or up to 100% non-spore forming bacterial strains.

In any of the compositions provided herein, the bacterial strains may be purified. In any of the compositions provided herein, the bacterial strains may be isolated. Any of the bacterial strains described herein may be isolated and/or purified, for example, from a source such as a culture or a microbiota sample (e.g., fecal matter). The bacterial strains used in the compositions provided herein generally are isolated from the microbiome of healthy individuals. However, bacterial strains can also be isolated from individuals that are considered not to be healthy. In some embodiments, the compositions include strains originating from multiple individuals.

As used herein, the term “isolated” refers to a bacterium or bacterial strain that has been separated from one or more undesired component, such as another bacterium or bacterial strain, one or more component of a growth medium, and/or one or more component of a sample, such as a fecal sample. In some embodiments, the bacteria are substantially isolated from a source such that other components of the source are not detected.

As also used herein, the term “purified” refers to a bacterial strain or composition comprising such that has been separated from one or more components, such as contaminants. In some embodiments, the bacterial strain is substantially free of contaminants. In some embodiments, one or more bacterial strains of a composition may be independently purified from one or more other bacteria produced and/or present in a culture or a sample containing the bacterial strain. In some embodiments, a bacterial strain is isolated or purified from a sample and then cultured under the appropriate conditions for bacterial replication, e.g., under anaerobic culture conditions. The bacteria that is grown under appropriate conditions for bacterial replication can subsequently be isolated/purified from the culture in which it is grown.

In some embodiments, the bacterial strains of the compositions provided herein are obligate anaerobes. In some embodiments, the bacterial strains of the compositions provided.

Aspects of the present disclosure are related to methods for suppressing undesired bacteria. As used herein, the term “suppressing” refers to any form of inhibiting undesired bacteria. In some embodiments, the methods described herein reduce/inhibit or prevent the colonization, replication, proliferation, and/or survival of the undesired bacteria (e.g., pathogenic organisms, multi-drug resistant organisms, for example carbapenem resistanct Enterobacteriaceae (CRE) and extended spectrum beta-lactamase (ESBL)-producing Enterobacteriaceae). In some embodiments, the methods described herein directly or indirectly induce death of the undesired bacteria (e.g., pathogenic organism, multi-drug resistant organisms, for example carbapenem resistanct Enterobacteriaceae (CRE) and extended spectrum beta-lactamase (ESBL)-producing Enterobacteriaceae).

Aspects of the present disclosure are related to methods for suppressing infection by a pathogenic organism in a subject. Aspects of the present disclosure are related to methods for reducing or preventing colonization by a pathogenic organism (e.g., bacterium, virus, fungus, or parasite) in a subject. Aspects of the present disclosure are related to methods for treating infection with a pathogenic organism. In some embodiments, the pathogenic organism is not multi-drug resistant. In some embodiments, the pathogenic organism resistant to one or more drugs (e.g., antibacterial, anti-viral, anti-fungal agent). In some embodiments, the pathogenic organism is multi-drug resistant. The methods described herein involve administering to a subject a therapeutically effective amount of any of the compositions described herein. As used herein, a “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human.

In some embodiments, any of the compositions described herein are effective in suppressing the replication, survival, and/or colonization of one or more pathogenic organism. In some instances, the pathogenic organism is susceptible to antibiotics, while in other instances, the pathogenic organism is resistant to antibiotics. In some embodiments, the pathogenic organism is a multi-drug resistant organism, which are described elsewhere herein. In some embodiments, the pathogenic organism is an oral microbiome bacterium. It should be noted that oral microbiome bacteria are not necessarily pathogenic, but may become so when located elsewhere, such as in the gastrointestinal tract. The amount of suppression of replication, survival, and/or colonization of the one or more pathogenic organism can be measured or identified using standard assays known in the art, some of which are further described and exemplified herein.

In some embodiments, administration of the compositions described herein reduces/inhibits or prevents the colonization, re-colonization, replication, proliferation, and/or survival of pathogenic organism, such as multi-drug resistant organisms. In some embodiments, administration of the compositions described herein allows for colonization of the gastrointestinal tract of the subject by the bacterial strain(s) of the compositions thereby preventing colonization by multi-drug resistant organisms.

In some embodiments, the pathogenic organism is a pathobiont, i.e., a potentially pathogenic organism which, under normal circumstances, lives as a symbiont.

In some embodiments, the subject is a carrier of a multi-drug resistant organism and is suffering from the effects of the infection. In some embodiments the subject is an asymptomatic carrier of a multi-drug resistant organism. In some embodiments, the subject has experienced recurrent or chronic colonization with a multi-drug resistant organism. In some embodiments, the subject is suffering from a first occurrence of a particular multi-drug resistant organism. In some embodiments, the subject is at risk of colonization with a multi-drug resistant organism, such as prior antibiotic use. In some embodiments, the subject has a risk factor associated with colonization with a multidrug resistant organism. In some embodiments, the subject has had a previous infection or colonization with a multi-drug resistant organism. In some embodiments, the subject has been treated with antibiotics which resulted in the recurrence of the multi-drug resistant organism.

In some embodiments, the subject is to undergo a procedure that puts the subject at a higher risk of colonization and the compositions are administered prophylactically. In some embodiments, the subject has a disease or disorder associated with use of a proton pump inhibitor, which may increase the likelihood of an oral bacterium migrating to the intestine. In some embodiments, the compositions provided herein are administered to a subject to lower the risk of becoming colonized with a multidrug resistant organism. In some embodiments, the bacterial compositions provided herein administered to a subject that is receiving a proton pump inhibitor.

Individuals may be at risk of acquiring a multi-drug resistant organism if they have recently received antimicrobials, are in an immunosuppressed state (e.g., on chemotherapy, have a malignancy, undergoing or received a transplant), have a chronic disease or inflammatory condition (such as diabetes, renal disease, etc.), are older, are undergoing hemodialysis, surgery or other invasive procedures, have indwelling device(s), and/or are living in a long-term care facility or are hospitalized. In some embodiments, the subject is colonized with a multi-drug resistant organism. Skin and mucosal colonization are common (Cassone et al., Curr Geriatr Rep. (2015) 4(1): 87-89), but multi-drug resistant organisms may also colonize the gastrointestinal (GI) tract and oral cavity, causing inflammation (Atarashi et al., Science (2017) 358, 359-365). Colonization can lead to significant infections, such as in the skin, lungs, urinary tract, or bloodstream, which may result in serious complications, including death (CDC, 2013). In some instances, multi-drug resistant organisms may be ingested, leading to consequences throughout the digestive system. In some embodiments, a multi-drug resistant organism may colonize the oral cavity.

In some embodiments, the multi-drug resistant organism is Vancomycin Resistant Enterococci (VRE), Carbapenem Resistant Enterobacteriaceae (CRE), Neisseria gonorrheae, Multidrug Resistant Acinetobacter, Campylobacter, Extended spectrum beta-lactamase (ESBL) producing Enterobacteriaceae, ESBL-producing Escherichia coli, Multidrug Resistant Pseudomonas aeruginosa, Salmonella, Drug resistant non-typhoid Salmonella, Drug resistant Salmonella Typhi, Drug resistant Shigella, Methicillin Resistant Staphylococcus aureus, Drug resistant Streptococcus pneumoniae, Drug resistant Tuberculosis, Vancomycin resistant Staphylococcus aureus, Erythromycin Resistant Group A Streptococcus, or Clindamycin resistant Group B Streptococcus. In some embodiments, the multi-drug resistant organism is Vancomycin Resistant Enterococci (VRE). In some embodiments, the multi-drug resistant organism is Carbapenem Resistant Enterobacteriaceae (CRE).

In some embodiments, the compositions described herein are effective in reducing or preventing colonization by more than one multi-drug resistant organism. In some embodiments, the compositions described herein are effective in treating infection of more than one multi-drug resistant organism. For example, in some embodiments, the compositions described herein are effective in reducing or preventing, or treating infection, with a carbapenem resistant Enterobacteriaceae (CRE) and Extended spectrum beta-lactamase (ESBL) producing Enterobacteriaceae,

Enterobacteriaceae is a broad family of bacteria that includes a number of bacterial pathogens. Bacteria belonging to the Enterobacteriaceae family are characterized as being Gram negative and are frequently found in the gut flora of the intestinal tract of humans and mammals but may also be found in water and soil. Non-limiting examples of bacterial genera belonging to the Enterobacteriaceae family include Alishewanella, Buttiauxella, Grimontella, Pectobacterium, Salmonella, Yokenella, Alterococcus, Cedecea, Hafnia, Phlomobacter, Samsonia, Aquamonas, Citrobacter, Klebsiella, Photorhabdus, Serratia, Aranicola, Cronobacter, Kluyvera, Poodoomaamaana, Shigella, Arsenophonus, Dickeya, Leclercia, Plesiomonas, Sodalis, Azotivirga, Edwardsiella, Leminorella, Pragia, Tatumella, Blochmannia, Enterobacter, Moellerella, Proteus, Trabulsiella, Brenneria, Erwinia, Morganella, Providencia, Wigglesworthia, Buchnera, Escherichia, Obesumbacterium Rahnella, Xenorhabdus, Budvicia, Ewingella, Pantoea, Raoultella, and Yersinia.

In some embodiments, the compositions described herein are effective in reducing or preventing, or treating infection, with a carbapenem resistant Enterobacteriaceae (CRE). As used herein, the terms “carbapenem resistant Enterobacteriaceae” or “CRE” refer to a bacterial strain or species that belongs to the Enterobacteriaceae family and has developed resistance to carbapenem antibiotics. In some embodiments, the CRE is also resistant to additional antibiotics in addition to carbapenem antibiotics.

In some embodiments, the Carbapenem Resistant Enterobacteriaceae is carbapenem-resistant Klebsiella pneumoniae. Examples of CRE Klebsiella pneumoniae is Klebsiella pneumoniae strain ATCC 700721.

In some embodiments, the compositions described herein are effective in reducing or preventing, or treating infection, with an extended spectrum beta-lactamase (ESBL) producing Enterobacteriaceae. As used herein, the terms “extended spectrum beta-lactamase Enterobacteriaceae” or “ESBL-Enterobacteriaceae” refer to a bacterial strain or species that belongs to the Enterobacteriaceae family and produce or are capable of producing beta-lactamase enzymes that breakdown or inactivate antibiotics. In some embodiments, the ESBL Enterobacteriaceae is resistant to one or more antibiotics (e.g., beta-lactam antibiotics).

In some embodiments, the ESBL Enterobacteriaceae is also resistant to additional antibiotics in addition to beta-lactam antibiotics.

In some embodiments, the ESBL-producing Enterobacteriaceae is ESBL-producing K. pneumoniae or ESBL-producing E. coli. In some embodiments, the ESBL-producing Enterobacteriaceae is ESBL-producing K. pneumoniae subsp. pneumoniae (ATCC 700721), ESBL-producing E. coli ATCC BAA 2777 adherent/invasive E. coli (AIEC), Shiga toxin-producing E. coli (STEC), Verocytotoxin-producing E. coli (VTEC), enterohemorrhagic E. coli (EHEC), enteropathogenic E. coli (EPEC), enteroaggregative E. coli (EAEC), enteroinvasive E. coli (EIEC), or diffusely adherent E. coli (DAEC).

In some embodiments, the CRE or ESBL-producing Enterobacteriaceae is Citrobacter freundii, Citrobacter koseri, Enterobacter cloacae, Enterobacter aerogenes, Enterobacter sakasakii, Escherichia coli, Escherichia albertii, Proteus mirabilis, Proteus vulgaris, Salmonella enterica, Serratia marcescens, Shigella dysenterii, Shigella flexneri, Shigella sonnei, Shigella boydii, Yersinia pestis, Yersinia enterocolitica, or Yersinia pseudotuberculosis.

Aspects of the present disclosure are related to methods for suppressing pathogenic organisms in a subject. In some embodiments, the pathogenic organism is susceptible to antibiotics (e.g., not a multi-drug resistant organism). Pathogenic organisms are organisms that are capable of causing disease in a subject and may be viruses, bacteria, fungi, protozoa, and worms. Non-limiting examples of pathogenic organisms of the present disclosure include Salmonella, Shigella, Staphylococcus, Streptococcus, Enterococcus, Enterobacteriaceae, Neisseria gonorrheae, Acinetobacter, Campylobacter, Clostridium, Listeria, Escherichia, Pseudomonas aeruginosa, Salmonella, Shigella, Staphylococcus aureus, Streptococcus pneumoniae, Tuberculosis, Group A Streptococcus, or Group B Streptococcus, Toxoplasma, Cyclospora, Giardia, Cryptosporidium, and Trichinella.

In some embodiments, pathogenic organisms in the subject are resistant to treatment with a drug. In some embodiments, pathogenic organisms in the subject are resistant to treatment with multiple drugs (e.g., multi-drug resistant). Drugs that may be used to treat pathogenic organisms of the present disclosure may include antibiotics, antivirals, antifungals, and antiparasitics.

In some embodiments, a pathogenic organism that is susceptible to a drug may acquire resistance to one or more drugs. In some embodiments, a pathogenic organism that is susceptible to antibiotics may acquire resistance to one or more antibiotics. In some embodiments, a pathogenic organism is resistant to one or more drugs (e.g., antibiotics) and may acquire resistance to one or more drugs (e.g., antibiotics). In some embodiments, the pathogenic organism is resistant to one or more drugs (e.g., antibiotics) and may become a multi-drug resistant organism by acquiring resistance to more than one drugs (e.g., antibiotics).

In general, there are multiple means by which an organism may acquire drug resistance. In some embodiments, the organism may acquire drug resistance by acquiring mutations in its genome that promote survival of the organism in the presence of at least one drug. In some embodiments, the organism acquires DNA from an organism that is resistant to one or more drugs (e.g., antibiotics), thereby becoming resistant to the drug (e.g., antibiotic). In some embodiments, the organism acquires a plasmid, such as a plasmid containing one or more drug (e.g., antibiotic) resistance genes, from an organism that is resistant to one or more drugs (e.g., antibiotics), thereby becoming resistant to the drug (e.g., antibiotic).

In some embodiments, administration of the compositions described herein reduces/inhibits or prevents intestinal colonization with oral microbiome bacteria. In some embodiments, administration of the compositions described herein reduces/inhibits or prevents the colonization, replication, proliferation, and/or survival of oral microbiome bacteria in the intestinal tract of the subject. In some embodiments, administration of the compositions described herein allows for colonization of the gastrointestinal tract of the subject by the bacterial strain(s) of the compositions thereby preventing colonization by oral microbiome bacteria.

In some embodiments, the subject is a carrier of an oral bacterium and is suffering from the effects of the infection. In some embodiments the subject is an asymptomatic carrier of an oral bacterium. In some embodiments, the subject has experienced recurrent or chronic colonization with an oral bacterium. In some embodiments, the subject is at risk of colonization with an oral bacterium. In some embodiments, the subject has a risk factor associated with colonization with an oral bacterium. In some embodiments, the subject is taking a proton pump inhibitor. In some embodiments, the subject has had a previous infection or colonization with an oral bacterium.

In some embodiments, the subject is to undergo a procedure that puts the subject at a higher risk of colonization and the compositions are administered prophylactically. In some embodiments, the compositions provided herein are administered to a subject to lower the risk of becoming colonized with an oral bacterium.

Over 700 bacterial species or phylotypes have been found in the oral cavity; however, over 50% have not yet been cultivated. A number of phyla have been identified in the oral microbiome Actinobacteria, Arachnia, Bacteroidetes, Bifidobacterium, Chlamydiae, Chloroflexi, Eubacterium, Euryarchaeota, Fusobacterium, Firmicutes, Fusobacteria, Lactobacillus, Leptotrichia, Peptococcus, Peptostreptococcus, Propionibacterium, Proteobacteria, Selenomonas, Spirochaetes, SRI, Synergistetes, Tenericutes, Treponema, TM7, and Veillonella (Dewhirst et al., J. Bacteriology (2010) 192(19): 5002-5010). Examples of oral microbiome bacteria include, without limitation, Streptococcus sanguis, Streptococcus salivarius, Streptococcus mitis, Streptococcus mutans, Treponema denticola, Eikenella corrodens, Streptococcus gordonii, Streptococcus oralis, Acinomyces maeslundii, and Bacteroides melaningenicus. A list of example species found in the human oral cavity can be found on the Human Oral Microbiome Database (homd.org). In some embodiments, the oral microbiome bacteria may be pathogenic. In some embodiments, the oral microbiome bacteria may be pathogenic if the bacteria gain access to another site of the body. In some embodiments, the oral microbiome bacteria are not pathogenic.

In some embodiments, administration of the compositions described herein reduces/inhibits or prevents intestinal colonization with oral microbiome bacteria. In some embodiments, the oral microbiome bacteria are Fusobacterium nucleatum (See e.g., Yoneda et al. J Gastrointest Dig Syst (2016) 6:2). In some embodiments, the oral microbiome bacteria are Campylobacter concisus (See e.g., Yoneda et al. J Gastrointest Dig Syst (2016) 6:2). In some embodiments, the oral microbiome bacteria are Streptococcus mutans (See e.g., Yoneda et al. J Gastrointest Dig Syst (2016) 6:2). Additional oral microbiome bacteria are described in Table S1A of Atarashi et al. (Atarashi et al., Science (2017) 358, 359-365), such as Rothia mucilaginosa, Neisseria subflava, Granulicatella para-adiacens, Streptococcus salivarius, Streptococcus mitis, Fusobacterium sp. 1_1_41FAA, Streptococcus oralis, Streptococcus salivarius, Neisseria subflava, Prevotella scopos, Veillonella parvula, Streptococcus sp. M143, Haemophilus parainfluenzae, Prevotella sp. CD3_34, Neisseria macacae, Prevotella histicola, Prevotella pallens, Streptococcus infantis, Streptococcus parasanguinis, Porphyromonas CW034, Streptococcus sp. oral strain T1-E5, Gemella sp. 933-88, Veillonella parvula, and Prevotella sp. C561.

In some embodiments, intestinal colonization with an oral microbiome bacterium induces a Th1 immune response in the subject. Examples of oral microbiome bacteria that may induce Th1 immune responses have been isolated, showing significant similarity (>96.3%) to the following species: Mogibacterium sp. CM96, Peptostreptococcus stomatis, Bifidobacterium sp. Group III-3, Slackia exigua, Veillonella denticariosi, Atopobium parvulum, Veillonella sp. 2011_oral_VSA_A3, Campylobacter concisus, Actinomyces odontolyticus, Solobacterium moorei, Enterococcus faecium, Bifidobacterium dentium, Veillonella parvela, Fusobacterium sp. 3_1_33, Klebsiella aeromobilis, and Klebsiella pneumoniae (see, e.g., Atarashi et al., Science (2017) 358: 359-365, Schirmer et al., Cell Host and Microbe (2018) 24: 600-610).

In some embodiments, the bacterium that induces a Th1 immune response (e.g., IBD) in a subject is a pathobiont. “Pathobiont” refers to a potentially pathological (disease-causing) organism which, under normal circumstances, lives as a symbiont. Examples of pathobionts include a bacterium that is associated with chronic inflammatory conditions (e.g., IBD). Non-limiting examples of pathobionts include Shigella spp., Campylobacter spp., Cryptosporidium spp., Salmonella spp., Escherichia coli strains (e.g., Enteropathogenic E. coli, Enteroaggregative E. coli, Enterotoxigenic E. coli), Veillonella dispar, Aggregatibacter segnis, Campylobacter, Lachnospiraceae, Veillonella parvula, Haemophilus parainfluenzae, Megasphaera, Escherichia coli, Enterobacteriaceae spp., Enterococcus spp., Fusobacterium spp., Gemella spp., Veillonella spp., Pasteurella spp., Neisseria spp., Haemophilus spp., Campylobacter spp., and Bifidobacterium spp.

In some embodiments, the methods may involve determining whether an oral bacterium is present in the subject. In some embodiments, the methods may involve determining whether an oral bacterium colonizes the oral cavity of the subject. In some embodiments, a subject may be at risk of intestinal colonization if the oral bacterium is present in the oral cavity of the subject. In some embodiments, the methods involve administering the combinations described herein to the subject, if an oral bacterium is detected in the oral cavity of the subject.

In some embodiments, the methods may involve determining whether an oral bacterium is present in the intestine of the subject. In some embodiments, the methods involve administering the combinations described herein to the subject, if an oral bacterium is detected in the intestine of the subject.

In some embodiments, the methods are for treating a disease or disorder associated with bacterial colonization in a subject. In some embodiments, the methods are for treating a disease or disorder associated with an immune response induced by bacteria in a subject. In some embodiments, the methods are for treating a disease or disorder associated with an undesired immune response induced by bacteria in a subject.

In some embodiments, the methods may involve determining whether the subject is colonized with bacteria. In some embodiments, the methods may involve determining whether the subject has or is experiencing an immune response induced by bacterial colonization. In some embodiments, a subject may be at risk of an immune response induced by bacterial colonization if the subject is colonized by the bacteria. In some embodiments, the methods involve administering the combinations described herein to the subject, if the subject is determined to be colonized by the bacteria. In some embodiments, the methods involve administering the combinations described herein to the subject, if the subject is determined to be experiencing or have experienced an immune response induced by bacterial colonization.

In some embodiments, the immune response induced by bacterial colonization is a Th1 immune response. As will be evident to one of skill in the art, Th1 immune responses are mediated the Th1 population of CD4+ cells. Th1 cells produce IFN-γ and other pro-inflammatory factors. The differentiation of CD4+ cells to Th1 cells is promoted by the presence of IL-2 and/or IL-12 and activation of the transcription factors STAT4 and T-bet. In some embodiments, the immune responses induced by bacterial colonization are Th1 pro-inflammatory responses. Any direct or indirect measure of Th1 immune response, such as the amount of IFN-γ or the number of Th1 cells, may be used to assess the level or extent of the immune response in a sample from a subject.

In some embodiments, the compositions provided herein are administered to a subject if the subject has an autoimmune disease. Examples of autoimmune diseases include, without limitation, inflammatory bowel disease (IBD), ulcerative colitis, Crohn's disease, sprue, autoimmune arthritis, rheumatoid arthritis, graft versus host disease, Type I diabetes, multiple sclerosis, osteoarthritis, juvenile chronic arthritis, Lyme arthritis, psoriatic arthritis, reactive arthritis, spondyloarthropathy, systemic lupus erythematosus, insulin dependent diabetes mellitus, thyroiditis, asthma, psoriasis, dermatitis scleroderma, atopic dermatitis, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, cachexia, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrome, adult (acute) respiratory distress syndrome, alopecia, alopecia areata, seronegative arthopathy, arthropathy, Reiter's disease, psoriatic arthropathy, chlamydia, yersinia and salmonella associated arthropathy, spondyloarhopathy, atheromatous disease/arteriosclerosis, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic cosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, insulin-dependent diabetes mellitus, sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, cosinophilic esophagitis, hypereosinophilic syndrome, eosinophilic gastroenteritis cutaneous lupus erythematosus, eosinophilic esophagitis, hypereosinophilic syndrome, and cosinophilic gastroenteritis, and diarrhea. In some embodiments, the autoimmune disease is inflammatory bowel disease (IBD). In some embodiments, the autoimmune disease is ulcerative colitis. In some embodiments, the autoimmune disease is Crohn's disease.

In some embodiments, the compositions provided herein are administered to a subject if the subject has non-alcoholic steatohepatitis (NASH), primary sclerosing cholangitis (PSC), non-alcoholic fatty liver disease (NAFLD), gastroesophageal reflux disease (GERD), or alcoholism.

In some embodiments, the compositions provided herein are administered to a subject if an immune response associated with bacterial colonization has been detected in the subject. In some embodiments, the methods involve determining whether the subject has an immune response induced by or associated with colonization with an undesired organism.

In some embodiments, the compositions provided herein are administered to a subject if the subject has a dysbiosis (e.g., has as microbiome associated with a disease state). In some embodiments, treatment with the compositions provided herein results in the change in the microbiome of the subject. In some embodiments, treatment with the compositions provided herein removes the dysbiosis in the subject resulting in a healthy microbiome. In some embodiments, treatment with the compositions provided herein removes the dysbiosis in the subject resulting in microbiome refractory or less susceptible to infection by a pathogen.

In some embodiments, the compositions and methods described herein are for the treatment and/or prevention of sepsis. In some embodiments, the compositions provided herein are administered to a subject if the subject has or is at risk for sepsis. In some embodiments, the compositions and methods described herein are for the treatment and/or prevention of sepsis in critically ill subjects, such as patients in an intensive care unit (ICU, also referred to as an intensive therapy unit or intensive treatment unit (ITU) or critical care unit (CCU)). In some embodiments, the compositions provided herein are administered to a subject if the subject is a critically ill subject and has or is at risk for sepsis. As will be evident to one of ordinary skill in the art, sepsis is a systemic inflammatory response that is typically due to infection with bacterium, fungus, virus, or protozoa. The severity of sepsis can range from mild (sepsis) to severe sepsis or even septic shock. Subjects of advanced age (e.g., over 65 years old), young age (e.g., under 1 year old), and/or having a compromised immune systems, including due to cancer, diabetes, burns, trauma, or other disease or disorder resulting in a critical illness, may be at increased risk of developing sepsis.

In some embodiments, the compositions and methods described herein are for the treatment and/or prevention of infection in critically ill subjects, such as patients in an intensive care unit (ICU). In some embodiments, the compositions provided herein are administered to a subject if the subject is a critically ill subject. In some embodiments, the subject may be a patient in an intensive care unit. In some embodiments, any of the compositions described herein may be administered to a subject to treat and/or prevent a bacterial infection, fungal infection, viral infection, or protozoan infection in a critically ill subject.

In some embodiments, the compositions and methods described herein are for the treatment and/or prevention of infection following a surgical procedure involving the gastrointestinal tract (i.e., gastrointestinal surgery). In some embodiments, the compositions and methods described herein are administered to a subject if the subject has undergone a surgical procedure involving the gastrointestinal tract. In some embodiments, the compositions and methods described herein are administered to a subject if the subject will be undergoing a surgical procedure involving the gastrointestinal tract (e.g., administered prior to the surgical procedure). In some embodiments, the subject has a disease or disorder associated with the gastrointestinal tract. In some embodiments, any of the compositions described herein may be administered to a subject to treat and/or prevent a bacterial infection, fungal infection, viral infection, or protozoan infection.

In some embodiments, the compositions and methods described herein are for the treatment and/or prevention of infection in subjects having cirrhosis (i.e., a cirrhosis patient). In some embodiments, the compositions and methods described herein are administered to a subject if the subject has or is at risk of cirrhosis. As will be evident to one of ordinary skill in the art, cirrhosis, also referred to as liver cirrhosis or hepatic cirrhosis, is a condition associated with liver dysfunction, predominantly due to long-term damage of the liver tissue and cells thereof. Cirrhosis may be caused by any of a variety of factors, for example, hepatitis (e.g., viral infection with Hepatitis B or Hepatitis C, autoimmune hepatitis), and excessive alcohol consumption. In some embodiments, any of the compositions described herein may be administered to a subject to treat and/or prevent a bacterial infection, fungal infection, viral infection, or protozoan infection in a cirrhosis patient.

In some embodiments, the compositions and methods described herein are for the treatment and/or prevention of infection in subjects who received a bone marrow transplant (i.e., bone marrow transplant patients). In some embodiments, the compositions and methods described herein are administered to a subject if the subject has undergone or will undergo a bone marrow transplant. In some embodiments, any of the compositions described herein may be administered to a subject to treat and/or prevent a bacterial infection, fungal infection, viral infection, or protozoan infection in a bone marrow transplant patient.

Any of the compositions described herein may be administered to a subject in a therapeutically effective amount or a dose of a therapeutically effective amount to treat or prevent a disease or disorder, for example associated with colonization with bacteria or an immune response associated with colonization with bacteria. The terms “treat” and “treatment” refer to reducing or alleviating one or more of the symptoms associated with colonization with bacteria or an immune response associated with colonization with bacteria. In some embodiments, any of the compositions described herein may be administered to a subject to prevent a disease or disorder. In some embodiments, any of the compositions described herein may be administered to a subject to prevent a Th1 related disease or disorder. In some embodiments, any of the compositions described herein may be administered to a subject to prevent IBD. The terms “prevent” and “prevention” encompass prophylactic administration and may reduce the incidence or likelihood of colonization with bacteria or an immune response associated with colonization with bacteria. For instance, in some embodiments, administration of the compositions provided herein result in a healthy microbiome that is refractory to pathogenic infection, thereby preventing the pathogenic infection or re-colonization with the pathogenic organism.

As used herein, a “therapeutically effective amount” may be used interchangeably with the term “effective amount.” A therapeutically effective amount or an effective amount of composition, such as a pharmaceutical composition, is any amount that results in a desired response or outcome in a subject, such as those described herein, including but not limited to reducing or preventing colonization with bacteria or an immune response associated with colonization with bacteria.

It should be appreciated that the term effective amount may be expressed as the number of bacteria or bacterial spores to be administered. It should further be appreciated that the bacteria can multiply once administered. Thus, administration of even a relatively small amount of bacteria may have therapeutic effects.

In some embodiments, the therapeutically effective amount of any of the compositions described herein is an amount sufficient to enhance survival of the subject, reduce or prevent bacterial colonization of the subject, and/or reduce or inhibit toxin production by the pathogenic infection. In some embodiments, colonization may be assessed by detecting and/or quantifying the bacteria in a sample from the subject, such as a fecal sample. In some embodiments, the therapeutically effective amount is an amount sufficient to reduce the colonization bacteria (e.g., pathogenic organisms, multi-drug resistant organisms, for example carbapenem resistant Enterobacteriaceae or extended spectrum beta-lactamase producing Enterobacteriaceae) in a fecal sample from the subject by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the bacterial burden in a subject that has not received any of the compositions described herein, or as compared to a fecal sample from the same subject that was collected prior to administration of any of the compositions.

In some embodiments, the compositions provided herein reduce an immune response associated with bacterial colonization or induced by bacterial colonization. In some embodiments, the therapeutically effective amount is an amount sufficient to reduce an immune response associated with bacterial colonization or induced by bacterial colonization by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 150-fold, 200-fold, 500-fold or more, as compared to the immune response associated with bacterial colonization or induced by bacterial colonization prior to administration of any of the compositions.

In some embodiments, the therapeutically effective amount is an amount sufficient to recolonize or repopulate the gastrointestinal tract of the subject with non-pathogenic bacteria. In some embodiments, the therapeutically effective amount is an amount sufficient to graft one or more of the bacterial strains of the composition in the gastrointestinal tract of the subject. In some embodiments, a fecal sample is obtained from the subject to assess the bacterial burden of undesired bacteria (e.g., pathogenic organisms, multi-drug resistant organisms, for example carbapenem resistant Enterobacteriaceae or extended spectrum beta-lactamase producing Enterobacteriaceae) and/or evaluate the efficacy of administration of the bacterial compositions described herein. In some embodiments, the microbiota of the subject (e.g., the identity and abundance of strains and/or species of the microbiota) may be assessed to determine a disease state of the subject and/or assess progress of the treatment. In some embodiments, the microbiota of the subject having a pathogenic infection is compared to the microbiota of a healthy subject, such as a subject that is not experiencing or has not experienced the pathogenic infection. In some embodiments, the microbiota of the subject having a pathogenic infection is compared to the microbiota of the same subject from a fecal sample obtained from the subject prior to the pathogenic infection.

In some embodiments, administration of the compositions provided herein results in a healthy microbiome that reduces or prevents colonization of the subject by any undesired organism. In some embodiments, administration of the compositions provided herein results in a healthy microbiome that reduces or prevents intestinal colonization of the subject by any undesired organism (e.g., pathogenic organisms, multi-drug resistant organisms, for example carbapenem resistant Enterobacteriaceae or extended spectrum beta-lactamase producing Enterobacteriaceae). In some embodiments, administration of the compositions provided herein results in a healthy microbiome that reduces an immune response associated with bacterial colonization, such as colonization with undesired bacteria. In some embodiments, administration of the compositions provided herein results in a healthy microbiome that reduces a Th1 immune response in the subject.

Any of the compositions described herein may be administered in combination with one or more additional compositions that can suppress a Th1 response and/or induces the accumulation and/or proliferation of regulatory T cells, and/or Th17 cells. In some embodiments, any of the compositions described herein may be administered in combination with a composition that induces the proliferation and/or accumulation of regulatory T cells (“Treg”).

In some embodiments, any of the compositions described herein may be administered in combination with a Treg inducing composition of bacterial strains, described for instance in Atarashi et al., Nature (2013) 500: 232-236. In some embodiments, the Treg inducing composition comprises at least 5, at least 10, at least 11, at least 12, at least 13, at least 14, at least 15, at least 16, or at least 17 of the following species: Clostridium saccharogumia, Flavonifractor plautii, Clostridium hathewayi, Blautia coccoides, Clostridium bolteae ATCC BAA-613, cf. Clostridium sp. MLG055, Clostridium indolis, Anaerotruncus colihominis, Ruminococcus sp. ID8, Clostridium asparagiforme DSM 15981, Clostridium symbiosum, Clostridium ramosum, Eubacterium contortum, Lachnospiraceae bacterium 5_1_57FAA, Lachnospiraceae bacterium 3_1_57FAA_CT1, Clostridiales bacterium 1_7_47FAA, and Lachnospiraceae bacterium A4. It should be appreciated that subsets of the bacterial compositions described above can also induce Treg cells. Examples of subsets bacteria that induce Treg cells are found for instance in Atarashi et al., Nature (2013) 500: 232-236 and corresponding Supplemental Information. In some embodiments, any of the compositions described herein may be administered in combination with any of the bacterial compositions as described in PCT Publication WO 2016/209806.

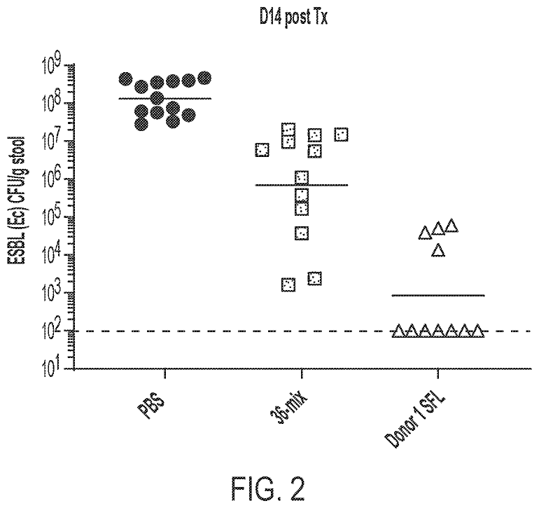

In some embodiments, any of the compositions described herein may be administered in combination with any of the bacterial compositions described in PCT Publication WO 2019/094837, which is incorporated herein by reference in its entirety. In some embodiments, any of the compositions described herein may be administered in combination with any of the bacterial compositions described in PCT Publication WO 2019/118515, which is incorporated herein by reference in its entirety. In some embodiments, any of the compositions described herein are administered in combination with a composition of 36 bacterial strains (e.g., the 36-mix, shown in FIG. 2).

As used herein, the phrase “induces proliferation and/or accumulation of regulatory T cells” refers to an effect of inducing the differentiation of immature T cells into regulatory T cells, which differentiation leads to the proliferation and/or the accumulation of regulatory T cells. Further, the meaning of “induces proliferation and/or accumulation of regulatory T cells” includes in vivo effects, in vitro effects, and/or ex vivo effects. In some embodiments, the proliferation and/or accumulation of regulatory T cells may be assessed by detecting and/or quantifying the number of cells that express markers of regulatory T cells (e.g., Foxp3 and CD4), for example by flow cytometry. In some embodiments, the proliferation and/or accumulation of regulatory T cells may be assessed by determining the activity of the regulatory T cells, such as the production of cytokines (e.g., IL-10).

In some embodiments, any of the compositions described herein may be administered in combination with a composition that induces the accumulation and/or proliferation of Th17 cells, see e.g., compositions disclosed in PCT Publication WO 2015/156419, which is incorporated herein by reference in its entirety.

In some embodiments, suppressing live bacterial product also repopulates the microbiota of the subject.

In some embodiments, administration of the compositions described herein results in an increase in the proliferation and/or accumulation of regulatory T cells (e.g., total Tregs or pathogenic organism-specific Tregs) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or pathogenic organism-specific Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 10⁴-fold, 10⁵-fold or more, as compared to the quantity of regulatory T cells in another subject (e.g., a reference subject) who did not receive the compositions.

In some embodiments, administration of the compositions described herein results in an increase the proliferation and/or accumulation of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the quantity of regulatory T cells in another subject (e.g., a reference subject) who did not receive the compositions.

The induction of Treg cells and corresponding decolonization of pathogenic organisms are intricately related. In some embodiments, administration of the compositions described herein results in an increase the proliferation and/or accumulation of regulatory T cells (e.g., total Tregs or pathogenic organism-specific Tregs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of regulatory T cells in the subject (or particular site in the subject) prior to administration of the compositions. In some embodiments, administration of the compositions described herein results in an increase the proliferation and/or accumulation of regulatory T cells (e.g., total Tregs or pathogenic organism-specific Tregs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of regulatory T cells in another subject (e.g., a reference subject) who did not receive the compositions.

In some embodiments, administration of the compositions described herein results in an increase in activity of regulatory T cells (e.g., total Tregs or pathogenic organism-specific Tregs) at a particular site (e.g., the gastrointestinal tract) in the subject. In some embodiments, administration of the compositions described herein results in an increase in activity of regulatory T cells (e.g., total Tregs or pathogenic organism-specific Tregs) by at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 10⁵-fold or more, as compared to the activity of regulatory T cells in the subject (or particular site in the subject) prior to administration of the compositions. In some embodiments, administration of the compositions described herein results in an increase in activity of regulatory T cells (e.g., total Tregs or pathogenic organism-specific Tregs) by at least 1.5-fold, 2-fold, 3-fold, 4-fold, 5-fold, 6-fold, 7-fold, 8-fold, 9-fold, 10-fold, 20-fold, 30-fold, 40-fold, 50-fold, 100-fold, 1000-fold, 104-fold, 10⁵-fold or more, as compared to the activity of regulatory T cells in another subject (e.g., a reference subject) who did not receive the compositions.

In some embodiments, administration of the compositions described herein results in an increase in the activity of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in the subject (or particular site in the subject) prior to administration of the compositions. In some embodiments, administration of the compositions described herein results in an increase in the activity of regulatory T cells (e.g., total Tregs or pathogenic organism-specific Tregs) by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 100%, 125%, 150% or more, as compared to the activity of regulatory T cells in another subject (e.g., a reference subject) who did not receive the compositions.

The abundance of regulatory T cells (e.g., total Tregs or pathogenic organism-specific Tregs) can be assessed by any method known in the art, for example by detecting a cellular marker indicative of regulatory T cells (e.g., FoxP3), assessing a direct or indirect activity of regulatory T cells, and/or by measuring the production of one or more cytokines produced by regulatory T cells (e.g., IL-10).

Some aspects compositions and methods described herein increase production of short chain fatty acids (e.g., in the gastrointestinal tract of the subject). In some embodiments, the methods involve administering to a subject one or more compositions containing bacterial strains that produce short chain fatty acids. SCFAs are abundant in healthy subjects (e.g., subjects not having a pathogenic organism infection) and decreased in subjects having pathogenic organism infections (e.g., Clostridium difficile infections and rCDIs). Fecal matter transplant (FMT) have been shown to increase SCFA following rCDI (Seckatz, et al., Anaerobe (2018) 53: 64-73.

SCFA produced in the gastrointestinal tract are thought to function as signaling molecules between the gut microbiota and the host organism, with the SCFA playing a in local, intermediary and peripheral metabolism of the host. Sec, e.g., Morrison, et al. Gut Microbes (2016) 7(3): 189-200. In some embodiments, a damaged gut mucosal barrier can be repaired by providing SCFA.

Examples of SCFA include, without limitation, formic acid, acetic acid, butyric acid, isobutyric acid, valeric acid, or isovaleric acid. In some embodiments, the SCFA is butyric acid (butyrate).

The production of SCFAs and corresponding decolonization of pathogenic organisms are intricately related. In some embodiments, administration of the compositions described herein results in an increase in the production and/or accumulation of SCFAs (e.g., total SCFAs or pathogenic organism-specific SCFAs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of SCFAs in the subject (or particular site in the subject) prior to administration of the compositions. In some embodiments, administration of the compositions described herein results in an increase in the production and/or accumulation of SCFAs (e.g., total SCFAs or pathogenic organism-specific SCFAs) by between 1% and 20%, 2% and 19%, 3% and 17%, 4% and 16%, 4% and 15%, 5% and 15%, 6% and 14%, 7% and 13%, 8% and 12%, 5% and 10%, 5% and 15%, 10% and 15%, or 8% and 15% as compared to the quantity of SCFAs in another subject (e.g., a reference subject) who did not receive the compositions.

In some embodiments, the compositions and methods described herein result in an increase in the amount of SCFAs produced in the gastrointestinal tract of the subject. In some embodiments, the SCFAs are increased by 10-fold to 500-fold following administration of the composition as described herein. In some embodiments, SCFAs are increased by 20-fold to 250-fold following administration of the compositions described herein. In some embodiments, SCFAs are increased by 100-fold to 500-fold following administration of the compositions described herein. In some embodiments, SCFAs are increased by at least 2-fold, 5-fold, 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise butyrate. In some embodiments, the level of butyrate (e.g., in the gastrointestinal tract of the subject) is increased by 10-fold to 500-fold following administration of the compositions described herein. In some embodiments, the level of butyrate is increased by 20-fold to 250-fold following administration of compositions described herein. In some embodiments, the level of butyrate is increased by 100-fold to 500-fold following administration of the compositions described herein. In some embodiments, the level of butyrate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise propionate. In some embodiments, the level of propionate (e.g., in the gastrointestinal tract of the subject) is increased by 10-fold to 500-fold following administration of the compositions described herein. In some embodiments, the level of propionate is increased by 20-fold to 250-fold following administration of compositions described herein. In some embodiments, the level of propionate is increased by 100-fold to 500-fold following administration of the compositions described herein. In some embodiments, the level of propionate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise acetate. In some embodiments, the level of acetate (e.g., in the gastrointestinal tract of the subject) is increased by 10-fold to 500-fold following administration of the compositions described herein. In some embodiments, the level of acetate is increased by 20-fold to 250-fold following administration of compositions described herein. In some embodiments, the level of acetate is increased by 100-fold to 500-fold following administration of the compositions described herein. In some embodiments, the level of acetate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, SCFAs comprise formate. In some embodiments, the level of acetate (e.g., in the gastrointestinal tract of the subject) is increased by 10-fold to 500-fold following administration of the compositions described herein. In some embodiments, the level of formate is increased by 20-fold to 250-fold following administration of compositions described herein. In some embodiments, the level of formate is increased by 100-fold to 500-fold following administration of the compositions described herein. In some embodiments, the level of formate is increased by at least 2-fold, 5-fold 10-fold, 20-fold, 30-fold, 40-fold, 50-fold, 60-fold, 70-fold, 80-fold, 90-fold, 100-fold, 200-fold, 300-fold, 400-fold, or 500-fold following administration of the compositions described herein.

In some embodiments, the subject is infected with a pathogenic organism. In some embodiments, the subject has a Clostridium difficile infection (CDI). In some embodiments, the CDI is recurrent (rCDI). rCDI is CDI that occurs more than once in the same subject and is associated with reduced short chain fatty acids (SCFAs), increased primary bile acids, and decreased secondary bile acids in the gut microbiota of the subject.

Bile acids are steroid acids that allow the digestion of dietary fats and oils by acting as surfactants that turn the fats and oils into micelles. Bile acids also act as hormones utilizing the farnesoid X receptor and GBPAR1. Primary bile acids are synthesized in the liver from cholesterol and a conjugated with either taurine or glycine prior to secretion. When the primary bile acids are secreted into the lumen of the intestine, bacteria partially dehydroxylate and remove the glycine or taurine groups, forming secondary bile acids.

Non-limiting examples of primary bile acids are cholic acid (CA), chenodeoxycholic acid (CDCA), glycocholic acid (GCA), glycochenodeoxycholic acid (GCDCA), glycodeoxycholic acid (GDCA), taurocholic acid (TCA), and turochenodeoxycholic acid (TCDCA). Non-limiting examples of secondary bile acids are deoxycholic acid (DCA), lithocholic acid (LCA), ursodeoxycholic acid (UDCA), taurodeoxycholic acid (TDCA), taurolithocholic acid (TLCA), and tauroursodeoxycholic acid (TUDCA).

Pathogenic organism infection, including Clostridium difficile infection and rCDI, are associated with increased primary bile acids and reduced secondary bile acids. The primary bile acids are reduced, and the secondary bile acids are increased following fecal matter transplant (FMT) (Seckatz, et al., Anaerobe (2018) 53: 64-73). In some embodiments, administration of the bacterial strains or a pharmaceutical composition as described herein reduces primary bile acids and/or increases secondary bile acids.

In some embodiments, the levels of primary bile acids are reduced by 10-fold to 100,000-fold following administration of the bacterial strains or the pharmaceutical composition. In some embodiments, the levels of primary bile acids are reduced by 10-fold to 1,000-fold following administration of the bacterial strains or the pharmaceutical composition. In some embodiments, the levels of primary bile acids are reduced 20-fold to 10,000-fold following administration of the bacterial strains or the pharmaceutical composition. In some embodiments, the levels of primary bile acids are reduced by 10-fold, 100-fold, 200-fold, 300-fold, 400-fold, 500-fold, 600-fold, 700-fold, 800-fold, 900-fold, 1,000-fold, 10,000-fold, 20,000-fold, 30,000-fold, 40,000-fold, 50,000-fold, 60,000-fold, 70,000-fold, 80,000-fold, 90,000-fold, or 100,000-fold following administration of the bacterial strains or the pharmaceutical composition.

In some embodiments, the levels of secondary bile salts are increased by 10-fold to 10,000-fold following administration of the bacterial strains or the pharmaceutical composition. In some embodiments, the levels of secondary bile acids are increased by 10-fold to 1,000-fold following administration of the bacterial strains or the pharmaceutical composition. In some embodiments, the levels of secondary bile acids are increased by 20-fold to 100-fold following administration of the bacterial strains or the pharmaceutical composition. In some embodiments, the levels of secondary bile acids are increased by 10-fold, 20-fold, 50-fold, 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, or 1,000-fold following administration of the bacterial strains or the pharmaceutical composition.

In general, short chain fatty acids (SCFAs) are fatty acids containing six or less carbon atoms. SCFAs are produced when dietary fiber is fermented in the intestine. Non-limiting examples of SCFAs include hexanoate, pentanoate, butyrate, propionate, acetate, and formate. SCFAs are primarily absorbed in the portal vein following lipid digestion, and can affect the production of lipids, energy, and vitamins. Further, SCFAs play a critical role in maintaining intestinal epithelial cell membrane integrity for preventing pathogenic organism (e.g., Clostridium difficile) infection.

In one aspect, the disclosure provides methods comprising administration of multiple doses of the pharmaceutical compositions. In some embodiments, the disclosure provides methods comprising administration of antibiotic (e.g., vancomycin) followed by multiple doses of the pharmaceutical compositions. In some embodiments, administration of multiple doses of the pharmaceutical compositions described herein provides enhanced colonization (engraftment) of one or more bacterial strains of the pharmaceutical compositions as compared to administration of a single dose of the pharmaceutical composition. In some embodiments, administration of multiple doses of the pharmaceutical compositions described herein provides enhanced recovery of one or more bacterial strains of the pharmaceutical compositions as compared to administration of a single dose of the pharmaceutical composition. In some embodiments, administration of multiple doses of the pharmaceutical compositions described herein provides increased abundance of one or more bacterial strains of the pharmaceutical compositions as compared to administration of a single dose of the pharmaceutical composition. In some embodiments, administration of multiple doses of the pharmaceutical compositions described herein provides an increase in the number of subjects that were colonized with of all of bacterial strains of the pharmaceutical compositions as compared to administration of a single dose of the pharmaceutical composition. In some embodiments, administration of multiple doses of the pharmaceutical compositions described herein provides durable colonization (e.g., up to 6 months) of one or more bacterial strains of the pharmaceutical compositions as compared to administration of a single dose of the pharmaceutical composition. In some embodiments, administration of multiple doses of the pharmaceutical compositions described herein provides durable colonization (e.g., up to 6 months) of all of the bacterial strains of the pharmaceutical compositions as compared to administration of a single dose of the pharmaceutical composition. It should further be appreciated that administration of multiple dose my results in a combination of the results described. Thus, for example, in some embodiments, administration of multiple doses of the pharmaceutical compositions described herein provides enhanced colonization (engraftment) and increased rate of recovery of one or more bacterial strains of the pharmaceutical compositions as compared to administration of a single dose of the pharmaceutical composition.

In some embodiments, administration of multiple doses of the pharmaceutical compositions described herein provides enhanced colonization (engraftment) of one or more bacterial strains of the pharmaceutical compositions as compared to administration of a single dose of the pharmaceutical composition. Administration of multiple doses of the pharmaceutical composition may result in enhanced colonization (engraftment) and an increased abundance of each of the bacterial strains of the pharmaceutical composition. In some embodiments, administration of a single dose of the pharmaceutical composition results in the same or a similar level of engraftment (e.g., total bacteria) as administration of multiple doses of the pharmaceutical composition, however the engraftment may be dominated by one bacterial strain or only a subset of the bacterial strains of the pharmaceutical compositions.

Any of the methods described herein may involve administering an antibiotic to the subject prior to administration of the pharmaceutical compositions described herein. In some embodiments, the antibiotic is vancomycin, fidaxomycin or ridinilazole. Non-limiting examples of antibiotics that may be used in any of the methods provided herein include cephalosporin antibiotics cephalexin, cefuroxime, cefadroxil, cefazolin, cephalothin, cefaclor, cefamandole, cefoxitin, cefprozil, ceftobiprole, clindamycin, ceftriaxone, cefotaxime, cefazolin, cefoperazone, cefuroxime, cefmetazole, fluoroquinolone, ciprofloxacin, Levaquin, floxin, tequin, avelox, norflox, tetracycline, minocycline, oxytetracycline, doxycycline, amoxicillin, ampicillin, penicillin V, dicloxacillin, benzylpenicillin, carbenicillin, vancomycin, and methicillin, ertapenem, doripenem, imipenem/cilastatin, meropenem, clavulanate, tazobactam, piperacillin, ceftriaxone, cefotaxime, cefazolin, fluoroquinolone, imipenem, meropenem, metronidazole, fidaxomyxin, or ridinilazole.

In some embodiments, any of the methods described herein may further comprise administering vancomycin to the subject prior to administration of the pharmaceutical compositions described herein. In some embodiments, the method does not comprise administering an antibiotic to the subject prior to administration of the pharmaceutical compositions described herein. In some embodiments, the method does not comprise administering vancomycin to the subject prior to administration of the pharmaceutical compositions described herein. Vancomycin administration has been found to alter the composition of human gut microbiota. See, e.g., Reijnders et al. Cell Metabolism (2016) 24(1): 63-72. Without wishing to be bound by any particular theory, it is thought that administration of vancomycin may aid engraftment of the bacterial strain(s) of the pharmaceutical compositions described herein, for example by removing other microbes present in the gastrointestinal tract.

In some embodiments, the antibiotic (e.g., vancomycin) is administered to the subject once, as a single dose. In some embodiments, the antibiotic (e.g., vancomycin) is administered to the subject in multiple doses. In some embodiments, the antibiotic (e.g., vancomycin) is administered to the subject in at least 2, 3, 4, 5, 6, 7, 8, 9, 10, 11, 12, 13, 14, 15 or more doses. The multiple doses of the antibiotic (e.g., vancomycin) may be administered to the subject at regular intervals prior to administering any of the pharmaceutical compositions described herein. In some embodiments, each of the multiple doses of the antibiotic (e.g., vancomycin) are administered on consecutive days (e.g., first dose on day 1, second dose of day 2, third dose on day 3, etc.). In some embodiments, the antibiotic (e.g., vancomycin) is administered to the subject for 2, 3, 4, 5, 6, 7, 8, 9, 10, 11, 12, 13, 14, 15 or more consecutive days. In some embodiments, the antibiotic (e.g., vancomycin) is administered to the subject each day for three consecutive days. In some embodiments, the antibiotic (e.g., vancomycin) administered to the subject each day for five consecutive days.

In some embodiments, the antibiotic (e.g., vancomycin) administered to the subject each day for seven consecutive days. In some embodiments, the antibiotic (e.g., vancomycin) administered to the subject for one day. In any of the embodiments described herein, a subject may be administered one or more doses of a first antibiotic followed by one or more doses of a second antibiotic.

In some embodiments, a single dose, or the first dose in a treatment regimen of multiple doses, is administered, the same day as the administration of the final dose of the antibiotic (e.g., vancomycin). In some embodiments, a single dose, or the first dose in a treatment regimen of multiple doses, is administered, the day after administration of the final dose of the antibiotic (e.g., vancomycin). In some embodiments, a single dose, or the first dose in a treatment regimen of multiple doses, is administered, two days after administration of the final dose of the antibiotic (e.g., vancomycin). In some embodiments, the methods provided herein allow for a wash out day between the final dose of the antibiotic (e.g., vancomycin) and the first dose of the pharmaceutical composition. In some embodiments, a single dose, or the first dose in a treatment regimen of multiple doses, is administered, three days, four days, five days, six days, ten days or more, after administration of the final dose of the antibiotic (e.g., vancomycin). In some embodiments, the methods provided herein allow for multiple wash out days between the final dose of the antibiotic (e.g., vancomycin) and the first dose of the pharmaceutical composition.

Each dose of the antibiotic (e.g., vancomycin) may be the same amount of the antibiotic or may be a different amount of the antibiotic. In some embodiments, the antibiotic (e.g., vancomycin) is administered in an amount sufficient to allow for colonization of one or more of the bacterial strains of the pharmaceutical compositions described herein. In some embodiments, the subject is administered between about 50 mg and 1 g, 100 mg and 750 mg, 100 mg and 500 mg. 200 mg and 750 mg. 200 mg and 500 mg, 300 mg and 750 mg, 300 mg and 500 mg, 100 mg and 400 mg, 100 mg and 300 mg, 100 mg and 200 mg, 200 mg and 400 mg. 200 mg and 300 mg, or 450 mg to 550 mg of the antibiotic per day. As will be appreciated by one of skill in the art, the total amount of vancomycin administered to the subject per day may be administered in a single dose or between multiple doses, which in sum results in the total amount of the antibiotic per day.

In some example, the subject is administered about 500 mg vancomycin per day prior to administration of any of the pharmaceutical compositions described herein. In some embodiments, 500 mg of vancomycin per day is administered in a single dose (e.g., 500 mg). In some embodiments, 500 mg of vancomycin per day is administered in multiple doses (e.g., 2, 3, 4, 5 or more), which in sum results in 500 mg vancomycin per day. In some embodiments, 500 mg vancomycin is administered in 4 doses of 125 mg vancomycin per day. In some embodiments, 500 mg of vancomycin is administered to the subject for one day. In some embodiments, 500 mg of vancomycin is administered to the subject per day for two days. In some embodiments, 500 mg vancomycin is administered to the subject per day for three days. In some embodiments, 500 mg vancomycin is administered to the subject per day for four days. In some embodiments, 500 mg vancomycin is administered to the subject per day for five days. In some embodiments, 500 mg vancomycin is administered to the subject per day for six days. In some embodiments, 500 mg vancomycin is administered to the subject per day for seven days. In some embodiments, 500 mg vancomycin is administered to the subject per day for eight days. In some embodiments, 500 mg vancomycin is administered to the subject per day for nine days. In some embodiments, 500 mg vancomycin is administered to the subject per day for ten days.

In some embodiments, the subject is administered about 250 mg vancomycin per day prior to administration of any of the pharmaceutical compositions described herein. In some embodiments, 250 mg vancomycin per day is administered in a single dose (e.g., 250 mg). In some embodiments, 250 mg vancomycin per day is administered in multiple doses (e.g., 2, 3, 4, 5 or more), which in sum results in 250 mg vancomycin per day. In some embodiments, 250 mg vancomycin is administered in 2 doses of 125 mg vancomycin per day. In some embodiments, 250 mg vancomycin is administered to the subject for one day. In some embodiments, 250 mg vancomycin is administered to the subject per day for two days. In some embodiments, 250 mg vancomycin is administered to the subject per day for three days. In some embodiments, 250 mg vancomycin is administered to the subject per day for four days. In some embodiments, 250 mg vancomycin is administered to the subject per day for five days. In some embodiments, 250 mg vancomycin is administered to the subject per day for six days. In some embodiments, 250 mg vancomycin is administered to the subject per day for seven days. In some embodiments, 250 mg vancomycin is administered to the subject per day for eight days. In some embodiments, 250 mg vancomycin is administered to the subject per day for nine days. In some embodiments, 250 mg vancomycin is administered to the subject per day for ten days.

In some embodiments, the subject is administered about 125 mg vancomycin per day prior to administration of any of the pharmaceutical compositions described herein. In some embodiments, the 125 mg vancomycin per day is administered in a single dose (e.g., 125 mg). In some embodiments, the 125 mg vancomycin per day is administered in multiple doses (e.g., 2, 3, 4, 5 or more), which in sum results in 125 mg vancomycin per day. In some embodiments, 125 mg vancomycin is administered to the subject for one day. In some embodiments, 125 mg vancomycin is administered to the subject per day for two days. In some embodiments, 125 mg vancomycin is administered to the subject per day for three days. In some embodiments, 125 mg vancomycin is administered to the subject per day for four days. In some embodiments, 125 mg vancomycin is administered to the subject per day for five days. In some embodiments, 125 mg vancomycin is administered to the subject per day for six days. In some embodiments, 125 mg vancomycin is administered to the subject per day for seven days. In some embodiments, 125 mg vancomycin is administered to the subject per day for eight days. In some embodiments, 125 mg vancomycin is administered to the subject per day for nine days. In some embodiments, 125 mg vancomycin is administered to the subject per day for ten days.

In some embodiments, the disclosure provides methods comprising administering one or more antibiotics to the subject and subsequently administering any of the bacterial compositions to the subject once, twice, 3 times, 4 times, 5 times, 6 times, 7 times, 8 times, 9 times, or at least 10 times, or more. In some embodiments, the disclosure provides methods comprising administering one or more antibiotics to the subject and subsequently administering any of the bacterial compositions described herein to the subject in multiple doses at a regular interval, such as every 2 weeks, every month, every 2 months, every 3 months, every 4 months, every 5 months, every 6 months, or more. In some embodiments, one dose of any of the compositions described herein is administered and a second dose of the composition is administered the following day (e.g., consecutive day). In some embodiments, one dose of any of the compositions described herein is administered and each of the additional doses of the composition are administered on consecutive days (e.g., first dose on day 1, second dose of day 2, third dose on day 3, etc.).

In one aspect, the disclosure provides methods comprising administering one or more antibiotics to the subject and subsequently administering any of the bacterial compositions as multiple daily doses of the pharmaceutical compositions. In some embodiments, the pharmaceutical compositions are administered on a daily basis for 2 days, 3 days, 4, days, 5, days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 1 month, 2 months, 3 months, 4 months, 5 months, 6 months, 7 months, 8 months, 9 months, 10 months, 11 months, 12 months or more. In some embodiments, the antibiotic (e.g., vancomycin) is administered according to a pulse tapered regime. See e.g., Sirbu et al., Clinical Infectious Diseases (2017) 65: 1396-1399.

In some embodiments, the antibiotic (e.g., vancomycin) is administered to the subject at least 1, 2, 3, 4, 5, 6, 7 days or more prior to administration of the pharmaceutical compositions described herein. In some embodiments, administration of antibiotic (e.g., vancomycin) is terminated at least one day (e.g., 1, 2, 3, 4, 5, or more) prior to administration of any of the pharmaceutical compositions described herein.

In some embodiments, additional antibiotics are administered in combination with the vancomycin regimes provided herein.

It should be appreciated, in some embodiments, that any of the vancomycin doses or administration regimens may be combined with any of the pharmaceutical composition doses or administration regimens provided herein.

In one aspect the disclosure provides methods comprising the administration of an antibiotic (e.g., vancomycin) followed by the administration of a pharmaceutical composition provided herein, wherein the administration of an antibiotic (e.g., vancomycin) is followed by the administration of a single dose or multiple doses of the pharmaceutical composition. In some embodiments, administration of an antibiotic (e.g., vancomycin) followed by the administration of a single dose or multiple doses of the pharmaceutical composition results in an increase in the abundance of bacterial strains of the pharmaceutical compositions in the microbiome of the subject (engraftment) compared to the administration of a pharmaceutical composition without the administration of the antibiotic. In some embodiments, administration of an antibiotic (e.g., vancomycin) followed by the administration of a single dose or multiple doses of the pharmaceutical composition results in an increase in the duration of the colonization of bacterial strains of the pharmaceutical composition in the microbiome of the subject (e.g., up to 6 months) compared to the administration of a pharmaceutical composition without the administration of the antibiotic.

In some embodiments, administration of an antibiotic (e.g., vancomycin) followed by the administration of a single dose or multiple doses of the pharmaceutical composition results in an increase in the rate of engraftment of the initial amount of the bacterial strains of the pharmaceutical composition in the microbiome of the subject by between ten- to one hundred-fold (e.g., within the first 48 hours) compared to the administration of a pharmaceutical composition without the administration of the antibiotic.

In some embodiments, administration of an antibiotic (e.g., vancomycin) followed by the administration of a single dose or multiple doses of the pharmaceutical composition results in a greater number (amount) of subjects having all of the bacterial strains of the pharmaceutical composition present in their microbiome as compared to compared to the administration of a pharmaceutical composition without the administration of the antibiotic.

In some embodiments, administration of an antibiotic (e.g., vancomycin) followed by the administration of multiple doses of the pharmaceutical composition results in an increase in the abundance of bacterial strains of the pharmaceutical composition in the microbiome of the subject (engraftment) compared to the administration of a single dose of the pharmaceutical composition. In some embodiments, the disclosure provides methods comprising the administration of a pharmaceutical composition provided herein, wherein the administration of multiple doses of the pharmaceutical composition increases the abundance of bacterial strains in the microbiota of the subject (engraftment) of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in an increase in the rate of engraftment of the initial amount of the bacterial strains of the pharmaceutical composition in the microbiome of the subject as compared to the administration of a single dose of the pharmaceutical composition. In some embodiments, the disclosure provides methods comprising the administration of a pharmaceutical composition provided herein, wherein the administration of multiple doses of the pharmaceutical composition increases the rate of engraftment of the initial amount of the bacterial strains of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in a higher abundance of the bacterial strains of the pharmaceutical composition in the microbiome of the subject as compared to the administration of a single dose of the pharmaceutical composition. In some embodiments, the disclosure provides methods comprising the administration of a pharmaceutical composition provided herein, wherein the administration of multiple doses of the pharmaceutical composition results in higher abundance of the bacterial strains of the pharmaceutical composition in the microbiome of the subject compared to the administration of a single dose of the pharmaceutical composition.

In some embodiments, administration of an antibiotic (e.g., vancomycin) followed by the administration of multiple doses of the pharmaceutical composition results in a greater number (amount) of subjects having all of the bacterial strains of the pharmaceutical composition present in their microbiome as compared to the administration of a single dose of the pharmaceutical composition. In some embodiments, the disclosure provides methods comprising the administration of a pharmaceutical composition provided herein, wherein the administration of multiple doses of the pharmaceutical results in a greater number (amount) of subject having all of the bacterial strains of the pharmaceutical composition in their microbiome as compared to the administration of a single dose of the pharmaceutical composition.

In some embodiments, administration of an antibiotic (e.g., vancomycin) followed by the administration of multiple doses of the pharmaceutical composition results in an accelerated recovery of the microbiome (e.g., increase in bacterial species of Bacteroidetes and/or Firmicutes, and/or decrease in Proteobacteria) as compared to the administration of a single dose of the pharmaceutical composition. In some embodiments, the disclosure provides methods comprising the administration of a pharmaceutical composition provided herein, wherein the administration of multiple doses of the pharmaceutical results in an accelerated recovery of the microbiome (e.g., increase in bacterial species of Bacteroidetes and/or Firmicutes, and/or decrease in Proteobacteria) as compared the administration of a single dose of the pharmaceutical composition.

In some embodiments, administration of an antibiotic (e.g., vancomycin) followed by the administration of a single dose or multiple doses of the pharmaceutical composition results in an accelerated recovery of the microbiome (e.g., increase in bacterial species of Bacteroidetes and/or Firmicutes, and/or decrease in Proteobacteria) as compared to the administration of an antibiotic (e.g., vancomycin) without the administration of a pharmaceutical composition.

In some embodiments, any of the methods described herein may further comprise administering a composition comprising taurine to the subject. In some embodiments, the composition comprising taurine is administered concomitantly with any of the pharmaceutical compositions comprising a purified bacterial mixture described herein. In some embodiments, the composition comprising taurine is administered prior to administration of any of the pharmaceutical compositions comprising a purified bacterial mixture described herein. In some embodiments, the composition comprising taurine is administered after administration of any of the pharmaceutical compositions comprising a purified bacterial mixture described herein. In some embodiments, the composition comprising taurine is administered prior to and after administration of any of the pharmaceutical compositions comprising a purified bacterial mixture described herein.

Taurine, also referred to as 2-aminoethanesulfonic acid, is an organic compound that is widely distributed in animal tissues and is a major constituent of bile and can be found conjugated to bile acids. Taurine may account for up to 0.1% of total human body weight and can be found in the large intestine, among other locations. The precise function of taurine in the physiological processes is not known, but without wishing to be bound by any particular theory, it is thought that taurine may help support muscle maintenance.

Taurine occurs naturally in fish and meat, and consumption in humans generally varies between 40 and 400 mg per day. However, in recent years, energy drinks for human consumption have included taurine. One example energy drink, marketed under the trademark Red Bull®, contains approximately 1000 mg of taurine per can (serving). High consumption of taurine is generally considered to be safe with no adverse effects noted from up to 3000 mg taurine per day.

Taurine administration has been found to alter the composition of mouse gut microbiota. It has recently been reported that infection may induce host taurine production and the expansion of taurine utilizers. Upon release of bile acids from the gallbladder into the gut, bacteria present in the gut may cleave taurine from bile acids, making the taurine available for use as an energy source. Bacteria, for example of the class Deltaproteobacteria, such as Biophila wadsworthia, utilize taurine in anaerobic respiration, and the expansion of Deltaproteobacteria following taurine administration is thought to aid in inhibiting intestinal colonization by Klebsiella pneumoniae and Citrobacter rodentium, a mouse model of pathogenic E. coli. Sec, e.g., Stacy et al. Cell. 2021 184(3):615-627.

As a result, gut microbiota from previously infected hosts were found to display enhanced resistance to infection. The microbiota from previously infected hosts has been associated with altered bile acid metabolism leading to the expansion of taxa that utilize the sulfonic acid, taurine. Indeed, it was reported that supplying exogenous taurine alone was sufficient to induce this alteration in microbiota function and enhance resistance. Mechanistically, taurine potentiates the microbiota's production of sulfide, an inhibitor of cellular respiration, which is key to host invasion by numerous pathogens. Sec, e.g., Stacy et al. Cell. 2021 184(3):615-627.

Without wishing to be bound by any particular theory, it is thought that administration of taurine may promote replication and/or aid engraftment of the bacterial strain(s) of the pharmaceutical compositions described herein, for example by serving as an energy source for bacteria present in the gastrointestinal tract or pharmaceutical composition.

In some embodiments, compositions comprising at least two of the strains selected from the group consisting of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri in combination with taurine may be effective at inhibiting infection and/or reducing/preventing colonization with a pathogen that is more than additive of either taurine alone or the bacterial compositions alone.

As described herein, any of the bacterial compositions may further comprise taurine. In such embodiments, the bacterial strain and taurine are administered to the subject at the same time. In some embodiments, taurine is administered to the subject in a separate composition, such as a pharmaceutical composition or food product, including beverage product.

In some embodiments, the composition comprises between about 40 mg to about 3000 mg taurine. In some embodiments, the composition comprises between about 100 mg to about 2000 mg taurine. In some embodiments, the composition comprises between about 500 mg to about 1500 mg taurine.

In some embodiments, the compositions comprising bacterial strains also comprise taurine. In some embodiments, the composition comprises about 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1100 mg, 1200 mg, 1300 mg, 1400 mg, 1500 mg, 1600 mg, 1700 mg, 1800 mg, 1900 mg, 2000 mg. 2100 mg, 2200 mg, 2300 mg, 2400 mg, 2500 mg, 2600 mg, 2700 mg, 2800 mg, 2900 mg, or 3000 mg taurine.

In some embodiments, the subject is administered between about 40 mg to about 3000 mg of taurine per day. In some embodiments, the subject is administered between about 100 mg to about 2000 mg taurine per day. In some embodiments, the subject is administered between about 500 mg to about 1500 mg taurine per day. Any of the daily amounts of taurine may be administered to the subject as a single dosage or as multiple dosages across the day.

In some embodiments, a composition comprising taurine is administered to the subject once, as a single dose. In some embodiments, the composition comprising taurine is administered to the subject in multiple doses. In some embodiments, the composition comprising taurine is administered to the subject in at least 2, 3, 4, 5, 6, 7, 8, 9, 10, 11, 12, 13, 14, 15 or more doses.

One or more doses of a composition comprising taurine may be administered to the subject prior to administering any of the pharmaceutical compositions containing purified bacterial strains described herein. In some embodiments, a composition comprising taurine is administered in multiple doses, such as on consecutive days (e.g., first dose on day 1, second dose of day 2, third dose on day 3, etc.) prior to administering any of the pharmaceutical compositions containing purified bacterial strains described herein. In some embodiments, a composition comprising taurine is administered to the subject for 2, 3, 4, 5, 6, 7, 8, 9, 10, 11, 12, 13, 14, 15, or more consecutive days prior to administering any of the pharmaceutical compositions containing purified bacterial strains described herein. In some embodiments, a composition comprising taurine is administered to the subject each day for three consecutive days prior to administering any of the pharmaceutical compositions containing purified bacterial strains described herein. In some embodiments, the composition comprising taurine is administered to the subject each day for five consecutive days prior to administering any of the pharmaceutical compositions containing purified bacterial strains described herein. In some embodiments, a composition comprising taurine is administered to the subject each day for seven consecutive days prior to administering any of the pharmaceutical compositions containing purified bacterial strains described herein. In some embodiments, a composition comprising taurine is administered to the subject for one day prior to administering any of the pharmaceutical compositions containing purified bacterial strains described herein.

One or more doses of a composition comprising taurine may be administered to the subject after administering any of the pharmaceutical compositions containing purified bacterial strains described herein. In some embodiments, a composition comprising taurine is administered in multiple doses, such as on consecutive days (e.g., first dose on day 1, second dose of day 2, third dose on day 3, etc.) after administering any of the pharmaceutical compositions containing purified bacterial strains described herein. In some embodiments, a composition comprising taurine is administered to the subject for 2, 3, 4, 5, 6, 7, 8, 9, 10, 11, 12, 13, 14, 15, or more consecutive days after administering any of the pharmaceutical compositions containing purified bacterial strains described herein. In some embodiments, a composition comprising taurine is administered to the subject each day for three consecutive days after administering any of the pharmaceutical compositions containing purified bacterial strains described herein. In some embodiments, the composition comprising taurine is administered to the subject each day for five consecutive days after administering any of the pharmaceutical compositions containing purified bacterial strains described herein. In some embodiments, a composition comprising taurine is administered to the subject each day for seven consecutive days after administering any of the pharmaceutical compositions containing purified bacterial strains described herein. In some embodiments, a composition comprising taurine is administered to the subject for one day after administering any of the pharmaceutical compositions containing purified bacterial strains described herein.

In some embodiments, a composition comprising taurine is administered the same day as the administration of any of the pharmaceutical compositions containing purified bacterial strains described herein. In some embodiments, a composition comprising taurine is administered at the same time (simultaneously) or substantially at the same time as the administration of any of the pharmaceutical compositions containing purified bacterial strains described herein. In some embodiments, a composition comprising taurine is administered within 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours of the administration of any of the pharmaceutical compositions containing purified bacterial strains described herein.

The composition comprising taurine may in any form for human administration, such as a food product/beverage, capsule, pill, tablet, or liquid composition.

In some embodiments, the methods described herein may involve subjecting the subject to a bowel lavage (bowel irrigation, whole bowel irrigation, gastrointestinal lavage, gastric lavage) prior to administration of the compositions described herein. In some embodiments, a bowel lavage may remove or aid in removing microbiota from the gastrointestinal tract of the subject, creating a niche for the bacterial strains of the compositions described herein. In some embodiments, the bowel lavage may be an oral bowel lavage or a rectal bowel lavage.

Methods of performing a bowel lavage are known in the art, and generally involve the rapid administration of large volumes of a solution, such as polyethylene glycol or a balanced electrolyte solution. A rectal bowel lavage can involve the administration of a solution or a suppository containing the pharmaceutical composition. A bowel lavage may be performed under doctor supervision, hospitalization, or at home.

Any of the compositions described herein, including the pharmaceutical compositions and food products comprising the compositions, may contain bacterial strains in any form, for example in an aqueous form, such as a solution or a suspension, embedded in a semi-solid form, in a powdered form or freeze dried form. In some embodiments, the composition or the bacterial strains of the composition are lyophilized. In some embodiments, a subset of the bacterial strains in a composition is lyophilized. Methods of lyophilizing compositions, specifically compositions comprising bacteria, are well known in the art. Sec, e.g., U.S. Pat. Nos. 3,261,761; 4,205,132; PCT Publications WO 2014/029578 and WO 2012/098358, herein incorporated by reference in their entirety. The bacteria may be lyophilized as a combination and/or the bacteria may be lyophilized separately and combined prior to administration. A bacterial strain may be combined with a pharmaceutical excipient prior to combining it with the other bacterial strain or multiple lyophilized bacteria may be combined while in lyophilized form and the mixture of bacteria, once combined may be subsequently be combined with a pharmaceutical excipient. In some embodiments, the bacterial strain is a lyophilized cake. In some embodiments, the compositions comprising the one or more bacterial strains are a lyophilized cake.

In some embodiments, one or more of the bacterial strains of the compositions, including pharmaceutical compositions and food products, has been spray-dried. In some embodiments, a subset of the bacterial strains is spray-dried. The process of spray-drying refers to production of dry powder from a liquid comprising bacterial compositions (Sec, e.g., Ledet, et al., Spray Draying of Pharmaceuticals in “Lyophilized Biologics and Vaccines” pages 273-294, Springer). In general, the process involves rapidly drying the bacterial compositions with a hot gas. A bacterial strain may be combined with a pharmaceutical excipient prior to combining it with the other bacterial strains or multiple spray-dried bacterial strains may be combined while in spray-dried form and the mixture of bacterial strains, once combined, may be subsequently combined with a pharmaceutical excipient.

The bacterial strains of the composition can be manufactured using fermentation techniques well known in the art. In some embodiments, the active ingredients are manufactured using anaerobic fermenters, which can support the rapid growth of anaerobic bacterial species. The anaerobic fermenters may be, for example, stirred tank reactors or disposable wave bioreactors. Culture media such as BL media and EG media, or similar versions of these media devoid of animal components, can be used to support the growth of the bacterial species. The bacterial product can be purified and concentrated from the fermentation broth by traditional techniques, such as centrifugation and filtration, and can optionally be dried and lyophilized by techniques well known in the art.

In some embodiments, the composition of bacterial strains may be formulated for administration as a pharmaceutical composition. The term “pharmaceutical composition” as used herein means a product that results from the mixing or combining of at least one active ingredient, such as any two or more purified bacterial strains described herein, and one or more inactive ingredients, which may include one or more pharmaceutically acceptable excipient.

An “acceptable” excipient refers to an excipient that must be compatible with the active ingredient and not deleterious to the subject to which it is administered. In some embodiments, the pharmaceutically acceptable excipient is selected based on the intended route of administration of the composition, for example a composition for oral or nasal administration may comprise a different pharmaceutically acceptable excipient than a composition for rectal administration. Examples of excipients include sterile water, physiological saline, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, and a solubilizer.

Pharmaceutical compositions disclosed herein can be prepared in accordance with methods well known and routinely practiced in the art (see e.g., Remington: The Science and Practice of Pharmacy, Mack Publishing Co. 20th ed. 2000). The pharmaceutical compositions described herein may further comprise any carriers or stabilizers in the form of a lyophilized formulation or an aqueous solution. Acceptable excipients, carriers, or stabilizers may include, for example, buffers, antioxidants, preservatives, polymers, chelating reagents, and/or surfactants. Pharmaceutical compositions are preferably manufactured under GMP conditions. The pharmaceutical compositions can be used orally, nasally or parenterally, for instance, in the form of capsules, tablets, pills, sachets, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like. In some embodiments, the pharmaceutical compositions can be used by injection, such as by intravenous, intramuscular, subcutaneous, or intradermal administration.

Aspects of the present disclosure relate to administration of compositions comprising taurine. In some embodiments, a composition comprising any of the purified bacterial strains described herein may further comprise taurine. In such embodiments, administration of the composition results in administration to the subject of both the purified bacterial strains and taurine. In some embodiments, a composition comprising any of the purified bacterial strains described herein does not contain taurine. In some embodiments, a composition that is not the composition comprising the purified bacterial strains comprises taurine. In some embodiments, the methods involve administering to a subject a composition comprising purified bacterial strains and administering to the subject a second composition comprising taurine.

In some embodiments, the compositions comprising bacterial strains are formulated for delivery to the intestines (e.g., the small intestine and/or the colon). In some embodiments, the compositions comprising bacterial strains are formulated with an enteric coating that increases the survival of the bacteria through the harsh environment in the stomach. The enteric coating is one which resists the action of gastric juices in the stomach so that the bacteria of the composition therein will pass through the stomach and into the intestines. The enteric coating may readily dissolve when in contact with intestinal fluids, so that the bacteria enclosed in the coating will be released in the intestinal tract. Enteric coatings may consist of polymer and copolymers well known in the art, such as commercially available EUDRAGIT (Evonik Industries). (See e.g., Zhang, AAPS PharmSciTech (2016) 17(1): 56-67).

The compositions comprising bacterial strains may also be formulated for rectal delivery to the intestine (e.g., the colon). Thus, in some embodiments, compositions comprising bacterial strains may be formulated for delivery by suppository, colonoscopy, endoscopy, sigmoidoscopy or enema. A pharmaceutical preparation or formulation and particularly a pharmaceutical preparation for oral administration, may include an additional component that enables efficient delivery of the compositions of the disclosure to the intestine (e.g., the colon). A variety of pharmaceutical preparations that allow for the delivery of the compositions to the intestine (e.g., the colon) can be used. Examples thereof include pH sensitive compositions, more specifically, buffered sachet formulations or enteric polymers that release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is between about 6.8 and about 7.5. Such a numeric value range is a range in which the pH shifts toward the alkaline side at a distal portion of the stomach, and hence is a suitable range for use in the delivery to the colon. It should further be appreciated that each part of the intestine (e.g., the duodenum, jejunum, ileum, cecum, colon and rectum), has different biochemical and chemical environment. For instance, parts of the intestines have different pHs, allowing for targeted delivery by compositions that have a specific pH sensitivity. Thus, the compositions provided herein may be formulated for delivery to the intestine or specific parts of the intestine (e.g., the duodenum, jejunum, ileum, cecum, colon and rectum) by providing formulations with the appropriate pH sensitivity. (See e.g., Villena et al., Int J Pharm (2015) 487 (1-2): 314-9).

Another embodiment of a pharmaceutical preparation useful for delivery of the compositions to the intestine (e.g., the colon) is one that ensures the delivery to the colon by delaying the release of the contents (e.g., the bacterial strains) by approximately 3 to 5 hours, which corresponds to the small intestinal transit time. In one embodiment of a pharmaceutical preparation for delayed release, a hydrogel is used as a shell. The hydrogel is hydrated and swells upon contact with gastrointestinal fluid, with the result that the contents are effectively released (released predominantly in the colon). Delayed release dosage units include drug-containing compositions having a material which coats or selectively coats a drug or active ingredient to be administered. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Additional examples of pharmaceutical compositions that allow for the delivery to the intestine (e.g., the colon) include bioadhesive compositions which specifically adhere to the colonic mucosal membrane (for example, a polymer described in the specification of U.S. Pat. No. 6,368,586) and compositions into which a protease inhibitor is incorporated for protecting particularly a biopharmaceutical preparation in the gastrointestinal tracts from decomposition due to an activity of a protease.

Another example of a system enabling the delivery to the intestine (e.g., the colon) is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

A further example of a system enabling the delivery of a composition to the intestine (e.g., the colon), is a composition that includes a coating that can be removed by an enzyme present in the gut (e.g., the colon), such as, for example, a carbohydrate hydrolase or a carbohydrate reductase. Such a system is not particularly limited, and more specific examples thereof include systems which use food components such as non-starch polysaccharides, amylose, xanthan gum, and azopolymers.

The compositions provided herein can also be delivered to specific target areas, such as the intestine, by delivery through an orifice (e.g., a nasal tube) or through surgery. In addition, the compositions provided herein that are formulated for delivery to a specific area (e.g., the cecum or the colon), may be administered by a tube (e.g., directly into the small intestine). Combining mechanical delivery methods such as tubes with chemical delivery methods such as pH specific coatings, allow for the delivery of the compositions provided herein to a desired target area (e.g., the cecum or the colon).

The compositions comprising bacterial strains are formulated into pharmaceutically acceptable dosage forms by conventional methods known to those of skill in the art. Dosage regimens are adjusted to provide the optimum desired response (e.g., the prophylactic or therapeutic effect). In some embodiments, the dosage form of the composition is a tablet, pill, capsule, powder, granules, solution, or suppository. In some embodiments, the pharmaceutical composition is formulated for oral administration. In some embodiments, the pharmaceutical composition is formulated such that the bacteria of the composition, or a portion thereof, remain viable after passage through the stomach of the subject. In some embodiments, the pharmaceutical composition is formulated for rectal administration, e.g. as a suppository. In some embodiments, the pharmaceutical composition is formulated for delivery to the intestine or a specific area of the intestine (e.g., the colon) by providing an appropriate coating (e.g., a pH specific coating, a coating that can be degraded by target area specific enzymes, or a coating that can bind to receptors that are present in a target area).

Dosages of the active ingredients in the pharmaceutical compositions disclosed herein can be varied so as to obtain an amount of the active ingredient which is effective to achieve the desired pharmaceutical response for a particular subject, composition, and mode of administration, without being toxic or having an adverse effect on the subject. The selected dosage level depends upon a variety of factors including the activity of the particular compositions employed, the route of administration, the time of administration, the duration of the treatment, other drugs, compounds and/or materials used in combination with the particular compositions employed, the age, sex, weight, condition, general health and prior medical history of the subject being treated, and like factors.

A physician, veterinarian or other trained practitioner, can start doses of the pharmaceutical composition at levels lower than that required to achieve the desired therapeutic effect and gradually increase the dosage until the desired effect is achieved. In general, effective doses of the compositions, for the prophylactic or therapeutic treatment of groups of people as described herein vary depending upon many different factors, including routes of administration, physiological state of the subject, whether the subject is human or an animal, other medications administered, and the therapeutic effect desired. Dosages need to be titrated to optimize safety and efficacy. In some embodiments, the dosing regimen entails oral administration of a dose of any of the compositions described herein. In some embodiments, the dosing regimen entails oral administration of multiple doses of any of the compositions described herein. In some embodiments, the composition is administered orally the subject once, twice, 3 times, 4 times, 5 times, 6 times, 7 times, 8 times, 9 times, or at least 10 times. In some embodiments, any of the compositions described herein are administered the subject in multiple doses at a regular interval, such as every 2 weeks, every month, every 2 months, every 3 months, every 4 months, every 5 months, every 6 months, or more.

The compositions, including the pharmaceutical compositions disclosed herein, include compositions that contain selected bacterial strains. The amount of bacteria, including the amount of bacteria cells of each of the bacterial strains, in the compositions, including pharmaceutical compositions, may be expressed in weight, number of bacteria and/or CFUs (colony forming units). In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more of each of the bacterial strains per dosage amount. In some embodiments, the compositions, including pharmaceutical compositions, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more total bacterial cells per dosage amount. It should further be appreciated that bacteria of each of the bacterial strains may be present in different amounts. Thus, for instance, as a non-limiting example, composition may include 10³ of bacteria A, 10⁴ of bacteria B and 10⁶ of bacteria C. In some embodiments, compositions, including pharmaceutical composition, comprise about 10, about 10², about 10³, about 10⁴, about 10⁵, about 10⁶, about 10⁷, about 10⁸, about 10⁹, about 10¹⁰, about 10¹¹, about 10¹², about 10¹³ or more bacterial cells or CFUs of each of the bacterial strains per dosage amount. In some embodiments, compositions, including pharmaceutical compositions, comprise about 10¹, about 10², about 10³, about 10⁴, about 10⁵, about 10⁶, about 10⁷, about 10⁸, about 10⁹, about 10¹⁰, about 10¹¹, about 10¹², about 10¹³ or more bacterial cells or CFUs in total for all of the bacterial strains combined per dosage amount. As discussed above, bacteria of each of the bacterial strains may be present in different amounts. In some embodiments, the compositions, including pharmaceutical compositions, contain about 10⁻⁷, about 10⁻⁶, about 10⁻⁵, about 10⁻⁴, about 10⁻³, about 10⁻², about 10⁻¹ or more grams of bacteria (bacterial cells or CFU) of each of the bacterial strains in the composition per dosage amount. In some embodiments, the compositions, including pharmaceutical compositions, contain about 10⁻⁷, about 10⁻⁶, about 10⁻⁵, about 10⁻⁴, about 10⁻³, about 10⁻², about 10⁻¹ or more grams of bacteria (bacterial cells or CFU) in total for all of the bacterial strains combined per dosage amount.

In some embodiments, the dosage amount is one administration device (e.g., one table, pill or capsule). In some embodiments, the dosage amount is the amount administered at one time, which may be in the form of more than one administration device (e.g., more than one table, pill or capsule). In some embodiment, the dosage amount is the amount that is administered in a particular period (e.g., one day or one week).

As described herein, any of the pharmaceutical compositions described herein may be administered once, as a single dose. In some embodiments, the pharmaceutical compositions described herein are administered in multiple doses. In some embodiments, each dose is administered in the form of one or more capsules. In some embodiments, each dose comprises administration of multiple capsules. In some embodiments, each dose is administered in the form of 1, 2, 3, 4, 5, 6, 7, 8, 9, 10, 11, 12, 13, 14, 15, or more capsules.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFU of each of the bacterial strains per capsule.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or CFU per capsule. In some embodiments, each capsule contains between 10⁷ and 10⁹, between 10⁷ and 10⁸, or between 10⁸ and 10⁹ total bacteria. In some embodiments, each capsule contains about 1.0×10⁷, 2.0×10⁷, 3.0×10⁷, 4.0×10⁷, 5.0×10⁷, 6.0×10⁷, 7.0×10⁷, 8.0×10⁷, 9.0×10⁷, 1.0×10⁸, 2.0×10⁸, 3.0×10⁸, 4.0×10⁸, 5.0×10⁸, 6.0×10⁸, 7.0×10⁸, 8.0×10⁸, 9.0×10⁸, 1.0×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 3.9×10⁹, 4.0×10⁹, 4.1×10⁹, 4.2×10⁹, 4.3×10⁹, 4.4×10⁹, 4.5×10⁹, 4.6×10⁹, 4.7×10⁹, 4.8×10⁹, 4.9×10⁹, 5.0×10⁹ total bacterial cells or CFU.

In some embodiments, each capsule contains between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFU of each bacterial strain per capsule.

In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFUs of each of the bacterial strains per dosage amount. In some embodiments, the pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or CFUs per dosage amount.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacteria or colony forming units per milliliter.

In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria. In some embodiments, the compositions disclosed herein contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or colony forming units of bacteria per milliliter.

In some embodiments, a composition that includes more than one bacterial strain may contain each bacterial strain in the same quantities, in terms of bacterial cells or CFUs. For example, a composition of the invention comprising Collinsella aerofaciens and Bifidobacterium longum may comprise 1×10⁸ CFU of Collinsella aerofaciens and 1×10⁸ CFU of Bifidobacterium longum per milliliter or may comprise 1×10⁸ Collinsella aerofaciens cells and 1×10⁸ Bifidobacterium longum cells per milliliter.

In some embodiments, the pharmaceutical composition comprises at least 1.6×10⁹ total CFUs. In some embodiments, the pharmaceutical composition comprises at least 1.6×10⁹ total CFUs and is administered as a single dose. In some embodiments, the pharmaceutical composition comprises at least 1.6×10⁹ total CFUs and is administered as multiple (e.g., 2, 3, 4, 5, or more) doses. In some embodiments, the pharmaceutical composition comprises at least 1.6×10⁹ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 4.0×10⁹ total CFUs. In some embodiments, the pharmaceutical composition comprises at least 4.0×10⁹ total CFUs and is administered as a single dose. In some embodiments, the pharmaceutical composition comprises at least 4.0×10⁹ total CFUs and is administered as multiple (e.g., 2, 3, 4, 5, or more) doses. In some embodiments, the pharmaceutical composition comprises at least 4.0×10¹⁰ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 8.0×10⁹ total CFUs. In some embodiments, the pharmaceutical composition comprises at least 8.0×10⁹ total CFUs and is administered as a single dose. In some embodiments, the pharmaceutical composition comprises at least 8.0×10⁹ total CFUs and is administered as multiple (e.g., 2, 3, 4, 5, or more) doses. In some embodiments, the pharmaceutical composition comprises at least 8.0×10⁹ total CFUs and is administered as two, three, four, five, six, seven, eight, nine, ten, eleven, twelve, thirteen, fourteen, fifteen or more doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 2.8×10¹⁰ total CFUs. In some embodiments, the pharmaceutical composition comprises at least 2.8×10¹⁰ total CFUs and is administered as a single dose. In some embodiments, the pharmaceutical composition comprises at least 2.8×10¹⁰ total CFUs and is administered as multiple (e.g., 2, 3, 4, 5, or more) doses. In some embodiments, the pharmaceutical composition comprises at least 2.8×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 2.8×10¹⁰ total CFUs and is administered as seven doses. In some embodiments, each of the multiple doses are administered at regular intervals. In some embodiments, each of the multiple doses are on consecutive days (e.g., first dose on day 1, second dose of day 2, third dose on day 3, etc.).

In some embodiments, the pharmaceutical composition comprises at least 4.0×10¹⁰ total CFUs. In some embodiments, the pharmaceutical composition comprises at least 4.0×10¹⁰ total CFUs and is administered as a single dose. In some embodiments, the pharmaceutical composition comprises at least 4.0×10¹⁰ total CFUs and is administered as multiple (e.g., 2, 3, 4, 5, or more) doses. In some embodiments, the pharmaceutical composition comprises at least 4.0×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 4.0×10¹⁰ total CFUs and is administered as five doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 4.0×10¹⁰ total CFUs and is administered as five doses, each of which are administered on five consecutive days.

In some embodiments, the pharmaceutical composition comprises at least 5.6×10¹⁰ total CFUs. In some embodiments, the pharmaceutical composition comprises at least 5.6×10¹⁰ total CFUs and is administered as a single dose. In some embodiments, the pharmaceutical composition comprises at least 5.6×10¹⁰ total CFUs and is administered as multiple (e.g., 2, 3, 4, 5, or more) doses. In some embodiments, the pharmaceutical composition comprises at least 5.6×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 5.6×10¹⁰ total CFUs and is administered as fourteen doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 5.6×10¹⁰ total CFUs and is administered as fourteen doses, each of which are administered on fourteen consecutive days.

In some embodiments, the pharmaceutical composition comprises at least 1.1×10¹¹ total CFUs. In some embodiments, the pharmaceutical composition comprises at least 1.1×10¹¹ total CFUs and is administered as a single dose. In some embodiments, the pharmaceutical composition comprises at least 1.1×10¹¹ total CFUs and is administered as multiple (e.g., 2, 3, 4, 5, or more) doses. In some embodiments, the pharmaceutical composition comprises at least 1.1×10¹¹ total CFUs and is administered as two, three, four, five, six, seven, eight, nine, ten, eleven, twelve, thirteen, fourteen, fifteen or more doses. In some embodiments, the pharmaceutical composition comprises at least 1.1×10¹¹ total CFUs and is administered as fourteen doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 1.1×10¹¹ total CFUs and is administered as fourteen doses, each of which are administered on fourteen consecutive days.

In some embodiments, the pharmaceutical composition comprises at least 2.1×10¹⁰ total CFUs. In some embodiments, the pharmaceutical composition comprises at least 2.1×10¹⁰ total CFUs and is administered as a single dose. In some embodiments, the pharmaceutical composition comprises at least 2.1×10¹⁰ total CFUs and is administered as multiple (e.g., 2, 3, 4, 5, or more) doses. In some embodiments, the pharmaceutical composition comprises at least 2.1×10¹⁰ total CFUs and is administered as two, three, four, five, six, seven, eight, nine, ten, eleven, twelve, thirteen, fourteen, fifteen or more doses. In some embodiments, the pharmaceutical composition comprises at least 2.1×10¹⁰ total CFUs and is administered as five doses. In some embodiments, each of the multiple doses are administered at regular intervals. In some embodiments, each of the multiple doses are on consecutive days (e.g., first dose on day 1, second dose of day 2, third dose on day 3, etc.). In some embodiments, the pharmaceutical composition comprises at least 2.1×10¹⁰ total CFUs and is administered as five doses, each of which are administered on five consecutive days.

As described herein, any of the pharmaceutical compositions described herein may be administered to a subject in one dose or in multiple doses (e.g., initial administration), which may be followed by one or more additional doses of any of the pharmaceutical compositions described herein. In some embodiments, any of pharmaceutical composition described herein may be administered to a subject in one dose or in multiple doses in an initial administration, followed by one or more additional doses of a pharmaceutical composition comprising the same one or more bacterial strains as the pharmaceutical composition of the initial administration. In some embodiments, any of pharmaceutical composition described herein may be administered to a subject in one dose or in multiple doses in an initial administration, followed by one or more additional doses of a pharmaceutical composition comprising more total bacteria (colony-forming units) relative to the initial administration of the pharmaceutical composition. In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition. In some embodiments, the initial administration includes at least 1, 2, 3, 4, 5, 6, 7, 8, 9, 10, 11, 12, 13, 14, 15 or more doses of any of the pharmaceutical compositions described herein. In some embodiments, the additional administration includes at least 1, 2, 3, 4, 5, 6, 7, 8, 9, 10, 11, 12, 13, 14, 15 or more doses of any of the pharmaceutical compositions described herein. In some embodiments, the initial administration comprises two doses of any of the pharmaceutical composition and the additional administration comprises three doses of any of the pharmaceutical compositions described herein.

In some embodiments, any of pharmaceutical composition described herein may be administered to a subject in one dose or in multiple doses in an initial administration, followed by one or more additional doses of a pharmaceutical composition comprising fewer total bacteria (colony-forming units) relative to the initial administration of the pharmaceutical composition. In such embodiments, the dose(s) of the initial administration may be referred to as a “high dose” and the dose(s) of the additional administration may be referred to as a “low dose. In some embodiments, the high dose is at least 1.1-fold, 1.2-fold, 1.3-fold, 1.4-fold, 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold or more higher than the low dose. In some embodiments, the high dose is 8.0×10⁹ CFUs. In some embodiments, the low dose is 1.6×10⁹ CFUs. In some embodiments, the initial administration comprises multiple doses (e.g., 2, 3, 4, 5 or more) of 8.0×10⁹ CFUs and the additional administration comprises multiple doses (e.g., 2, 3, 4, 5 or more) of 1.6×10⁹ CFUs. In some embodiments, the low dose is 1.6×10⁹ CFUs. In some embodiments, the initial administration comprises two doses of 8.0×10⁹ CFUs and the additional administration comprises three doses of 1.6×10⁹ CFUs.

In some embodiments, the one or more additional administrations is performed on the day following the initial administration (e.g., consecutive days). In some embodiments, the one or more additional administrations is performed at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks or longer following the initial administration. In some embodiments, the one or more additional administrations is performed at least 6 weeks after the initial administration. In some embodiments, the one or more additional administrations is performed at least 12 weeks after the initial administration.

In some embodiments, the compositions, including pharmaceutical compositions, contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² bacterial cells or CFUs of each of the bacterial strains per dosage amount. In some embodiments, the compositions, including pharmaceutical compositions contain between 10 and 10¹³, between 10² and 10¹³, between 10³ and 10¹³, between 10⁴ and 10¹³, between 10⁵ and 10¹³, between 10⁶ and 10¹³, between 10⁷ and 10¹³, between 10⁸ and 10¹³, between 10⁹ and 10¹³, between 10¹⁰ and 10¹³, between 10¹¹ and 10¹³, between 10¹² and 10¹³, between 10 and 10¹², between 10² and 10¹², between 10³ and 10¹², between 10⁴ and 10¹², between 10⁵ and 10¹², between 10⁶ and 10¹², between 10⁷ and 10¹², between 10⁸ and 10¹², between 10⁹ and 10¹², between 10¹⁰ and 10¹², between 10¹¹ and 10¹², between 10 and 10¹¹, between 10² and 10¹¹, between 10³ and 10¹³, between 10⁴ and 10¹³, between 10⁵ and 10¹³, between 10⁶ and 10¹³, between 10⁷ and 10¹¹, between 10⁸ and 10¹¹, between 10⁹ and 10¹¹, between 10¹⁰ and 10¹¹, between 10 and 10¹⁰, between 10² and 10¹⁰, between 10³ and 10¹⁰, between 10⁴ and 10¹⁰, between 10⁵ and 10¹⁰, between 10⁶ and 10¹⁰, between 10⁷ and 10¹⁰, between 10⁸ and 10¹⁰, between 10⁹ and 10¹⁰, between 10 and 10⁹, between 10² and 10⁹, between 10³ and 10⁹, between 10⁴ and 10⁹, between 10⁵ and 10⁹, between 10⁶ and 10⁹, between 10⁷ and 10⁹, between 10⁸ and 10⁹, between 10 and 10⁸, between 10² and 10⁸, between 10³ and 10⁸, between 10⁴ and 10⁸, between 10⁵ and 10⁸, between 10⁶ and 10⁸, between 10⁷ and 10⁸, between 10 and 10⁷, between 10² and 10⁷, between 10³ and 10⁷, between 10⁴ and 10⁷, between 10⁵ and 10⁷, between 10⁶ and 10⁷, between 10 and 10⁶, between 10² and 10⁶, between 10³ and 10⁶, between 10⁴ and 10⁶, between 10⁵ and 10⁶, between 10 and 10⁵, between 10² and 10⁵, between 10³ and 10⁵, between 10⁴ and 10⁵, between 10 and 10⁴, between 10² and 10⁴, between 10³ and 10⁴, between 10 and 10³, between 10² and 10³, or between 10 and 10² total bacterial cells or CFUs per dosage amount.

In some embodiments, the compositions, including pharmaceutical compositions, contain between 10⁻⁷ and 10⁻¹, between 10⁻⁶ and 10⁻¹, between 10⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁵ and 10⁻², between 10⁻⁴ and 10⁻², between 10⁻³ and 10⁻², between 10⁻⁷ and 10⁻³, between 10⁻⁶ and 10⁻³, between 10⁵ and 10⁻³, between 10⁻⁴ and 10⁻³, between 10⁻⁷ and 10⁻⁴, between 10⁻⁶ and 10⁻⁴, between 10⁻⁵ and 10⁻⁴, between 10⁻⁷ and 10⁻⁵, between 10⁻⁶ and 10⁵, or between 10⁻⁷ and 10⁶ grams of bacteria of each of the bacterial strains in the composition per dosage amount. In some embodiments, the compositions, including pharmaceutical compositions, disclosed herein contain between 10⁻⁷ and 10⁻¹, between 10⁻⁶ and 10⁻¹, between 10⁵ and 10⁻¹, between 10⁻⁴ and 10⁻¹, between 10⁻³ and 10⁻¹, between 10⁻² and 10⁻¹, between 10⁻⁷ and 10⁻², between 10⁻⁶ and 10⁻², between 10⁻⁵ and 10⁻², between 10⁻⁴ and 10⁻², between 10⁻³ and 10⁻², between 10⁻⁷ and 10⁻³, between 10⁶ and 10⁻³, between 10⁻⁵ and 10⁻³, between 10⁻⁴ and 10⁻³, between 10⁻⁷ and 10⁻⁴, between 10⁶ and 10⁴, between 10⁻⁵ and 10⁴, between 10⁻⁷ and 10⁵, between 10⁻⁶ and 10⁵, or between 10⁻⁷ and 10⁻⁶ grams of all of the bacteria combined (total) per dosage amount.

Aspects of the present disclosure also provide food products comprising any of the compositions described herein and a nutrient. Also with the scope of the present disclosure are food products comprising any of the bacterial strains described herein and a nutrient. In some embodiments, the food product may further comprise taurine. Food products are, in general, intended for the consumption of a human or an animal. Any of the bacterial strains described herein may be formulated as a food product. In some embodiments, the bacterial strains are formulated as a food product in spore form. In some embodiments, the bacterial strains are formulated as a food product in vegetative form. In some embodiments, the food product comprises both vegetative bacteria and bacteria in spore form. The compositions disclosed herein can be used in a food or beverage, such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed.

Non-limiting examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

Food products containing bacterial strains described herein may be produced using methods known in the art and may contain the same amount of bacteria (e.g., by weight, amount or CFU) as the pharmaceutical compositions provided herein. Selection of an appropriate amount of bacteria in the food product may depend on various factors, including for example, the serving size of the food product, the frequency of consumption of the food product, the specific bacterial strains contained in the food product, the amount of water in the food product, and/or additional conditions for survival of the bacteria in the food product.

Examples of food products which may be formulated to contain any of the bacterial strains described herein include, without limitation, a beverage, a drink, a bar, a snack, a dairy product, a confectionery product, a cereal product, a ready-to-eat product, a nutritional formula, such as a nutritional supplementary formulation, a food or beverage additive.

TABLE 1
Examples of bacterial species of the
bacterial strains disclosed herein

| 47-mix (Closely related | | SEQ | |
|---|---|---|---|
| species based on 16S | Additional closely related | ID | |
| sequence) | species | NO: | Strain |
| Collinsella aerofaciens_F | Collinsella aerofaciens | 1 | 1 |
| Bacteroides xylanisolvens | | 2 | 2 |
| Alistipes putredinis | | 3 | 3 |
| Alistipes shahii | | 4 | 4 |
| Eubacterium_E hallii | Eubacterium hallii | 5 | 5 |
| Blautia_A obeum | Blautia obeum | 6 | 6 |
| Odoribacter splanchnicus | | 7 | 7 |
| Bacteroides cellulosilyticus | | 8 | 8 |
| Bacteroides uniformis | | 9 | 9 |
| Bacteroides_B vulgatus | Bacteroides vulgatus | 10 | 10 |
| Bacteroides fragilis | | 11 | 11 |
| Bacteroides caccae | | 12 | 12 |
| Paeniclostridium sordellii | Clostridium sordellii | 13 | 13 |
| Blautia producta | | 14 | 14 |
| Absiella innocuum | Erysipelotrichaceae | 15 | 15 |
| | bacterium | | |
| Clostridium_M | Clostridium clostridioforme | 16 | 16 |
| clostridioforme | | | |
| Paraclostridium massiliensis | Clostridium bifermentans | 17 | 17 |
| Dorea longicatena B | Dorea longicatena | 18 | 18 |
| Bacteroides thetaiotaomicron | | 19 | 19 |
| Parabacteroides distasonis | | 20 | 20 |
| Escherichia coli | | 21 | 21 |
| Bifidobacterium longum | | 22 | 22 |
| Absiella innocuum | Clostridium innoccum | 23 | 23 |
| Fusobacterium_A sp | Fusobacterium mortiferum | 24 | 24 |
| Parabacteroides merdae | | 25 | 25 |
| Coprococcus_B comes | Coprococcus comes | 26 | 26 |
| Agathobacter rectale | Eubacterium rectale | 27 | 27 |
| Erysipelatoclostridium | | 28 | 28 |
| ramosum | | | |
| Clostridium_{—}M citroniae | Clostridium citroniae | 29 | 29 |
| Bacteroides faecis | | 30 | 30 |
| Bacteroides ovatus | | 31 | 31 |
| Bifidobacterium | | 32 | 32 |
| pseudocatenulatum | | | |
| Bacteroides caccae | | 33 | 33 |
| Bifidobacterium adolescentis | | 34 | 34 |
| Agathobaculum sp | Agathobaculum sp, | 35 | 35 |
| MUR_P3C8a | Agathobaculum | | |
| | butyriciproducens | | |
| Phascolarctobacterium | Phascolarctobacterium | 36 | 36 |
| faecium MUR_P6E9 | faecium | | |
| Akkermansia muciniphila | | 37 | 37 |
| Barnesiella intestinihomins | | 38 | 38 |
| Bilophila wadsworthia | | 39 | 39 |
| Blautia faecis | | 40 | 40 |
| Blautia wexlerae | | 41 | 41 |
| Blautia luti | | 42 | 42 |
| Butyricimonas synergistica | | 43 | 43 |
| Clostridium bolteae | | 44 | 44 |
| Parasutterella | | 45 | 45 |
| excrementihominis | | | |
| Prevotella copri | | 46 | 46 |
| Ruminococcus faecis | | 47 | 47 |

SEQUENCES

SEQ ID NO: 1 Collinsella aerofaciens_F Strain 1
AGAGTTCGATCCTGGCTCAGGATGAACGCTGGCGGCGCGCCTAACACATGCAAGT
CGAACGGCACCCCTCTCCGGAGGGAAGCGAGTGGCGAACGGCTGAGTAACACGTG
GAGAACCTGCCCCCTCCCCCGGGATAGCCGCCCGAAAGGACGGGTAATACCGGAT
ACCCCGGGGTGCCGCATGGCACCCCGGCTAAAGCCCCGACGGGAGGGGATGGCT
CCGCGGCCCATCAGGTAGACGGCGGGGTGACGGCCCACCGTGCCGACAACGGGT
AGCCGGGTTGAGAGACCGACCGGCCAGATTGGGACTGAGACACGGCCCAGACTCC
TACGGGAGGCAGCAGTGGGGAATCTTGCGCAATGGGGGGAACCCTGACGCAGCGA
CGCCGCGTGCGGGACGGAGGCCTTCGGGTCGTAAACCGCTTTCAGCAGGGAAGAG
TCAAGACTGTACCTGCAGAAGAAGCCCCGGCTAACTACGTGCCAGCAGCCGCGGT
AATACGTAGGGGGCGAGCGTTATCCGGATTCATTGGGCGTAAAGCGCGCGTAGGC
GGCCCGGCAGGCCGGGGGTCGAAGCGGGGGGCTCAACCCCCCGAAGCCCCCGGA
ACCTCCGCGGCTTGGGTCCGGTAGGGGAGGGTGGAACACCCGGTGTAGCGGTGG
AATGCGCAGATATCGGGTGGAACACCGGTGGCGAAGGCGGCCCTCTGGGCCGAGA
CCGACGCTGAGGCGCGAAAGCTGGGGGAGCGAACAGGATTAGATACCCTGGTAGT
CCCAGCCGTAAACGATGGACGCTAGGTGTGGGGGGACGATCCCCCCGTGCCGCAG
CCAACGCATTAAGCGTCCCGCCTGGGGAGTACGGCCGCAAGGCTAAAACTCAAAG
GAATTGACGGGGGCCCGCACAAGCAGCGGAGCATGTGGCTTAATTCGAAGCAACG
CGAAGAACCTTACCAGGGCTTGACATATGGGTGAAGCGGGGGAGACCCCGTGGCC
GAGAGGAGCCCATACAGGTGGTGCATGGCTGTCGTCAGCTCGTGTCGTGAGATGTT
GGGTTAAGTCCCGCAACGAGCGCAACCCCCGCCGCGTGTTGCCATCGGGTGATGC
CGGGAACCCACGCGGGACCGCCGCCGTCAAGGCGGAGGAGGGGGGGGACGACGT
CAAGTCATCATGCCCCTTATGCCCTGGGCTGCACACGTGCTACAATGGCCGGTACA
GAGGGATGCCACCCCGCGAGGGGGAGCGGATCCCGGAAAGCCGGCCCCAGTTCG
GATTGGGGGCTGCAACCCGCCCCCATGAAGTCGGAGTTGCTAGTAATCGCGGATC
AGCATGCCGCGGTGAATGCGTTCCCGGGCCTTGTACACACCGCCCGTCACACCAC
CCGAGTCGTCTGCACCCGAAGTCGCCGGCCCAACCGAGAGGGGGGAGGCGCCGA
AGGTGTGGAGGGTGAGGGGGGTGAAGTCGTAACAAGGTA
SEQ ID NO: 2 Bacteroides xylanisolvens Strain 2
AGAGTTTGATCCTGGCTCAGGATGAACGCTAGCTACAGGCTTAACACATGCAAGTC
GAGGGGCAGCATTTTAGTTTGCTTGCAAACTAAAGATGGCGACCGGCGCACGGGTG
AGTAACACGTATCCAACCTGCCGATAACTCGGGGATAGCCTTTCGAAAGAAAGATTA
ATATCCGATAGTATATTAAAACCGCATGGTTTTACTATTAAAGAATTTCGGTTATCGA
TGGGGATGCGTTCCATTAGTTTGTTGGCGGGGTAACGGCCCACCAAGACTACGATG
GATAGGGGTTCTGAGAGGAAGGTCCCCCACATTGGAACTGAGACACGGTCCAAACT
CCTACGGGAGGCAGCAGTGAGGAATATTGGTCAATGGACGAGAGTCTGAACCAGC
CAAGTAGCGTGAAGGATGACTGCCCTATGGGTTGTAAACTTCTTTTATATGGGAATA
AAGTATTCCACGTGTGGGATTTTGTATGTACCATATGAATAAGGATCGGCTAACTCC
GTGCCAGCAGCCGCGGTAATACGGAGGATCCGAGCGTTATCCGGATTTATTGGGTT
TAAAGGGAGCGTAGGTGGATTGTTAAGTCAGTTGTGAAAGTTTGCGGCTCAACCGT
AAAATTGCAGTTGAAACTGGCAGTCTTGAGTACAGTAGAGGTGGGCGGAATTCGTG
GTGTAGCGGTGAAATGCTTAGATATCACGAAGAACTCCGATTGCGAAGGCAGCTCA
CTAGACTGCAACTGACACTGATGCTCGAAAGTGTGGGTATCAAACAGGATTAGATA
CCCTGGTAGTCCACACAGTAAACGATGAATACTCGCTGTTTGCGATATACAGTAAGC
GGCCAAGCGAAAGCATTAAGTATTCCACCTGGGGAGTACGCCGGCAACGGTGAAA
CTCAAAGGAATTGACGGGGGCCCGCACAAGCGGAGGAACATGTGGTTTAATTCGAT
GATACGCGAGGAACCTTACCCGGGCTTAAATTGCATTTGAATAATCTGGAAACAGGT
TAGCCGCAAGGCAAATGTGAAGGTGCTGCATGGTTGTCGTCAGCTCGTGCCGTGAG
GTGTCGGCTTAAGTGCCATAACGAGCGCAACCCTTATCTTTAGTTACTAACAGGTTA
TGCTGAGGACTCTAGAGAGACTGCCGTCGTAAGATGTGAGGAAGGTGGGGATGAC
GTCAAATCAGCACGGCCCTTACGTCCGGGGCTACACACGTGTTACAATGGGGGGTA
CAGAAGGCAGCTACCTGGCGACAGGATGCTAATCCCAAAAACCTCTCTCAGTTCGG
ATCGAAGTCTGCAACCCGACTTCGTGAAGCTGGATTCGCTAGTAATCGCGCATCAG
CCATGGCGCGGTGAATACGTTCCCGGGCCTTGTACACACCGCCCGTCAAGCCATG
AAAGCCGGGGGTACCTGAAGTACGTAACCGCAAGGAGCGTCCTAGGGTAAAACTG
GTAATTGGGGCTAAGTCGTAACAAGGTAGCCGTACCGGAAGGTGCGGCTGGAACA
CCT
SEQ ID NO: 3 Alistipes putredinis Strain 3
AGAGTTTGATCCTGGCTCAGGATGAACGCTAGCGGCAGGCTTAACACATGCAAGTC
GAGGGGCAGCATAATGGATAGCAATATCTATGGTGGCGACCGGCGCACGGGTGCG
TAACGCGTATGCAACCTACCTTTAACAGGGGGATAACACTGAGAAATTGGTACTAAT
ACCCCATAATATCATAGAAGGCATCTTTTATGGTTGAAAATTCCGATGGTTAGAGAT
GGGCATGCGTTGTATTAGCTAGTTGGTGGGGTAACGGCTCACCAAGGCGACGATAC
ATAGGGGGACTGAGAGGTTAACCCCCCACACTGGTACTGAGACACGGACCAGACT
CCTACGGGAGGCAGCAGTGAGGAATATTGGTCAATGGACGCAAGTCTGAACCAGC
CATGCCGCGTGCAGGATGACGGCTCTATGAGTTGTAAACTGCTTTTGTACGAGGGT
AAACGCAGATACGTGTATCTGTCTGAAAGTATCGTACGAATAAGGATCGGCTAACTC
CGTGCCAGCAGCCGCGGTAATACGGAGGATTCAAGCGTTATCCGGATTTATTGGGT
TTAAAGGGTGCGTAGGCGGTTTGATAAGTTAGAGGTGAAATTTCGGGGCTCAACCC
TGAACGTGCCTCTAATACTGTTGAGCTAGAGAGTAGTTGCGGTAGGCGGAATGTAT
GGTGTAGCGGTGAAATGCTTAGAGATCATACAGAACACCGATTGCGAAGGCAGCTT
ACCAAACTATATCTGACGTTGAGGCACGAAAGCGTGGGGAGCAAACAGGATTAGAT
ACCCTGGTAGTCCACGCAGTAAACGATGATAACTCGTTGTCGGCGATACACAGTCG
GTGACTAAGCGAAAGCGATAAGTTATCCACCTGGGGAGTACGTTCGCAAGAATGAA
ACTCAAAGGAATTGACGGGGGCCCGCACAAGCGGAGGAACATGTGGTTTAATTCGA
TGATACGCGAGGAACCTTACCCGGGCTTGAAAGTTAGCGACGATTCTTGAAAGAGG
ATTTCCCTTCGGGGCGCGAAACTAGGTGCTGCATGGTTGTCGTCAGCTCGTGCCGT
GAGGTGTCGGGTTAAGTCCCATAACGAGCGCAACCCCTACCGTTAGTTGCCATCAG
GTGAAGCTGGGCACTCTGGGGGGACTGCCGGTGTAAGCCGAGAGGAAGGTGGGG
ATGACGTCAAATCAGCACGGCCCTTACGTCCGGGGCTACACACGTGTTACAATGGT
AGGTACAGAGGGCAGCTACCCAGCGATGGGATGCGAATCTCGAAAGCCTATCTCAG
TTCGGATTGGAGGCTGAAACCCGCCTCCATGAAGTTGGATTCGCTAGTAATCGCGC
ATCAGCCATGGCGCGGTGAATACGTTCCCGGGCCTTGTACACACCGCCCGTCAAG
CCATGGGAGCCGGGGGTGCCTGAAGTTCGTGACCGCAAGGAGCGACCTAGGGCAA
AACTGGTGACTGGGGCTAAGTCGTAACAAGGTA
SEQ ID NO: 4 Alistipes shahii Strain 4
AGAGTTTGATCCTGGCTCAGGATGAACGCTAGCGGCAGGCCTAACACATGCAAGTC
GAGGGGCAGCACGGTGTAGCAATACACTGGTGGCGACCGGCGCACGGGTGCGTAA
CGCGTATGCAACCTACCCATAACAGGGGGATAACACTGAGAAATTGGTACTAATAC
CCCATAACATCAGGACCGGCATCGGTTCTGGTTGAAAACTCCGGTGGTTATGGATG
GGCATGCGTTGTATTAGCTGGTTGGTGAGGTAACGGCTCACCAAGGCAACGATACA
TAGGGGGACTGAGAGGTTAACCCCCCACATTGGTACTGAGACACGGACCAAACTCC
TACGGGAGGCAGCAGTGAGGAATATTGGTCAATGGACGCAAGTCTGAACCAGCCAT
GCCGCGTGCAGGAAGACGGCTCTATGAGTTGTAAACTGCTTTTGTACGAGAGTAAA
CGCTCTTACGTGTAAGAGCCTGAAAGTATCGTACGAATAAGGATCGGCTAACTCCG
TGCCAGCAGCCGCGGTAATACGGAGGATCCAAGCGTTATCCGGATTTATTGGGTTT
AAAGGGTGCGTAGGCGGTTTGATAAGTTAGAGGTGAAATACCGGTGCTTAACACCG
GAACTGCCTCTAATACTGTTGAACTAGAGAGTAGTTGCGGTAGGCGGAATGTATGG
TGTAGCGGTGAAATGCTTAGAGATCATACAGAACACCGATTGCGAAGGCAGCTTAC
CAAACTATATCTGACGTTGAGGCACGAAAGCGTGGGGAGCAAACAGGATTAGATAC
CCTGGTAGTCCACGCAGTAAACGATGATAACTCGCTGTCGGCGATACACAGTCGGC
GGCTAAGCGAAAGCGATAAGTTATCCACCTGGGGAGTACGTTCGCAAGAATGAAAC
TCAAAGGAATTGACGGGGGCCCGCACAAGCGGAGGAACATGTGGTTTAATTCGATG
ATACGCGAGGAACCTTACCCGGGCTTGAAAGTTACTGACGATTCTGGAAACAGGAT
TTCCCTTCGGGGCAGGAAACTAGGTGCTGCATGGTTGTCGTCAGCTCGTGCCGTGA
GGTGTCGGGTTAAGTCCCATAACGAGCGCAACCCCTACCGTTAGTTGCCATCAGGT
CAAGCTGGGCACTCTGGGGGGACTGCCGGTGTAAGCCGAGAGGAAGGTGGGGATG
ACGTCAAATCAGCACGGCCCTTACGTCCGGGGCTACACACGTGTTACAATGGTAGG
TACAGAGGGCAGCTACCCAGTGATGGGATGCGAATCTCGAAAGCCTATCTCAGTTC
GGATCGGAGGCTGAAACCCGCCTCCGTGAAGTTGGATTCGCTAGTAATCGCGCATC
AGCCATGGCGCGGTGAATACGTTCCCGGGCCTTGTACACACCGCCCGTCAAGCCA
TGGAAGCTGGGGGTGCCTGAAGTTCGTGACCGCAAGGAGCGACCTAGGGCAAAAC
CGGTGACTGGGGCTAAGTCGTAACAAGGTAGCCGTACCGGAAGGTGCGGCTGGAA
CACCT
SEQ ID NO: 5 Eubacterium_E hallii Strain 5
CTCAGGATGAACGCTAGCTACAGGCTTAACACATGCAAGTCGAGGGGCAGCATTCC
AGTTTGCTTGCAAACTGGAGATGGCGACCGGCGCACGGGTGAGTAACACGTATCCA
ACCTGCCGATAACTCGGGGATAGCCTTTCGAAAGAAAGATTAATACCCGATGGCAT
AATAGAACCGCATGGTTTGATTATTAAAGAATTTCGGTTATCGATGGGGATGCGTTC
CATTAGGCAGTTGGTGGGGTAACGGCCCACCAAACCTTCGATGGATAGGGGTTCTG
AGAGGAAGGTCCCCCACATTGGAACTGAGACACGGTCCAAACTCCTACGGGAGGC
AGCAGTGAGGAATATTGGTCAATGGACGAGAGTCTGAACCAGCCAAGTAGCGTGAA
GGATGACTGCCCTATGGGTTGTAAACTTCTTTTATATGGGAATAAAGTGGTCCACGT
GTGGATTTTTGTATGTACCATATGAATAAGGATCGGCTAACTCCGTGCCAGCAGCCG
CGGTAATACGGAGGATCCGAGCGTTATCCGGATTTATTGGGTTTAAAGGGAGCGTA
GGTGGACAGTTAAGTCAGTTGTGAAAGTTTGCGGCTCAACCGTAAAATTGCAGTTG
ATACTGGCTGTCTTGAGTACAGTAGAGGGGGGGGGAATTCGTGGTGTAGCGGTGAA
ATGCTTAGATATCACGAAGAACTCCGATTGCGAAGGCAGCTCACTGGACTGCAACT
GACACTGATGCTCGAAAGTGTGGGTATCAAACAGGATTAGATACCCTGGTAGTCCA
CACAGTAAACGATGAATACTCGCTGTTTGCGATATACAGTAAGCGGCCAAGCGAAA
GCATTAAGTATTCCACCTGGGGAGTACGCCGGCAACGGTGAAACTCAAAGGAATTG
ACGGGGGCCCGCACAAGCGGAGGAACATGTGGTTTAATTCGATGATACGCGAGGA
ACCTTACCCGGGCTTAAATTGCATTTGAATATATTGGAAACAGTATAGTCGTAAGAC
AAATGTGAAGGTGCTGCATGGTTGTCGTCAGCTCGTGCCGTGAGGTGTCGGCTTAA
GTGCCATAACGAGCGCAACCCTTATCTTTAGTTACTAACAGGTCATGCTGAGGACTC
TGGAGAGACTGCCGTCGTAAGATGTGAGGAAGGTGGGGATGACGTCAAATCAGCA
CGGCCCTTACGTCCGGGGCTACACACGTGTTACAATGGGGGGTACAGAAGGCCGC
TACCTGGTGACAGGATGCTAATCCCAAAAGCCTCTCTCAGTTCGGATCGAAGTCTG
CAACCCGACTTCGTGAAGCTGGATTCGCTAGTAATCGCGCATCAGCCATGGCGCGG
TGAATACGTTCCCGGGCCTTGTACACACCGCCCGTCAAGCCATGAAAGCCGGGGG
TACCTGAAGTACGTAACCGCAAGGAGCGTCCTAGGGTAAAACTGGTAATTGGGGCT
AAGTCGTAACAAGGTA
SEQ ID NO: 6 Blautia_A obeum Strain 6
AGAGTTTGATCCTGGCTCAGGATGAACGCTGGGGGCGTGCTTAACACATGCAAGTC
GAACGGGAAACCTTTTATTGAAGCTTCGGCAGATTTAGCTGGTTTCTAGTGGCGGA
CGGGTGAGTAACGCGTGGGTAACCTGCCTTATACAGGGGGATAACAACCAGAAATG
GTTGCTAATACCGCATAAGCGCACAGGACCGCATGGTCCGGTGTGAAAAACTCCGG
TGGTATAAGATGGACCCGCGTTGGATTAGCTAGTTGGCAGGGTAACGGCCTACCAA
GGCGACGATCCATAGCCGGCCTGAGAGGGTGAACGGCCACATTGGGACTGAGACA
CGGCCCAGACTCCTACGGGAGGCAGCAGTGGGGAATATTGCACAATGGGGGAAAC
CCTGATGCAGCGACGCCGCGTGAAGGAAGAAGTATCTCGGTATGTAAACTTCTATC
AGCAGGGAAGATAGTGACGGTACCTGACTAAGAAGCCCCGGCTAACTACGTGCCA
GCAGCCGCGGTAATACGTAGGGGGCAAGCGTTATCCGGATTTACTGGGTGTAAAG
GGAGCGTAGACGGACTGGCAAGTCTGATGTGAAAGGCGGGGGCTCAACCCCTGGA
CTGCATTGGAAACTGTTAGTCTTGAGTGCCGGAGAGGTAAGCGGAATTCCTAGTGT
AGCGGTGAAATGCGTAGATATTAGGAGGAACACCAGTGGCGAAGGCGGCTTACTG
GACGGTAACTGACGTTGAGGCTCGAAAGCGTGGGGAGCAAACAGGATTAGATACC
CTGGTAGTCCACGCCGTAAACGATGAATACTAGGTGTTGGGGAGCAAAGCTCTTCG
GTGCCGCCGCAAACGCATTAAGTATTCCACCTGGGGAGTACGTTCGCAAGAATGAA
ACTCAAAGGAATTGACGGGGACCCGCACAAGCGGTGGAGCATGTGGTTTAATTCGA
AGCAACGCGAAGAACCTTACCAAGTCTTGACATCCCTCTGACCGTTCCTTAACCGG
AACTTTCCTTCGGGACAGGGGAGACAGGTGGTGCATGGTTGTCGTCAGCTCGTGTC
GTGAGATGTTGGGTTAAGTCCCGCAACGAGCGCAACCCCTATCCCCAGTAGCCAGC
AGTCCGGCTGGGCACTCTGAGGAGACTGCCAGGGATAACCTGGAGGAAGGCGGGG
ATGACGTCAAATCATCATGCCCCTTATGATTTGGGCTACACACGTGCTACAATGGCG
TAAACAAAGGGAAGCA-
AGCCTGCGAAGGTAAGCAAATCCCAAAAATAACGTCCCAGTTCGGACTGCAGTCTG
CAACTCGACTGCACGAAGCTGGAATCGCTAGTAATCGCGGATCAGAATGCCGCGGT
GAATACGTTCCCGGGTCTTGTACACACCGCCCGTCACACCATGGGAGTCAGTAACG
CCCGAAGTCAGTGACCTAACTGCAAAGAAGGAGCTGCCGAAGGGGGGACCGATGA
CTGGGGTGAAGTCGTAACAAGGTA
SEQ ID NO: 7 Odoribacter splanchnicus Strain 7
AGAGTTTGATCCTGGCTCAGGATGAACGCTAGCGACAGGCTTAACACATGCAAGTC
GAGGGGCATCATGAGGTAGCAATACCTTGATGGCGACCGGCGCACGGGTGAGTAA
CGCGTATGCAACCTGCCTGATACCGGGGTATAGCCCATGGAAACGTGGATTAACAC
CCCATAGTACTTTTATCCTGCATGGGATGTGAGTTAAATGTTTAAGGTATCGGATGG
GCATGCGTCCTATTAGTTAGTTGGGGGGGTAACAGCCCACCAAGACGATGATAGGT
AGGGGTTCTGAGAGGAAGGTCCCCCACATTGGAACTGAGACACGGTCCAAACTCCT
ACGGGAGGCAGCAGTGAGGAATATTGGTCAATGGACGAGAGTCTGAACCAGCCAA
GTCGCGTGAGGGAAGACTGCCCTATGGGTTGTAAACCTCTTTTATAAGGGAAGAAT
AAGTTCTACGTGTAGAATGATGCCTGTACCTTATGAATAAGCATCGGCTAACTCCGT
GCCAGCAGCCGCGGTAATACGGAGGATGCGAGCGTTATCCGGATTTATTGGGTTTA
AAGGGTGCGTAGGCGGTTTATTAAGTTAGTGGTTAAATATTTGAGCTAAACTCAATT
GTGCCATTAATACTGGTAAACTGGAGTACAGACGAGGTAGGCGGAATAAGTTAAGT
AGCGGTGAAATGCATAGATATAACTTAGAACTCCGATAGCGAAGGCAGCTTACCAG
ACTGTAACTGACGCTGATGCACGAGAGCGTGGGTAGCGAACAGGATTAGATACCCT
GGTAGTCCACGCCGTAAACGATGCTCACTGGTTCTGTGCGATATATTGTACGGGAT
TAAGCGAAAGTATTAAGTGAGCCACCTGGGGAGTACGTCGGCAACGATGAAACTCA
AAGGAATTGACGGGGGCCCGCACAAGCGGAGGAACATGTGGTTTAATTCGATGATA
CGCGAGGAACCTTACCTGGGTTTAAATGGGAAATGTCGTATTTGGAAACAGATATTC
TCTTCGGAGCGTTTTTCAAGGTGCTGCATGGTTGTCGTCAGCTCGTGCCGTGAGGT
GTCGGGTTAAGTCCCATAACGAGCGCAACCCTTACCGTTAGTTGCTAGCATGTAAT
GATGAGCACTCTAACGGGACTGCCACCGTAAGGTGAGAGGAAGGCGGGGATGACG
TCAAATCAGCACGGCCCTTACACCCAGGGCTACACACGTGTTACAATGGCCGGTAC
AGAGGGCCGCTACCAGGTGACTGGATGCCAATCTCAAAAGCCGGTCGTAGTTCGG
ATTGGAGTCTGTAACCCGACTCCATGAAGTTGGATTCGCTAGTAATCGCGCATCAG
CCATGGCGCGGTGAATACGTTCCCGGGCCTTGTACACACCGCCCGTCAAGCCATG
GAAGCCGGGGGTGCCTGAAGTCCGTAACCGCGAGGATCGGCCTAGGGCAAAACTG
GTAACTGGGGCTAAGTCGTAACAAGGTAGCCGTACCGGAAGGTGCGGCTGGAACA
CCT
SEQ ID NO: 8 Bacteroides cellulosilyticus Strain 8
AGAGTTTGATCCTGGCTCAGGATGAACGCTAGCTACAGGCTTAACACATGCAAGTC
GAGGGGCAGCATGACCTAGCAATAGGTTGATGGCGACCGGCGCACGGGTGAGTAA
CACGTATCCAACCTACCGGTTATTCCGGGATAGCCTTTCGAAAGAAAGATTAATACC
GGATAGTATAACGAGAAGGCATCTTTTTGTTATTAAAGAATTTCGATAACCGATGGG
GATGCGTTCCATTAGTTTGTTGGCGGGGTAACGGCCCACCAAGACATCGATGGATA
GGGGTTCTGAGAGGAAGGTCCCCCACATTGGAACTGAGACACGGTCCAAACTCCTA
CGGGAGGCAGCAGTGAGGAATATTGGTCAATGGACGAGAGTCTGAACCAGCCAAG
TAGCGTGAAGGATGACTGCCCTATGGGTTGTAAACTTCTTTTATATGGGAATAAAGT
GAGCCACGTGTGGCTTTTTGTATGTACCATACGAATAAGGATCGGCTAACTCCGTG
CCAGCAGCCGCGGTAATACGGAGGATCCGAGCGTTATCCGGATTTATTGGGTTTAA
AGGGAGCGTAGGCGGACTATTAAGTCAGCTGTGAAAGTTTGCGGCTCAACCGTAAA
ATTGCAGTTGATACTGGTCGTCTTGAGTGCAGTAGAGGTAGGCGGAATTCGTGGTG
TAGCGGTGAAATGCTTAGATATCACGAAGAACTCCGATTGCGAAGGCAGCTTACTG
GACTGTAACTGACGCTGATGCTCGAAAGTGTGGGTATCAAACAGGATTAGATACCC
TGGTAGTCCACACAGTAAACGATGAATACTCGCTGTTTGCGATATACGGCAAGCGG
CCAAGCGAAAGCATTAAGTATTCCACCTGGGGAGTACGCCGGCAACGGTGAAACTC
AAAGGAATTGACGGGGGCCCGCACAAGCGGAGGAACATGTGGTTTAATTCGATGAT
ACGCGAGGAACCTTACCCGGGCTTAAATTGCAAATGAATATAGTGGAAACATTATAG
CCGCAAGGCATTTGTGAAGGTGCTGCATGGTTGTCGTCAGCTCGTGCCGTGAGGTG
TCGGCTTAAGTGCCATAACGAGCGCAACCCTTATCTTTAGTTACTAACAGGTCATGC
TGAGGACTCTAGAGAGACTGCCGTCGTAAGATGTGAGGAAGGTGGGGATGACGTC
AAATCAGCACGGCCCTTACGTCCGGGGCTACACACGTGTTACAATGGGGGGTACAG
AAGGCAGCTACACAGCGATGTGATGCTAATCCCAAAAGCCTCTCTCAGTTCGGATT
GGAGTCTGCAACCCGACTCCATGAAGCTGGATTCGCTAGTAATCGCGCATCAGCCA
CGGCGCGGTGAATACGTTCCCGGGCCTTGTACACACCGCCCGTCAAGCCATGAAA
GCCGGGGGTACCTGAAGTCCGTAACCGTAAGGAGCGGCCTAGGGTAAAACTGGTA
ATTGGGGCTAAGTCGTA
SEQ ID NO: 9 Bacteroides uniformis Strain 9
CTGGCTCAGGATGAACGCTAGCTACAGGCTTAACACATGCAAGTCGAGGGGCAGCA
TGAACTTAGCTTGCTAAGTTTGATGGCGACCGGCGCACGGGTGAGTAACACGTATC
CAACCTGCCGATGACTCGGGGATAGCCTTTCGAAAGAAAGATTAATACCCGATGGC
ATAGTTCTTCCGCATGGTAGAACTATTAAAGAATTTCGGTCATCGATGGGGATGCGT
TCCATTAGGTTGTTGGCGGGGTAACGGCCCACCAAGCCTTCGATGGATAGGGGTTC
TGAGAGGAAGGTCCCCCACATTGGAACTGAGACACGGTCCAAACTCCTACGGGAG
GCAGCAGTGAGGAATATTGGTCAATGGACGAGAGTCTGAACCAGCCAAGTAGCGTG
AAGGATGACTGCCCTATGGGTTGTAAACTTCTTTTATACGGGAATAAAGTGAGGCAC
GTGTGCCTTTTTGTATGTACCGTATGAATAAGGATCGGCTAACTCCGTGCCAGCAGC
CGCGGTAATACGGAGGATCCGAGCGTTATCCGGATTTATTGGGTTTAAAGGGAGCG
TAGGCGGACGCTTAAGTCAGTTGTGAAAGTTTGCGGCTCAACCGTAAAATTGCAGT
TGATACTGGGTGTCTTGAGTACAGTAGAGGCAGGCGGAATTCGTGGTGTAGCGGTG
AAATGCTTAGATATCACGAAGAACTCCGATTGCGAAGGCAGCTTGCTGGACTGTAA
CTGACGCTGATGCTCGAAAGTGTGGGTATCAAACAGGATTAGATACCCTGGTAGTC
CACACAGTAAACGATGAATACTCGCTGTTTGCGATATACAGTAAGCGGCCAAGCGA
AAGCGTTAAGTATTCCACCTGGGGAGTACGCCGGCAACGGTGAAACTCAAAGGAAT
TGACGGGGGCCCGCACAAGCGGAGGAACATGTGGTTTAATTCGATGATACGCGAG
GAACCTTACCCGGGCTTGAATTGCAACTGAATGATGTGGAGACATGTCAGCCGCAA
GGCAGTTGTGAAGGTGCTGCATGGTTGTCGTCAGCTCGTGCCGTGAGGTGTCGGC
TTAAGTGCCATAACGAGCGCAACCCTTATCGATAGTTACCATCAGGTTATGCTGGG
GACTCTGTCGAGACTGCCGTCGTAAGATGTGAGGAAGGTGGGGATGACGTCAAATC
AGCACGGCCCTTACGTCCGGGGCTACACACGTGTTACAATGGGGGGTACAGAAGG
CAGCTACACGGCGACGTGATGCTAATCCCTAAAGCCTCTCTCAGTTCGGATTGGAG
TCTGCAACCCGACTCCATGAAGCTGGATTCGCTAGTAATCGCGCATCAGCCACGGC
GCGGTGAATACGTTCCCGGGCCTTGTACACACCGCCCGTCAAGCCATGAAAGCCG
GGGGTACCTGAAGTGCGTAACCGCGAGGAGCGCCCTAGGGTAAAACTGGTGATTG
GGGCTAAGTCGTAACAAGGTA
SEQ ID NO: 10 Bacteroides_B vulgatus Strain 10
ATGAAGAGTTTGATCCTGGCTCAGGATGAACGCTAGCTACAGGCTTAACACATGCA
AGTCGAGGGGCAGCATGGTCTTAGCTTGCTAAGGCCGATGGCGACCGGCGCACGG
GTGAGTAACACGTATCCAACCTGCCGTCTACTCTTGGACAGCCTTCTGAAAGGAAG
ATTAATACAAGATGGCATCATGAGTCCGCATGTTCACATGATTAAAGGTATTCCGGT
AGACGATGGGGATGCGTTCCATTAGATAGTAGGCGGGGTAACGGCCCACCTAGTCT
TCGATGGATAGGGGTTCTGAGAGGAAGGTCCCCCACATTGGAACTGAGACACGGTC
CAAACTCCTACGGGAGGCAGCAGTGAGGAATATTGGTCAATGGGCGAGAGCCTGA
ACCAGCCAAGTAGCGTGAAGGATGACTGCCCTATGGGTTGTAAACTTCTTTTATAAA
GGAATAAAGTCGGGTATGGATACCCGTTTGCATGTACTTTATGAATAAGGATCGGCT
AACTCCGTGCCAGCAGCCGCGGTAATACGGAGGATCCGAGCGTTATCCGGATTTAT
TGGGTTTAAAGGGAGCGTAGATGGATGTTTAAGTCAGTTGTGAAAGTTTGCGGCTC
AACCGTAAAATTGCAGTTGATACTGGATATCTTGAGTGCAGTTGAGGCAGGCGGAA
TTCGTGGTGTAGCGGTGAAATGCTTAGATATCACGAAGAACTCCGATTGCGAAGGC
AGCCTGCTAAGCTGCAACTGACATTGAGGCTCGAAAGTGTGGGTATCAAACAGGAT
TAGATACCCTGGTAGTCCACACGGTAAACGATGAATACTCGCTGTTTGCGATATACA
GCAAGCGGCCAAGCGAAAGCGTTAAGTATTCCACCTGGGGAGTACGCCGGCAACG
GTGAAACTCAAAGGAATTGACGGGGGCCCGCACAAGCGGAGGAACATGTGGTTTAA
TTCGATGATACGCGAGGAACCTTACCCGGGCTTAAATTGCAGATGAATTACGGTGA
AAGCCGTAAGCCGCAAGGCATCTGTGAAGGTGCTGCATGGTTGTCGTCAGCTCGTG
CCGTGAGGTGTCGGCTTAAGTGCCATAACGAGCGCAACCCTTGTTGTCAGTTACTA
ACAGGTTCCGCTGAGGACTCTGACAAGACTGCCATCGTAAGATGTGAGGAAGGTGG
GGATGACGTCAAATCAGCACGGCCCTTACGTCCGGGGCTACACACGTGTTACAATG
GGGGGTACAGAGGGCCGCTACCACGCGAGTGGATGCCAATCCCAAAAACCTCTCT
CAGTTCGGACTGGAGTCTGCAACCCGACTCCACGAAGCTGGATTCGCTAGTAATCG
CGCATCAGCCACGGCGCGGTGAATACGTTCCCGGGCCTTGTACACACCGCCCGTC
AAGCCATGGGAGCCGGGGGTACCTGAAGTGCGTAACCGCGAGGAGCGCCCTAGG
GTAAAACTGGTGACTGGGGCTAAGTCGTAACAAGGTAGCCGTACCGGAAG
SEQ ID NO: 11 Bacteroides fragilis Strain 11
ATGAAGAGTTTGATCCTGGCTCAGGATGAACGCTAGCTACAGGCTTAACACATGCA
AGTCGAGGGGCATCAGGAAGAAAGCTTGCTTTCTTTGCTGGCGACCGGCGCACGG
GTGAGTAACACGTATCCAACCTGCCCTTTACTCGGGGATAGCCTTTCGAAAGAAAG
ATTAATACCCGATGGCATAATGATTCCGCATGGTTTCATTATTAAAGGATTCCGGTA
AAGGATGGGGATGCGTTCCATTAGGTTGTTGGTGAGGTAACGGCTCACCAAGCCTT
CGATGGATAGGGGTTCTGAGAGGAAGGTCCCCCACATTGGAACTGAGACACGGTC
CAAACTCCTACGGGAGGCAGCAGTGAGGAATATTGGTCAATGGGCGCTAGCCTGAA
CCAGCCAAGTAGCGTGAAGGATGAAGGCTCTATGGGTCGTAAACTTCTTTTATATAA
GAATAAAGTGCAGTATGTATACTGTTTTGTATGTATTATATGAATAAGGATCGGCTAA
CTCCGTGCCAGCAGCCGCGGTAATACGGAGGATCCGAGCGTTATCCGGATTTATTG
GGTTTAAAGGGAGCGTAGGTGGACTGGTAAGTCAGTTGTGAAAGTTTGCGGCTCAA
CCGTAAAATTGCAGTTGATACTGTCAGTCTTGAGTACAGTAGAGGTGGGCGGAATT
CGTGGTGTAGCGGTGAAATGCTTAGATATCACGAAGAACTCCGATTGCGAAGGCAG
CTCACTGGACTGCAACTGACACTGATGCTCGAAAGTGTGGGTATCAAACAGGATTA
GATACCCTGGTAGTCCACACAGTAAACGATGAATACTCGCTGTTTGCGATATACAGT
AAGCGGCCAAGCGAAAGCATTAAGTATTCCACCTGGGGAGTACGCCGGCAACGGT
GAAACTCAAAGGAATTGACGGGGGCCCGCACAAGCGGAGGAACATGTGGTTTAATT
CGATGATACGCGAGGAACCTTACCCGGGCTTAAATTGCAGTGGAATGATGTGGAAA
CATGTCAGTGAGCAATCACCGCTGTGAAGGTGCTGCATGGTTGTCGTCAGCTCGTG
CCGTGAGGTGTCGGCTTAAGTGCCATAACGAGCGCAACCCTTATCTTTAGTTACTAA
CAGGTTATGCTGAGGACTCTAGAGAGACTGCCGTCGTAAGATGTGAGGAAGGTGG
GGATGACGTCAAATCAGCACGGCCCTTACGTCCGGGGCTACACACGTGTTACAATG
GGGGGTACAGAAGGCAGCTAGCGGGTGACCGTATGCTAATCCCAAAAGCCTCTCTC
AGTTCGGATCGAAGTCTGCAACCCGACTTCGTGAAGCTGGATTCGCTAGTAATCGC
GCATCAGCCACGGCGCGGTGAATACGTTCCCGGGCCTTGTACACACCGCCCGTCA
AGCCATGGGAGCCGGGGGTACCTGAAGTACGTAACCGCAAGGATCGTCCTAGGGT
AAAACTGGTGACTGGGGCTAAGTCGTAACAAGGTAGCCGTACCGGAAGGTGCGGC
TGGAACACCTCCTT
SEQ ID NO: 12 Bacteroides caccae Strain 12
TGGCTCAGGATGAACGCTAGCTACAGGCTTAACACATGCAAGTCGAGGGGCATCAG
TTTGGTTTGCTTGCAAACCAAAGCTGGCGACCGGCGCACGGGTGAGTAACACGTAT
CCAACCTGCCTCATACTCGGGGATAGCCTTTCGAAAGAAAGATTAATATCCGATAGC
ATATATTTCCCGCATGGGTTTTATATTAAAGAAATTCGGTATGAGATGGGGATGCGT
TCCATTAGTTTGTTGGGGGGGTAACGGCCCACCAAGACTACGATGGATAGGGGTTC
TGAGAGGAAGGTCCCCCACATTGGAACTGAGACACGGTCCAAACTCCTACGGGAG
GCAGCAGTGAGGAATATTGGTCAATGGACGCGAGTCTGAACCAGCCAAGTAGCGTG
AAGGATGACTGCCCTATGGGTTGTAAACTTCTTTTATATGGGAATAAAGTTGTCCAC
GTGTGGATTTTTGTATGTACCATATGAATAAGGATCGGCTAACTCCGTGCCAGCAGC
CGCGGTAATACGGAGGATCCGAGCGTTATCCGGATTTATTGGGTTTAAAGGGAGCG
TAGGCGGATTGTTAAGTCAGTTGTGAAAGTTTGCGGCTCAACCGTAAAATTGCAGTT
GATACTGGCAGTCTTGAGTGCAGTAGAGGTGGGCGGAATTCGTGGTGTAGCGGTG
AAATGCTTAGATATCACGAAGAACTCCGATTGCGAAGGCAGCTCACTGGAGTGTAA
CTGACGCTGATGCTCGAAAGTGTGGGTATCAAACAGGATTAGATACCCTGGTAGTC
CACACAGTAAACGATGAATACTCGCTGTTTGCGATATACAGTAAGCGGCCAAGCGA
AAGCATTAAGTATTCCACCTGGGGAGTACGCCGGCAACGGTGAAACTCAAAGGAAT
TGACGGGGGCCCGCACAAGCGGAGGAACATGTGGTTTAATTCGATGATACGCGAG
GAACCTTACCCGGGCTTAAATTGCAAATGAATTATGGGGAAACCCATAGGCCGCAA
GGCATTTGTGAAGGTGCTGCATGGTTGTCGTCAGCTCGTGCCGTGAGGTGTCGGCT
TAAGTGCCATAACGAGCGCAACCCTTATCTTCAGTTACTAACAGGTCATGCTGAGGA
CTCTGGAGAGACTGCCGTCGTAAGATGTGAGGAAGGTGGGGATGACGTCAAATCA
GCACGGCCCTTACGTCCGGGGCTACACACGTGTTACAATGGGGGGTACAGAAGGC
CGCTACCTGGTGACAGGATGCCAATCCCAAAAACCTCTCTCAGTTCGGATCGAAGT
CTGCAACCCGACTTCGTGAAGCTGGATTCGCTAGTAATCGCGCATCAGCCATGGCG
CGGTGAATACGTTCCCGGGCCTTGTACACACCGCCCGTCAAGCCATGAAAGCCGG
GGGTACCTGAAGTACGTAACCGCAAGGAGCGTCCTAGGGTAAAACTGGTAATTGGG
GCTAAGTCGTAACAAGGTA
SEQ ID NO: 13 Paeniclostridium sordellii Strain 13
GTTTGATCCTGGCTCAGGATGAACGCTGGCGGCGTGCCTAACACATGCAAGTCGAG
CGAACCCTTCGGGGTGAGCGGCGGACGGGTGAGTAACGCGTGGGTAACCTGCCCT
GTACACACGGATAACATACCGAAAGGTATGCTAATACGGGATAATATATGAGAGTCG
CATGGCTTTTGTATCAAAGCTCTGGCGGTACAGGATGGACCCGCGTCTGATTAGCT
AGTTGGTAAGGTAACGGCTTACCAAGGCAACGATCAGTAGCCGACCTGAGAGGGTG
ATCGGCCACATTGGAACTGAGACACGGTCCAAACTCCTACGGGAGGCAGCAGTGG
GGAATATTGCACAATGGGCGAAAGCCTGATGCAGCAACGCCGCGTGAGCGATGAA
GGCCTTCGGGTCGTAAAGCTCTGTCCTCAAGGAAGATAATGACGGTACTTGAGGAG
GAAGCCCCGGCTAACTACGTGCCAGCAGCCGCGGTAATACGTAGGGGGCTAGCGT
TATCCGGAATTACTGGGCGTAAAGGGTGCGTAGGCGGTCTTTCAAGCCAGAAGTGA
AAGGCTACGGCTCAACCGTAGTAAGCTTTTGGAACTGTAGGACTTGAGTGCAGGAG
AGGAGAGTGGAATTCCTAGTGTAGCGGTGAAATGCGTAGATATTAGGAGGAACACC
AGTAGCGAAGGCGGCTCTCTGGACTGTAACTGACGCTGAGGCACGAAAGCGTGGG
GAGCAAACAGGATTAGATACCCTGGTAGTCCACGCCGTAAACGATGAGTACTAGGT
GTCGGGGGTTACCCCCCTCGGTGCCGCAGCTAACGCATTAAGTACTCCGCCTGGG
AAGTACGCTCGCAAGAGTGAAACTCAAAGGAATTGACGGGGACCCGCACAAGTAGC
GGAGCATGTGGTTTAATTCGAAGCAACGCGAAGAACCTTACCTAAGCTTGACATCC
CACTGACCTCTCCCTAATCGGAGATTTCCCTTCGGGGACAGTGGTGACAGGTGGTG
CATGGTTGTCGTCAGCTCGTGTCGTGAGATGTTGGGTTAAGTCCCGCAACGAGCGC
AACCCTTGCCTTTAGTTGCCAGCATTAAGTTGGGCACTCTAGAGGGACTGCCGAGG
ATAACTCGGAGGAAGGTGGGGATGACGTCAAATCATCATGCCCCTTATGCTTAGGG
CTACACACGTGCTACAATGGGTGGTACAGAGGGTTGCCAAGCCGTGAGGTGGAGC
TAATCCCTTAAAGCCATTCTCAGTTCGGATTGTAGGCTGAAACTCGCCTACATGAAG
CTGGAGTTACTAGTAATCGCAGATCAGAATGCTGCGGTGAATGCGTTCCCGGGTCT
TGTACACACCGCCCGTCACACCATGGGAGTTGGGGGCGCCCGAAGCCGGTTAGCT
AACCTTTTAGGAAGCGGCCGTCGAAGGTGAAACCAATGACTGGGGTGAAGTCGTAA
CAAGGTAGCCGTATCGGAAGGTGCGGCTGGATCACCTCCTTT
SEQ ID NO: 14 Blautia producta Strain 14
AGAGTTTGATCCTGGCTCAGGATGAACGCTGGCGGCGTGCTTAACACATGCAAGTC
GAGCGAAGCACTAAGACAGATTTCTTCGGATTGAAGTCTTTGTGACTGAGCGGCGG
ACGGGTGAGTAACGCGTGGGTAACCTGCCTCATACAGGGGGATAACAGTTAGAAAT
GACTGCTAATACCGCATAAGCGCACAGGACCGCATGGTCTGGTGTGAAAAACTCCG
GTGGTATGAGATGGACCCGCGTCTGATTAGCTAGTTGGAGGGGTAACGGCCCACC
AAGGCGACGATCAGTAGCCGGCCTGAGAGGGTGAACGGCCACATTGGGACTGAGA
CACGGCCCAGACTCCTACGGGAGGCAGCAGTGGGGAATATTGCACAATGGGGGAA
ACCCTGATGCAGCGACGCCGCGTGAAGGAAGAAGTATCTCGGTATGTAAACTTCTA
TCAGCAGGGAAGAAAATGACGGTACCTGACTAAGAAGCCCCGGCTAACTACGTGCC
AGCAGCCGCGGTAATACGTAGGGGGCAAGCGTTATCCGGATTTACTGGGTGTAAAG
GGAGCGTAGACGGAAGAGCAAGTCTGATGTGAAAGGCTGGGGCTTAACCCCAGGA
CTGCATTGGAAACTGTTGTTCTAGAGTGCCGGAGAGGTAAGCGGAATTCCTAGTGT
AGCGGTGAAATGCGTAGATATTAGGAGGAACACCAGTGGCGAAGGCGGCTTACTG
GACGGTAACTGACGTTGAGGCTCGAAAGCGTGGGGAGCAAACAGGATTAGATACC
CTGGTAGTCCACGCCGTAAACGATGAATACTAGGTGTCGGGTGGCAAAGCCATTCG
GTGCCGCAGCAAACGCAATAAGTATTCCACCTGGGGAGTACGTTCGCAAGAATGAA
ACTCAAAGGAATTGACGGGGACCCGCACAAGCGGTGGAGCATGTGGTTTAATTCGA
AGCAACGCGAAGAACCTTACCAAGTCTTGACATCCCTCTGACCGTCCCGTAACGGG
GGCTTCCCTTCGGGGCAGAGGAGACAGGTGGTGCATGGTTGTCGTCAGCTCGTGT
CGTGAGATGTTGGGTTAAGTCCCGCAACGAGCGCAACCCTTATCCTTAGTAGCCAG
CACATGATGGTGGGCACTCTAGGGAGACTGCCGGGGATAACCCGGAGGAAGGCGG
GGACGACGTCAAATCATCATGCCCCTTATGATTTGGGCTACACACGTGCTACAATG
GCGTAAACAAAGGGAAGCGAGACAGCGATGTTGAGCGAATCCCAAAAATAACGTCC
CAGTTCGGACTGCAGTCTGCAACTCGACTGCACGAAGCTGGAATCGCTAGTAATCG
CGGATCAGAATGCCGCGGTGAATACGTTCCCGGGTCTTGTACACACCGCCCGTCAC
ACCATGGGAGTCAGTAACGCCCGAAGTCAGTGACCTAACCGAAAGGAAGGAGCTG
CCGAAGGGGGGACCGATAACTGGGGTGAAGTCGTAACAAGGTA
SEQ ID NO: 15 Absiella innocuum Strain 15
ATGGAGAGTTTGATCCTGGCTCAGGATGAACGCTGGCGGCATGCCTAATACATGCA
AGTCGAACGAAGT-
TTCGAGGAAGCTTGCTTCCAAAGAGACTTAGTGGCGAACGGGTGAGTAACACGTAG
GTAACCTGCCCATGTGTCCGGGATAACTGCTGGAAACGGTAGCTAAAACCGGATAG
GTATACAGAGCGCATGCTCAGTATATTAAAGCGCCCATCAAGGCGTGAACATGGAT
GGACCTGCGGCGCATTAGCTAGTTGGTGAGGTAACGGCCCACCAAGGCGATGATG
CGTAGCCGGCCTGAGAGGGTAAACGGCCACATTGGGACTGAGACACGGCCCAAAC
TCCTACGGGAGGCAGCAGTAGGGAATTTTCGTCAATGGGGGAAACCCTGAACGAG
CAATGCCGCGTGAGTGAAGAAGGTCTTCGGATCGTAAAGCTCTGTTGTAAGTGAAG
AACGGCTCATAGAGGAAATGCTATGGGAGTGACGGTAGCTTACCAGAAAGCCACGG
CTAACTACGTGCCAGCAGCCGCGGTAATACGTAGGTGGCAAGCGTTATCCGGAATC
ATTGGGCGTAAAGGGTGCGTAGGTGGCGTACTAAGTCTGTAGTAAAAGGCAATGGC
TCAACCATTGTAAGCTATGGAAACTGGTATGCTGGAGTGCAGAAGAGGGCGATGGA
ATTCCATGTGTAGCGGTAAAATGCGTAGATATATGGAGGAACACCAGTGGCGAAGG
CGGTCGCCTGGTCTGTAACTGACACTGAGGCACGAAAGCGTGGGGAGCAAATAGG
ATTAGATACCCTAGTAGTCCACGCCGTAAACGATGAGAACTAAGTGTTGGAGGAATT
CAGTGCTGCAGTTAACGCAATAAGTTCTCCGCCTGGGGAGTATGCACGCAAGTGTG
AAACTCAAAGGAATTGACGGGGGCCCGCACAAGCGGTGGAGTATGTGGTTTAATTC
GAAGCAACGCGAAGAACCTTACCAGGCCTTGACATGGAAACAAATACCCTAGAGAT
AGGGGGATAATTATGGATCACACAGGTGGTGCATGGTTGTCGTCAGCTCGTGTCGT
GAGATGTTGGGTTAAGTCCCGCAACGAGCGCAACCCTTGTCGCATGTTACCAGCAT
CAAGTTGGGGACTCATGCGAGACTGCCGGTGACAAACCGGAGGAAGGTGGGGATG
ACGTCAAATCATCATGCCCCTTATGGCCTGGGCTACACACGTACTACAATGGCGAC
CACAAAGAGCAGCGACACAGTGATGTGAAGCGAATCTCATAAAGGTCGTCTCAGTT
CGGATTGAAGTCTGCAACTCGACTTCATGAAGTCGGAATCGCTAGTAATCGCAGAT
CAGCATGCTGCGGTGAATACGTTCTCGGGCCTTGTACACACCGCCCGTCAAACCAT
GGGAGTCAGTAATACCCGAAGCCGGTGGCATAACCGTAAGGAGTGAGCCGTCGAA
GGTAGGACCGA
SEQ ID NO: 16 Clostridium_M clostridioforme Strain 16
AGAGTTTGATCCTGGCTCAGGATGAACGCTGGCGGCGTGCCTAACACATGCAAGTC
GAACGAAGCAATTAAGATGAAGTTTTCGGATGGAATCTTGATTGACTGAGTGGCGG
ACGGGTGAGTAACGCGTGGATAACCTGCCTCACACTGGGGGATAACAGTTAGAAAT
GACTGCTAATACCGCATAAGCGCACAGTGCCGCATGGCAGTGTGTGAAAAACTCCG
GTGGTGTGAGATGGATCCGCGTCTGATTAGCCAGTTGGCGGGGTAACGGCCCACC
AAAGCGACGATCAGTAGCCGACCTGAGAGGGTGACCGGCCACATTGGGACTGAGA
CACGGCCCAAACTCCTACGGGAGGCAGCAGTGGGGAATATTGCACAATGGGCGAA
AGCCTGATGCAGCGACGCCGCGTGAGTGAAGAAGTATTTCGGTATGTAAAGCTCTA
TCAGCAGGGAAGAAAATGACGGTACCTGACTAAGAAGCCCCGGCTAACTACGTGCC
AGCAGCCGCGGTAATACGTAGGGGGCAAGCGTTATCCGGATTTACTGGGTGTAAAG
GGAGCGTAGACGGCGAAGCAAGTCTGAAGTGAAAACCCGGGGCTCAACCCTGGGA
CTGCTTTGGAAACTGTTTTGCTAGAGTGTCGGAGAGGTAAGTGGAATTCCTAGTGTA
GCGGTGAAATGCGTAGATATTAGGAGGAACACCAGTGGCGAAGGCGGCTTACTGG
ACGATAACTGACGTTGAGGCTCGAAAGCGTGGGGAGCAAACAGGATTAGATACCCT
GGTAGTCCACGCCGTAAACGATGAATGCTAGGTGTTGGGGGGCAAAGCCCTTCGG
TGCCGCCGCAAACGCAGTAAGCATTCCACCTGGGGAGTACGTTCGCAAGAATGAAA
CTCAAAGGAATTGACGGGGACCCGCACAAGCGGTGGAGCATGTGGTTTAATTCGAA
GCAACGCGAAGAACCTTACCAAGTCTTGACATCCCCCTGACGGGCCGGTAACGCG
GCCTTTCCTTCGGGACAGGGGAGACAGGTGGTGCATGGTTGTCGTCAGCTCGTGT
CGTGAGATGTTGGGTTAAGTCCCGCAACGAGCGCAACCCTTATCCTTAGTAGCCAG
CAGGTAGAGCCGGGCACTCTAGGGAGACTGCCAGGGATAACCTGGAGGAAGGTGG
GGATGACGTCAAATCATCATGCCCCTTATGATTTGGGCTACACACGTGCTACAATGG
CGTAAACAAAGGGAAGCGAGACAGTGATGTGGAGCAAATCCCAAAAATAACGTCCC
AGTTCGGACTGTAGTCTGCAACCCGACTACACGAAGCTGGAATCGCTAGTAATCGC
GAATCAGAATGTCGCGGTGAATACGTTCCCGGGTCTTGTACACACCGCCCGTCACA
CCATGGGAGTCAGCAACGCCCGAAGTCAGTGACCCAAC-CGAAAG-
GAGGGAGCTGCCGAAGGGGGGGCAGGTAACTGGGGTGAAGTCGTAACAAGGTAGC
CGTA
SEQ ID NO: 17 Paraclostridium massiliensis Strain 17
AGAGTTTGATCCTGGCTCAGGATGAACGCTGGCGGCGTGCCTAACACATGCAAGTC
GAGCGATCTCTTCGGAGAGAGCGGCGGACGGGTGAGTAACGCGTGGGTAACCTGC
CCTGTACACACGGATAACATACCGAAAGGTATACTAATACGGGATAACATACGAAAG
TCGCATGGCTTTTGTATCAAAGCTCCGGCGGTACAGGATGGACCCGCGTCTGATTA
GCTAGTTGGTAAGGTAATGGCTTACCAAGGCAACGATCAGTAGCCGACCTGAGAGG
GTGATCGGCCACACTGGAACTGAGACACGGTCCAGACTCCTACGGGAGGCAGCAG
TGGGGAATATTGCACAATGGGCGAAAGCCTGATGCAGCAACGCCGCGTGAGCGAT
GAAGGCCTTCGGGTCGTAAAGCTCTGTCCTCAAGGAAGATAATGACGGTACTTGAG
GAGGAAGCCCCGGCTAACTACGTGCCAGCAGCCGCGGTAATACGTAGGGGGCTAG
CGTTATCCGGAATTACTGGGCGTAAAGGGTGCGTAGGTGGTTTTTTAAGTCAGAAG
TGAAAGGCTACGGCTCAACCGTAGTAAGCTTTTGAAACTAGAGAACTTGAGTGCAG
GAGAGGAGAGTAGAATTCCTAGTGTAGCGGTGAAATGCGTAGATATTAGGAGGAAT
ACCAGTAGCGAAGGCGGCTCTCTGGACTGTAACTGACACTGAGGCACGAAAGCGT
GGGGAGCAAACAGGATTAGATACCCTGGTAGTCCACGCCGTAAACGATGAGTACTA
GGTGTCGGGGGTTACCCCCCTCGGTGCCGCAGCTAACGCATTAAGTACTCCGCCT
GGGAAGTACGCTCGCAAGAGTGAAACTCAAAGGAATTGACGGGGACCCGCACAAG
TAGCGGAGCATGTGGTTTAATTCGAAGCAACGCGAAGAACCTTACCTAAGCTTGAC
ATCCCACTGACCTCTCCCTAATCGGAGATTTCCCTTCGGGGACAGTGGTGACAGGT
GGTGCATGGTTGTCGTCAGCTCGTGTCGTGAGATGTTGGGTTAAGTCCCGCAACGA
GCGCAACCCTTGCCTTTAGTTGCCAGCATTAAGTTGGGCACTCTAGAGGGACTGCC
GAGGATAACTCGGAGGAAGGTGGGGATGACGTCAAATCATCATGCCCCTTATGCTT
AGGGCTACACACGTGCTACAATGGGTGGTACAGAGGGTTGCCAAGCCGCGAGGTG
GAGCTAATCCCTTAAAGCCATTCTCAGTTCGGATTGTAGGCTGAAACTCGCCTACAT
GAAGCTGGAGTTACTAGTAATCGCAGATCAGAATGCTGCGGTGAATGCGTTCCCGG
GTCTTGTACACACCGCCCGTCACACCATGGAAGTTGGGGGCGCCCGAAGCCGGTT
AGCTAACCTTTTAGGAAGCGGCCGTCGAAGGTGAAACCAATGACTGGGGTGAAGTC
GTAACAAGGTAGCCGTATCGGAAGGTGCGGCTGGATCACCT
SEQ ID NO: 18 Dorea longicatena B Strain 18
AGAGTTTGATCCTGGCTCAGGATGAACGCTGGCGGCGTGCTTAACACATGCAAGTC
GAGCGAAGCACTTAAGTCTGA-TTCTTCGGA-
TGAAGACTTTTGTGACTGAGCGGCGGACGGGTGAGTAACGCGTGGGTAACCTGCC
TCATACAGGGGGATAACAGTTAGAAATGACTGCTAATACCGCATAAGACCACGGTA
CCGCATGGTACAGTGGTAAAAACTCCGGTGGTATGAGATGGACCCGCGTCTGATTA
GGTAGTTGGTGGGGTAACGGCCTACCAAGCCGACGATCAGTAGCCGACCTGAGAG
GGTGACCGGCCACATTGGGACTGAGACACGGCCCAGACTCCTACGGGAGGCAGCA
GTGGGGAATATTGCACAATGGAGGAAACTCTGATGCAGCGACGCCGCGTGAAGGA
TGAAGTATTTCGGTATGTAAACTTCTATCAGCAGGGAAGAAAATGACGGTACCTGAC
TAAGAAGCCCCGGCTAACTACGTGCCAGCAGCCGCGGTAATACGTAGGGGGCAAG
CGTTATCCGGATTTACTGGGTGTAAAGGGAGCGTAGACGGCACGGCAAGCCAGAT
GTGAAAGCCCGGGGCTCAACCCCGGGACTGCATTTGGAACTGCTG-
AGCTAGAGTGTCGGAGAGGCAAGTGGAATTCCTAGTGTAGCGGTGAAATGCGTAGA
TATTAGGAGGAACACCAGTGGCGAAGGCGGCTTGCTGGACGATGACTGACGTTGA
GGCTCGAAAGCGTGGGGAGCAAACAGGATTAGATACCCTGGTAGTCCACGCCGTA
AACGATGACTGCTAGGTGTCGGGTGGCAAAGCCATTCGGTGCCGCAGCTAACGCA
ATAAGCAGTCCACCTGGGGAGTACGTTCGCAAGAATGAAACTCAAAGGAATTGACG
GGGACCCGCACAAGCGGTGGAGCATGTGGTTTAATTCGAAGCAACGCGAAGAACC
TTACCTGATCTTGACATCCCGATGACCG-
CTTCGTAATGGAAGCTTTTCTTCGGAACATCGGTGACAGGTGGTGCATGGTTGTCG
TCAGCTCGTGTCGTGAGATGTTGGGTTAAGTCCCGCAACGAGCGCAACCCCTATCT
TCAGTAGCCAGCAGGTTAAGCTGGGCACTCTGGAGAGACTGCCAGGGATAACCTG
GAGGAAGGTGGGGATGACGTCAAATCATCATGCCCCTTATGACCAGGGCTACACAC
GTGCTACAATGGCGTAAACAAAGAGAAGCGA-
ACTCGCGAGGGTAAGCAAATCTCAAAAATAACGTCTCAGTTCGGATTGTAGTCTGCA
ACTCGACTACATGAAGCTGGAATCGCTAGTAATCGCAGATCAGAATGCTGCGGTGA
ATACGTTCCCGGGTCTTGTACACACCGCCCGTCACACCATGGGAGTCAGTAACGCC
CGAAGTCAGTGACCCAACCGTAAGGAGGGAGCTGCCGAAGGTGGGACCGATAACT
GGGGTGAAGTCGTAACAAGGTAGCCGTATCGGAAGGTGCGGCTGGATCACCTCCT
T
SEQ ID NO: 19 Bacteroides thetaiotaomicron Strain 19
ATGAAGAGTTTGATCCTGGCTCAGGATGAACGCTAGCTACAGGCTTAACACATGCA
AGTCGAGGGGCAGCATTTCAGTTTGCTTGCAAACTGGAGATGGCGACCGGCGCAC
GGGTGAGTAACACGTATCCAACCTGCCGATAACTCGGGGATAGCCTTTCGAAAGAA
AGATTAATACCCGATGGCATAAT-
AGAACCGCATGGTTTTTTTATTAAAGAATTTCGGTTATCGATGGGGATGCGTTCCAT
TAGGCAGTTGGTGAGGTAACGGCTCACCAAACCTTCGATGGATAGGGGTTCTGAGA
GGAAGGTCCCCCACATTGGAACTGAGACACGGTCCAAACTCCTACGGGAGGCAGC
AGTGAGGAATATTGGTCAATGGACG-
AGAGTCTGAACCAGCCAAGTAGCGTGAAGGATGACTGCCCTATGGGTTGTAAACTT
CTTTTATATGGGAATAAAGTTTTCCACGTGTGGAATTTTGTATGTACCATATGAATAA
GGATCGGCTAACTCCGTGCCAGCAGCCGCGGTAATACGGAGGATCCGAGCGTTAT
CCGGATTTATTGGGTTTAAAGGGAGCGTAGGTGGACAGTTAAGTCAGTTGTGAAAG
TTTGCGGCTCAACCGTAAAATTGCAGTTGATACTGGCTGTCTTGAGTACAGTAGAGG
TGGGCGGAATTCGTGGTGTAGCGGTGAAATGCTTAGATATCACGAAGAACTCCGAT
TGCGAAGGCAGCTCACTGGACTGCAACTGACACTGATGCTCGAAAGTGTGGGTATC
AAACAGGATTAGATACCCTGGTAGTCCACACAGTAAACGATGAATACTCGCTGTTTG
CGATATACAGTAAGCGGCCAAGCGAAAGCATTAAGTATTCCACCTGGGGAGTACGC
CGGCAAC-
GGTGAAACTCAAAGGAATTGACGGGGGCCCGCACAAGCGGAGGAACATGTGGTTT
AATTCGATGATACGCGAGGAACCTTACCCGGGCTTAAATTGCATTTGAATATATTGG
AAACAGTATAGTCGTAAGACAAATGTGAAGGTGCTGCATGGTTGTCGTCAGCTCGT
GCCGTGAGGTGTCGGCTTAAGTGCCATAACGAGCGCAACCCTTATCTTTAGTTACTA
ACAGGTCATGCTGAGGACTCTAGAGAGACTGCCGTCGTAAGATGTGAGGAAGGTG
GGGATGACGTCAAATCAGCACGGCCCTTACGTCCGGGGCTACACACGTGTTACAAT
GGGGGGTACAGAAGGCAGCTACCTGGTGACAGGATGCTAATCCCAAAAGCCTCTCT
CAGTTCGGATCGAAGTCTGCAACCCGACTTCGTGAAGCTGGATTCGCTAGTAATCG
CGCATCAGCCATGGCGCGGTGAATACGTTCCCGGGCCTTGTACACACCGCCCGTC
AAGCCATGAAAGCCGGGGGTACCTGAAGTACGTAACCGCAAGGAGCGTCCTAGGG
TAAAACTGGTAATTGGGGC
SEQ ID NO: 20 Parabacteroides distasonis Strain 20
CGAAGAGTTTGATCCTGGCTCAGGATGAACGCTAGCGACAGGCTTAACACATGCAA
GTCGAGGGGCAGCACAG-GTAGCAATAC-
CGGGTGGCGACCGGCGCACGGGTGAGTAACGCGTATGCAACTTGCCTATCAGAGG
GGGATAACCCGGCGAAAGTCGGACTAATACCGCATGAAGCAGGGATCCCGCATGG
GAATATTTGCTAAAGATTCATCGCTGATAGATAGGCATGCGTTCCATTAGGCAGTTG
GCGGGGTAACAGCCCACCAAACCGACGATGGATAGGGGTTCTGAGAGGAAGGTCC
CCCACATTGGTACTGAGACACGGACCAAACTCCTACGGGAGGCAGCAGTGAGGAAT
ATTGGTCAATGGCCGAGAGGCTGAACCAGCCAAGTCGCGTGAGGGATGAAGGTTC
TATGGATCGTAAACCTCTTTTATAAGGGAATAAAGTGCGGGACGTGTCCCGTTTTGT
ATGTACCTTATGAATAAGGATCGGCTAACTCCGTGCCAGCAGCCGCGGTAATACGG
AGGATCCGAGCGTTATCCGGATTTATTGGGTTTAAAGGGTGCGTAGGCGGCCTTTT
AAGTCAGCGGTGAAAGTCTGTGGCTCAACCATAGAATTGCCGTTGAAACTGGGGGG
CTTGAGTATGTTTGAGGCAGGCGGAATGCGTGGTGTAGCGGTGAAATGCATAGATA
TCACGCAGAACCCCGATTGCGAAGGCAGCCTGCCAAGCCATGACTGACGCTGATG
CACGAAAGCGTGGGGATCAAACAGGATTAGATACCCTGGTAGTCCACGCAGTAAAC
GATGATCACTAGCTGTTTGCGATACACTGTAAGCGGCACAGCGAAAGCGTTAAGTG
ATCCACCTGGGGAGTACGCCGGCAACGGTGAAACTCAAAGGAATTGACGGGGGCC
CGCACAAGCGGAGGAACATGTGGTTTAATTCGATGATACGCGAGGAACCTTACCCG
GGTTTGAACGCATTCGGACCGAGGTGGAAACACCTTTTCTAGCAATAGCCGTTTGC
GAGGTGCTGCATGGTTGTCGTCAGCTCGTGCCGTGAGGTGTCGGCTTAAGTGCCAT
AACGAGCGCAACCCTTGCCACTAGTTACTAACAGGTAAAGCTGAGGACTCTGGTGG
GACTGCCAGCGTAAGCTGCGAGGAAGGCGGGGATGACGTCAAATCAGCACGGCCC
TTACATCCGGGGCGACACACGTGTTACAATGGCGTGGACAAAGGGAAGCCACCTG
GCGACAGGGAGCGAATCCCCAAACCACGTCTCAGTTCGGATCGGAGTCTGCAACC
CGACTCCGTGAAGCTGGATTCGCTAGTAATCGCGCATCAGCCATGGCGCGGTGAAT
ACGTTCCCGGGCCTTGTACACACCGCCCGTCAAGCCATGGGAGCCGGGGGTACCT
GAAGTCCGTAACCGCGAGGATCGGCCTAGGGTAAAACTGGTGACTGGGGCTAAGT
CGTAACAAG
SEQ ID NO: 21 Escherichia coli Strain 21
TTGAAGAGTTTGATCATGGCTCAGATTGAACGCTGGCGGCAGGCCTAACACATGCA
AGTCGAACGGTAACAGGAAGCAGCTTGCTGCTTTGCTGACGAGTGGCGGACGGGT
GAGTAATGTCTGGGAAACTGCCTGATGGAGGGGGATAACTACTGGAAACGGTAGCT
AATACCGCATAACGTCGCAAGACCAAAGAGGGGGACCTTCGGGCCTCTTGCCATCG
GATGTGCCCAGATGGGATTAGCTAGTAGGTGGGGTAACGGCTCACCTAGGCGACG
ATCCCTAGCTGGTCTGAGAGGATGACCAGCCACACTGGAACTGAGACACGGTCCAG
ACTCCTACGGGAGGCAGCAGTGGGGAATATTGCACAATGGGCGCAAGCCTGATGC
AGCCATGCCGCGTGTATGAAGAAGGCCTTCGGGTTGTAAAGTACTTTCAGCGGGGA
GGAAGGGAGTAAAGTTAATACCTTTGCTCATTGACGTTACCCGCAGAAGAAGCACC
GGCTAACTCCGTGCCAGCAGCCGCGGTAATACGGAGGGTGCAAGCGTTAATCGGA
ATTACTGGGCGTAAAGCGCACGCAGGCGGTTTGTTAAGTCAGATGTGAAATCCCCG
GGCTCAACCTGGGAACTGCATCTGATACTGGCAAGCTTGAGTCTCGTAGAGGGGG
GTAGAATTCCAGGTGTAGCGGTGAAATGCGTAGAGATCTGGAGGAATACCGGTGGC
GAAGGCGGCCCCCTGGACGAAGACTGACGCTCAGGTGCGAAAGCGTGGGGAGCAA
ACAGGATTAGATACCCTGGTAGTCCACGCCGTAAACGATGTCGACTTGGAGGTTGT
GCCCTTGAGGCGTGGCTTCCGGAGCTAACGCGTTAAGTCGACCGCCTGGGGAGTA
CGGCCGCAAGGTTAAAACTCAAATGAATTGACGGGGGCCCGCACAAGCGGTGGAG
CATGTGGTTTAATTCGATGCAACGCGAAGAACCTTACCTGGTCTTGACATCCACGGA
AGTTTTCAGAGATGAGAATGTGCCTTCGGGAACCGTGAGACAGGTGCTGCATGGCT
GTCGTCAGCTCGTGTTGTGAAATGTTGGGTTAAGTCCCGCAACGAGCGCAACCCTT
ATCCTTTGTTGCCAGCGGTCCGGCCGGGAACTCAAAGGAGACTGCCAGTGATAAAC
TGGAGGAAGGTGGGGATGACGTCAAGTCATCATGGCCCTTACGACCAGGGCTACA
CACGTGCTACAATGGCGCATACAAAGAGAAGCGACCTCGCGAGAGCAAGCGGACC
TCATAAAGTGCGTCGTAGTCCGGATTGGAGTCTGCAACTCGACTCCATGAAGTCGG
AATCGCTAGTAATCGTGGATCAGAATGCCACGGTGAATACGTTCCCGGGCCTTGTA
CACACCGCCCGTCACACCATGGGAGTGGGTTGCAAAAGAAGTAGGTAGCTTAACCT
TCGGGAGGGCGCTTACCACTTTGTGATTCATGACTGGGGTGAAGTCGTAACAAGGT
AACCGTAGGGGAACCTGCGGTTGGATCACCTCCTT
SEQ ID NO: 22 Bifidobacterium longum Strain 22
TGGCTCAGGATGAACGCTGGCGGCGTGCTTAACACATGCAAGTCGAACGGGATCC
ATCAGGCTTTGCTTGGTGGTGAGAGTGGCGAACGGGTGAGTAATGCGTGACCGAC
CTGCCCCATACACCGGAATAGCTCCTGGAAACGGGTGGTAATGCCGGATGCTCCAG
TTGATCGCATGGTCTTCTGGGAAAGCTTTCGCGGTATGGGATGGGGTCGCGTCCTA
TCAGCTTGACGGCGGGGTAACGGCCCACCGTGGCTTCGACGGGTAGCCGGCCTGA
GAGGGCGACCGGCCACATTGGGACTGAGATACGGCCCAGACTCCTACGGGAGGCA
GCAGTGGGGAATATTGCACAATGGGCGCAAGCCTGATGCAGCGACGCCGCGTGAG
GGATGGAGGCCTTCGGGTTGTAAACCTCTTTTATCGGGGAGCAAGCGAGAGTGAGT
TTACCCGTTGAATAAGCACCGGCTAACTACGTGCCAGCAGCCGCGGTAATACGTAG
GGTGCAAGCGTTATCCGGAATTATTGGGCGTAAAGGGCTCGTAGGCGGTTCGTCGC
GTCCGGTGTGAAAGTCCATCGCTTAACGGTGGATCCGCGCCGGGTACGGGGGGGC
TTGAGTGCGGTAGGGGAGACTGGAATTCCCGGTGTAACGGTGGAATGTGTAGATAT
CGGGAAGAACACCAATGGCGAAGGCAGGTCTCTGGGCCGTTACTGACGCTGAGGA
GCGAAAGCGTGGGGAGCGAACAGGATTAGATACCCTGGTAGTCCACGCCGTAAAC
GGTGGATGCTGGATGTGGGGCCCGTTCCACGGGTTCCGTGTCGGAGCTAACGCGT
TAAGCATCCCGCCTGGGGAGTACGGCCGCAAGGCTAAAACTCAAAGAAATTGACGG
GGGCCCGCACAAGCGGCGGAGCATGCGGATTAATTCGATGCAACGCGAAGAACCT
TACCTGGGCTTGACATGTTCCCGACGGTCGTAGAGATACGGCTTCCCTTCGGGGCG
GGTTCACAGGTGGTGCATGGTCGTCGTCAGCTCGTGTCGTGAGATGTTGGGTTAAG
TCCCGCAACGAGCGCAACCCTCGCCCCGTGTTGCCAGCGGATTATGCCGGGAACT
CACGGGGGACCGCCGGGGTTAACTCGGAGGAAGGTGGGGATGACGTCAGATCATC
ATGCCCCTTACGTCCAGGGCTTCACGCATGCTACAATGGCCGGTACAACGGGATGC
GACGCGGCGACGCGGAGCGGATCCCTGAAAACCGGTCTCAGTTCGGATCGCAGTC
TGCAACTCGACTGCGTGAAGGCGGAGTCGCTAGTAATCGCGAATCAGCAACGTCGC
GGTGAATGCGTTCCCGGGCCTTGTACACACCGCCCGTCAAGTCATGAAAGTGGGCA
GCACCCGAAGCCGGTGGCCTAACCCCTTGTGGGATGGAGCCGTCTAAGGTGAGGC
TCGTGATTGGGACTAAGTCGTAACAAGGTAGCCGTACCGGAAGGTGCGGCTGGATC
ACCTCCTT
SEQ ID NO: 23 Absiella innocuum Strain 23
ATGGAGAGTTTGATCCTGGCTCAGGATGAACGCTGGCGGCATGCCTAATACATGCA
AGTCGAACGAAGT-
TTCGAGGAAGCTTGCTTCCAAAGAGACTTAGTGGCGAACGGGTGAGTAACACGTAG
GTAACCTGCCCATGTGTCCGGGATAACTGCTGGAAACGGTAGCTAAAACCGGATAG
GTATACAGAGCGCATGCTCAGTATATTAAAGCGCCCATCAAGGCGTGAACATGGAT
GGACCTGCGGCGCATTAGCTAGTTGGTGAGGTAACGGCCCACCAAGGCGATGATG
CGTAGCCGGCCTGAGAGGGTAAACGGCCACATTGGGACTGAGACACGGCCCAAAC
TCCTACGGGAGGCAGCAGTAGGGAATTTTCGTCAATGGGGGAAACCCTGAACGAG
CAATGCCGCGTGAGTGAAGAAGGTCTTCGGATCGTAAAGCTCTGTTGTAAGTGAAG
AACGGCTCATAGAGGAAATGCTATGGGAGTGACGGTAGCTTACCAGAAAGCCACGG
CTAACTACGTGCCAGCAGCCGCGGTAATACGTAGGTGGCAAGCGTTATCCGGAATC
ATTGGGCGTAAAGGGTGCGTAGGTGGCGTACTAAGTCTGTAGTAAAAGGCAATGGC
TCAACCATTGTAAGCTATGGAAACTGGTATGCTGGAGTGCAGAAGAGGGCGATGGA
ATTCCATGTGTAGCGGTAAAATGCGTAGATATATGGAGGAACACCAGTGGCGAAGG
CGGTCGCCTGGTCTGTAACTGACACTGAGGCACGAAAGCGTGGGGAGCAAATAGG
ATTAGATACCCTAGTAGTCCACGCCGTAAACGATGAGAACTAAGTGTTGGAGGAATT
CAGTGCTGCAGTTAACGCAATAAGTTCTCCGCCTGGGGAGTATGCACGCAAGTGTG
AAACTCAAAGGAATTGACGGGGGCCCGCACAAGCGGTGGAGTATGTGGTTTAATTC
GAAGCAACGCGAAGAACCTTACCAGGCCTTGACATGGAAACAAATACCCTAGAGAT
AGGGGGATAATTATGGATCACACAGGTGGTGCATGGTTGTCGTCAGCTCGTGTCGT
GAGATGTTGGGTTAAGTCCCGCAACGAGCGCAACCCTTGTCGCATGTTACCAGCAT
CAAGTTGGGGACTCATGCGAGACTGCCGGTGACAAACCGGAGGAAGGTGGGGATG
ACGTCAAATCATCATGCCCCTTATGGCCTGGGCTACACACGTACTACAATGGCGGC
CACAAAGAGCAGCGACACAGTGATGTGAAGCGAATCTCATAAAGGTCGTCTCAGTT
CGGATTGAAGTCTGCAACTCGACTTCATGAAGTCGGAATCGCTAGTAATCGCAGAT
CAGCATGCTGCGGTGAATACGTTCTCGGGCCTTGTACACACCGCCCGTCAAACCAT
GGGAGTCAGTAATACCCGAAGCCGGTGGCATAACCGTAAGGAGTGAGCCGTCGAA
GGTAGGACCGA
SEQ ID NO: 24 Fusobacterium_A sp Strain 24
AGAGTTTGATCCTGGCTCAGGATGAACGCTGACAGAATGCTTAACACATGCAAGTC
TA-
CTTGATCCTTCGGGTGATGGTGGGGGACGGGTGAGTAACGCGTAAAGAACTTGCCC
TGCAGTCTGGGACAACATTTGGAAACGAATGCTAATACCGGATATTATGT-
ATTTCTCGCATGAGTTTTACATGAAAGCTATATGCGCTGCAGGAGAGCTTTGCGTCC
TATTAGCTAGTTGGTGAGGTAACGGCTCACCAAGGCCATGATAGGTAGCCGGCCTG
AGAGGGTGAACGGCCACAAGGGGACTGAGACACGGCCCTTACTCCTACGGGAGGC
AGCAGTGGGGAATATTGGACAATGGACCAAAAGTCTGATCCAGCAATTCTGTGTGC
ACGATGAAGTTTTTCGGAATGTAAAGTGCTTTCAGTTGGGACGAAGTAAGTGACGGT
ACCAACAGAAGAAGCGACGGCTAAATACGTGCCAGCAGCCGCGGTAATACGTATGT
CGCAAGCGTTATCCGGATTTATTGGGCGTAAAGCGCGTC-
TAGGCGGTTTGGTAAGTCTGATGTGAAAATGCGGGGCTCAACTCCGTATTGCGTTG
GAAACTG-
CTAAACTAGAGTACTGGAGAGGTGGGGGGAACTACAAGTGTAGAGGTGAAATTCGT
AGATATTTGTAGGAATGCCGATGGGGAAGCCAGCCCACTGGACAGATACTGACGCT
AAAGCGCGAAAGCGTGGGTAGCAAACAGGATTAGATAC-----
CCTGGTAGTCCACGCCGTAAACGATGATTACTAGGTGTTGGGGGTCGAACCTCAGC
GCCCAAGCTAACGCGATAAGTAATCCGCCTGGGGAGTACGTACGCAAGTATGAAAC
TCAAAGGAATTGACGGGGACCCGCACAAGCGGTGGAGCATGTGGTTTAATTCGACG
CAACGCGAGGAACCTTACCAGCGTTTGACATCCTAAGAAATTAGCAGAGATGCTTTT
GTGCCCCTTCGGGGGAACTTAGTGACAGGTGGTGCATGGCTGTCGTCAGCTCGTG
TCGTGAGATGTTGGGTTAAGTCCCGCAACGAGCGCAACCCCTTTCGTATGTTGCCA
TCATTAAGTTGGGCACTCATGCGATACTGCCTGCGATGAGCAGGAGGAAGGTGGG
GATGACGTCAAGTCATCATGCCCCTTATACGCTGGGCTACACACGTGCTACAATGG
GTAGTACAGAGAGTCGCAAACCTGCGAGGGGGAGCTAATCTCAGAAAACTATTCTC
AGTTCGGATTGTACTCTGCAACTCGAGTACATGAAGTTGGAATCGCTAGTAATCGCA
AATCAGCTATGTTGCGGTGAATACGTTCTCGGGTCTTGTACACACCGCCCGTCACA
CCACGAGAGTTGGTTGCACCTGAAGTAGCAGGCCTAACCGCAAGGAGGGATGCTC
CGAGGGTGTGATTAGCGATTGGGGTGAAGTCGTAACAAGGTATCCGTACGGGAAC
GTGCGGATGGATCACCTCCTT
SEQ ID NO: 25 Parabacteroides merdae Strain 25
AGAGTTTGATCCTGGCTCAGGATGAACGCTAGCGACAGGCTTAACACATGCAAGTC
GAGGGGCAGCATGATTTGTAGCAATACAGATTGATGGCGACCGGCGCACGGGTGA
GTAACGCGTATGCAACTTACCTATCAGAGGGGGATAGCCCGGCGAAAGTCGGATTA
ATACCCCATAAAACAGGGGTCCCGCATGGGAATATTTGTTAAAGATTCATCGCTGAT
AGATAGGCATGCGTTCCATTAGGCAGTTGGGGGGTAACGGCCCACCAAACCGAC
GATGGATAGGGGTTCTGAGAGGAAGGTCCCCCACATTGGTACTGAGACACGGACC
AAACTCCTACGGGAGGCAGCAGTGAGGAATATTGGTCAATGGCCGAGAGGCTGAA
CCAGCCAAGTCGCGTGAAGGAAGAAGGATCTATGGTTTGTAAACTTCTTTTATAGGG
GAATAAAGTGGAGGACGTGTCCTTTTTTGTATGTACCCTATGAATAAGCATCGGCTA
ACTCCGTGCCAGCAGCCGCGGTAATACGGAGGATGCGAGCGTTATCCGGATTTATT
GGGTTTAAAGGGTGCGTAGGTGGTGATTTAAGTCAGCGGTGAAAGTTTGTGGCTCA
ACCATAAAATTGCCGTTGAAACTGGGTTACTTGAGTGTGTTTGAGGTAGGCGGAAT
GCGTGGTGTAGCGGTGAAATGCATAGATATCACGCAGAACTCCGATTGCGAAGGCA
GCTTACTAAACCATAACTGACACTGAAGCACGAAAGCGTGGGGATCAAACAGGATT
AGATACCCTGGTAGTCCACGCAGTAAACGATGATTACTAGGAGTTTGCGATACAATG
TAAGCTCTACAGCGAAAGCGTTAAGTAATCCACCTGGGGAGTACGCCGGCAACGGT
GAAACTCAAAGGAATTGACGGGGGCCCGCACAAGCGGAGGAACATGTGGTTTAATT
CGATGATACGCGAGGAACCTTACCCGGGTTTGAACGTAGTCTGACCGGAGTGGAAA
CACTCTTTCTAGCAATAGCAGATTACGAGGTGCTGCATGGTTGTCGTCAGCTCGTG
CCGTGAGGTGTCGGCTTAAGTGCCATAACGAGCGCAACCCTTATCACTAGTTACTA
ACAGGTGAAGCTGAGGACTCTGGTGAGACTGCCAGCGTAAGCTGTGAGGAAGGTG
GGGATGACGTCAAATCAGCACGGCCCTTACATCCGGGGCGACACACGTGTTACAAT
GGCATGGACAAAGGGCAGCTACCTGGTGACAGGATGCTAATCTCCAAACCATGTCT
CAGTTCGGATCGGAGTCTGCAACTCGACTCCGTGAAGCTGGATTCGCTAGTAATCG
CGCATCAGCCATGGCGCGGTGAATACGTTCCCGGGCCTTGTACACACCGCCCGTC
AAGCCATGGGAGCCGGGGGTACCTGAAGTCCGTAACCGCAAGGATCGGCCTAGGG
TAAAACTGGTGACTGGGGCTAAGTCGTAACAAGGTA
SEQ ID NO: 26 Coprococcus_B comes Strain 26
GAGAGTTTGATCCTGGCTCAGGATGAACGCTGGC-GG-
CGTGCTTAACACATGCAAGTCGAACGAAGCACTTACCTTTGA-TTCTTCGGA-
TGAAGGTTTTTG-
TGACTGAGTGGGGGACGGGTGAGTAACGCGTGGGTAACCTGCCTCATACAGGGGG
ATAACAGTTAGAAATGACTGCTAATACCGCATAAGACCACAGAGCTGCATGGCTCA
GTGGGAAAAACTCCGGTGGTATGAGATGGACCCGCGTCTGATTAGGTAGTTGGTGG
GGTAACGGCCTACCAAGCCAACGATCAGTAGCCGACCTGAGAGGGTGACCGGCCA
CATTGGGACTGAGACACGGCCCAAACTCCTACGGGAGGCAGCAGTGGGGAATATT
GCACAATGGGGGAAACCCTGATGCAGCGACGCCGCGTGAGCGAAGAAGTATTTCG
GTATGTAAAGCTCTATCAGCAGGGAAGAAAATGACGGTACCTGACTAAGAAGCACC
GGCTAAATACGTGCCAGCAGCCGCGGTAATACGTATGGTGCAAGCGTTATCCGGAT
TTACTGGGTGTAAAGGGAGCGTAGACGGCTGTGTAAGTCTGAAGTGAAAGCCCGG
GGCTCAACCCCGGGACTGCTTTGGAAACTATGCAGCTAGAGTGTCGGAGAGGTAAG
TGGAATTCCCAGTGTAGCGGTGAAATGCGTAGATATTGGGAGGAACACCAGTGGCG
AAGGCGGCTTACTGGACGATGACTGACGTTGAGGCTCGAAAGCGTGGGGAGCAAA
CAGGATTAGATACCCTGGTAGTCCACGCCGTAAACGATGACTACTAGGTGTCGGGG
AGCAGAGCTCTTCGGTGCCGCAGCAAACGCAATAAGTAGTCCACCTGGGGAGTAC
GTTCGCAAGAATGAAACTCAAAGGAATTGACGGGGACCCGCACAAGCGGTGGAGC
ATGTGGTTTAATTCGAAGCAACGCGAAGAACCTTACCTGCTCTTGACATCCCGGTGA
CCGG-
CATGTAATGATGCCTTTTCTTCGGAACACCGGTGACAGGTGGTGCATGGTTGTCGT
CAGCTCGTGTCGTGAGATGTTGGGTTAAGTCCCGCAACGAGCGCAACCCTTATCTT
CAGTAGCCAGCAATTCGGATGGGCACTCTGGAGAGACTGCCAGGGATAACCTGGA
GGAAGGTGGGGATGACGTCAAATCATCATGCCCCTTATGAGCAGGGCTACACACGT
GCTACAATGACGTAAACAAAGGGAAGCGAGCCTGCGAGGGTAAGCAAATCTCAAAA
ATAACGTCTCAGTTCGGATTGTAGTCTGCAACTCGACTACATGAAGCTGGAATCGCT
AGTAATCGCGAATCAGCATGTCGCGGTGAATACGTTCCCGGGTCTTGTACACACCG
CCCGTCACACCATGGGAGTTGGTAACGCCCGAAGTCAGTGACCCAACCGTAAGGA
GGGAGCTGCCGAAGGTGGGACCGATAACTGGGGTGAAGTCGTAACAAGGTAGCCG
TATCGGAAGGTGCGGCTGGATCACCTCC
SEQ ID NO: 27 Agathobacter rectale Strain 27
AGAGTTTGATCCTGGCTCAGGATGAACGCTGGCGGCGTGCTTAACACATGCAAGTC
GAACGAAGCACTTTATTTGATTTCCTTCGGGACTGATTATTTTGTGACTGAGTGGCG
GACGGGTGAGTAACGCGTGGGTAACCTGCCTTGTACAGGGGGATAACAGTTGGAA
ACGGCTGCTAATACCGCATAAGCGCACGGCATCGCATGATGCAGTGTGAAAAACTC
CGGTGGTATAAGATGGACCCGCGTTGGATTAGCTAGTTGGTGAGGTAACGGCCCAC
CAAGGCGACGATCCATAGCCGACCTGAGAGGGTGACCGGCCACATTGGGACTGAG
ACACGGCCCAAACTCCTACGGGAGGCAGCAGTGGGGAATATTGCACAATGGGCGA
AAGCCTGATGCAGCGACGCCGCGTGAGCGAAGAAGTATTTCGGTATGTAAAGCTCT
ATCAGCAGGGAAGATAATGACGGTACCTGACTAAGAAGCACCGGCTAAATACGTGC
CAGCAGCCGCGGTAATACGTATGGTGCAAGCGTTATCCGGATTTACTGGGTGTAAA
GGGAGCGCAGGCGGTGCGGCAAGTCTGATGTGAAAGCCCGGGGCTCAACCCCGG
TACTGCATTGGAAACTGTCGTACTAGAGTGTCGGAGGGGTAAGCGGAATTCCTAGT
GTAGCGGTGAAATGCGTAGATATTAGGAGGAACACCAGTGGCGAAGGCGGCTTACT
GGACGATAACTGACGCTGAGGCTCGAAAGCGTGGGGAGCAAACAGGATTAGATAC
CCTGGTAGTCCACGCCGTAAACGATGAATACTAGGTGTTGGGAAGCATTGCTTCTC
GGTGCCGTCGCAAACGCAGTAAGTATTCCACCTGGGGAGTACGTTCGCAAGAATGA
AACTCAAAGGAATTGACGGGGACCCGCACAAGCGGTGGAGCATGTGGTTTAATTCG
AAGCAACGCGAAGAACCTTACCAAGTCTTGACATCCTTCTGACCGGTACTTAACCGT
ACCTTCTCTTCGGAGCAGGAGTGACAGGTGGTGCATGGTTGTCGTCAGCTCGTGTC
GTGAGATGTTGGGTTAAGTCCCGCAACGAGCGCAACCCTTATCTTTAGTAGCCAGC
GGTTCGGCCGGGCACTCTAGAGAGACTGCCAGGGATAACCTGGAGGAAGGCGGGG
ATGACGTCAAATCATCATGCCCCTTATGACTTGGGCTACACACGTGCTACAATGGC
GTAAACAAAGGGAAGCAAAGCTGTGAAGCCGAGCAAATCTCAAAAATAACGTCTCA
GTTCGGACTGTAGTCTGCAACCCGACTACACGAAGCTGGAATCGCTAGTAATCGCA
GATCAGAATGCTGCGGTGAATACGTTCCCGGGTCTTGTACACACCGCCCGTCACAC
CATGGGAGTTGGGAATGCCCGAAGCCAGTGACCTAACCGAAAGGAAGGAGCTGTC
GAAGGCAGGCTCGATAACTGGGGTGAAGTCGTAACAAGGTAGCCGTATCGGAAGG
TGCGGCTGGATCACCT
SEQ ID NO: 28 Erysipelatoclostridium ramosum Strain 28
GAGAGTTTGATCCTGGCTCAGGATGAACGCTGGCGGCGTGCCTAATACATGCAAGT
CGAACGCGAGCACTTGTGCTCGAGTGGCGAACGGGTGAGTAATACATAAGTAACCT
GCCCTAGACAGGGGGATAACTATTGGAAAC-
GATAGCTAAGACCGCATAGGTACGGACACTGCATGGTGACCGTATTAAAAGTGCCT
CAAAGCACTGGTAGAGGATGGACTTATGGCGCATTAGCTGGTTGGCGGGGTAACG
GCCCACCAAGGCGACGATGCGTAGCCGACCTGAGAGGGTGACCGGCCACACTGGG
ACTGAGACACGGCCCAGACTCCTACGGGAGGCAGCAGTAGGGAATTTTCGGCAAT
GGGGGAAACCCTGACCGAGCAACGCCGCGTGAAGGAAGAAGGTTTTCGGATTGTA
AACTTCTGTTATAAAGGAAGAACGGGGGCTACAGGAAATGGTAGCCGAGTGACGGT
ACTTTA--
TTAGAAAGCCACGGCTAACTACGTGCCAGCAGCCGCGGTAATACGTAGGTGGCAAG
CGTTATCCGGAATTATTGGGCGTAAAGAGGGAGCAGGGGGCAGCAAGGGTCTGTG
GTGAAAGCCTGAAGCTTAACTTCAGTAAGCCATAGAAACCAGGCAGCTAGAGTGCA
GGAGAGGATCGTGGAATTCCATGTGTAGCGGTGAAATGCGTAGATATATGGAGGAA
CACCAGTGGCGAAGGCGACGATCTGGCCTGCAACTGACGCTCAGTCCCGAAAGCG
TGGGGAGCAAATAGGATTAGATACCCTAGTAGTCCACGCCGTAAACGATGAGTACT
AAGTGTTGGATGTCAAAGTTCAGTGCTGCAGTTAACGCAATAAGTACTCCGCCTGA
GTAGTACGTTCGCAAGAATGAAACTCAAAGGAATTGACGGGGGCCCGCACAAGCG
GTGGAGCATGTGGTTTAATTCGAAGCAACGCGAAGAACCTTACCAGGTCTTGACAT
ACTCATAAAGGCTCCAGAGATGGAGAGATAGCTATATGAGATACAGGTGGTGCATG
GTTGTCGTCAGCTCGTGTCGTGAGATGTTGGGTTAAGTCCCGCAACGAGCGCAACC
CTTATCGTTAGTTACCATCATTAAGTTGGGGACTCTAGCGAGACTGCCAGTGACAAG
CTGGAGGAAGGCGGGGATGACGTCAAATCATCATG-CCCTTATGACCTGGGCT--
ACACGTGCTACAATGGATGGTGCAGAGAGAGGCGAGCCGCG-
GGCGAAACAAAAGCCCATAAAA-CCATT-
TCAGTTCCGGATTGTAGTCTGCAACTCGACTTTATTACATGAGAGTTGGAATCGCTA
GTAATCGCGAATCAGCATGTCGCGGTTTGAATACGTTCTCGGGCCTT-
ACCGCCCGTCACACCAA-AGGATTGATAACAC--G--GC-GGTGG-
CTAACCGCAAGGAAAG-
GCTGTCTAAGGTGGGATTGATGATTGGGGTAAAGTCGTAACGAAGGTATCCCTACG
GGAACGTGGGGATGGATCACCTCC
SEQ ID NO: 29 Clostridium_M citroniae Strain 29
AGAGTTTGATCCTGGCTCAGGATGAACGCTGGCGGCGTGCCTAACACATGCAAGTC
GAACGAAGCAATTAGAAAGAAGTTTTCGGATGGATTTCTGATTGACTGAGTGGCGG
ACGGGTGAGTAACGCGTGGATAACCTGCCTCACACTGGGGGATAACAGTTAGAAAT
GGCTGCTAATACCGCATAAGCGCACAGTACCGCATGGTACGGTGTGAAAAACTCCG
GTGGTGTGAGATGGATCCGCGTCTGATTAGTTAGTTGGCGGGGTAACGGCCCACCA
AGACAGCGATCAGTAGCCGACCTGAGAGGGTGACCGGCCACATTGGGACTGAGAC
ACGGCCCAAACTCCTACGGGAGGCAGCAGTGGGGAATATTGCACAATGGGCGAAA
GCCTGATGCAGCGACGCCGCGTGAGTGAAGAAGTATTTCGGTATGTAAAGCTCTAT
CAGCAGGGAAGAAACTGACGGTACCTGACTAAGAAGCCCCGGCTAACTACGTGCCA
GCAGCCGCGGTAATACGTAGGGGGCAAGCGTTATCCGGATTTACTGGGTGTAAAG
GGAGCGTAGACGGCGAAGCAAGTCTGGAGTGAAAACCCAGGGCTCAACCCTGGGA
CTGCTTTGGAAACTGTTTTGCTAGAGTGTCGGAGAGGTAAGTGGAATTCCTAGTGTA
GCGGTGAAATGCGTAGATATTAGGAGGAACACCAGTGGCGAAGGCGGCTTACTGG
ACGATAACTGACGTTGAGGCTCGAAAGCGTGGGGAGCAAACAGGATTAGATACCCT
GGTAGTCCACGCCGTAAACGATGAATGCTAGGTGTTGGGGGGCAAAGCCCTTCGG
TGCCGTCGCAAACGCAATAAGCATTCCACCTGGGGAGTACGTTCGCAAGAATGAAA
CTCAAAGGAATTGACGGGGACCCGCACAAGCGGTGGAGCATGTGGTTTAATTCGAA
GCAACGCGAAGAACCTTACCAAGTCTTGACATCCCCCTGACCGGTCAGTAAAGTGA
CCTTTCCTTCGGGACAGGGGAGACAGGTGGTGCATGGTTGTCGTCAGCTCGTGTC
GTGAGATGTTGGGTTAAGTCCCGCAACGAGCGCAACCCTTATCCTTAGTAGCCAGC
AGGTAAAGCTGGGCACTCTAGGGAGACTGCCAGGGATAACCTGGAGGAAGGTGGG
GATGACGTCAAATCATCATGCCCCTTATGATTTGGGCTACACACGTGCTACAATGGC
GTAAACAAAGGGAAGCGACCCTGCGAAGGCAAGCAAATCCCAAAAATAACGTCCCA
GTTCGGACTGTAGTCTGCAACCCGACTACACGAAGCTGGAATCGCTAGTAATCGCG
AATCAGAATGTCGCGGTGAATACGTTCCCGGGTCTTGTACACACCGCCCGTCACAC
CATGGGAGTCAGCAACGCCCGAAGTCAGTGACCCAAC-CGAAAG-
GAGGGAGCTGCCGAAGGCGGGGCAGGTAACTGGGGTGAAGTCGTAACAAGGTAGC
CGTA
SEQ ID NO: 30 Bacteroides faecis Strain 30
ATGAAGAGTTTGATCCTGGCTCAGGATGAACGCTAGCTACAGGCTTAACACATGCA
AGTCGAGGGGCATCAGGAAGAAAGCTTGCTTTCTTTGCTGGCGACCGGCGCACGG
GTGAGTAACACGTATCCAACCTGCCCTTTACTCGGGGATAGCCTTTCGAAAGAAAG
ATTAATACCCGATGGCATAATGATTCCGCATGGTTTCATTATTAAAGGATTCCGGTA
AAGGATGGGGATGCGTTCCATTAGGTTGTTGGTGAGGTAACGGCTCACCAAGCCTT
CGATGGATAGGGGTTCTGAGAGGAAGGTCCCCCACATTGGAACTGAGACACGGTC
CAAACTCCTACGGGAGGCAGCAGTGAGGAATATTGGTCAATGGGCGATAGCCTGAA
CCAGCCAAGTAGCGTGAAGGATGACTGCCCTATGGGTTGTAAACTTCTTTTATAAAG
GAATAAAGTCGGGTATGCATACCCGTTTGCATGTACTTTATGAATAAGGATCGGCTA
ACTCCGTGCCAGCAGCCGCGGTAATACGGAGGATCCGAGCGTTATCCGGATTTATT
GGGTTTAAAGGGAGCGTAGGTGGA-
TGTTTAAGTCAGTTGTGAAAGTTTGCGGCTCAACCGTAAAATTGCAGTTGATACTGG
CTGTCTTGAGTGCAGTTGAGGCAGGCGGAATTCGTGGTGTAGCGGTGAAATGCTTA
GATATCACGAAGAACTCCGATTGCGAAGGCAGCCTGCTAAGCTGCAACTGACATTG
AGGCTCGAAAGTGTGGGTATCAAACAGGATTAGATACCCTGGTAGTCCACACGGTA
AACGATGAATACTCGCTGTTTGCGATATACGGCAAGCGGCCAAGCGAAAGCGTTAA
GTATTCCACCTGGGGAGTACGCCGGCAACGGTGAAACTCAAAGGAATTGACGGGG
GCCCGCACAAGCGGAGGAACATGTGGTTTAATTCGATGATACGCGAGGAACCTTAC
CCGGGCTTAAATTGCAGTGGAATGATGTGGAAACATGTCAGTGAGCAATCACCGCT
GTGAAGGTGCTGCATGGTTGTCGTCAGCTCGTGCCGTGAGGTGTCGGCTTAAGTGC
CATAACGAGCGCAACCCTTATCTTTAGTTACTAACAGGTGATGCTGAGGACTCTGGA
GAGACTGCCATCGTAAGATGTGAGGAAGGTGGGGATGACGTCAAATCAGCACGGC
CCTTACGTCCGGGGCTACACACGTGTTACAATGGGAGGTACAGAAGGCAGCTACCC
GGCGACGGGATGCCAATCCCCAAAACCTCTCTCAGT
SEQ ID NO: 31 Bacteroides ovatus Strain 31
ATGAAGAGTTTGATCCTGGCTCAGGATGAACGCTAGCTACAGGCTTAACACATGCA
AGTCGAGGGGCAGCATGGTCTTAGCTTGCTAAGGCTGATGGCGACCGGCGCACGG
GTGAGTAACACGTATCCAACCTGCCGTCTACTCTTGGCCAGCCTTCTGAAAGGAAG
ATTAATCCAGGATGGGATCATGAGTTCACATGTCCGCATGATTAAAGGTATTTTCCG
GTAGACGATGGGGATGCGTTCCATTAGATAGTTGGCGGGGTAACGGCCCACCAAAC
CGACGATGGATAGGGGTTCTGAGAGGAAGGTCCCCCACATTGGTACTGAGACACG
GACCAAACTCCTACGGGAGGCAGCAGTGAGGAATATTGGTCAATGGGCGAGAGCC
TGAACCAGCCAAGTAGCGTGAAGGATGACTGCCCTATGGGTTGTAAACTTCTTTTAT
AAGGGAATAAAGTCGGGTATGCATACCCGTTTGCATGTACTTTATGAATAAGGATCG
GCTAACTCCGTGCCAGCAGCCGCGGTAATACGGAGGATCCGAGCGTTATCCGGATT
TATTGGGTTTAAAGGGAGCGTAGGTGGACGTTTAAGTCAGTTGTGAAAGTTTGCGG
CTCAACCGTAAAATTGCAGTTGATACTGGATGTCTTGAGTGCAGTTGAGGCAGGCG
GAATTCGTGGTGTAGCGGTGAAATGCTTAGATATCACGAAGAACTCCGATTGCGAA
GGCAGCCTGCTAAGCTGCAACTGACATTGAGGCTCGAAAGTGTGGGTATCAAACAG
GATTAGATACCCTGGTAGTCCACACGGTAAACGATGAATACTCGCTGTTTGCGATAT
ACGGCAAGCGGCCAAGCGAAAGCGTTAAGTATTCCACCTGGGGAGTACGCCGGCA
ACGGTGAAACTCAAAGGAATTGACGGGGGCCCGCACAAGCGGAGGAACATGTGGT
TTAATTCGATGATACGCGAGGAACCTTACCCGGGCTTAAATTGCAGACGAATTACGA
GGAAACTTGTAAGCCGCAAGGCGTCTGTGAAGGTGCTGCATGGTTGTCGTCAGCTC
GTGCCGTGAGGTGTCGGCTTAAGTGCCATAACGAGCGCAACCCTTATCTTTAGTTA
CTAACAGGTTATGCTGAGGACTCTAGAG-
AGACTGCCATCGTAAGATGTGAGGAAGGTGGGGATGACGTCAAATCAGCACGGCC
CTTACGTCCGGGGCTACACACGTGTTACAATGGGGGGTACAGAGGGCCGCTACCA
CGCGAGTGGATGCCAATCCCTAAAACCCCTCTCAGTTCGGACTGGAGTCTGCAACC
CGACTCCACGAAGCTGGATTCGCTAGTAATCGCGCATCAGCCACGGCGCGGTGAAT
ACGTTCCCGGGCCTTGTACACACCGCCCGTCAAGCCATGGGAGCCGGGGGTACCT
GAAGTGCGTAACCGCGAGGATCGCCCTAGGGTAAAACTGGTGACTGGGGCTAAGT
CGTAACAAGGTAGCCGTACCGGAAG
SEQ ID NO: 32 Bifidobacterium pseudocatenulatum Strain 32
TTCGATTCTGGCTCAGGATGAACGCTGGCGGCGTGCTTAACACATGCAAGTCGAAC
GGGATCCATCAGGCTTTGCTTGGTGGTGAGAGTGGCGAACGGGTGAGTAATGCGT
GACCGACCTGCCCCATACACCGGAATAGCTCCTGGAAACGGGTGGTAATGCCGGA
TGCTCCGACTCCTCGCATGGGGTGTCGGGAAAGATTTCATCGGTATGGGATGGGGT
CGCGTCCTATCAGGTAGTCGGGGGGGTAACGGCCCACCGAGCCTACGACGGGTAG
CCGGCCTGAGAGGGCGACCGGCCACATTGGGACTGAGATACGGCCCAGACTCCTA
CGGGAGGCAGCAGTGGGGAATATTGCACAATGGGCGCAAGCCTGATGCAGCGACG
CCGCGTGCGGGATGACGGCCTTCGGGTTGTAAACCGCTTTTGATCGGGAGCAAGC
CTTCGGGTGAGTGTACCTTTCGAATAAGCACCGGCTAACTACGTGCCAGCAGCCGC
GGTAATACGTAGGGTGCAAGCGTTATCCGGAATTATTGGGCGTAAAGGGCTCGTAG
GCGGTTCGTCGCGTCCGGTGTGAAAGTCCATCGCTTAACGGTGGATCTGCGCCGG
GTACGGGGGGGCTGGAGTGCGGTAGGGGAGACTGGAATTCCCGGTGTAACGGTGG
AATGTGTAGATATCGGGAAGAACACCAATGGCGAAGGCAGGTCTCTGGGCCGTTAC
TGACGCTGAGGAGCGAAAGCGTGGGGAGCGAACAGGATTAGATACCCTGGTAGTC
CACGCCGTAAACGGTGGATGCTGGATGTGGGGCCCGTTCCACGGGTTCCGTGTCG
GAGCTAACGCGTTAAGCATCCCGCCTGGGGAGTACGGCCGCAAGGCTAAAACTCA
AAGAAATTGACGGGGGCCCGCACAAGCGGCGGAGCATGCGGATTAATTCGATGCA
ACGCGAAGAACCTTACCTGGGCTTGACATGTTCCCGACAGCCGTAGAGATATGGCC
TCCCTTCGGGGGGGGTTCACAGGTGGTGCATGGTCGTCGTCAGCTCGTGTCGTGA
GATGTTGGGTTAAGTCCCGCAACGAGCGCAACCCTCGCCCTGTGTTGCCAGCACGT
CATGGTGGGAACTCACGGGGGACCGCCGGGGTCAACTCGGAGGAAGGTGGGGAT
GACGTCAGATCATCATGCCCCTTACGTCCAGGGCTTCACGCATGCTACAATGGCCG
GTACAACGGGATGCGACACGGCGACGTGGAGCGGATCCCTGAAAACCGGTCTCAG
TTCGGATTGGAGTCTGCAACCCGACTCCATGAAGGGGGAGTCGCTAGTAATCGCGG
ATCAGCAACGCCGCGGTGAATGCGTTCCCGGGCCTTGTACACACCGCCCGTCAAG
TCATGAAAGTGGGTAGCACCCGAAGCCGGTGGCCTAACCCTTTGTGGATGGAGCC
GTCTAAGGTGAGACTCGTGATTGGGACTAAGTCGTAACAAGGTAGCCGTACCGGAA
GGTGCGGCTGGATCACC
SEQ ID NO: 33 Anaerostipes caccae Strain 33
GCTTAACACATG-
CAAGTCGAACGAAGCATTTAGGATTGAAGTTTTCGGATGGATTTCCTATATGACTGA
GTGGCGGACGGGTGAGTAACGCGTGGGGAACCTGCCCTATACAGGGGGATAACAG
CTGGAAACGGCTGCTAATACCGCATAAGCGCACAGAATCGCATGATTCAGTGTGAA
AAGCCCTGGCAGTATAGGATGGTCCCGCGTCTGATTAGCTGGTTGGTGAGGTAACG
GCTCACCAAGGCGACGATCAGTAGCCGGCTTGAGAGAGTGAACGGCCACATTGGG
ACTGAGACACGGCCCAAACTCCTACGGGAGGCAGCAGTGGGGAATATTGCACAAT
GGGGG-
AAACCCTGATGCAGCGACGCCGCGTGAGTGAAGAAGTATTTCGGTATGTAAAGCTC
TATCAGCAGGGAAGAAAACAGACGGTACCTGACTAAGAAGCCCCGGCTAACTACGT
GCCAGCAGCCGCGGTAATACGTAGGGGGCAAGCGTTATCCGGAATTACTGGGTGT
AAAGGGTGCGTAGGTGGCATGGTAAGTCAGAAGTGAAAGCCCGGGGCTTAACCCC
GGGACTGCTTTTGAAACTGTCATGCTGGAGTGCAGGAGAGGTAAGCGGAATTCCTA
GTGTAGCGGTGAAATGCGTAGATATTAGGAGGAACACCAGTGGCGAAGGCGGCTTA
CTGGACTGTCACTGACACTGATGCACGAAAGCGTGGGGAGCAAACAGGATTAGATA
CCCTGGTAGTCCACGCCGTAAACGATGAATACTAGGTGTCGGGGCCGTAGAGGCTT
CGGTGCCGCAGCAAACGCAGTAAGTATTCCACCTGGGGAGTACGTTCGCAAGAATG
AAACTCAAAGGAATTGACGGGGACCCGCACAAGCGGTGGAGCATGTGGTTTAATTC
GAAGCAACGCGAAGAACCTTACCTGGTCTTGACATCCCAATGACCGAACCTTAACC
GGTTTTTTCTTTCGAGACATTGGAGACAGGTGGTGCATGGTTGTCGTCAGCTCGTGT
CGTGAGATGTTGGGTTAAGTCCCGCAACGAGCGCAACCCCTATCTTTAGTAGCCAG
CATTTAAGGTGGGCACTCTAGAGAGACTGCCAGGGATAACCTGGAGGAAGGTGGG
GACGACGTCAAATCATCATGCCCCTTATGGCCAGGGCTACACACGTGCTACAATGG
CGTAAACAAAGGGAAGCGAAGTCGTGAGGCGAAGCAAATCCCAGAAATAACGTCTC
AGTTCGGATTGTAGTCTGCAACTCGACTACATGAAGCTGGAATCGCTAGTAATCGTG
AATCAGAATGTCACGGTGAATACGTTCCCGGGTCTTGTACACACCGCCCGTCACAC
CATGGGAGTCAGTAACGCCCGAAGTCAGTGACCCAACCGCAAGGAGGGAGCTGCC
GAAGGTGGGACCGATAACTGGGGTGAAGTCGTAACAAGG
SEQ ID NO: 34 Bifidobacterium adolescentis Strain 34
TGTGGAGGGTTCGATTCTGGCTCAGGATGAACGCTGGCGGCGTGCTTAACACATGC
AAGTCGAACGGGATC-CCAGGAGCTTGCTCCTGG-
GTGAGAGTGGCGAACGGGTGAGTAATGCGTGACCGACCTGCCCCATACACCGGAA
TAGCTCCTGGAAACGGGTGGTAATGCCGGATGCTCCAGTTGGATGCATGTCCTTCT
GGGAAAGATTC-
ATCGGTATGGGATGGGGTCGCGTCCTATCAGCTTGATGGGGGGGTAACGGCCCAC
CATGGCTTCGACGGGTAGCCGGCCTGAGAGGGCGACCGGCCACATTGGGACTGAG
ATACGGCCCAGACTCCTACGGGAGGCAGCAGTGGGGAATATTGCACAATGGGCGC
AAGCCTGATGCAGCGACGCCGCGTGCGGGATGACGGCCTTCGGGTTGTAAACCGC
TTTTGACTGGGAGCAAGCCCTTCGGGGTGAGTGTACCTTTCGAATAAGCACCGGCT
AACTACGTGCCAGCAGCCGCGGTAATACGTAGGGTGCAAGCGTTATCCGGAATTAT
TGGGCGTAAAGGGCTCGTAGGCGGTTCGTCGCGTCCGGTGTGAAAGTCCATCGCT
TAACGGTGGATCCGCGCCGGGTACGGGGGGCTTGAGTGCGGTAGGGGAGACTG
GAATTCCCGGTGTAACGGTGGAATGTGTAGATATCGGGAAGAACACCAATGGCGAA
GGCAGGTCTCTGGGCCGTCACTGACGCTGAGGAGCGAAAGCGTGGGGAGCGAACA
GGATTAGATACCCTGGTAGTCCACGCCGTAAACGGTGGATGCTGGATGTGGGGAC
CATTCCACGGTCTCCGTGTCGGAGCCAACGCGTTAAGCATCCCGCCTGGGGAGTA
CGGCCGCAAGGCTAAAACTCAAAGAAATTGACGGGGGCCCGCACAAGCGGCGGAG
CATGCGGATTAATTCGATGCAACGCGAAGAACCTTACCTGGGCTTGACATGTTCCC
GACAGCCCCAGAGATGGGGCCTCCCTTCGGGGGGGGTTCACAGGTGGTGCATGGT
CGTCGTCAGCTCGTGTCGTGAGATGTTGGGTTAAGTCCCGCAACGAGCGCAACCCT
CGCCCTGTGTTGCCAGCACGTCGTGGTGGGAACTCACGGGGGACCGCCGGGGTCA
ACTCGGAGGAAGGTGGGGATGACGTCAGATCATCATGCCCCTTACGTCCAGGGCTT
CACGCATGCTACAATGGCCGGTACAACGGGATGCGACACT-
GTGAGGTGGAGCGGATCCCTTAAAACCGGTCTCAGTTCGGATTGGAGTCTGCAACC
CGACTCCATGAAGGGGAGTCGCTAGTAATCGCGGATCAGCAACGCCGCGGTGAA
TGCGTTCCCGGGCCTTGTACACACCGCCCGTCAAGTCATGAAAGTGGGTAGCACCC
GAAGCCGGTGGCCCAACCTTTTTGGGGGGAGCCGTCTAAGGTGAGACTCGTGATT
GGGACTAAGTCGTAACAAGGTAGCCGTACCGGAAGGTGCGGCTGGATCACCTCCTT
T
SEQ ID NO: 35 Agathobaculum sp MUR_P3C8a Strain 35
AGTTTGATCCTGGCTCAGGATGAACGCTGGCGGCGTGCCTAACACATGCAAGTCGA
ACGGGGTTATTTTGGAAAATCCTTCGGGATTGGAATTCTT--
AACCTAGTGGCGGACGGGTGAGTAACGCGTGAGCAATCTGCCTTTAAGAGGGGGA
TAACAGTCGGAAACGGCTGCTAATACCGCATAAAGCATTGAATTCGCATGTTTTCGA
TGCCAAAGGAGCAATCCGCTTTTAGATGAGCTCGCGTCTGATTAGCTAGTTGGCGG
GGTAACGGCCCACCAAGGCGACGATCAGTAGCCGGACTGAGAGGTTGAACGGCCA
CATTGGGACTGA-GACACGGCCCAGACTCCTA-
CGGGAGGCAGCAGTGGGGAATATTGCGCAATGGGGGGAACCCTGACGCAGCAACG
CCGCGTGATTGAAGAAGGCCTTCGGGTTGTAAAGATCTTTAATCAGGGACGAAAC--
ATGACGGTACCTGAAGAATAAGCTCCGGCTAACTACGTGCCAGCAGCCGCGGTAAT
ACGTAGGGAGCAAGCGTTATCCGGATTTACTGGGTGTAAAGGGCGCGCAGGGGGG
CCGGCAAGTTGGAAGTGAAATCCGGGGGCTTAACCCCCGAACTGCTTTCAAAACTG
CTGGTCTTGAGTGATGGAGAGGCAGGCGGAATTCCGTGTGTAGCGGTGAAATGCG
TAGATATACGGAGGAACACCAGTGGCGAAGGCGGCCTGCTGGACATTAACTGACG
CTGAGGCGCGAAAGCGTGGGGAGCAAACAGGATTAGATACCCTGGTAGTCCACGC
CGTAAACGATGGATACTAGGTGTGGGAGGTATTGACCCCTTCCGTGCCGCAGTTAA
CACAATAAGTATCCCACCTGGGGAGTACGGCCGCAAGGTTGAAACTCAAAGGAATT
GACGGGGGCCCGCACAAGCAGTGGAGTATGTGGTTTAATTCGAAGCAACGCG-
AAGAACCTTACCAGGCCTTGACATCCCGATGACCGGTCTAGAGATAGACCTTCTCTT
CGGAGCATCGGAGACAGGTGGTGCATGGTTGTCGTCAGCTCGTGTCGTGAGATGTT
GGGTTAAGTCCCGCAACGAGCGCAACCCTTAC-
GGTTAGTTGATACGCAAGATCACTCTAGCCGGACTGCCGTTGACAAAACGGAGGAA
GGTGGGGACGACGTCAAATCATCATGCCCCTTATGGCCTGGGCTACACACGTACTA
CAATGGCAGTCATACAGAGGGAAGCAAAATCGCGAGGTGGAGCAAATCCCTAAAAG
CTGTCCCAGTTCAGATTGCAGGCTGCAACCCGCCTGCATGAAGTCGGAATTGCTAG
TAATCGCGGATCAGCATGCCGCGGTGAATACGTTCCCGGGCCTTGTACACACCGCC
CGTCACACCATGAGAGCCGTCAATACCCGAAGTCCGTAGCCTAACCGTAAGGAGGG
CGCGGCCGAAGGTAGGGGTGGTAATTAGGGTGAAGTCGT
SEQ ID NO: 36 Phascolarctobacterium faecium MUR_P6E9 Strain 36
ATCCTGGCTCAGGACGAACGCTGGCGGCGTGCCTAACACATGCAAGTCGAACGGA
GAATTTTATTTCGGTAGAATTCTTAGTGGCGAAGGGGTGAGTAACGCGTAGGCAAC
CTGCCCTTTAGACGGGGACAACATTCCGAAAGGAGTGCTAATACCGGATGTGATCA
TCGTGCCGCATGGCAGGATGAAGAAAGATGGCCTCTACAAGTAAGCTATCGCTAAA
GGATGGGCCTGCGTCTGATTAGCTAGTTGGTAGTGTAACGGACTACCAAGGCGATG
ATCAGTAGCCGGTCTGAGAGGATGAACGGCCACATTGGGACTGAGACACGGCCCA
AACTCCTACGGGAGGCAGCAGTGGGGAATCTTCCGCAATGGACGAAAGTCTGACG
GAGCAACGCCGCGTGAGTGATGAAGGATTTCGGTCTGTAAAGCTCTGTTGTTTATG
ACGAACGTGCAGTGTGTGAACAATGCATTGCAATGACGGTAGTAAACGAGGAAGCC
ACGGCTAACTACGTGCCAGCAGCCGCGGTAATACGTAGGTGGCGAGCGTTGTCCG
GAATTATTGGGCGTAAAGAGCATGTAGGCGGCTTAATAAGTCGAGCGTGAAAATGC
GGGGCTCAACCCCGTATGGCGCTGGAAACTGTTAGGCTTGAGTGCAGGAGAGGAA
AGGGGAATTCCCAGTGTAGCGGTGAAATGCGTAGATATTGGGAGGAACACCAGTGG
CGAAGGCGCCTTTCTGGACTGTGTCTGACGCTGAGATGCGAAAGCCAGGGTAGCG
AACGGGATTAGATACCCCGGTAGTCCTGGCCGTAAACGATGGGTACTAGGTGTAGG
AGGTATCGACCCCTTCTGTGCCGGAGTTAACGCAATAAGTACCCCGCCTGGGGAGT
ACGGCCGCAAGGTTGAAACTCAAAGGAATTGACGGGGGCCCGCACAAGCGGTGGA
GTATGTGGTTTAATTCGACGCAACGCGAAGAACCTTACCAAGGCTTGACATTGATTG
AACGCTCTAGAGATAGAGCTTTCCCTTCGGGGACAAGAAAACAGGTGGTGCATGGC
TGTCGTCAGCTCGTGTCGTGAGATGTTGGGTTAAGTCCCGCAACGAGCGCAACCCC
TATCCTATGTTACCAGCAAGTAAAGTTGGGGACTCATGGGAGACTGCCAGGGACAA
CCTGGAGGAAGGCGGGGATGACGTCAAGTCATCATGCCCCTTATGTCTTGGGCTAC
ACACGTACTACAATGGTCGGAAACAGAGGGAAGCGAAGCCGCGAGGCAGAGCAAA
CCCCAGAAACCCGATCTCAGTTCGGATCGCAGGCTGCAACCCGCCTGCGTGAAGT
CGGAATCGCTAGTAATCGCAGGTCAGCATACTGCGGTGAATACGTTCCCGGGCCTT
GTACACACCGCCCGTCACACCACGAAAGTTGGTAACACCCGAAGCCGGTGAGGTAA
CCTATAAGGAGCCAGCCGTCTAAGGTGGGGCCGATGATTGGGGTGAAGTCGTAAC
AAGGTAGC
SEQ ID NO: 37 Akkermansia muciniphila Strain 37
AGAGTTTGATTCTGGCTCAGAACGAACGCTGGGGGCGTGGATAAGACATGCAAGTC
GAACGAGAGAATTGCTAGCTTGCTAATAATTCTCTAGTGGCGCACGGGTGAGTAAC
ACGTGAGTAACCTGCCCCCAAGAGTGGGATAGCCCCGGGAAACTGGGATTAATACC
GCATAAAATCGCAAGATTAAAGCAGCAATGCGCTTGGGGATGGGCTCGCGTCCTAT
TAGTTAGTTGGTGAGGTAACGGCTCACCAAGGCGATGACGGGTAGCCGGTCTGAG
AGGATGTCCGGCCACACTGGAACTGAGACACGGTCCAGACACCTACGGGTGGCAG
CAGTCGAGAATCATTCACAATGGGGGAAACCCTGATGGTGCGACGCCGCGTGGGG
GAATGAAGGTCTTCGGATTGTAAACCCCTGTCATGTGGGAGCAAATTAAAAAGATAG
TACCACAAGAGGAAGAGACGGCTAACTCTGTGCCAGCAGCCGCGGTAATACAGAG
GTCTCAAGCGTTGTTCGGAATCACTGGGCGTAAAGCGTGCGTAGGCGGTTTCGTAA
GTCGTGTGTGAAAGGCGGGGGCTCAACCCCCGGACTGCACATGATACTGCGAGAC
TAGAGTAATGGAGGGGGAACCGGAATTCTCGGTGTAGCAGTGAAATGCGTAGATAT
CGAGAGGAACACTCGTGGCGAAGGGGGGTTCCTGGACATTAACTGACGCTGAGGC
ACGAAGGCCAGGGGAGCGAAAGGGATTAGATACCCCTGTAGTCCTGGCAGTAAAC
GGTGCACGCTTGGTGTGCGGGGAATCGACCCCCTGCGTGCCGGAGCTAACGCGTT
AAGCGTGCCGCCTGGGGAGTACGGTCGCAAGATTAAAACTCAAAGAAATTGACGGG
GACCCGCACAAGCGGTGGAGTATGTGGCTTAATTCGATGCAACGCGAAGAACCTTA
CCTGGGCTTGACATGTAATGAACAACATGTGAAAGCATGCGACTCTTCGGAGGCGT
TACACAGGTGCTGCATGGCCGTCGTCAGCTCGTGTCGTGAGATGTTTGGTTAAGTC
CAGCAACGAGCGCAACCCCTGTTGCCAGTTACCAGCACGTAAAGGTGGGGACTCT
GGCGAGACTGCCCAGATCAACTGGGAGGAAGGTGGGGACGACGTCAGGTCAGTAT
GGCCCTTATGCCCAGGGCTGCACACGTACTACAATGCCCAGTACAGAGGGGGCCG
AAGCCGCGAGGCGGAGGAAATCCTAAAAACTGGGCCCAGTTCGGACTGTAGGCTG
CAACCCGCCTACACGAAGCCGGAATCGCTAGTAATGGCGCATCAGCTACGGCGCC
GTGAATACGTTCCCGGGTCTTGTACACACCGCCCGTCACATCATGGAAGCCGGTCG
CACCCGAAGTATCTGAAGCCAACCGCAAGGAGGCAGGGTCCTAAGGTGAGACTGG
TAACTGGGATGAAGTCGTAACAAGGTAGCCGTAGGGGAACCTGCGGCTGGATCAC
CTCCTTT
SEQ ID NO: 38 Barnesiella intestinihomins Strain 38
GATGAACGCTAGCGACAGGCCTAACACATGCAAGTCGAGGGGCAGCGAAGAGGTA
GCAATACCTCTGTCGGCGACCGGCGCACGGGTGAGTAACACGTATGCAATCCACCT
GTAACAGGGGGATAACCCGGAGAAATCCGGACTAATACCCCATAATATGGGCGCTC
CGCATGGAGAGTTCATTAAAGAGAGCAATTTTGGTTACAGACGAGCATGCGCTCCA
TTAGCCAGTTGGGGGGGTAACGGCCCACCAAAGCGACGATGGATAGGGGTTCTGA
GAGGAAGGTCCCCCACATTGGAACTGAGACACGGTCCAAACTCCTACGGGAGGCA
GCAGTGAGGAATATTGGTCAATGGTCGGCAGACTGAACCAGCCAAGTCGCGTGAG
GGAAGACGGCCCTACGGGTTGTAAACCTCTTTTGTCGGAGAGTAAAGTACGCTACG
TGTAGCGTATTGCAAGTATCCGAAGAAAAAGCATCGGCTAACTCCGTGCCAGCAGC
CGCGGTAATACGGAGGATGCGAGCGTTATCCGGATTTATTGGGTTTAAAGGGTGCG
TAGGCGGCACGCCAAGTCAGCGGTGAAATTTCCGGGCTCAACCCGGAGTGTGCCG
TTGAAACTGGCGAGCTAGAGTGCACAAGAGGCAGGCGGAATGCGTGGTGTAGCGG
TGAAATGCATAGATATCACGCAGAACCCCGATTGCGAAGGCAGCCTGCTAGGGTGA
AACAGACGCTGAGGCACGAAAGCGTGGGTATCGAACAGGATTAGATACCCTGGTAG
TCCACGCAGTAAACGATGAATACTAACTGTTTGCGATACAATGTAAGCGGTACAGCG
AAAGCGTTAAGTATTCCACCTGGGGAGTACGCCGGCAACGGTGAAACTCAAAGGAA
TTGACGGGGGCCCGCACAAGCGGAGGAACATGTGGTTTAATTCGATGATACGCGA
GGAACCTTACCCGGGCTCAAACGCAGGGGGAATGTCGGTGAAAGCCGGCAGCTAG
TAATAGTCACCTGCGAGGTGCTGCATGGTTGTCGTCAGCTCGTGCCGTGAGGTGTC
GGCTTAAGTGCCATAACGAGCGCAACCCCTATCGACAGTTACTAACGGGTGAAGCC
GAGGACTCTGTCGAGACTGCCGGCGCAAGCCGCGAGGAAGGTGGGGATGACGTCA
AATCAGCACGGCCCTTACGTCCGGGGCGACACACGTGTTACAATGGCAGGTACAGA
AGGCAGCCAGTCAGCAATGACGCGCGAATCCCGAAAACCTGTCTCAGTTCGGATTG
GAGTCTGCAACCCGACTCCATGAAGCTGGATTCGCTAGTAATCGCGCATCAGCCAT
GGCGCGGTGAATACGTTCCCGGGCCTTGTACACACCGCCCGTCAAGCCATGGAAG
CCGGGAGTACCTGAAGCATGCAACCGCAAGGAGCGTACGAAGGTAATACCGGTAA
CTGGGGCTAAGTCGTAACAAGGTAGCCGTACCGGAAGG
SEQ ID NO: 39 Bilophila wadsworthia Strain 39
AGAGTTTGATTCTGGCTCAGATTGAACGCTGGGGGCGTGCTTAACACATGCAAGTC
GAACGTGAAAG-TCCTTCGGGGC-
GAGTAAAGTGGCGCACGGGTGAGTAACGCGTGGATAATCTACCCTTAAGATGGGGA
TAACGGCTGGAAACGGTCGCTAATACCGAATACGCTCCCGATTTTATCGTTGGGGG
GAAAGATGGCCTCTGCTTGCAAGCTATCGCTTAAGGATGAGTCCGCGTCCCATTAG
CTAGTTGGCGGGGTAACGGCCCACCAAGGCAACGATGGGTAGCCGGTCTGAGAGG
ATGACCGGCCACACTGGAACTGGAACACGGTCCAGACTCCTACGGGAGGCAGCAG
TGGGGAATATTGCGCAATGGGCGAAAGCCTGACGCAGCGACGCCGCGTGAGGGAT
GAAGGTTCTCGGATCGTAAACCTCTGTCAGGGGGGAAGAAACCCCCTCGTGTGAAT
AATGCGAG-
GGCTTGACGGTACCCCCAAAGGAAGCACCGGCTAACTCCGTGCCAGCAGCCGCGG
TAATACGGAGGGTGCAAGCGTTAATCGGAATCACTGGGCGTAAAGCGCACGTAGGC
GGCTTG-
GTAAGTCAGGGGTGAAATCCCACAGCCCAACTGTGGAACTGCCTTTGATACTGC-
CAGGCTTGAGTACCGGAGAGGGTGGCGGAATTCCAGGTGTAGGAGTGAAATCCGT
AGATATCTGGAGGAACACCGGTGGCGAAGGCGGCCACCTGGAC-
GGTAACTGACGCTGAGGTGCGAAAGCGTGGGTAGCAAACAGGATTAGATACCCTG
GTAGTCCACGCTGTAAACGATGGGTGCTGGGTG-CTGGGATGTATGTC-
TCGGTGCCGTAGCTAACGCGATAAGCACCCCGCCTGGGGAGTACGGTCGCAAGGC
TGAAACTCAAAGAAATTGACGGGGGCCCGCACAAGCGGTGGAGTATGTGGTTTAAT
TCGATGCAACGCGAAGAACCTTACCCAGGCTTGACATCTAGGGAACCCTTCGGAAA
TGAAGGGGTGCCCTTCGGGGAGCCCTAAGACAGGTGCTGCATGGCTGTCGTCAGC
TCGTGCCGTGAGGTGTTGGGTTAAGTCCCGCAACGAGCGCAACCCCTATCTTCAGT
TGCCAGCAGGTAAGGCTGGGCACTCTGGAGAGACCGCCCCGGTCAACGGGGAGGA
AGGTGGGGACGACGTCAAGTCATCATGGCCCTTACGCCTGGGGCTACACACGTACT
ACAATGGCGCGCACAAAGGGTAGCGAGACCGCGAGGTGGAGCCAATCCCAAAAAA
CGCGTCCCAGTCCGGATTGGAGTCTGCAACTCGACTCCATGAAGTCGGAATCGCTA
GTAATTCGAGATCAGCATGCTCGGGTGAATGCGTTCCCGGGCCTTGTACACACCGC
CCGTCACACCACGAAAGTCGGTTTTACCCGAAGCCGGTGAGCTAACTCGCAAGAGA
AGCAGCCGTCTACGGTAGGGCCGATGATTGGGGTGAAGTCGTAACAAGGTAGCCG
TAGGGGAACCTGCGGCTGGATCACCTCCTT
SEQ ID NO: 40 Blautia faecis Strain 40
AGAGTTTGATCCTGGCTCAGGATGAACGCTGGCGGCGTGCTTAACACATGCAAGTC
GAACGGGAAATACTTTATTGAAACTTCGGTCGATTTAATTTATTTCTAGTGGCGGAC
GGGTGAGTAACGCGTGGGTAACCTGCCTTATACCGGGGGATAACAGCCAGAAATG
GCTGCTAATACCGCATAAGCGCACAGAACCGCATGGTTCGGTGTGAAAAACTCCGG
TGGTATAAGATGGACCCGCGTTGGATTAGCTAGTTGGCAGGGCAGCGGCCTACCAA
GGCGACGATCCATAGCCGGCCTGAGAGGGTGAACGGCCACATTGGGACTGAGACA
CGGCCCAGACTCCTACGGGAGGCAGCAGTGGGGAATATTGCACAATGGGGGAAAC
CCTGATGCAGCGACGCCGCGTGAAGGAAGAAGTATCTCGGTATGTAAACTTCTATC
AGCAGGGAAGATAATGACGGTACCTGACTAAGAAGCCCCGGCTAACTACGTGCCAG
CAGCCGCGGTAATACGTAGGGGGCAAGCGTTATCCGGATTTACTGGGTGTAAAGG
GAGCGTAGACGGCGCAGCAAGTCTGATGTGAAAGGCAGGGGCTTAACCCCTGGAC
TGCATTGGAAACTGCTGTGCTTGAGTGCCGGAGGGGTAAGCGGAATTCCTAGTGTA
GCGGTGAAATGCGTAGATATTAGGAGGAACACCAGTGGCGAAGGCGGCTTACTGG
ACGGTAACTGACGTTGAGGCTCGAAAGCGTGGGGAGCAAACAGGATTAGATACCCT
GGTAGTCCACGCCGTAAACGATGAATACTAGGTGTCAGGGAGCACAGCTCTTTGGT
GCCGCCGCAAACGCATTAAGTATTCCACCTGGGGAGTACGTTCGCAAGAATGAAAC
TCAAAGGAATTGACGGGGACCCGCACAAGCGGTGGAGCATGTGGTTTAATTCGAAG
CAACGCGAAGAACCTTACCAAATCTTGACATCCCTCTGACCGGGACTTAACCGTCC
CTTTCCTTCGGGACAGGGGAGACAGGTGGTGCATGGTTGTCGTCAGCTCGTGTCGT
GAGATGTTGGGTTAAGTCCCGCAACGAGCGCAACCCCTATCCTTAGTAGCCAGCAC
G-
ATGGTGGGCACTCTGAGGAGACTGCCAGGGATAACCTGGAGGAAGGCGGGGATGA
CGTCAAATCATCATGCCCCTTATGATTTGGGCTACACACGTGCTACAATGGCGTAAA
CAAAGGGAAGCGAACCTGTGAGGGGGGCAAATCTCAAAAATAACGTCCCAGTTCG
GACTGCAGTCTGCAACTCGACTGCACGAAGCTGGAATCGCTAGTAATCGCGGATCA
GAATGCCGCGGTGAATACGTTCCCGGGTCTTGTACACACCGCCCGTCACACCATGG
GAGTCAGTAACGCCCGAAGTCAGTGACCTAACCGCAAGGGAGGAGCTGCCGAAGG
CGGGACCGATGACTGGGGTGAAGTCGTAACAAGGTA
SEQ ID NO: 41 Blautia wexlerae Strain 41
AGAGTTTGATCCTGGCTCAGGATGAACGCTGGGGGCGTGCTTAACACATGCAAGTC
GAACGGGAATTACTTTATTGAAGCTTCGGCAGATTTAGCTGGTTTCTAGTGGCGGAC
GGGTGAGTAACGCGTGGGTAACCTGCCTTGTACAGGGGGATAACAGTCAGAAATGA
CTGCTAATACCGCATAAGCGCACAGGACCGCATGGTCCGGTGTGAAAAACTCCGGT
GGTATAAGATGGACCCGCGTTGGATTAGCTTGTTGGTGGGGTAACGGCCCACCAAG
GCGACGATCCATAGCCGGCCTGAGAGGGTGAACGGCCACATTGGGACTGAGACAC
GGCCCAGACTCCTACGGGAGGCAGCAGTGGGGAATATTGCACAATGGGGGAAACC
CTGATGCAGCGACGCCGCGTGAAGGAAGAAGTATCTCGGTATGTAAACTTCTATCA
GCAGGGAAGAAAATGACGGTACCTGACTAAGAAGCCCCGGCTAACTACGTGCCAG
CAGCCGCGGTAATACGTAGGGGGCAAGCGTTATCCGGATTTACTGGGTGTAAAGG
GAGCGTAGACGGATGGA-
CAAGTCTGATGTGAAAGGCTGGGGCTCAACCCCGGGACTGCATTGGAAACTGCCC
GTCTTGAGTGCCGGAGAGGTAAGCGGAATTCCTAGTGTAGCGGTGAAATGCGTAGA
TATTAGGAGGAACACCAGTGGCGAAGGCGGCTTACTGGACGGTAACTGACGTTGAG
GCTCGAAAGCGTGGGGAGCAAACAGGATTAGATACCCTGGTAGTCCACGCGGTAAA
CGATGAATGCTAGGTGTCGGGTGACAAAGTCATTCGGTGCCGCCGCAAACGCATTA
AGCATTCCACCTGGGGAGTACGTTCGCAAGAATGAAACTCAAAGGAATTGACGGGG
ACCCGCACAAGCGGTGGAGCATGTGGTTTAATTCGAAGCAACGCGAAGAACCTTAC
CAAGTCTTGACATCCCTCTGACCGGAACTTAACCGTTCCTTCCCTTCGGGGCAGAG
GAGACAGGTGGTGCATGGTTGTCGTCAGCTCGTGTCGTGAGATGTTGGGTTAAGTC
CCGCAACGAGCGCAACCCCTATCCCCAGTAGCCAGCATTTAAGGTGGGCACTCTGG
GGAGACTGCCAGGGATAACCTGGAGGAAGGGGGGGATGACGTCAAATCATCATGC
CCCTTATGATTTGGGCTACACACGTGCTACAATGGCGTAAACAAAGGGAAGCGAAC
CCGCGAGGGTGGGCAAATCCCAAAAATAACGTCCCAGTTCGGACTGTAGTCTGCAA
CCCGACTACACGAAGCTGGAATCGCTAGTAATCGCGGATCAGAATGCCGCGGTGAA
TACGTTCCCGGGTCTTGTACACACCGCCCGTCACACCATGGGAGTCAGTAACGCCC
GAAGTCAGTGACCTAACCGTAAGGAGGGAGCTGCCGAAGGCGGGACCGATGACTG
GGGTGAAGTCGTAACAAGGTA
SEQ ID NO: 42 Blautia luti Strain 42
CTGGCTCAGGATGAACGCTGGGGGCGTGCTTAACACATGCAAGTCGAACGGGAAAT
ATTTCATTGAGACTTCGGTGGATTTGATCTATTTCTAGTGGGGGACGGGTGAGTAAC
GCGTGGGTAACCTGCCTTATACAGGGGGATAACAGTCAGAAATGGCTGCTAATACC
GCATAAGCGCACAGAGCTGCATGGCTCAGTGTGAAAAACTCCGGTGGTATAAGATG
GACCCGCGTTGGATTAGCTGGTTGGTGGGGTAACGGCCCACCAAGGCGACGATCC
ATAGCCGGCCTGAGAGGGTGAACGGCCACATTGGGACTGAGACACGGCCCAGACT
CCTACGGGAGGCAGCAGTGGGGAATATTGCACAATGGGGGAAACCCTGATGCAGC
GACGCCGCGTGAAGGAAGAAGTATCTCGGTATGTAAACTTCTATCAGCAGGGAAGA
TAGTGACGGTACCTGACTAAGAAGCCCCGGCTAACTACGTGCCAGCAGCCGCGGT
AATACGTAGGGGGCAAGCGTTATCCGGATTTACTGGGTGTAAAGGGAGCGTAGACG
GTGTGGCAAGTCTGATGTGAAAGGCATGGGCTCAACCTGTGGACTGCATTGGAAAC
TGTCATACTTGAGTGCCGGAGGGGTAAGCGGAATTCCTAGTGTAGCGGTGAAATGC
GTAGATATTAGGAGGAACACCAGTGGCGAAGGCGGCTTACTGGACGGTAACTGAC
GTTGAGGCTCGAAAGCGTGGGGAGCAAACAGGATTAGATACCCTGGTAGTCCACG
CCGTAAACGATGAATACTAGGTGTCGGGGAGTATGGCTCTTCGGTGCCGTCGCAAA
CGCAGTAAGTATTCCACCTGGGGAGTACGTTCGCAAGAATGAAACTCAAAGGAATT
GACGGGGACCCGCACAAGCGGTGGAGCATGTGGTTTAATTCGAAGCAACGCGAAG
AACCTTACCAAGTCTTGACATCCGCCTGACCGGTCCTTAACCGGACCTTTCCTTCGG
GACAGGAGAGACAGGTGGTGCATGGTTGTCGTCAGCTCGTGTCGTGAGATGTTGG
GTTAAGTCCCGCAACGAGCGCAACCCCTATCCTCAGTAGCCAGCAT-
TTAAGGTGGGCACTCTGGGGAGACTGCCAGGGATAACCTGGAGGAAGGCGGGGAT
GACGTCAAATCATCATGCCCCTTATGATTTGGGCTACACACGTGCTACAATGGCGTA
AACAAAGGGAAGCGAGATCGTGAGATGGAGCAAATCCCAAAAATAACGTCCCAGTT
CGGACTGTAGTCTGCAACCCGACTACACGAAGCTGGAATCGCTAGTAATCGCGGAT
CAGAATGCCGCGGTGAATACGTTCCCGGGTCTTGTACACACCGCCCGTCACACCAT
GGGAGTCAGTAACGCCCGAAGTCAGTGACCTAACTGCAAAGAAGGAGCTGCCGAA
GGCGGGACCGATGACTGGGGTGAAGTCGTAACAAGGTA
SEQ ID NO: 43 Butyricimonas synergistica Strain 43
AGAGTTTGATCCTGGCTCAGGATGAACGCTAGCGACAGGCTTAACACATGCAAGTC
GAGGGGCAGCACGGTGTAGCAATACACTGGTGGCGACCGGCGCACGGGTGAGTAA
CACGTGTGCAACCAACCCCGTACCGGGAGATAACCCGCGGAAACGTGGACTAACAT
CCCATAAGACTCTAGAGCCGCATGGCTCTGGATTTAAAATTCCGGTGGTACGGGAC
GGGCACGCGCGACATTAGGTAGTTGGCGGGGTAACGGCCCACCAAGCCGACGATG
TCTAGGGGTTCTGAGAGGAAGGTCCCCCACACTGGAACTGAGACACGGTCCAGACT
CCTACGGGAGGCAGCAGTGAGGAATATTGGTCAATGGGCGCGAGCCTGAACCAGC
CAAGTCGCGTGAGGGAAGAATGGTCTATGGCCTGTAAACCTCTTTTGTCAAGGAAG
AATAAAAGGT-ACGTGTACCTTC-
TTGCCAGTACTTGACGAATAAGCATCGGCTAACTCCGTGCCAGCAGCCGCGGTAAT
ACGGGGGATGCGAGCGTTATCCGGATTTATTGGGTTTAAAGGGCGCGTAGGGGGG
ACGCCAAGTCAGCGGTAAAAGACTGCAGCTAAACTGTAGCACGCCGTTGAAACTGG
CGACCTGGAGACGAGACGAGGGAGGCGGAACAAGTGAAGTAGCGGTGAAATGCTT
AGATATCACTTGGAACCCCGATAGCGAAGGCAGCTTCCCAGGCTCGATCTGACGCT
GATGCGCGAGAGCGTGGGTAGCGAACAGGATTAGATACCCTGGTAGTCCACGCCG
TAAACGATGCTCACTGGATCTTGGCGATACACTGCCAGGGTTCAAGCGAAAGTATT
AAGTGAGCCACCTGGGGAGTACGTCGGCAACGATGAAACTCAAAGGAATTGACGG
GGGCCCGCACAAGCGGAGGAACATGTGGTTTAATTCGATGATACGCGAGGAACCTT
ACCCGGGTTTAAATGTATTTTGCATTTCTTGGAAACAGGAATTCCCTTCGGGGCTAG
ATACAAGGTGCTGCATGGTTGTCGTCAGCTCGTGCCGTGAGGTGTCGGGTTAAGTC
CCATAACGAGCGCAACCCCTATCGCCAGTTGCCATCGGTTGAAGCCGGGCACTCTG
TCGAGACTGCCACCGTAAGGTGCGAGGAAGGGGGGGATGACGTCAAATCAGCACG
GCCCTTACACCCGGGGCGACACACGTGTTACAATGGCCGGTACAGAGGGCAGCCA
CGGGGTGACCCGGAGCGAATCTCTAAAGCCGGTCGTAGTTCGGACTGGAGTCTGC
AACCCGACTCCACGAAGTTGGATTCGCTAGTAATCGCGCATCAGCCATGGCGCGGT
GAATACGTTCCCGGGCCTTGTACACACCGCCCGTCAAGCCATGGAAGCCGGGAGT
ACCTGAAGATCGTGACCGCGAGGAACGGGCTAGGGTAATACCGGTAACTGGGGCT
AAGTCGTAACAAGGTA
SEQ ID NO: 44 Clostridium bolteae Strain 44
AGAGTTTGATCCTGGCTCAGGATGAACGCTGGCGGCGTGCCTAACACATGCAAGTC
GAACGAAGCAATT-AAAGGAAGTTTTCGGATGGA-
ATTTGATTGACTGAGTGGCGGACGGGTGAGTAACGCGTGGATAACCTGCCTCACAC
TGGGGGATAACAGTTAGAAATGACTGCTAATACCGCATAAGCGCACAGTACCGCAT
GGTACGGTGTGAAAAACTCCGGTGGTGTGAGATGGATCCGCGTCTGATTAGCCAGT
TGGCGGGGTAACGGCCCACCAAAGCGACGATCAGTAGCCGACCTGAGAGGGTGAC
CGGCCACATTGGGACTGAGACACGGCCCAAACTCCTACGGGAGGCAGCAGTGGGG
AATATTGCACAATGGGCGAAAGCCTGATGCAGCGACGCCGCGTGAGTGAAGAAGTA
TTTCGGTATGTAAAGCTCTATCAGCAGGGAAGAAAATGACGGTACCTGACTAAGAA
GCCCCGGCTAACTACGTGCCAGCAGCCGCGGTAATACGTAGGGGGCAAGCGTTAT
CCGGATTTACTGGGTGTAAAGGGAGCGTAGACGGCGAAGCAAGTCTGAAGTGAAAA
CCCAGGGCTCAACCCTGGGACTGCTTTGGAAACTGTTTTGCTAGAGTGTCGGAGAG
GTAAGTGGAATTCCTAGTGTAGCGGTGAAATGCGTAGATATTAGGAGGAACACCAG
TGGCGAAGGCGGCTTACTGGACGATAACTGACGTTGAGGCTCGAAAGCGTGGGGA
GCAAACAGGATTAGATACCCTGGTAGTCCACGCCGTAAACGATGAATGCTAGGTGT
TGGGGGGCAAAGCCCTTCGGTGCCGTCGCAAACGCAGTAAGCATTCCACCTGGGG
AGTACGTTCGCAAGAATGAAACTCAAAGGAATTGACGGGGACCCGCACAAGCGGTG
GAGCATGTGGTTTAATTCGAAGCAACGCGAAGAACCTTACCAAGTCTTGACATCCTC
TTGGCCGGCGTGTAACGGCGCCTTCCCTTCGGGGCAAGAGAGACAGGTGGTGCAT
GGTTGTCGTCAGCTCGTGTCGTGAGATGTTGGGTTAAGTCCCGCAACGAGCGCAAC
CCTTATCCTTAGTAGCCAGCAGGTAGAGCTGGGCACTCTAGGGAGACTGCCAGGGA
TAACCTGGAGGAAGGTGGGGATGACGTCAAATCATCATGCCCCTTATGATTTGGGC
TACACACGTGCTACAATGGCGTAAACAAAGGGAAGCAAGACAGTGATGTGGAGCAA
ATCCCAAAAATAACGTCCCAGTTCGGACTGTAGTCTGCAACCCGACTACACGAAGC
TGGAATCGCTAGTAATCGCGAATCAGAATGTCGCGGTGAATACGTTCCCGGGTCTT
GTACACACCGCCCGTCACACCATGGGAGTCAGCAACGCCCGAAGTCAGTGACCCA
ACTCGCAAGAGAGGGAGCTGCCGAAGGCGGGGCAGGTAACTGGGGTGAAGTCGTA
ACAAGGTAGCCGTA
SEQ ID NO: 45 Parasutterella excrementihominis Strain 45
ATTGAACGCTGGCGGAACGCTTTACACATGCAAGTCGAACGGTAACGTGGGGAGGA
GCTTGCTCCACCCCGACGACGAGTGGCGAACGGGTGAGTAATACATCGGAACGTG
TCCGCTTGTGGGGGACAACCAGCCGAAAGGTTGGCTAATACCGCATGAGTTCTACG
GAAGAAAGAGGGGGACCCGCAAGGGCCTCTCGCGAGCGGAGCGGCCGATGACTG
ATTAGCCGGTTGGTGAGGTAACGGCTCACCAAAGCAACGATCAGTAGCTGGTCTGA
GAGGACGACCAGCCACACTGGGACTGAGACACGGCCCAGACTCCTACGGGAGGCA
GCAGTGGGGAATTTTGGACAATGGGCGCAAGCCTGATCCAGCTATTCCGCGTGTGG
GATGAAGGCCCTCGGGTTGTAAACCACTTTTGTAGAGAACGAAAAGACACCATCGA
ATAAATGGTGTTGCTGACGGTACTCTAAGAATAAGCACCGGCTAACTACGTGCCAG
CAGCCGCGGTAATACGTAGGGTGCGAGCGTTAATCGGAATTACTGGGCGTAAAGG
GTGCGCAGGCGGTTGAGTAAGACAGATGTGAAATCCCCGAGCTTAACTCGGGAATG
GCATATGTGACTGCTCGACTAGAGTGTGTCAGAGGGAGGTGGAATTCCACGTGTAG
CAGTGAAATGCGTAGATATGTGGAAGAACACCGATGGCGAAGGCAGCCTCCTGGG
ACATAACTGACGCTCAGGCACGAAAGCGTGGGGAGCAAACAGGATTAGATACCCTG
GTAGTCCACGCCCTAAACGATGTTAACTAGTTGTTGGGATGTAACAATCTCAGTAAC
GCAGCCAACGCGAGAAGTTAACCGCCTGGGAAGTACGGTCGCAAGACTAAAACTCA
AAGGAATTGACGGGGACCCGCACAAGCGGTGGATGATGTGGATTAATTCGATGCAA
CGCGAAAAACCTTACCTACCCTTGACATGTCAGGAAGCTCTTGTAATGAGAGCGTG
CCCGCAAGGGAGCCTGAACACAGGTGCTGCATGGCTGTCGTCAGCTCGTGTCGTG
AGATGTTGGGTTAAGTCCCGCAACGAGCGCAACCCTTGTCACTAGTTGCTACGAAA
GGGCACTCTAGTGAGACTGCCGGTGACAAACCGGAGGAAGGTGGGGATGACGTCA
AGTCCTCATGGCCCTTATGGGTAGGGCTTCACACGTCATACAATGGTCGGAACAGA
GGGCAGCGAAGCCGTGAGGCGGAGCCAATCCCAGAAAACCGATCGTAGTCCGGAT
TGCAGTCTGCAACTCGACTGCATGAAGTCGGAATCGCTAGTAATCGCGGATCAGCA
TGCCGCGGTGAATACGTTCCCGGGTCTTGTACACACCGCCCGTCAAACAATGGGAG
TGGTGTTTACCAGAAGTCGTTAGCCTAACCGCAAGGAGGGCGGCGACCACGGTGA
GCACCGTGACTAATGTTAAGTCGTAACAAGGTAGCCGTACCGGAAGG
SEQ ID NO: 46 Prevotella copri Strain 46
AGAGTTTGATCCTGGCTCAGGATGAACGCTAGCTACAGGCTTAACACATGCAAGTC
GAGGGGAAACGATATTGGAAGCTTGCTTCCGATAGGCGTCGACCGGCGCACGGGT
GAGTAACGCGTATCCAACCTGCCCACCACTTGGGGATAACCTTGCGAAAGTAAGAC
TAATACCCAATGATATCTCTAGAAGACATCTGAAAGAGATTAAAGATTTATCGGTGAT
GGATGGGGATGCGTCTGATTAGCTTGTTGGCGGGGTAACGGCCCACCAAGGCGAC
GATCAGTAGGGGTTCTGAGAGGAAGGTCCCCCACATTGGAACTGAGACACGGTCCA
AACTCCTACGGGAGGCAGCAGTGAGGAATATTGGTCAATGGGCGAGAGCCTGAAC
CAGCCAAGTAGCGTGCAGGATGACGGCCCTATGGGTTGTAAACTGCTTTTATAAGG
GAATAAAGTGAGAGTCGTGACTCTTTTTGCATGTACCTTATGAATAAGGACCGGCTA
ATTCCGTGCCAGCAGCCGCGGTAATACGGAAGGTCCGGGCGTTATCCGGATTTATT
GGGTTTAAAGGGAGCGTAGGCCGGAGATTAAGCGTGTTGTGAAATGTAGACGCTCA
ACGTCTGCACTGCAGCGCGAACTGGTTTCCTTGAGTACGCACAAAGTGGGCGGAAT
TCGTGGTGTAGCGGTGAAATGCTTAGATATCACGAAGAACTCCGATTGCGAAGGCA
GCTCACTGGAGCGCAACTGACGCTGAAGCTCGAAAGTGCGGGTATCGAACAGGATT
AGATACCCTGGTAGTCCGCACGGTAAACGATGGATGCCCGCTGTTGGTCTGAATAG
GTCAGCGGCCAAGCGAAAGCATTAAGCATCCCACCTGGGGAGTACGCCGGCAACG
GTGAAACTCAAAGGAATTGACGGGGGCCCGCACAAGCGGAGGAACATGTGGTTTAA
TTCGATGATACGCGAGGAACCTTACCCGGGCTTGAATTGCAGAGGAAGGATTTGGA
GACAATGACGCCCTTCGGGGCCTCTGTGAAGGTGCTGCATGGTTGTCGTCAGCTCG
TGCCGTGAGGTGTCGGCTTAAGTGCCATAACGAGCGCAACCCCTCTCCTTAGTTGC
CATCAGGTTAAGCTGGGCACTCTGGGGGCACTGCCACCGTAAGGTGTGAGGAAGG
TGGGGATGACGTCAAATCAGCACGGCCCTTACGTCCGGGGCTACACACGTGTTACA
ATGGCAGGTACAGAGAGATGGTCCCTTGCAAAACGGATCAAATCCTTAAAGCCTGT
CTCAGTTCGGACTGGGGTCTGCAACCCGACCCCACGAAGCTGGATTCGCTAGTAAT
CGCGCATCAGCCATGGCGCGGTGAATACGTTCCCGGGCCTTGTACACACCGCCCG
TCAAGCCATGAAAGCCGGGGGCGCCTAAAGTCCGTGACCGTAAGGAGCGGCCTAG
GGCGAAACTGGTAATTGGGGCTAAGTCGTAACAAGGTA
SEQ ID NO: 47 Ruminococcus faecis Strain 47
TTTGATCCTGGCTCAGGATGAACGCTGGGGGCGTGCTTAACACATGCAAGTCGAAC
GAAGCACCTTGATTTGA-TTCTTCGG-
ATGAAGATCCTGGTGACTGAGTGGCGGACGGGTGAGTAACGCGTGGGTAACCTGC
CTCATACAGGGGGATAACAGTTAGAAATGACTGCTAATACCGCATAAGACCACAGC
ACCGCATGGTGCAGGGGTAAAAACTCCGGTGGTATGAGATGGACCCGCGTCTGATT
AGGTAGTTGGTGGGGTAACGGCCTACCAAGCCGACGATCAGTAGCCGACCTGAGA
GGGTGACCGGCCACATTGGGACTGAGACACGGCCCAAACTCCTACGGGAGGCAGC
AGTGGGGAATATTGCACAATGGGGGAAACCCTGATGCAGCGACGCCGCGTGAGCG
ATGAAGTATTTCGGTATGTAAAGCTCTATCAGCAGGGAAGAAAATGACGGTACCTGA
CTAAGAAGCACCGGCTAAATACGTGCCAGCAGCCGCGGTAATACGTATGGTGCAAG
CGTTATCCGGATTTACTGGGTGTAAAGGGAGCGTAGACGGAGTGGCAAGTCTGATG
TGAAAACCCGGGGCTCAACCCCGGGACTGCATTGGAAACT--
GTCAATCTAGAGTACCGGAGAGGTAAGCGGAATTCCTAGTGTAGCGGTGAAATGCG
TAGATATTAGGAGGAACACCAGTGGCGAAGGCGGCTTACTGGACGGTAACTGACGT
TGAGGCTCGAAAGCGTGGGGAGCAAACAGGATTAGATACCCTGGTAGTCCACGCC
GTAAACGATGACTACTAGGTGTCGGGCAGCAAAGCTGTTCGGTGCCGCAGCAAACG
CAATAAGTAGTCCACCTGGGGAGTACGTTCGCAAGAATGAAACTCAAAGGAATTGA
CGGGGACCCGCACAAGCGGTGGAGCATGTGGTTTAATTCGAAGCAACGCGAAGAA
CCTTACCTGCTCTTGACATCTCCCTGACCGGCAAGTAATGTTGCCTTTCCTTCGGGA
CAG-
GGATGACAGGTGGTGCATGGTTGTCGTCAGCTCGTGTCGTGAGATGTTGGGTTAAG
TCCCGCAACGAGCGCAACCCCTATCTTTAGTAGCCAGCGGTCAGGCCGGGCACTCT
AGAGAGACTGCCAGGGATAACCTGGAGGAAGGTGGGGATGACGTCAAATCATCAT
GCCCCTTATGAGCAGGGCTACACACGTGCTACAATGGCGTAAACAAAGGGAGGCA
GAACCGCGAGGTCGAGCAAATCCCAAAAATAACGTCTCAGTTCGGATTGTAGTCTG
CAACTCGACTACATGAAGCTGGAATCGCTAGTAATCGCGAATCAGAATGTCGCGGT
GAATACGTTCCCGGGTCTTGTACACACCGCCCGTCACACCATGGGAGTCAGTAACG
CCCGAAGTCAGTGACCCAACCGTAAGGAGGGAGCTGCCGAAGGTGGGACCGATAA
CTGGGGTGAAGTCGTAACAAGGTAGCCGTATCGGAAGGTGCGGCTGGATCACCTC
CTTT
SEQ ID NO: 48 Bacteroides caccae
TGGCTCAGGATGAACGCTAGCTACAGGCTTAACACATGCAAGTCGAGGGGCATCAG
TTTGGTTTGCTTGCAAACCAAAGCTGGCGACCGGCGCACGGGTGAGTAACACGTAT
CCAACCTGCCTCATACTCGGGGATAGCCTTTCGAAAGAAAGATTAATATCCGATAGC
ATATATTTCCCGCATGGGTTTTATATTAAAGAAATTCGGTATGAGATGGGGATGCGT
TCCATTAGTTTGTTGGGGGGGTAACGGCCCACCAAGACTACGATGGATAGGGGTTC
TGAGAGGAAGGTCCCCCACATTGGAACTGAGACACGGTCCAAACTCCTACGGGAG
GCAGCAGTGAGGAATATTGGTCAATGGACGCGAGTCTGAACCAGCCAAGTAGCGTG
AAGGATGACTGCCCTATGGGTTGTAAACTTCTTTTATATGGGAATAAAGTTGTCCAC
GTGTGGATTTTTGTATGTACCATATGAATAAGGATCGGCTAACTCCGTGCCAGCAGC
CGCGGTAATACGGAGGATCCGAGCGTTATCCGGATTTATTGGGTTTAAAGGGAGCG
TAGGCGGATTGTTAAGTCAGTTGTGAAAGTTTGCGGCTCAACCGTAAAATTGCAGTT
GATACTGGCAGTCTTGAGTGCAGTAGAGGTGGGCGGAATTCGTGGTGTAGCGGTG
AAATGCTTAGATATCACGAAGAACTCCGATTGCGAAGGCAGCCACTGGAGTGTAAC
TGACGCTGATGCTCGAAAGTGTGGGTATCAAACAGGATTAGATACCCTGGTAGTCC
ACACAGTAAACGATGAATACTCGCTGTTTGCGATATACAGTAAGCGGCCAAGCGAA
AGCATTAAGTATTCCACCTGGGGAGTACGCCGGCAACGGTGAAACTCAAAGGAATT
GACGGGGGCCCGCACAAGCGGAGGAACATGTGGTTTAATTCGATGATACGCGAGG
AACCTTACCCGGGCTTAAATTGCAAATGAATTATGGGGAAACCCATAGGCCGCAAG
GCATTTGTGAAGGTGCTGCATGGTTGTCGTCAGCTCGTGCCGTGAGGTGTCGGCTT
AAGTGCCATAACGAGCGCAACCCTTATCTTCAGTTACTAACAGGTCATGCTGAGGA
CTCTGGAGAGACTGCCGTCGTAAGATGTGAGGAAGGTGGGGATGACGTCAAATCA
GCACGGCCCTTACGTCCGGGGCTACACACGTGTTACAATGGGGGGTACAGAAGGC
CGCTACCTGGTGACAGGATGCCAATCCCAAAAACCTCTCTCAGTTCGGATCGAAGT
CTGCAACCCGACTTCGTGAAGCTGGATTCGCTAGTAATCGCGCATCAGCCATGGCG
CGGTGAATACGTTCCCGGGCCTTGTACACACCGCCCGTCAAGCCATGAAAGCCGG
GGGTACCTGAAGTACGTAACCGCAAGGAGCGTCCTAGGGTAAAACTGGTAATTGGG
GCTAAGTCGTAACAAGGTA
SEQ ID NO: 49 Bacteroides cellulosilyticus/ Bacteroides intestinalis
AGAGTTTGATCCTGGCTCAGGATGAACGCTAGCTACAGGCTTAACACATGCAAGTC
GAGGGGCAGCATGACCTAGCAATAGGTTGATGGCGACCGGCGCACGGGTGAGTAA
CACGTATCCAACCTACCGGTTATTCCGGGATAGCCTTTCGAAAGAAAGATTAATACC
GGATAGTATAACGAGAAGGCATCTTTTTGTTATTAAAGAATTTCGATAACCGATGGG
GATGCGTTCCATTAGTTTGTTGGCGGGGTAACGGCCCACCAAGACATCGATGGATA
GGGGTTCTGAGAGGAAGGTCCCCCACATTGGAACTGAGACACGGTCCAAACTCCTA
CGGGAGGCAGCAGTGAGGAATATTGGTCAATGGACGAGAGTCTGAACCAGCCAAG
TAGCGTGAAGGATGACTGCCCTATGGGTTGTAAACTTCTTTTATATGGGAATAAAGT
GAGCCACGTGTGGCTTTTTGTATGTACCATACGAATAAGGATCGGCTAACTCCGTG
CCAGCAGCCGCGGTAATACGGAGGATCCGAGCGTTATCCGGATTTATTGGGTTTAA
AGGGAGCGTAGGCGGACTATTAAGTCAGCTGTGAAAGTTTGCGGCTCAACCGTAAA
ATTGCAGTTGATACTGGTCGTCTTGAGTGCAGTAGAGGTAGGCGGAATTCGTGGTG
TAGCGGTGAAATGCTTAGATATCACGAAGAACTCCGATTGCGAAGGCAGCTTACTG
GACTGTAACTGACGCTGATGCTCGAAAGTGTGGGTATCAAACAGGATTAGATACCC
TGGTAGTCCACACAGTAAACGATGAATACTCGCTGTTTGCGATATACGGCAAGCGG
CCAAGCGAAAGCATTAAGTATTCCACCTGGGGAGTACGCCGGCAACGGTGAAACTC
AAAGGAATTGACGGGGGCCCGCACAAGCGGAGGAACATGTGGTTTAATTCGATGAT
ACGCGAGGAACCTTACCCGGGCTTAAATTGCAAATGAATATAGTGGAAACATTATAG
CCGCAAGGCATTTGTGAAGGTGCTGCATGGTTGTCGTCAGCTCGTGCCGTGAGGTG
TCGGCTTAAGTGCCATAACGAGCGCAACCCTTATCTTTAGTTACTAACAGGTCATGC
TGAGGACTCTAGAGAGACTGCCGTCGTAAGATGTGAGGAAGGTGGGGATGACGTC
AAATCAGCACGGCCCTTACGTCCGGGGCTACACACGTGTTACAATGGGGGGTACAG
AAGGCAGCTACACAGCGATGTGATGCTAATCCCAAAAGCCTCTCTCAGTTCGGATT
GGAGTCTGCAACCCGACTCCATGAAGCTGGATTCGCTAGTAATCGCGCATCAGCCA
CGGCGCGGTGAATACGTTCCCGGGCCTTGTACACACCGCCCGTCAAGCCATGAAA
GCCGGGGGTACCTGAAGTCCGTAACCGTAAGGAGCGGCCTAGGGTAAAACTGGTA
ATTGGGGCTAAGTCGTA
SEQ ID NO: 50 Bacteroides faecis
CTCAGGATGAACGCTAGCTACAGGCTTAACACATGCAAGTCGAGGGGCAGCATTTC
AGTTTGCTTGCAAACTGGAGATGGCGACCGGCGCACGGGTGAGTAACACGTATCCA
ACCTGCCGATAACTCGGGGATAGCCTTTCGAAAGAAAGATTAATACCCGATGGCAT
AATAGAACCGCATGGTTTTTTTATTAAAGAATTTCGGTTATCGATGGGGATGCGTTC
CATTAGGCAGTTGGTGAGGTAACGGCTCACCAAACCTTCGATGGATAGGGGTTCTG
AGAGGAAGGTCCCCCACATTGGAACTGAGACACGGTCCAAACTCCTACGGGAGGC
AGCAGTGAGGAATATTGGTCAATGGACGAGAGTCTGAACCAGCCAAGTAGCGTGAA
GGATGACTGCCCTATGGGTTGTAAACTTCTTTTATATGGGAATAAAGTTTTCCACGT
GTGGAATTTTGTATGTACCATATGAATAAGGATCGGCTAACTCCGTGCCAGCAGCC
GCGGTAATACGGAGGATCCGAGCGTTATCCGGATTTATTGGGTTTAAAGGGAGCGT
AGGTGGACAGTTAAGTCAGTTGTGAAAGTTTGCGGCTCAACCGTAAAATTGCAGTT
GATACTGGCTGTCTTGAGTACAGTAGAGGTGGGGGGAATTCGTGGTGTAGCGGTGA
AATGCTTAGATATCACGAAGAACTCCGATTGCGAAGGCAGCTCACTGGACTGCAAC
TGACACTGATGCTCGAAAGTGTGGGTATCAAACAGGATTAGATACCCTGGTAGTCC
ACACAGTAAACGATGAATACTCGCTGTTTGCGATATACAGTAAGCGGCCAAGCGAA
AGCATTAAGTATTCCACCTGGGGAGTACGCCGGCAACGGTGAAACTCAAAGGAATT
GACGGGGGCCCGCACAAGCGGAGGAACATGTGGTTTAATTCGATGATACGCGAGG
AACCTTACCCGGGCTTAAATTGCATTTGAATATATTGGAAACAGTATAGTCGTAAGA
CAAATGTGAAGGTGCTGCATGGTTGTCGTCAGCTCGTGCCGTGAGGTGTCGGCTTA
AGTGCCATAACGAGCGCAACCCTTATCTTTAGTTACTAACAGGTCATGCTGAGGACT
CTAGAGAGACTGCCGTCGTAAGATGTGAGGAAGGTGGGGATGACGTCAAATCAGCA
CGGCCCTTACGTCCGGGGCTACACACGTGTTACAATGGGGGGTACAGAAGGCAGC
TACCTGGTGACAGGATGCTAATCCCAAAAGCCTCTCTCAGTTCGGATCGAAGTCTG
CAACCCGACTTCGTGAAGCTGGATTCGCTAGTAATCGCGCATCAGCCATGGCGCGG
TGAATACGTTCCCGGGCCTTGTACACACCGCCCGTCAAGCCATGAAAGCCGGGGG
TACCTGAAGTACGTAACCGCAAGGAGCGTCCTAGGGTAAAACTGGTAATTGGGGCT
AAGTCGTAACAAGGTA
SEQ ID NO: 51 Bacteroides ovatus
CGATATCCGGATTTATTGGAGTTT-
AAGGGAGCGTAGGTGGATTGTTAAGTCAGTTGTGAAAGTTTGCGGCTCAACCGTAA
AATTGCAGTTGAAACTGGCAGTCTTGAGTACAGTAGAGGTGGGCGGAATTCGTGGT
GTAGCGGTGAAATGCTTAGATATCACGAAGAACTCCGATTGCGAAGGCAGCTCACT
AGACTGTCACTGACACTGATGCTCGAAAGTGTGGGTATCAAACAGGATTAGATACC
CTGGTAGTCCACACAGTAAACGATGAATACTCGCTGTTTGCGATATACAGTAAGCG
GCCAAGCGAAAGCATTAAGTATTCCACCTGGGGAGTACGCCGGCAACGGTGAAACT
CAAAGGAATTGACGGGGGCCCGCACAAGCGGAGGAACATGTGGTTTAATTCGATGA
TACGCGAGGAACCTTACCCGGGCTTAAATTGCAACAGAATATATTGGAAACAGTATA
GCCGTAAGGCTGTTGTGAAGGTGCTGCATGGTTGTCGTCAGCTCGTGCCGTGAGGT
GTCGGCTTAAGTGCCATAACGAGCGCAACCCTTATCTTTAGTTACTAACAGGTKATG
CTGAGGACTCTAGAGAGACTGCCGTCGTAAGATGTGAGGAAGGTGGGGATGACGT
CAAATCAGCACGGCCCTTACGTCCGGGGCTACACACGTGTTACAATGGGGGGTACA
GAAGGCSGCTACCTGGTGACAGGATGCTAATCCCAAAAACCTCTCTCAGTTCGGAT
CGAAGTCTGCAACCCGACTTCGTGAAGCTGGATTCGCTAGTAATCGCGCATCAGCC
ATGGCGCGGTGAATACGTTCCCGGGCCTTGTACACACCGCCCGTCAAGCCATGAAA
GCCGGGGGTACCTGAAGTACGTAACCGCAAGGAGCGTCCTAGGGGTAAAACTGGT
AATTGGGGC
SEQ ID NO: 52 Bacteroides thetaiotaomicron
TTTAAGGGAGCGTAGGTGGACAGTTAAGTCAGTTGTGAAAGTTTGCGGCTCAACCG
TAAAATTGCAGTTGATACTGGCTGTCTTGAGTACAGTAGAGGTGGGCGGAATTCGT
GGTGTAGCGGTGAAATGCTTAGATATCACGAAGAACTCCGATTGCGAAGGCAGCTC
ACTGGACTGCAACTGACACTGATGCTCGAAAGTGTGGGTATCAAACAGGATTAGAT
ACCCTGGTAGTCCACACAGTAAACGATGAATACTCGCTGTTTGCGATATACAGTAAG
CGGCCWAGCGAAAGCATTAAGTATTCCACCTGGGGAGTACGCCGGCAACGGTGAA
ACTCAAAGGAATTGACGGGGGCCCGCACAAGCGGAGGAACATGTGGTTTAATTCGA
TGATACGCGAGGAACCTTACCCGGGCTTAAATTGCAWWTGAATAWWYTGGAAACA
GKWTAGYCGYAAGRCAWWTGTGAAGGTGCTGCATGGTTGTCGTCAGCTCGTGCCG
TGAGGTGTCGGCTTAAGTGCCATAACGAGCGCAACCCTTATCTTTAGTTACTAACAG
GTCATGCTGAGGACTCTAGAGAGACTGCCGTCGTAAGATGTGAGGAAGGTGGGGA
TGACGTCAAATCAGCACGGCCCTTACGTCCGGGGCTACACACGTGTTACAATGGGG
GGTACAGAAGGCAGCTACCTGGTGACAGGATGCTAATCCCAAAAGCCTCTCTCAGT
TCGGATCGAAGTCTGCAACCCGACTTCGTGAAGCTGGATTCGCTAGTAATCGCGCA
TCAGCCATGGCGCGGTGAATACGTTCCCGGGCCTTGTACACACCGCCCGTCAAGC
CATGAAAGCCGGGGGTACCTGAAGTACGTAACCGCA
SEQ ID NO: 53 Bacteroides uniformis
CTGGCTCAGGATGAACGCTAGCTACAGGCTTAACACATGCAAGTCGAGGGGCAGCA
TGAACTTAGCTTGCTAAGTTTGATGGCGACCGGCGCACGGGTGAGTAACACGTATC
CAACCTGCCGATGACTCGGGGATAGCCTTTCGAAAGAAAGATTAATACCCGATGGC
ATAGTTCTTCCGCATGGTAGAACTATTAAAGAATTTCGGTCATCGATGGGGATGCGT
TCCATTAGGTTGTTGGCGGGGTAACGGCCCACCAAGCCTTCGATGGATAGGGGTTC
TGAGAGGAAGGTCCCCCACATTGGAACTGAGACACGGTCCAAACTCCTACGGGAG
GCAGCAGTGAGGAATATTGGTCAATGGACGAGAGTCTGAACCAGCCAAGTAGCGTG
AAGGATGACTGCCCTATGGGTTGTAAACTTCTTTTATACGGGAATAAAGTGAGGCAC
GTGTGCCTTTTTGTATGTACCGTATGAATAAGGATCGGCTAACTCCGTGCCAGCAGC
CGCGGTAATACGGAGGATCCGAGCGTTATCCGGATTTATTGGGTTTAAAGGGAGCG
TAGGCGGACGCTTAAGTCAGTTGTGAAAGTTTGCGGCTCAACCGTAAAATTGCAGT
TGATACTGGGTGTCTTGAGTACAGTAGAGGCAGGCGGAATTCGTGGTGTAGCGGTG
AAATGCTTAGATATCACGAAGAACTCCGATTGCGAAGGCAGCTTGCTGGACTGTAA
CTGACGCTGATGCTCGAAAGTGTGGGTATCAAACAGGATTAGATACCCTGGTAGTC
CACACAGTAAACGATGAATACTCGCTGTTTGCGATATACAGTAAGCGGCCAAGCGA
AAGCGTTAAGTATTCCACCTGGGGAGTACGCCGGCAACGGTGAAACTCAAAGGAAT
TGACGGGGGCCCGCACAAGCGGAGGAACATGTGGTTTAATTCGATGATACGCGAG
GAACCTTACCCGGGCTTGAATTGCAACTGAATGATGTGGAGACATGTCAGCCGCAA
GGCAGTTGTGAAGGTGCTGCATGGTTGTCGTCAGCTCGTGCCGTGAGGTGTCGGC
TTAAGTGCCATAACGAGCGCAACCCTTATCGATAGTTACCATCAGGTTATGCTGGG
GACTCTGTCGAGACTGCCGTCGTAAGATGTGAGGAAGGTGGGGATGACGTCAAATC
AGCACGGCCCTTACGTCCGGGGCTACACACGTGTTACAATGGGGGGTACAGAAGG
CAGCTACACGGCGACGTGATGCTAATCCCTAAAGCCTCTCTCAGTTCGGATTGGAG
TCTGCAACCCGACTCCATGAAGCTGGATTCGCTAGTAATCGCGCATCAGCCACGGC
GCGGTGAATACGTTCCCGGGCCTTGTACACACCGCCCGTCAAGCCATGAAAGCCG
GGGGTACCTGAAGTGCGTAACCGCGAGGAGCGCCCTAGGGTAAAACTGGTGATTG
GGGCTAAGTCGTAACAAGGTA
SEQ ID NO: 54 Bacteroides vulgatus
ATGAAGAGTTTGATCCTGGCTCAGGATGAACGCTAGCTACAGGCTTAACACATGCA
AGTCGAGGGGCAGCATGGTCTTAGCTTGCTAAGGCCGATGGCGACCGGCGCACGG
GTGAGTAACACGTATCCAACCTGCCGTCTACTCTTGGACAGCCTTCTGAAAGGAAG
ATTAATACAAGATGGCATCATGAGTTCACATGTTCACATGATTAAAGGTATTCCGGT
AGACGATGGGGATGCGTTCCATTAGATAGTAGGGGGGGTAACGGCCCACCTAGTCT
TCGATGGATAGGGGTTCTGAGAGGAAGGTCCCCCACATTGGAACTGAGACACGGTC
CAAACTCCTACGGGAGGCAGCAGTGAGGAATATTGGTCAATGGGCGAGAGCCTGA
ACCAGCCAAGTAGCGTGAAGGATGACTGCCCTATGGGTTGTAAACTTCTTTTATAAA
GGAATAAAGTCGGGTATGCATACCCGTTTGCATGTACTTTATGAATAAGGATCGGCT
AACTCCGTGCCAGCAGCCGCGGTAATACGGAGGATCCGAGCGTTATCCGGATTTAT
TGGGTTTAAAGGGAGCGTAGATGGATGTTTAAGTCAGTTGTGAAAGTTTGCGGCTC
AACCGTAAAATTGCAGTTGATACTGGATATCTTGAGTGCAGTTGAGGCAGGCGGAA
TTCGTGGTGTAGCGGTGAAATGCTTAGATATCACGAAGAACTCCGATTGCGAAGGC
AGCCTGCTAAGCTGCAACTGACATTGAGGCTCGAAAGTGTGGGTATCAAACAGGAT
TAGATACCCTGGTAGTCCACACGGTAAACGATGAATACTCGCTGTTTGCGATATACG
GCAAGCGGCCAAGCGAAAGCGTTAAGTATTCCACCTGGGGAGTACGCCGGCAACG
GTGAAACTCAAAGGAATTGACGGGGGCCCGCACAAGCGGAGGAACATGTGGTTTAA
TTCGATGATACGCGAGGAACCTTACCCGGGCTTAAATTGCAGATGAATTACGGTGA
AAGCCGTAAGCCGCAAGGCATCTGTGAAGGTGCTGCATGGTTGTCGTCAGCTCGTG
CCGTGAGGTGTCGGCTTAAGTGCCATAACGAGCGCAACCCTTGTTGTCAGTTACTA
ACAGGTTCCGCTGAGGACTCTGACAAGACTGCCATCGTAAGATGTGAGGAAGGTGG
GGATGACGTCAAATCAGCACGGCCCTTACGTCCGGGGCTACACACGTGTTACAATG
GGGGGTACAGAGGGCCGCTACCACGCGAGTGGATGCCAATCCCAAAAACCTCTCT
CAGTTCGGACTGGAGTCTGCAACCCGACTCCACGAAGCTGGATTCGCTAGTAATCG
CGCATCAGCCACGGCGCGGTGAATACGTTCCCGGGCCTTGTACACACCGCCCGTC
AAGCCATGGGAGCCGGGGGTACCTGAAGTGCGTAACCGCGAGGAGCGCCCTAGG
GTAAAACTGGTGACTGGGGCTAAGTCGTAACAAGGTAGCCGTACCGGAAG
SEQ ID NO: 55 Bifidobacterium adolescentis
GGGCTCGTAGKCGGTTCGTCGCGTCCGGTGTGAAAGTCCAYCGCTTAACGGTGGA
TCCGCGCCGGGTACGGGCGGGCTTGAGTGCGGTAGGGGAGACTGGAATTCCCGGT
GTAACGGTGGAATGTGTAGATATCGGGAAGAACACCAATGGCGAAGGCAGGTCTCT
GGGCCGTCACTGACGCTGAGGAGCGAAAGCGTGGGGAGCGAACAGGATTAGATAC
CCTGGTAGTCCACGCCGTAAACGGTGGATGCTGGATGTGGGGACCATTCCACGGT
CTCCGTGTCGGAGCCAACGCGTTAAGCATCCCGCCTGGGGAGTACGGCCGCAAGG
CTAAAACTCAAAGAAATTGACGGGGGCCCGCACAAGCGGCGGAGCATGCGGATTA
ATTCGATGCAACGCGAAGAACCTTACCTGGGCTTGACATGTTCCCGACAGCCCCAG
AGATGGGGCCTCCCTTCGGGGGGGGTTCACAGGTGGTGCATGGTCGTCGTCAGCT
CGTGTCGTGAGATGTTGGGTTAAGTCCCGCAACGAGCGCAACCCTCGCCCTGTGTT
GCCAGCACGTCGTGGTGGGAACTCACGGGGGACCGCCGGGGTCAACTCGGAGGA
AGGTGGGGATGACGTCAGATCATCATGCCCCTTACGTCCAGGGCTTCACGCATGCT
ACAATGGCCGGTACAACGGGATGCGACACT-
GTGAGGTGGAGCGGATCCCTTAAAACCGGTCTCAGTTCGGATTGGAGTCTGCAACC
CGACTCCATGAAGGCGGAGTCGCTAGTAATCGCGGATCAGCAACGCCGCGGTGAA
TGCGTTCCCGGGCCTTGTACACACCGCCCGTCAAGTCATGAAAGTGGGTAGCACCC
GAAGCCGGTGGCCCATCCTTTTTGGGG
SEQ ID NO: 56 Bifidobacterium longum
TGGCTCAGGATGAACGCTGGGGGCGTGCTTAACACATGCAAGTCGAACGGGATCC
ATCAGGCTTTGCTTGGTGGTGAGAGTGGCGAACGGGTGAGTAATGCGTGACCGAC
CTGCCCCATACACCGGAATAGCTCCTGGAAACGGGTGGTAATGCCGGATGCTCCAG
TTGATCGCATGGTCTTCTGGGAAAGCTTTCGCGGTATGGGATGGGGTCGCGTCCTA
TCAGCTTGACGGGGGGGTAACGGCCCACCGTGGCTTCGACGGGTAGCCGGCCTGA
GAGGGCGACCGGCCACATTGGGACTGAGATACGGCCCAGACTCCTACGGGAGGCA
GCAGTGGGGAATATTGCACAATGGGCGCAAGCCTGATGCAGCGACGCCGCGTGAG
GGATGGAGGCCTTCGGGTTGTAAACCTCTTTTATCGGGGAGCAAGCGAGAGTGAGT
TTACCCGTTGAATAAGCACCGGCTAACTACGTGCCAGCAGCCGCGGTAATACGTAG
GGTGCAAGCGTTATCCGGAATTATTGGGCGTAAAGGGCTCGTAGGCGGTTCGTCGC
GTCCGGTGTGAAAGTCCATCGCTTAACGGTGGATCCGCGCCGGGTACGGGGGGGC
TTGAGTGCGGTAGGGGAGACTGGAATTCCCGGTGTAACGGTGGAATGTGTAGATAT
CGGGAAGAACACCAATGGCGAAGGCAGGTCTCTGGGCCGTTACTGACGCTGAGGA
GCGAAAGCGTGGGGAGCGAACAGGATTAGATACCCTGGTAGTCCACGCCGTAAAC
GGTGGATGCTGGATGTGGGGCCCGTTCCACGGGTTCCGTGTCGGAGCTAACGCGT
TAAGCATCCCGCCTGGGGAGTACGGCCGCAAGGCTAAAACTCAAAGAAATTGACGG
GGGCCCGCACAAGCGGCGGAGCATGCGGATTAATTCGATGCAACGCGAAGAACCT
TACCTGGGCTTGACATGTTCCCGACGGTCGTAGAGATACGGCTTCCCTTCGGGGCG
GGTTCACAGGTGGTGCATGGTCGTCGTCAGCTCGTGTCGTGAGATGTTGGGTTAAG
TCCCGCAACGAGCGCAACCCTCGCCCCGTGTTGCCAGCGGATTATGCCGGGAACT
CACGGGGGACCGCCGGGGTTAACTCGGAGGAAGGTGGGGATGACGTCAGATCATC
ATGCCCCTTACGTCCAGGGCTTCACGCATGCTACAATGGCCGGTACAACGGGATGC
GACGCGGCGACGCGGAGCGGATCCCTGAAAACCGGTCTCAGTTCGGATCGCAGTC
TGCAACTCGACTGCGTGAAGGCGGAGTCGCTAGTAATCGCGAATCAGCAACGTCGC
GGTGAATGCGTTCCCGGGCCTTGTACACACCGCCCGTCAAGTCATGAAAGTGGGCA
GCACCCGAAGCCGGTGGCCTAACCCCTTGTGGGATGGAGCCGTCTAAGGTGAGGC
TCGTGATTGGGACTAAGTCGTAACAAGGTAGCCGTACCGGAAGGTGCGGCTGG
SEQ ID NO: 57 Bifidobacterium pseudocatenulatum
GGTTCGTCGCGTCCGGTGTGAAAGTCCATCGTTTAACGGTGGATCTGCGCCGGGTA
CGGGGGGGCTGGAGTGCGGTAGGGGAGACTGGAATTCCCGGTGTAACGGTGGAAT
GTGTAGATATCGGGAAGAACACCAATGGCGAAGGCAGGTCTCTGGGCCGTTACTGA
CGCTGAGGAGCGAAAGCGTGGGGAGCGAACAGGATTAGATACCCTGGTAGTCCAC
GCCGTAAACGGTGGATGCTGGATGTGGGGCCCGTTCCACGGGTTCCGTGWCGGAG
CTAACGCGTTAAGCATCCCGCCTGGGGAGTACGGCCGCAAGGCTAAAACWMAAAK
AAATTGACGGGGGCCCGCACAAGCGGCGGAGCATGCGGATTAATTCGATGCAACG
CGAAGAACCTTACCTGGGCTTGACATGTTCCCGACAGCCGTAGAGATATGGCCTCC
CTTCGGGGGGGGTTCACAGGTGGTGCATGGTCGTCGTCAGCTCGTGTCGTGAGAT
GTTGGGTTAAGTCCCGCAACGAGCGCAACCCTCGCCCTGTGTTGCCAGCACGTCAT
GGTGGGAACTCACGGGGGACCGCCGGGGTCAACTCGGAGGAAGGTGGGGATGAC
GTCAGATCATCATGCCCCTTACGTTCAGGGCTTCACGCATGCTACAATGGCCGGTA
CAACGGGATGCGACACGGCGACGTG
SEQ ID NO: 58 Blautia coccoides/ Blautia producta
TGTGACTGAGCGGCGGACGGGTGAGTAACGCGTGGGTAACCTGCCTCATACAGGG
GGATAACAGTTAGAAATGACTGCTAATACCGCATAAGCGCACAGGACCGCATGGTC
TGGTGTGAAAAACTCCGGTGGTATGAGATGGACCCGCGTCTGATTAGCTAGTTGGA
GGGGTAACGGCCCACCAAGGCGACGATCAGTAGCCGGCCTGAGAGGGTGAACGG
CCACATTGGGACTGAGACACGGCCCAGACTCCTACGGGAGGCAGCAGTGGGGAAT
ATTGCACAATGGGGGAAACCCTGATGCAGCGACGCCGCGTGAAGGAAGAAGTATCT
CGGTATGTAAACTTCTATCAGCAGGGAAGAAAATGACGGTACCTGACTAAGAAGCC
CCGGCTAACTACGTGCCAGCAGCCGCGGTAATACGTAGGGGGCAAGCGTTATCCG
GATTTACTGGGTGTAAAGGGAGCGTAGACGGAAGAGCAAGTCTGATGTGAAAGGCT
GGGGCTTAACCCCAGGACTGCATTGGAAACTGTTGTTCTAGAGTGCCGGAGAGGTA
AGCGGAATTCCTAGTGTAGCGGTGAAATGCGTAGATATTAGGAGGAACACCAGTGG
CGAAGGCGGCTTACTGGACGGTAACTGACGTTGAGGCTCGAAAGCGTGGGGAGCA
AACAGGATTAGATACCCTGGTAGTCCACGCCGTAAACGATGAATACTAGGTGTCGG
GTGGCAAAGCCATTCGGTGCCGCAGCAAACGCAATAAGTATTCCACCTGGGGAGTA
CGTTCGCAAGAATGAAACTCAAAGGAATTGACGGGGACCCGCACAAGCGGTGGAG
CATGTGGTTTAATTCGAAGCAACGCGAAGAACCTTACCAAGTCTTGACATCCCTCTG
ACCGTCCCGTAACGGGGGCTTCCCTTCGGGGCAGAGGAGACAGGTGGTGCATGGT
TGTCGTCAGCTCGTGTCGTGAGATGTTGGGTTAAGTCCCGCAACGAGCGCAACCCT
TATCCTTAGTAGCCAGCACATGATGGTGGGCACTCTAGGGAGACTGCCGGGGATAA
CCCGGAGGAAGGCGGGGACGACGTCAAATCATCATGCCCCTTATGATTTGGGCTAC
ACACGTGCTACAATGGCGTAAACAAAGGGAAGCGAGACAGCGATGTTGAGCGAATC
CCAAAAATAACGTCCCAGTTCGGACTGCAGTCTGCAACTCGACTGCACGAAGCTGG
AATCGCTAGTAATCGCGGATCAGAATGCCGCGGTGAATACGTTCCCGGGTCTTGTA
CACACCGCCCGTCACACCATGGGAGTCAGTAACGCCCGAAGTCAGTGACCTAACC
GAAAGGAAGGAGCTGCCGAAGGGGGGACCGATAACTGGGGTGAAGTCGTAACAAG
GTA
SEQ ID NO: 59 Clostridium citroniae
TCCGGATTTACTGGAGTAGT-
AAGGGAGCGTAGACGGCGAAGCAAGTCTGGAGTGAAAACCCAGGGCTCAACCCTG
GGACTGCTTTGGAAACTGTTTTGCTAGAGTGTCGGAGAGGTAAGTGGAATTCCTAG
TGTAGCGGTGAAATGCGTAGATATTAGGAGGAACACCAGTGGCGAAGGGGCTTAC
TGGACGATAACTGACGTTGAGGCTCGAAAGCGTGGGGAGCAAACAGGATTAGATAC
CCTGGTAGTCCACGCCGTAAACGATGAATGCTAGGTGTTGGGGGGCAAAGCCCTTC
GGTGCCGTCGCAAACGCAATAAGCATTCCACCTGGGGAGTACGTTCGCAAGAATGA
AACTCAAAGGAATTGACGGGGACCCGCACAAGCGGTGGAGCATGTGGTTTAATTCG
AAGCAACGCGAAGAACCTTACCAAGTCTTGACATCCCCCTGACCGGTCAGTAAAGT
GACCTTTCCTTCGGGACAGGGGAGACAGGTGGTGCATGGTTGTCGTCAGCTCGTGT
CGTGAGATGTTGGGTTAAGTCCCGCAACGAGCGCAACCCTTATCCTTAGTAGCCAG
CAGGTAAAGCTGGGCACTCTAGGGAGACTGCCAGGGATAACCTGGAGGAAGGTGG
GGATGACGTCAAATCATCATGCCCCTTATGATTTGGGCTACACACGTGCTACAATGG
CGTAAACAAAGGGAAGCGACCCTGCGAAGGCAAGCAAATCCCAAAAATAACGTCCC
AGTTCGGACTGTAGTCTGCAACCCGACTACACGAAGCTGGAATCGCTAGTAATCGC
GAATCAGAATGTCGCGGTGAATACGTTCCCGGGTCTTGTACACACCGCCCGTCACA
CCATGGGAGTCAGCAACGCCCGAAGTCAGTGACCCAACCGAAAGGAGGGAGCTGC
CGAA
SEQ ID NO: 60 Clostridium clostridioforme
TAATACCGCATAAGCGCACAGTGCCGCATGGCAGTGTGTGAAAAACTCCGGTGGTG
TGAGATGGATCCGCGTCTGATTAGCCAGTTGGGGGGGTAACGGCCCACCAAAGCG
ACGATCAGTAGCCGACCTGAGAGGGTGACCGGCCACATTGGGACTGAGACACGGC
CCAAACTCCTACGGGAGGCAGCAGTGGGGAATATTGCACAATGGGCGAAAGCCTG
ATGCAGCGACGCCGCGTGAGTGAAGAAGTATTTCGGTATGTAAAGCTCTATCAGCA
GGGAAGAAAATGACGGTACCTGACTAAGAAGCCCCGGCTAACTACGTGCCAGCAG
CCGCGGTAATACGTAGGGGGCAAGCGTTATCCGGATTTACTGGGTGTAAAGGGAG
CGTAGACGGCGAAGCAAGTCTGAAGTGAAAACCCGGGGCTCAACCCTGGGACTGC
TTTGGAAACTGTTTTGCTAGAGTGTCGGAGAGGTAAGTGGAATTCCTAGTGTAGCG
GTGAAATGCGTAGATATTAGGAGGAACACCAGTGGCGAAGGCGGCTTACTGGACGA
TAACTGACGTTGAGGCTCGAAAGCGTGGGGAGCAAACAGGATTAGATACCCTGGTA
GTCCACGCCGTAAACGATGAATGCTAGGTGTTGGGGGGCAAAGCCCTTCGGTGCC
GCCGCAAACGCAGTAAGCATTCCACCTGGGGAGTACGTTCGCAAGAATGAAACTCA
AAGGAATTGACGGGGACCCGCACAAGCGGTGGAGCATGTGGTTTAATTCGAAGCAA
CGCGAAGAACCTTACCAAGTCTTGACATCCCCCTGACGGGCCGGTAACGCGGCCTT
TCCTTCGGGACAGGGGAGACAGGTGGTGCATGGTTGTCGTCAGCTCGTGTCGTGA
GATGTTGGGTTAAGTCCCGCAACGAGCGCAACCCTTATCCTTAGTAGCCAGCAGGT
AGAGCCGGGCACTCTAGGGAGACTGCCAGGGATAACCTGGAGGAAGGTGGGGATG
ACGTCAAATCATCATGCCCCTTATGATTTGGGCTACACACGTGCTACAATGGCGTAA
ACAAAGGGAAGCGAGACAGTGATGTGGAGCAAATCCCAAAAATAACGTCCCAGTTC
GGACTGTAGTCTGCAACCCGACTACACGAAGCTGGAATCGCTAGTAATCGCGAATC
AGAATGTCGCGGTGAATACGTTCCCGGGTCTTGTACACACCGCCCGTCACACCATG
GGAGTCAGCAACGCCCGAAGTCAGTGACCCAACCGAAAGGAGGGAGCTGCCGAAG
GCGGGGCAGGTAACTGGGGTGAAGTCGT
SEQ ID NO: 61 Clostridium innocuum
ATGGAGAGTTTGATCCTGGCTCAGGATGAACGCTGGCGGCATGCCTAATACATGCA
AGTCGAACGAAGT-
TTCGAGGAAGCTTGCTTCCAAAGAGACTTAGTGGCGAACGGGTGAGTAACACGTAG
GTAACCTGCCCATGTGTCCGGGATAACTGCTGGAAACGGTAGCTAAAACCGGATAG
GTATACAGAGCGCATGCTCAGTATATTAAAGCGCCCATCAAGGCGTGAACATGGAT
GGACCTGCGGCGCATTAGCTAGTTGGTGAGGTAACGGCCCACCAAGGCGATGATG
CGTAGCCGGCCTGAGAGGGTAAACGGCCACATTGGGACTGAGACACGGCCCAAAC
TCCTACGGGAGGCAGCAGTAGGGAATTTTCGTCAATGGGGGAAACCCTGAACGAG
CAATGCCGCGTGAGTGAAGAAGGTCTTCGGATCGTAAAGCTCTGTTGTAAGTGAAG
AACGGCTCATAGAGGAAATGCTATGGGAGTGACGGTAGCTTACCAGAAAGCCACGG
CTAACTACGTGCCAGCAGCCGCGGTAATACGTAGGTGGCAAGCGTTATCCGGAATC
ATTGGGCGTAAAGGGTGCGTAGGTGGCGTACTAAGTCTGTAGTAAAAGGCAATGGC
TCAACCATTGTAAGCTATGGAAACTGGTATGCTGGAGTGCAGAAGAGGGCGATGGA
ATTCCATGTGTAGCGGTAAAATGCGTAGATATATGGAGGAACACCAGTGGCGAAGG
CGGTCGCCTGGTCTGTAACTGACACTGAGGCACGAAAGCGTGGGGAGCAAATAGG
ATTAGATACCCTAGTAGTCCACGCCGTAAACGATGAGAACTAAGTGTTGGAGGAATT
CAGTGCTGCAGTTAACGCAATAAGTTCTCCGCCTGGGGAGTATGCACGCAAGTGTG
AAACTCAAAGGAATTGACGGGGGCCCGCACAAGCGGTGGAGTATGTGGTTTAATTC
GAAGCAACGCGAAGAACCTTACCAGGCCTTGACATGGAAACAAATACCCTAGAGAT
AGGGGGATAATTATGGATCACACAGGTGGTGCATGGTTGTCGTCAGCTCGTGTCGT
GAGATGTTGGGTTAAGTCCCGCAACGAGCGCAACCCTTGTCGCATGTTACCAGCAT
CAAGTTGGGGACTCATGCGAGACTGCCGGTGACAAACCGGAGGAAGGTGGGGATG
ACGTCAAATCATCATGCCCCTTATGGCCTGGGCTACACACGTACTACAATGGCGGC
CACAAAGAGCAGCGACACAGTGATGTGAAGCGAATCTCATAAAGGTCGTCTCAGTT
CGGATTGAAGTCTGCAACTCGACTTCATGAAGTCGGAATCGCTAGTAATCGCAGAT
CAGCATGCTGCGGTGAATACGTTCTCGGGCCTTGTACACACCGCCCGTCAAACCAT
GGGAGTCAGTAATACCCGAAGCCGGTGGCATAACCGTAAGGAGTGAGCCGTCGAA
GGTAGGACCGA
SEQ ID NO: 62 Clostridium innocuum / Erysipelotrichaceae bacterium 6_1_45
ATGGAGAGTTTGATCCTGGCTCAGGATGAACGCTGGGGGCATGCCTAATACATGCA
AGTCGAACGAAGT-
TTCGAGGAAGCTTGCTTCCAAAGAGACTTAGTGGCGAACGGGTGAGTAACACGTAG
GTAACCTGCCCATGTGTCCGGGATAACTGCTGGAAACGGTAGCTAAAACCGGATAG
GTATACAGAGCGCATGCTCAGTATATTAAAGCGCCCATCAAGGCGTGAACATGGAT
GGACCTGCGGCGCATTAGCTAGTTGGTGAGGTAACGGCCCACCAAGGCGATGATG
CGTAGCCGGCCTGAGAGGGTAAACGGCCACATTGGGACTGAGACACGGCCCAAAC
TCCTACGGGAGGCAGCAGTAGGGAATTTTCGTCAATGGGGGAAACCCTGAACGAG
CAATGCCGCGTGAGTGAAGAAGGTCTTCGGATCGTAAAGCTCTGTTGTAAGTGAAG
AACGGCTCATAGAGGAAATGCTATGGGAGTGACGGTAGCTTACCAGAAAGCCACGG
CTAACTACGTGCCAGCAGCCGCGGTAATACGTAGGTGGCAAGCGTTATCCGGAATC
ATTGGGCGTAAAGGGTGCGTAGGTGGCGTACTAAGTCTGTAGTAAAAGGCAATGGC
TCAACCATTGTAAGCTATGGAAACTGGTATGCTGGAGTGCAGAAGAGGGCGATGGA
ATTCCATGTGTAGCGGTAAAATGCGTAGATATATGGAGGAACACCAGTGGCGAAGG
CGGTCGCCTGGTCTGTAACTGACACTGAGGCACGAAAGCGTGGGGAGCAAATAGG
ATTAGATACCCTAGTAGTCCACGCCGTAAACGATGAGAACTAAGTGTTGGAGGAATT
CAGTGCTGCAGTTAACGCAATAAGTTCTCCGCCTGGGGAGTATGCACGCAAGTGTG
AAACTCAAAGGAATTGACGGGGGCCCGCACAAGCGGTGGAGTATGTGGTTTAATTC
GAAGCAACGCGAAGAACCTTACCAGGCCTTGACATGGAAACAAATACCCTAGAGAT
AGGGGGATAATTATGGATCACACAGGTGGTGCATGGTTGTCGTCAGCTCGTGTCGT
GAGATGTTGGGTTAAGTCCCGCAACGAGCGCAACCCTTGTCGCATGTTACCAGCAT
CAAGTTGGGGACTCATGCGAGACTGCCGGTGACAAACCGGAGGAAGGTGGGGATG
ACGTCAAATCATCATGCCCCTTATGGCCTGGGCTACACACGTACTACAATGGCGAC
CACAAAGAGCAGCGACACAGTGATGTGAAGCGAATCTCATAAAGGTCGTCTCAGTT
CGGATTGAAGTCTGCAACTCGACTTCATGAAGTCGGAATCGCTAGTAATCGCAGAT
CAGCATGCTGCGGTGAATACGTTCTCGGGCCTTGTACACACCGCCCGTCAAACCAT
GGGAGTCAGTAATACCCGAAGCCGGTGGCATAACCGTAAGGAGTGAGCCGTCGAA
GGTAGGACCGA
SEQ ID NO: 63 Clostridium sordellii
ACACATGCAAGTCGAGCGAACCCTTCGGGGTGAGCGGCGGACGGGTGAGTAACGC
GTGGGTAACCTGCCCTGTACACACGGATAACATACCGAAAGGTATGCTAATACGGG
ATRAYATATGAGAGTCGCATGGCTTTTGTATCAAAGCTCCGGCGGTACAGGATGGA
CCCGCGTCTGATTAGCTAGTTGGTAAGGTAACGGCTTACCAAGGCAACGATCAGTA
GCCGACCTGAGAGGGTGATCGGCCACATTGGAACTGAGACACGGTCCAAACTCMT
ACGGGAGGCAGCAGTGGGGAATATTGCACAATGGGCGAAAGCCTGATGCAGCAAC
GCCGCGTGAGCGATGAAGGCCTTCGGGTCGTAAAGCTCTGTCCTCAAGGAAGATAA
TGACGGTACTTGAGGAGGAAGCCCCGGCTAACTACGTGCCAGCAGCCGCGGTAAT
ACGTAGGGGGCTAGCGTTATCCGGAATTACTGGGCGTAAAGGGTGCGTAGGCGGT
CTTTCAAGCCAGAAGTGAAAGGCTACGGCTCAACCGTAGTAAGCTTTTGGAACTGT
AGGACTTGAGTGCAGGAGAGGAGAGTGGAATTCCTAGTGTAGCGGTGAAATGCGTA
GATATTAGGAGGAACACCAGTAGCGAAGGCGGCTCTCTGGACTGTAACTGACGCTG
AGGCACGAAAGCGTGGGGAGCAAACAGGATTAGATACCCTGGTAGTCCACGCCGT
AAACGATGAGTACTAGGTGTCGGGGGTTACCCCCCTCGGTGCCGCAGCTAACGCAT
TAAGTACTCCGCCTGGGAAGTACGCTCGCAAGAGTGAAACTCAAAGGAATTGACGG
GGACCCGCACAAGTAGCGGAGCATGTGGTTTAATTCGAAGCAACGCGAAGAACCTT
ATCTAARCTTGACATC
SEQ ID NO: 64 Coprococcus comes
ACRGGAGGCAGCAGTGGGGAATATTGCACAATGGGGGAAACCCTGATGCAGCGAC
GCCRCGTGAGCGAAGAAGTATTKCGGTATGTAAAGCTCTATCAGCAGGGAAGAAAA
TGACGGTACCTGACTAAGAAGCACCGGCTAAATACGTGCCAGCAGCCGCGGTAATA
CGTATGGTGCAAGCGTTATCCGGATTTACTGGGTGTAAAGGGAGCGTAGACGGCTG
TGTAAGTCTGAAGTGAAAGGGGGGGGCTCCCCCCCGGGGACTGCTTTGGAAACTAT
GCAGCTAGACTGTCGGACAGGTAAGTGGAATTCCCAGTGTAGCGGTGAAATGCGTA
GATATTGGGAGGAACAGCAKTGGGTAAGGCTSCTTACAGGACRAT
SEQ ID NO: 65 Dorea longicatena
TAACGCGTGGGTAACCTGCCTCATACAGGGGGATAACAGTTAGAAATGACTGCTAA
TACCGCATAAGACCACGGTACCGCATGGTACAGTGGTAAAAACTCCGGTGGTATGA
GATGGACCCGCGTCTGATTAGGTAGTTGGTGGGGTAACGGCCTACCAAGCCGACG
ATCAGTAGCCGACCTGAGAGGGTGACCGGCCACATTGGGACTGAGACACGGCCCA
GACTCCTACGGGAGGCAGCAGTGGGGAATATTGCACAATGGAGGAAACTCTGATGC
AGCGACGCCGCGTGAAGGATGAAGTATTTCGGTATGTAAACTTCTATCAGCAGGGA
AGAAAATGACGGTACCTGACTAAGAAGCCCCGGCTAACTACGTGCCAGCAGCCGC
GGTAATACGTAGGGGGCAAGCGTTATCCGGATTTACTGGGTGTAAAGGGAGCGTAG
ACGGCACGGCAAGCCAGATGTG-
AAAGCCCGGGGCTCAACCCCGGGACTGCATTTGGAACTGCTGAGCTAGAGTGTCG
GAGAGGCAAGTGGAATTCCTAGTGTAGCGGTGAAATGCGTAGATATTAGGAGGAAC
ACCAGTGGCGAAGGCGGCTTGCTGGACGATGACTGACGTTGAGGCTCGAAAGCGT
GGGGAGCAAACAGGATTAGATACCCTGGTAGTCCACGCCGTAAACGATGACTGCTA
GGTGTCGGGTGGCAAAGCCATTCGGTGCCGCAGCTAACGCAATAAGCAGTCCACC
TGGGGAGTACGTTCGCAAGAATGAAACTCAAAGGAATTGACGGGGACCCGCACAAG
CGGTGGAGCATGTGGTTTAATTCGAAGCAACGCGAAGAACCTTACCTGATCTTGAC
ATCCCGATGACCGCTTCGTAATGGAAGCTTTTCTTCGGAACATCGGTGACAGGTGG
TGCATGGTTGTCGTCAGCTCGTGTCGTGAGATGTTGGGTTAAGTCCCGCAACGAGC
GCAACCCCTATCTTCAGTAGCCAGCAGGTTAAGCTGGGCACTCTGGAGAGACTGCC
AGGGATAACCTGGAGGAAGGTGGGGATGACGTCAAATCATCATGCCCCTTATGACC
AGGGCTACACACGTGCTACAATGGCGTAAACAAAGAGAAGCGAACTCGCGAGGGTA
AGCAAATCTCAAAAATAACGTCTCAGTTCGGATTGTAGTCTGCAACTCGACTACATG
AAGCTGGAATCGCTAGTAATCGCAGATCAGAATGCTGCGGTGAATACGTTCCCGGG
TCTTGTACACACCGCCCGTCACACCATGGGAGTCAGTAACGCCCGAAGTCAGTGAC
CCAACCGTAAGG
SEQ ID NO: 66 Erysipelatoclostridium ramosum
GAGGGAGCAGGCGGCAGCAAGGGTCTGTGGTGAAAGCCTGAAGTTAAACTTCAGT
AAGCCATAGAAACCAGGCAGCTAGAGTGCAGGAGAGGAKCGTGGAATTCCATGTGT
AGCGGTGAAATGCGTAGATATATGGAGGAACACCAGTGGCGAAGGCGACGATCTG
GCCTGCAACTGACGCTCAGTCCCGAAAGCGTGGGGAGCAAATAGGATTAGATACCC
TAGTAGTCCACGCCGTAAACGATGAGTACTRAGTGTTGGATGTCAAAGTTCAGTGCT
GCAGTTAACGCAATAAGTACTCCGCCTGAGTAGTACGTTCGCAAGAATGAAACTCAA
AGGAATTGACGGGGGCCCGCACAAGCGGTGGAGCATGTGGTTTAATTCGAAGCAA
CGCGAAGAACCTTACCAGGTCTTGACATACTCATAAAGGCTCCAGAGATGGAGAGA
TAGCTATATGAGATACAGGTGGTGCATGGTTGTCGTCAGCTCGTGTCGTGAGATGT
TGGGTTAAGTCCCGCAACGAGCGCAACCCTTATCGTTAGTTACCATCATTAAGTTGG
GGACTCTAGCGAGACTGCCAGTGACAAGCTGGAGGAARGCGGGGATGACGTCAAA
TCATCATGCCCCTTATGACCTGGGCTACACACGTGCTACAATGGATGGTGCAGAGG
GAAGCGAAGCCGCGAGGTGAAGCAAAACCCATAAAACCATTCTCAGTTCGGATTGT
AGTCTGCARCTCGACTACATGAAGTTGGAATCGCTAGTAATCGCGAATCARCATGTC
GCGATGAATAMGTTCTCGGGCCTT
SEQ ID NO: 67 Eubacterium rectale
AGAGTTTGATCCTGGCTCAGGATGAACGCTGGCGGCGTGCTTAACACATGCAAGTC
GAACGAAGCACTTTATTTGATTTCCTTCGGGACTGATTATTTTGTGACTGAGTGGCG
GACGGGTGAGTAACGCGTGGGTAACCTGCCTTGTACAGGGGGATAACAGTTGGAA
ACGGCTGCTAATACCGCATAAGCGCACGGCATCGCATGATGCAGTGTGAAAAACTC
CGGTGGTATAAGATGGACCCGCGTTGGATTAGCTAGTTGGTGAGGTAACGGCCCAC
CAAGGCGACGATCCATAGCCGACCTGAGAGGGTGACCGGCCACATTGGGACTGAG
ACACGGCCCAAACTCCTACGGGAGGCAGCAGTGGGGAATATTGCACAATGGGCGA
AAGCCTGATGCAGCGACGCCGCGTGAGCGAAGAAGTATTTCGGTATGTAAAGCTCT
ATCAGCAGGGAAGATAATGACGGTACCTGACTAAGAAGCACCGGCTAAATACGTGC
CAGCAGCCGCGGTAATACGTATGGTGCAAGCGTTATCCGGATTTACTGGGTGTAAA
GGGAGCGCAGGCGGTGCGGCAAGTCTGATGTGAAAGCCCGGGGCTCAACCCCGG
TACTGCATTGGAAACTGTCGTACTAGAGTGTCGGAGGGGTAAGCGGAATTCCTAGT
GTAGCGGTGAAATGCGTAGATATTAGGAGGAACACCAGTGGCGAAGGCGGCTTACT
GGACGATAACTGACGCTGAGGCTCGAAAGCGTGGGGAGCAAACAGGATTAGATAC
CCTGGTAGTCCACGCCGTAAACGATGAATACTAGGTGTTGGGAAGCATTGCTTCTC
GGTGCCGTCGCAAACGCAGTAAGTATTCCACCTGGGGAGTACGTTCGCAAGAATGA
AACTCAAAGGAATTGACGGGGACCCGCACAAGCGGTGGAGCATGTGGTTTAATTCG
AAGCAACGCGAAGAACCTTACCAAGTCTTGACATCCTTCTGACCGGTACTTAACCGT
ACCTTCTCTTCGGAGCAGGAGTGACAGGTGGTGCATGGTTGTCGTCAGCTCGTGTC
GTGAGATGTTGGGTTAAGTCCCGCAACGAGCGCAACCCTTATCTTTAGTAGCCAGC
GGTTCGGCCGGGCACTCTAGAGAGACTGCCAGGGATAACCTGGAGGAAGGCGGGG
ATGACGTCAAATCATCATGCCCCTTATGACTTGGGCTACACACGTGCTACAATGGC
GTAAACAAAGGGAAGCAAAGCTGTGAAGCCGAGCAAATCTCAAAAATAACGTCTCA
GTTCGGACTGTAGTCTGCAACCCGACTACACGAAGCTGGAATCGCTAGTAATCGCA
GATCAGAATGCTGCGGTGAATACGTTCCCGGGTCTTGTACACACCGCCCGTCACAC
CATGGGAGTTGGGAATGCCCGAAGCCAGTGACCTAACCGAAAGGAAGGAGCTGTC
GAAGGCAGGCTCGATAACTGGGGTGAAGTCGTAACAAGGTAGCCGTATCGGAAGG
TGCGGCTGGATCACCT
SEQ ID NO: 68 Odoribacter splanchnicus
AGAGTTTGATCCTGGCTCAGGATGAACGCTAGCGACAGGCTTAACACATGCAAGTC
GAGGGGCATCATGAGGTAGCAATACCTTGATGGCGACCGGCGCACGGGTGAGTAA
CGCGTATGCAACCTGCCCGATACCGGGGTATAGCCCATGGAAACGTGGATTAACAC
CCCATAGTACTTTTATCCTGCATGGGATGTGAGTTAAATGTTTAAGGTATCGGATGG
GCATGCGTCCTATTAGTTAGTTGGCGGGGTAACAGCCCACCAAGACGATGATAGGT
AGGGGTTCTGAGAGGAAGGTCCCCCACATTGGAACTGAGACACGGTCCAAACTCCT
ACGGGAGGCAGCAGTGAGGAATATTGGTCAATGGACGAGAGTCTGAACCAGCCAA
GTCGCGTGAGGGAAGACTGCCCTATGGGTTGTAAACCTCTTTTATAAGGGAAGAAT
AAGTTCTACGTGTAGAATGATGCCTGTACCTTATGAATAAGCATCGGCTAACTCCGT
GCCAGCAGCCGCGGTAATACGGAGGATGCGAGCGTTATCCGGATTTATTGGGTTTA
AAGGGTGCGTAGGCGGTTTATTAAGTTAGTGGTTAAATATTTGAGCTAAACTCAATT
GTGCCATTAATACTGGTAAACTGGAGTACAGACGAGGTAGGCGGAATAAGTTAAGT
AGCGGTGAAATGCATAGATATAACTTAGAACTCCGATAGCGAAGGCAGCTTACCAG
ACTGTAACTGACGCTGATGCACGAGAGCGTGGGTAGCGAACAGGATTAGATACCCT
GGTAGTCCACGCCGTAAACGATGCTCACTGGTTCTGTGCGATATATTGTACGGGAT
TAAGCGAAAGTATTAAGTGAGCCACCTGGGGAGTACGTCGGCAACGATGAAACTCA
AAGGAATTGACGGGGGCCCGCACAAGCGGAGGAACATGTGGTTTAATTCGATGATA
CGCGAGGAACCTTACCTGGGTTTAAATGGGAAATGTCGTATTTGGAAACAGATATTC
TCTTCGGAGCGTTTTTCAAGGTGCTGCATGGTTGTCGTCAGCTCGTGCCGTGAGGT
GTCGGGTTAAGTCCCATAACGAGCGCAACCCTTACCGTTAGTTGCTAGCATGTAAT
GATGAGCACTCTAACGGGACTGCCACCGTAAGGTGAGAGGAAGGCGGGGATGACG
TCAAATCAGCACGGCCCTTACACCCAGGGCTACACACGTGTTACAATGGCCGGTAC
AGAGGGCCGCTACCAGGTGACTGGATGCCAATCTCAAAAGCCGGTCGTAGTTCGG
ATTGGAGTCTGTAACCCGACTCCATGAAGTTGGATTCGCTAGTAATCGCGCATCAG
CCATGGCGCGGTGAATACGTTCCCGGGCCTTGTACACACCGCCCGTCAAGCCATG
GAAGCCGGGGGTGCCTGAAGTCCGTAACCGCGAGGATCGGCCTAGGGCAAAACTG
GTAACTGGGGCTAAGTCGTAACAAGGTA
SEQ ID NO: 69 Parabacteroides distasonis
CGAGGGGCAGCRCAGGAGT-TAGCAATAC-
CSGGTGGCGACCGGCGCACGGGTGAGTAACGCGTATGCAACTTRCCTATCAGAGG
GGGATAACCCGGCGAAAGTCGGACTAATACCGCATGAAGCAGGGATYCCGCATGG
GRATATTTGCTAAAGATTCATCGCTGATAGATAGGCATGCGTTCCATTAGGCAGTTG
GCGGGGTAACRGCCCACCAAACCGACGATGGATAGGGGTTCTGAGAGGAAGGTCC
CCCACATTGGTACTGAGACACGGACCAAACTCCTACGGGAGGCAGCAGTGAGGAAT
ATTGGTCAATGGSCGWRAGSCTGAACCAGCCAAGTCGCGTGAGGGATGAAGGTTC
TATGGATCGTAAACCTCTTTTATAAGGGAATAAAGTGCGGGACGTGTCCYRTTTTGT
ATGTACCTTATGAATAAGGATCGGCTAACTCCGTGCCAGCAGCCGCGGTAATACGG
AGGATCCGAGCGTTATCCGGATTTATTGGGTTTAAAGGGTGCGTAGGCGGCCTTTT
AAGTCAGCGGTGAAAGTCTGTGGCTCAACCATAGAATTGCCGTTGAAACTGGGGGG
CTTGAGTATGTTTGAGGCAGGCGGAATGCGTGGTGTAGCGGTGAAATGCATAGATA
TCACGCAGAACCCCGWTTGCGAAGGCAGCCTGCCAAGCCGTAACTGACGCGGATG
CACGAAAGCGTGGGGATCAAACAGGATTAGATACCCTGGTA
SEQ ID NO: 70 Parabacteroides merdae
CATGCAAGTCGAGGGGCAGCATGATTTGTAGCAATACAGATTGATGGCGACCGGCG
CACGGGTGAGTAACGCGTATGCAACTTACCTATCAGAGGGGGATAGCCCGGCGAA
AGTCGGATTAATACCCCATAAAACAGGGGTCCCGCATGGGAATATTTGTTAAAGATT
CATCGCTGATAGATAGGCATGCGTTCCATTAGGCAGTTGGCGGGGTAACGGCCCAC
CAAACCGACGATGGATAGGGGTTCKGAGAGGAAGGTCCCCCACATTGGTACTGAGA
CACGGACCAAACTCCTACGGGAGGCAGCAGTGAGGAATATTGGTCAATGGCCGAG
AGGCTGAACCAGCCAAGTCGCGTGAAGGAAGAAGGATCTATGGTTTGTAAACTTCT
TTTATAGGGGAATAAAGTGGAGGACGTGTCCTTTTTTGTATGTACCCTATGAATAAG
CATCGGCTAACTCCGTGMSARCMGCCGCGGGAATACGGAAGATGCAGAGCGTTAT
CCGGATWTATTGGGGTTA
SEQ ID NO: 71 Bacteroides xylanisolvens
CATGCAAGTCGAGGGGCAGCATTTTAGTTTGCTTGCAAACTAAAGATGGCGACCGG
CGCACGGGTGAGTAACACGTATCCAACCTGCCGATAACTCGGGGATAGCCTTTCGA
AAGAAAGATTAATATCCGATAGTATATTAAAACCGCATGGTTTTACTATTAAAGAATT
TCGGTTATCGATGGGGATGCGTTCCATTAGTTTGTTGGCGGGGTAACGGCCCACCA
AGACTACGATGGATAGGGGTTCTGAGAGGAAGGTCCCCCACATTGGAACTGAGACA
CGGTCCAAACTCCTACGGGAGGCAGCAGTGAGGAATATTGGTCAATGGACGAGAGT
CTGAACCAGCCAAGTAGCGTGAAGGATGACTGCCCTATGGGTTGTAAACTTCTTTTA
TATGGGAATAAAGTATTCCACGTGTGGGATTTTGTATGTACCATATGAATAAGGATC
GGCTAACTCCGTGCCAGCAGCCGCGGTAATACGGAGGATCCGAGCGTTATCCGGA
TTTATTGGGTTTAAAGGGAGCGTAGGTGGATTGTTAAGTCAGTTGTGAAAGTTTGCG
GCTCAACCGTAAAATTGCAGTTGAAACTGGCAGTCTTGAGTACAGTAGAGGTGGGC
GGAATTCGTGGTGTAGCGGTGAAATGCTTAGATATCACGAAGAACTCCGATTGCGA
AGGCAGCTCACTAGACTGCAACTGACACTGATGCTCGAAAGTGTGGGTATCAAACA
GGATTAGATACCCTGGTAGTCCACACAGTAAACGATGAATACTCGCTGTTTGCGATA
TACAGTAAGCGGCCAAGCGAAAGCATTAAGTATTCCACCTGGGGAGTACGCCGGCA
ACGGTGAAACTCAAAGGAATTGACGGGGGCCCGCACAAGCGGAGGAACATGTGGT
TTAATTCGATGATACGCGAGGAACCTTACCCGGGCTTAAATTGCATTTGAATAATCT
GGAAACAGGTTAGCCGCAAGGCAAATGTGAAGGTGCTGCATGGTTGTCGTCAGCTC
GTGCCGTGAGGTGTCGGCTTAAGTGCCATAACGAGCGCAACCCTTATCTTTAGTTA
CTAACAGGTTATGCTGAGGACTCTAGAGAGACTGCCGTCGTAAGATGTGAGGAAGG
TGGGGATGACGTCAAATCAGCACGGCCCTTACGTCCGGGGCTACACACGTGTTACA
ATGGGGGGTACAGAAGGCAGCTACCTGGCGACAGGATGCTAATCCCAAAAACCTCT
CTCAGTTCGGATCGAAGTCTGCAACCCGACTTCGTGAAGCTGGATTCGCTAGTAAT
CGCGCATCAGCCATGGCGCGGTGAATACGTTCCCGGGCCTTGTACACACCGCCCG
TCAAGCCATGAAAGCCGGGG
SEQ ID NO: 72 Blautia obeum
GGCGTGCTTAACACATGCAAGTCGAACGGGAAACCTTTTATTGAAGCTTCGGCAGA
TTTAG-
CTGGTTTCTAGTGGCGGACGGGTGAGTAACGCGTGGGTAACCTGCCTTATACAGGG
GGATAACAACCAGAAATGGTTGCTAATACCGCATAAGCGCACAGGACCGCATGGTC
CGGTGTGAAAAACTCCGGTGGTATAAGATGGACCCGCGTTGGATTAGCTAGTTGGC
AGGGTAACGGCCTACCAAGGCGACGATCCATAGCCGGCCTGAGAGGGTGAACGGC
CACATTGGGACTGAGACACGGCCCAGACTCCTACGGGAGGCAGCAGTGGGGAATA
TTGCACAATGGGGGAAACCCTGATGCAGCGACGCCGCGTGAAGGAAGAAGTATCT
CGGTATGTAAACTTCTATCAGCAGGGAAGATAGTGACGGTACCTGACTAAGAAGCC
CCGGCTAACTACGTGCCAGCAGCCGCGGTAATACGTAGGGGGCAAGCGTTATCCG
GATTTACTGGGTGTAAAGGGAGCGTAGACGGACTGGCAAGTCTGATGTGAAAGGCG
GGGGCTCAACCCCTGGACTGCATTGGAAACTGTTAGTCTTGAGTGCCGGAGAGGTA
AGCGGAATTCCTAGTGTAGCGGTGAAATGCGTAGATATTAGGAGGAACACCAGTGG
CGAAGGCGGCTTACTGGACGGTAACTGACGTTGAGGCTCGAAAGCGTGGGGAGCA
AACAGGATTAGATACCCTGGTAGTCCACGCCGTAAACGATGAATACTAGGTGTTGG
GGAGCAAAGCTCTTCGGTGCCGCCGCAAACGCATTAAGTATTCCACCTGGGGAGTA
CGTTCGCAAGAATGAAACTCAAAGGAATTGACGGGGACCCGCACAAGCGGTGGAG
CATGTGGTTTAATTCGAAGCAACGCGAAGAACCTTACCAAGTCTTGACATCCCTCTG
ACCGTTCCTTAACCGGAACTTTCCTTCGGGACAGGGGAGACAGGTGGTGCATGGTT
GTCGTCAGCTCGTGTCGTGAGATGTTGGGTTAAGTCCCGCAACGAGCGCAACCCCT
ATCCCCAGTAGCCAGCAGTCCGGCTGGGCACTCTGAGGAGACTGCCAGGGATAAC
CTGGAGGAAGGCGGGGATGACGTCAAATCATCATGCCCCTTATGATTTGGGCTACA
CACGTGCTACAATGGCGTAAACAAAGGGAAGCAAGCCTGCGAAGGTAAGCAAATCC
CAAAAATAACGTCCCAGTTCGGACTGCAGTCTGCAACTCGACTGCACGAAGCTGGA
ATCGCTAGTAATCGCGGATCAGAATGCCGCGGTGAATACGTTCCCGGGTCTTGTAC
ACACCGCCCGTCACACCATGGGAGTCAGTAACGCCCGAAGTCAGTGACCTAACTGC
SEQ ID NO: 73 Alistipes putredinis
AGAGTTTGATCCTGGCTCAGGATGAACGCTAGCGGCAGGCTTAACACATGCAAGTC
GAGGGGCAGCATAATGGATAGCAATATCTATGGTGGCGACCGGCGCACGGGTGCG
TAACGCGTATGCAACCTACCTTTAACAGGGGGATAACACTGAGAAATTGGTACTAAT
ACCCCATAATATCATAGAAGGCATCTTTTATGGTTGAAAATTCCGATGGTTAGAGAT
GGGCATGCGTTGTATTAGCTAGTTGGTGGGGTAACGGCTCACCAAGGCGACGATAC
ATAGGGGGACTGAGAGGTTAACCCCCCACACTGGTACTGAGACACGGACCAGACT
CCTACGGGAGGCAGCAGTGAGGAATATTGGTCAATGGACGCAAGTCTGAACCAGC
CATGCCGCGTGCAGGATGACGGCTCTATGAGTTGTAAACTGCTTTTGTACGAGGGT
AAACGCAGATACGTGTATCTGTCTGAAAGTATCGTACGAATAAGGATCGGCTAACTC
CGTGCCAGCAGCCGCGGTAATACGGAGGATTCAAGCGTTATCCGGATTTATTGGGT
TTAAAGGGTGCGTAGGCGGTTTGATAAGTTAGAGGTGAAATTTCGGGGCTCAACCC
TGAACGTGCCTCTAATACTGTTGAGCTAGAGAGTAGTTGCGGTAGGCGGAATGTAT
GGTGTAGCGGTGAAATGCTTAGAGATCATACAGAACACCGATTGCGAAGGCAGCTT
ACCAAACTATATCTGACGTTGAGGCACGAAAGCGTGGGGAGCAAACAGGATTAGAT
ACCCTGGTAGTCCACGCAGTAAACGATGATAACTCGTTGTCGGCGATACACAGTCG
GTGACTAAGCGAAAGCGATAAGTTATCCACCTGGGGAGTACGTTCGCAAGAATGAA
ACTCAAAGGAATTGACGGGGGCCCGCACAAGCGGAGGAACATGTGGTTTAATTCGA
TGATACGCGAGGAACCTTACCCGGGCTTGAAAGTTAGCGACGATTCTTGAAAGAGG
ATTTCCCTTCGGGGCGCGAAACTAGGTGCTGCATGGTTGTCGTCAGCTCGTGCCGT
GAGGTGTCGGGTTAAGTCCCATAACGAGCGCAACCCCTACCGTTAGTTGCCATCAG
GTGAAGCTGGGCACTCTGGGGGGACTGCCGGTGTAAGCCGAGAGGAAGGTGGGG
ATGACGTCAAATCAGCACGGCCCTTACGTCCGGGGCTACACACGTGTTACAATGGT
AGGTACAGAGGGCAGCTACCCAGCGATGGGATGCGAATCTCGAAAGCCTATCTCAG
TTCGGATTGGAGGCTGAAACCCGCCTCCATGAAGTTGGATTCGCTAGTAATCGCGC
ATCAGCCATGGCGCGGTGAATACGTTCCCGGGCCTTGTACACACCGCCCGTCAAG
CCATGGGAGCCGGGGGTGCCTGAAGTTCGTGACCGCAAGGAGCGACCTAGGGCAA
AACTGGTGACTGGGGCTAAGTCGTAACAAGGTA
SEQ ID NO: 74 Collinsella aerofaciens
AGAGTTCGATCCTGGCTCAGGATGAACGCTGGCGGCGCGCCTAACACATGCAAGT
CGAACGGCACCTATCTTCGGATAGAAGCGAGTGGCGAACGGCTGAGTAACACGTG
GAGAACCTGCCCCCTCCCCCGGGATAGCCGCCCGAAAGGACGGGTAATACCGGAT
ACCCCGGGGTGCCGCATGGCACCCCGGCTAAAGCCCCGACGGGAGGGGATGGCT
CCGCGGCCCATCAGGTAGACGGCGGGGTGACGGCCCACCGTGCCGACAACGGGT
AGCCGGGTTGAGAGACCGACCGGCCAGATTGGGACTGAGACACGGCCCAGACTCC
TACGGGAGGCAGCAGTGGGGAATCTTGCGCAATGGGGGGAACCCTGACGCAGCGA
CGCCGCGTGCGGGACGGAGGCCTTCGGGTCGTAAACCGCTTTCAGCAGGGAAGAG
TCAAGACTGTACCTGCAGAAGAAGCCCCGGCTAACTACGTGCCAGCAGCCGCGGT
AATACGTAGGGGGCGAGCGTTATCCGGATTCATTGGGCGTAAAGCGCGCGTAGGC
GGCCCGGCAGGCCGGGGGTCGAAGCGGGGGGCTCAACCCCCCGAAGCCCCCGGA
ACCTCCGCGGCTTGGGTCCGGTAGGGGAGGGTGGAACACCCGGTGTAGCGGTGG
AATGCGCAGATATCGGGTGGAACACCGGTGGCGAAGGCGGCCCTCTGGGCCGAGA
CCGACGCTGAGGCGCGAAAGCTGGGGGAGCGAACAGGATTAGATACCCTGGTAGT
CCCAGCCGTAAACGATGGACGCTAGGTGTGGGGGGACGATCCCCCCGTGCCGCAG
CCAACGCATTAAGCGTCCCGCCTGGGGAGTACGGCCGCAAGGCTAAAACTCAAAG
GAATTGACGGGGGCCCGCACAAGCAGCGGAGCATGTGGCTTAATTCGAAGCAACG
CGAAGAACCTTACCAGGGCTTGACATATGGGTGAAGCGGGGGAGACCCCGTGGCC
GAGAGGAGCCCATACAGGTGGTGCATGGCTGTCGTCAGCTCGTGTCGTGAGATGTT
GGGTTAAGTCCCGCAACGAGCGCAACCCCCGCCGCGTGTTGCCATCGGGTGATGC
CGGGAACCCACGCGGGACCGCCGCCGTCAAGGCGGAGGAGGGGGGGGACGACGT
CAAGTCATCATGCCCCTTATGCCCTGGGCTGCACACGTGCTACAATGGCCGGTACA
GAGGGATGCCACCCCGCGAGGGGGAGCGGATCCCGGAAAGCCGGCCCCAGTTCG
GATTGGGGGCTGCAACCCGCCCCCATGAAGTCGGAGTTGCTAGTAATCGCGGATC
AGCATGCCGCGGTGAATGCGTTCCCGGGCCTTGTACACACCGCCCGTCACACCAC
CCGAGTCGTCTGCACCCGAAGTCGCCGGCCCAACCGAGAGGGGGGAGGCGCCGA
AGGTGTGGAGGGTGAGGGGGGTGAAGTCGTAACAAGGTA
SEQ ID NO: 75 Bacteroides faecis /Eubacterium hallii
CTCAGGATGAACGCTAGCTACAGGCTTAACACATGCAAGTCGAGGGGCAGCATTCC
AGTTTGCTTGCAAACTGGAGATGGCGACCGGCGCACGGGTGAGTAACACGTATCCA
ACCTGCCGATAACTCGGGGATAGCCTTTCGAAAGAAAGATTAATACCCGATGGCAT
AATAGAACCGCATGGTTTGATTATTAAAGAATTTCGGTTATCGATGGGGATGCGTTC
CATTAGGCAGTTGGTGGGGTAACGGCCCACCAAACCTTCGATGGATAGGGGTTCTG
AGAGGAAGGTCCCCCACATTGGAACTGAGACACGGTCCAAACTCCTACGGGAGGC
AGCAGTGAGGAATATTGGTCAATGGACGAGAGTCTGAACCAGCCAAGTAGCGTGAA
GGATGACTGCCCTATGGGTTGTAAACTTCTTTTATATGGGAATAAAGTGGTCCACGT
GTGGATTTTTGTATGTACCATATGAATAAGGATCGGCTAACTCCGTGCCAGCAGCCG
CGGTAATACGGAGGATCCGAGCGTTATCCGGATTTATTGGGTTTAAAGGGAGCGTA
GGTGGACAGTTAAGTCAGTTGTGAAAGTTTGCGGCTCAACCGTAAAATTGCAGTTG
ATACTGGCTGTCTTGAGTACAGTAGAGGGGGGGGAATTCGTGGTGTAGCGGTGAA
ATGCTTAGATATCACGAAGAACTCCGATTGCGAAGGCAGCTCACTGGACTGCAACT
GACACTGATGCTCGAAAGTGTGGGTATCAAACAGGATTAGATACCCTGGTAGTCCA
CACAGTAAACGATGAATACTCGCTGTTTGCGATATACAGTAAGCGGCCAAGCGAAA
GCATTAAGTATTCCACCTGGGGAGTACGCCGGCAACGGTGAAACTCAAAGGAATTG
ACGGGGGCCCGCACAAGCGGAGGAACATGTGGTTTAATTCGATGATACGCGAGGA
ACCTTACCCGGGCTTAAATTGCATTTGAATATATTGGAAACAGTATAGTCGTAAGAC
AAATGTGAAGGTGCTGCATGGTTGTCGTCAGCTCGTGCCGTGAGGTGTCGGCTTAA
GTGCCATAACGAGCGCAACCCTTATCTTTAGTTACTAACAGGTCATGCTGAGGACTC
TGGAGAGACTGCCGTCGTAAGATGTGAGGAAGGTGGGGATGACGTCAAATCAGCA
CGGCCCTTACGTCCGGGGCTACACACGTGTTACAATGGGGGGTACAGAAGGCCGC
TACCTGGTGACAGGATGCTAATCCCAAAAGCCTCTCTCAGTTCGGATCGAAGTCTG
CAACCCGACTTCGTGAAGCTGGATTCGCTAGTAATCGCGCATCAGCCATGGCGCGG
TGAATACGTTCCCGGGCCTTGTACACACCGCCCGTCAAGCCATGAAAGCCGGGGG
TACCTGAAGTACGTAACCGCAAGGAGCGTCCTAGGGTAAAACTGGTAATTGGGGCT
AAGTCGTAACAAGGTA
SEQ ID NO: 76 Alistipes shahii
ACATAGGGGGWSTGWKAGGTTWRCCSCCCACATTSRTACTGAGMCA-
TGAWCMAACTCTMTACGGGARGSAGSAGTGAGGAATATTGGTCRRTGGACGCAAG
TCTGAACCAGCCATGCCGSGTGCRGGAAGACGGCTCKATGAGTKGKAAACTGCTTT
TGTACRARRGTAAACGCTCTTACGTGTAAGAGCCTGAAAGTATSGTACRAATGAGGA
TCGGCTAACTCCGTGCCAGCAGCCGCGGTAATACGGAGGATCCAAGCGTTATCCG
GATTTATTGGGTTTAAAGGGTGCGTAGGGGGGTTGATAAAGTTAGRGG
SEQ ID NO: 77 Anaerostipes caccae
GCTT-ACACATG-
CAAGTCGAACGAAGCATTTARGATTGAAGTTTTCGGATGGATTTCCTATATGACTGA
GTGGCGGACGGGTGAGTAACGCGTGGGGAACCTGCCCTATACAGGGGGATAACAG
CTGGAAACGGCTGCTAATACCGCATAAGCGCACAGAATCGCATGATTCAGTGTGAA
AAGCCCTGGCAGTATAGGATGGTCCCGCGTCTGATTAGCTGGTTGGTGAGGTAACG
GCTCACCAAGGCGACGATCAGTAGCCGGCTTGAGAGAGTGAACGGCCACATTGGG
ACTGAGACACGGCCCAAACTCCTACGGGAGGCAGCAGTGGGGAATATTGCACAAT
GGGGG-
AAACCCTGATGCAGCGACGCCGCGTGAGTGAAGAAGTATTTCGGTATGTAAAGCTC
TATCAGCAGGGAAGAAAACAGACGGTACCTGACTAAGAAGCCCCGGCTAACTACGT
GCCAGCAGCCGCGGTAATACGTAGGGGGCAAGCGTTATCCGGAATTACTGGGTGT
AAAGGGTGCGTAGGTGGCATGGTAAGTCAGAAGTGAAAGCCCGGGGCTTAACCCC
GGGACTGCTTTTGAAACTGTCATGCTGGAGTGCAGGAGAGGTAAGCGGAATTCCTA
GTGTAGCGGTGAAATGCGTAGATATTAGGAGGAACACCAGTGGCGAAGGCGGCTTA
CTGGACTGTCACTGACACTGATGCACGAAAGCGTGGGGAGCAAACAGGATTAGATA
CCCTGGTAGTCCACGCCGTAAACGATGAATACTAGGTGTCGGGGCCGTAGAGGCTT
CGGTGCCGCAGCAAA
SEQ ID NO: 78 Phascolarctobacterium faecium
CGGAGAATTTTCATTTCGGTAGAATTCTTAGTGGCGAACGGGTGAGTAACGCGTAG
GCAACCTGCCCTTTAGACGGGGACAACATTCCGAAAGGAGTGCTAATACCGGATGT
GATCATCGTGCCGCATGGCAGGATGAAGAAAGATGGCCTCTACAAGTAAGCTATCG
CTAAAGGATGGGCCTGCGTCTGATTAGCTAGTTGGTAGTGTAACGGACTACCAAGG
CGATGATCAGTAGCCGGTCTGAGAGGATGAACGGCCACATTGGGACTGAGACACG
GCCCAAACTCCTACGGGAGGCAGCAGTGGGGAATCTTCCGCAATGGACGAAAGTC
TGACAGAGCAACGCCGCGTGAGTGATGAAGGATTTCGGTCTGTAAAGCTCTGTTGT
TTATGACGAACGTGCAGTGTGTGAACAATGCATTGCAATGACGGTAGTAAACGAGG
AAGCCACGGCTAACTACGTGCCAGCAGCCGCGGTAATACGTAGGTGGCGAGCGTT
GTCCGGAATTATTGGGCGTAAAGAGCATGTAGGCGGCTTAATAAGTCGAGCGTGAA
AAATGCGGGGCTCAACCCCGTATGGCGCTGGAAACTGTTAGGCTTGAGTGCAGGA
GAGGAAAGGGGAATTCCCAGTGTAGCGGTGAAATGCGTAGATATTGGGAGGAACAC
CAGTGGCGAAGGCGCCTTTCTGGACTGTGTTTGACGCTGAGATGCGAAAGCCAGG
GTAGC
SEQ ID NO: 79 Agathobaculum butyriciproducens
TAGTGGGGGACGGGTGAGTAACGCGTGAGCAATCTGCCTTTAAGAGGGGGATAAC
AGTCGGAAACGGCTGCTAATACCGCATAAAGCATTGAATTCGCATGTTTTCGATGCC
AAAGGAGCAATCCGCTTTTAGATGAGCTCGCGTCTGATTAGCTAGTTGGGGGGTA
ACGGCCCACCAAGGCGACGATCAGTAGCCGGACTGAGAGGTTGAACGGCCACATT
GGGACTGAGACACGGCCCAGACTCCTACGGGAGGCAGCAGTGGGGAATATTGCGC
AATGGGGGRAACCCTGACGCAGCAACGCCGCGTGATTGAAGAAGGCCTTCGGGTT
GTAAAGATCTTTAATCAGGGACGAA--
AMATGACGGTACCTGAAGAATAAGCTCCGGCTAACTACGTGCCAGCAGCCGCGGTA
ATACGTAGGGAGCAAGCGTTATCCGGATTTACTGGGTGTAAAGGGCGCGCAGGCG
GGCCGGCAAGTTGGAAGTGAAATCCGGGGGCTTAACCCCCGAACTGCTTTCAAAAC
TGCTGGTCTTGAGTGATGGAGAGGCAGGCGGAATTCCGTGTGTAGCGGTGAAATG
CGTAGATATACGGAGGAACACCAGTGGCGAAGGCGGCCTGCTGGACATTAACTGA
CGCTGAGGCGCGAAAGCGTGGGGAGCAAACAGGATTAGATACCCTGGTAGTCCAC
GCCGTAAACGATGGATA
SEQ ID NO: 80 Bacteroides fragilis
ATGAAGAGTTTGATCCTGGCTCAGGATGAACGCTAGCTACAGGCTTAACACATGCA
AGTCGAGGGGCATCAGGAAGAAAGCTTGCTTTCTTTGCTGGCGACCGGCGCACGG
GTGAGTAACACGTATCCAACCTGCCCTTTACTCGGGGATAGCCTTTCGAAAGAAAG
ATTAATACCCGATGGCATAATGATTCCGCATGGTTTCATTATTAAAGGATTCCGGTA
AAGGATGGGGATGCGTTCCATTAGGTTGTTGGTGAGGTAACGGCTCACCAAGCCTT
CGATGGATAGGGGTTCTGAGAGGAAGGTCCCCCACATTGGAACTGAGACACGGTC
CAAACTCCTACGGGAGGCAGCAGTGAGGAATATTGGTCAATGGGCGCTAGCCTGAA
CCAGCCAAGTAGCGTGAAGGATGAAGGCTCTATGGGTCGTAAACTTCTTTTATATAA
GAATAAAGTGCAGTATGTATACTGTTTTGTATGTATTATATGAATAAGGATCGGCTAA
CTCCGTGCCAGCAGCCGCGGTAATACGGAGGATCCGAGCGTTATCCGGATTTATTG
GGTTTAAAGGGAGCGTAGGTGGACTGGTAAGTCAGTTGTGAAAGTTTGCGGCTCAA
CCGTAAAATTGCAGTTGATACTGTCAGTCTTGAGTACAGTAGAGGTGGGCGGAATT
CGTGGTGTAGCGGTGAAATGCTTAGATATCACGAAGAACTCCGATTGCGAAGGCAG
CTCACTGGACTGCAACTGACACTGATGCTCGAAAGTGTGGGTATCAAACAGGATTA
GATACCCTGGTAGTCCACACAGTAAACGATGAATACTCGCTGTTTGCGATATACAGT
AAGCGGCCAAGCGAAAGCATTAAGTATTCCACCTGGGGAGTACGCCGGCAACGGT
GAAACTCAAAGGAATTGACGGGGGCCCGCACAAGCGGAGGAACATGTGGTTTAATT
CGATGATACGCGAGGAACCTTACCCGGGCTTAAATTGCAGTGGAATGATGTGGAAA
CATGTCAGTGAGCAATCACCGCTGTGAAGGTGCTGCATGGTTGTCGTCAGCTCGTG
CCGTGAGGTGTCGGCTTAAGTGCCATAACGAGCGCAACCCTTATCTTTAGTTACTAA
CAGGTTATGCTGAGGACTCTAGAGAGACTGCCGTCGTAAGATGTGAGGAAGGTGG
GGATGACGTCAAATCAGCACGGCCCTTACGTCCGGGGCTACACACGTGTTACAATG
GGGGGTACAGAAGGCAGCTAGCGGGTGACCGTATGCTAATCCCAAAAGCCTCTCTC
AGTTCGGATCGAAGTCTGCAACCCGACTTCGTGAAGCTGGATTCGCTAGTAATCGC
GCATCAGCCACGGCGCGGTGAATACGTTCCCGGGCCTTGTACACACCGCCCGTCA
AGCCATGGGAGCCGGGGGTACCTGAAGTACGTAACCGCAAGGATCGTCCTAGGGT
AAAACTGGTGACTGGGGCTAAGTCGTAACAAGGTAGCCGTACCGGAAGGTGCGGC
TGGAACACCTCCTT
SEQ ID NO: 81 Fusobacterium mortiferum
TGGCTCAGGATGAACGCTGACAGAATGCTTAACACATGCAAGTCTACTTGATCCTTC
GGGTGATGGTGGCGGACGGGTGAGTAACGCGTAAAGAACTTGCCCTGCAGTCTGG
GACAACATTTGGAAACGAATGCTAATACCGGATATTATGT-
ATTTCTCGCATGAGTTTTACATGAAAGCTATATGCGCTGCAGGAGAGCTTTGCGTCC
TATTAGCTAGTTGGTGAGGTAACGGCTCACCAAGGCCATGATAGGTAGCCGGCCTG
AGAGGGTGAACGGCCACAAGGGGACTGAGACACGGCCCTTACTCCTACGGGAGGC
AGCAGTGGGGAATATTGGACAATGGACCAAAAGTCTGATCCAGCAATTCTGTGTGC
ACGATGAAGTTTTTCGGAATGTAAAGTGCTTTCAGTTGGGACGAAGTAAGTGACGGT
ACCAACAGAAGAAGCGACGGCTAAATACGTGCCAGCAGCCGCGGTAATACGTATGT
CGCAAGCGTTATCCGGATTTATTGGGCGTAAAGCGCGTCTAGGCGGTTTGGTAAGT
CTGATGTGAAAATGCGGGGCTCAACTCCGTATTGCGTTGGAAACTGCTAAACTAGA
GTACTGGAGAGGTGGGGGGAACTACAAGTGTAGAGGTGAAATTCGTAGATATTTGT
AGGAATGCCGATGGGGAAGCCAGCCCACTGGACAGATACTGACGCTAAAGCGCGA
AAGCGTGGGTAGCAAACAGGATTAGATACCCTGGTAGTCCACGCCGTAAACGATGA
TTACTAGGTGTTGGGGGTCGAACCTCAGCGCCCAAGCTAACGCGATAAGTAATCCG
CCTGGGGAGTACGTACGCAAGTATGAAACTCAAAGGAATTGACGGGGACCCGCACA
AGCGGTGGAGCATGTGGTTTAATTCGACGCAACGCGAGGAACCTTACCAGCGTTTG
ACATCCTAAGAAATTAGCAGAGATGCTTTTGTGCCCCTTCGGGGGAACTTAGTGACA
GGTGGTGCATGGCTGTCGTCAGCTCGTGT
SEQ ID NO: 82 Paraclostridium benzoelyticum/ Clostridium bifermantans
AGAGTTTGATCCTGGCTCAGGATGAACGCTGGGGGCGTGCCTAACACATGCAAGTC
GAGCGATCTCTTCGGAGAGAGCGGCGGACGGGTGAGTAACGCGTGGGTAACCTGC
CCTGTACACACGGATAACATACCGAAAGGTATACTAATACGGGATAACATACGAAAG
TCGCATGGCTTTTGTATCAAAGCTCCGGGGGTACAGGATGGACCCGCGTCTGATTA
GCTAGTTGGTAAGGTAATGGCTTACCAAGGCAACGATCAGTAGCCGACCTGAGAGG
GTGATCGGCCACACTGGAACTGAGACACGGTCCAGACTCCTACGGGAGGCAGCAG
TGGGGAATATTGCACAATGGGCGAAAGCCTGATGCAGCAACGCCGCGTGAGCGAT
GAAGGCCTTCGGGTCGTAAAGCTCTGTCCTCAAGGAAGATAATGACGGTACTTGAG
GAGGAAGCCCCGGCTAACTACGTGCCAGCAGCCGCGGTAATACGTAGGGGGCTAG
CGTTATCCGGAATTACTGGGCGTAAAGGGTGCGTAGGTGGTTTTTTAAGTCAGAAG
TGAAAGGCTACGGCTCAACCGTAGTAAGCTTTTGAAACTAGAGAACTTGAGTGCAG
GAGAGGAGAGTAGAATTCCTAGTGTAGCGGTGAAATGCGTAGATATTAGGAGGAAT
ACCAGTAGCGAAGGCGGCTCTCTGGACTGTAACTGACACTGAGGCACGAAAGCGT
GGGGAGCAAACAGGATTAGATACCCTGGTAGTCCACGCCGTAAACGATGAGTACTA
GGTGTCGGGGGTTACCCCCCTCGGTGCCGCAGCTAACGCATTAAGTACTCCGCCT
GGGAAGTACGCTCGCAAGAGTGAAACTCAAAGGAATTGACGGGGACCCGCACAAG
TAGCGGAGCATGTGGTTTAATTCGAAGCAACGCGAAGAACCTTACCTAAGCTTGAC
ATCCCACTGACCTCTCCCTAATCGGAGATTTCCCTTCGGGGACAGTGGTGACAGGT
GGTGCATGGTTGTCGTCAGCTCGTGTCGTGAGATGTTGGGTTAAGTCCCGCAACGA
GCGCAACCCTTGCCTTTAGTTGCCAGCATTAAGTTGGGCACTCTAGAGGGACTGCC
GAGGATAACTCGGAGGAAGGTGGGGATGACGTCAAATCATCATGCCCCTTATGCTT
AGGGCTACACACGTGCTACAATGGGTGGTACAGAGGGTTGCCAAGCCGCGAGGTG
GAGCTAATCCCTTAAAGCCATTCTCAGTTCGGATTGTAGGCTGAAACTCGCCTACAT
GAAGCTGGAGTTACTAGTAATCGCAGATCAGAATGCTGCGGTGAATGCGTTCCCGG
GTCTTGTACACACCGCCCGTCACACCATGGAAGTTGGGGGCGCCCGAAGCCGGTT
AGCTAACCTTTTAGGAAGCGGCCGTCGAAGGTGAAACCAATGACTGGGGTGAAGTC
GTAACAAGGTAGCCGTATCGGAAGGTGCGGCTGGATCACCT
SEQ ID NO: 83 Escherichia fergusonii / Escherichia coli
TTGAAGAGTTTGATCATGGCTCAGATTGAACGCTGGCGGCAGGCCTAACACATGCA
AGTCGAACGGTAACAGGAAGCAGCTTGCTGCTTTGCTGACGAGTGGCGGACGGGT
GAGTAATGTCTGGGAAACTGCCTGATGGAGGGGGATAACTACTGGAAACGGTAGCT
AATACCGCATAACGTCGCAAGACCAAAGAGGGGGACCTTCGGGCCTCTTGCCATCG
GATGTGCCCAGATGGGATTAGCTAGTAGGTGGGGTAACGGCTCACCTAGGCGACG
ATCCCTAGCTGGTCTGAGAGGATGACCAGCCACACTGGAACTGAGACACGGTCCAG
ACTCCTACGGGAGGCAGCAGTGGGGAATATTGCACAATGGGCGCAAGCCTGATGC
AGCCATGCCGCGTGTATGAAGAAGGCCTTCGGGTTGTAAAGTACTTTCAGCGGGGA
GGAAGGGAGTAAAGTTAATACCTTTGCTCATTGACGTTACCCGCAGAAGAAGCACC
GGCTAACTCCGTGCCAGCAGCCGCGGTAATACGGAGGGTGCAAGCGTTAATCGGA
ATTACTGGGCGTAAAGCGCACGCAGGCGGTTTGTTAAGTCAGATGTGAAATCCCCG
GGCTCAACCTGGGAACTGCATCTGATACTGGCAAGCTTGAGTCTCGTAGAGGGGG
GTAGAATTCCAGGTGTAGCGGTGAAATGCGTAGAGATCTGGAGGAATACCGGTGGC
GAAGGCGGCCCCCTGGACGAAGACTGACGCTCAGGTGCGAAAGCGTGGGGAGCAA
ACAGGATTAGATACCCTGGTAGTCCACGCCGTAAACGATGTCGACTTGGAGGTTGT
GCCCTTGAGGCGTGGCTTCCGGAGCTAACGCGTTAAGTCGACCGCCTGGGGAGTA
CGGCCGCAAGGTTAAAACTCAAATGAATTGACGGGGGCCCGCACAAGCGGTGGAG
CATGTGGTTTAATTCGATGCAACGCGAAGAACCTTACCTGGTCTTGACATCCACGGA
AGTTTTCAGAGATGAGAATGTGCCTTCGGGAACCGTGAGACAGGTGCTGCATGGCT
GTCGTCAGCTCGTGTTGTGAAATGTTGGGTTAAGTCCCGCAACGAGCGCAACCCTT
ATCCTTTGTTGCCAGCGGTCCGGCCGGGAACTCAAAGGAGACTGCCAGTGATAAAC
TGGAGGAAGGTGGGGATGACGTCAAGTCATCATGGCCCTTACGACCAGGGCTACA
CACGTGCTACAATGGCGCATACAAAGAGAAGCGACCTCGCGAGAGCAAGCGGACC
TCATAAAGTGCGTCGTAGTCCGGATTGGAGTCTGCAACTCGACTCCATGAAGTCGG
AATCGCTAGTAATCGTGGATCAGAATGCCACGGTGAATACGTTCCCGGGCCTTGTA
CACACCGCCCGTCACACCATGGGAGTGGGTTGCAAAAGAAGTAGGTAGCTTAACCT
TCGGGAGGGCGCTTACCACTTTGTGATTCATGACTGGGGTGAAGTCGTAACAAGGT
AACCGTAGGGGAACCTGCGGTTGGATCACCTCCTT

The invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Unless otherwise defined herein, scientific and technical terms used in connection with the present disclosure shall have the meanings that are commonly understood by those of ordinary skill in the art. Further, unless otherwise required by context, singular terms shall include pluralities and plural terms hall include the singular. The methods and techniques of the present disclosure are generally performed according to conventional methods well-known in the art. Generally, nomenclatures used in connection with, and techniques of biochemistry, enzymology, molecular and cellular biology, microbiology, virology, cell or tissue culture, genetics and protein and nucleic chemistry described herein are those well-known and commonly used in the art. The methods and techniques of the present disclosure are generally performed according to conventional methods well known in the art and as described in various general and more specific references that are cited and discussed throughout the present specification unless otherwise indicated.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teaching that is referenced hereinabove. However, the citation of any reference is not intended to be an admission that the reference is prior art.

### EXAMPLES

### Example 1: Efficacy of Bacterial Compositions to Decolonize ESBL

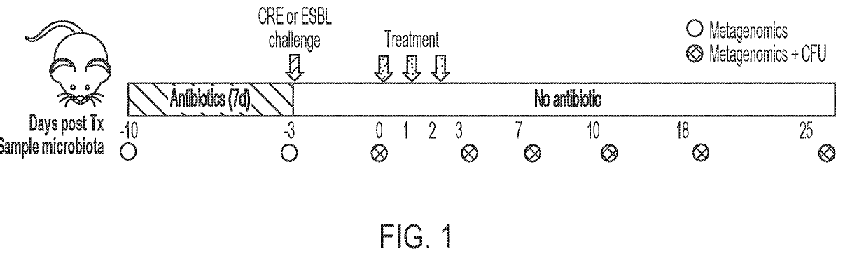

As shown in FIG. 1, mice were treated with antibiotics (e.g., 0.5 g/L ampicillin) for 7 days prior to challenge with CRE or ESBL-producing Enterobacteriaceae challenge (Escherichia coli ATCC BAA-2777, “ESBL”). On day-3, mice were challenged with CRE or ESBL. On days 0.1, and 2, mice were administered treatment (e.g., PBS (control), a 36-mix live biotherapeutic product (36-mix), or stool fraction library from a human donor (Donor 1 SFL)). Fecal samples (pellets) were collected at various time points post-treatment to quantify colony forming units (CFU) as measure of ESBL colonization levels. The efficacy of a treatment in reducing CRE or ESBL colonization was assessed.

As shown in FIG. 2, the 36-mix and SFL derived from Donor 1 were capable of reducing ESBL colonization levels, however the SFL derived from Donor 1 reduce ESBL colonization to lower levels. Several of the mice treated with the 35-mix exhibited a greater reduction in ESBL colonization. Stools from the mice treated with the 36-mix and having the lowest levels of ESBL colonization were subjected to metagenomic sequencing, which identified several mouse-derived taxa that were present in these mice compared to mice that received the 36-mix and higher levels of ESBL colonization.

Additional bacterial strains were evaluated for combination with strains of the 36-mix. A bacterial mixture comprising the strains present in the 36-mix and 11 additional strains, for a total of 47 bacterial strains (47-mix), was prepared. See, FIG. 3 and Table 1.

### Example 2: Efficacy of Bacterial Compositions Comprising 47 Bacterial Strains to Decolonize CRE and ESBL

Mice were treated with antibiotics (e.g., 0.5 g/L ampicillin) for 7 days prior to challenge with carbapenem-resistant Klebsiella pneumoniae challenge (Klebsiella pneuomoniae ATCC BAA-2814, “CRE”) or ESBL-producing Enterobacteriaceae challenge (Escherichia coli ATCC BAA-2777, “ESBL”) (FIG. 1). On day-3, mice were challenged with CRE or ESBL. On days 0, 1, and 2, mice were administered treatment (e.g., PBS (control), or the 47-mix live biotherapeutic product (47-mix). Fecal samples (pellets) were collected at various time points post-treatment to quantify colony forming units (CFU) as measure of CRE or ESBL colonization levels.

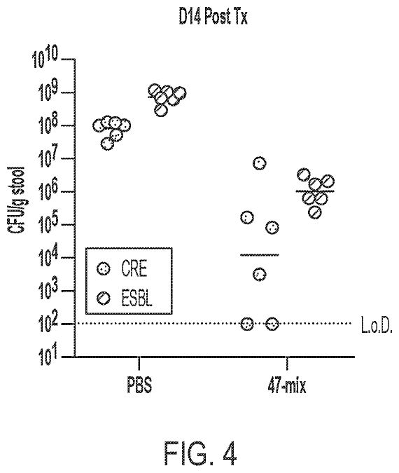

Colonization levels of CRE and ESBL in mice are shown in FIG. 4. Administration of the 47-mix bacterial composition resulted in a reduction of both CRE and ESBL by at least 3-logs as compared to mice that received the control (PBS).

### Example 3: Efficacy of Bacterial Compositions Comprising 47 Bacterial Strains to Decolonize ESBL or CRE

Mice were treated with antibiotics (e.g., 0.5 g/L ampicillin) for 7 days prior to challenge with carbapenem-resistant Klebsiella pneumoniae challenge (Klebsiella pneuomoniae ATCC BAA-2814, “CRE”) or ESBL-producing Enterobacteriaceae challenge (Escherichia coli ATCC BAA-2777, “ESBL”) (FIG. 1). On day-3, mice were challenged with CRE or ESBL. On days 0, 1, and 2, mice were administered treatment (e.g., PBS (control), the 47-mix live biotherapeutic product (47-mix), stool fraction library derived by culturing stool from a second human donor (Donor 2 SFL), or a bacterial mixture obtained by introducing fecal matter from a first human donor (Donor 1) into a mouse and obtaining stool from the mouse to prepare a fecal matter transplant (Donor 1-inoculated mouse FMT).

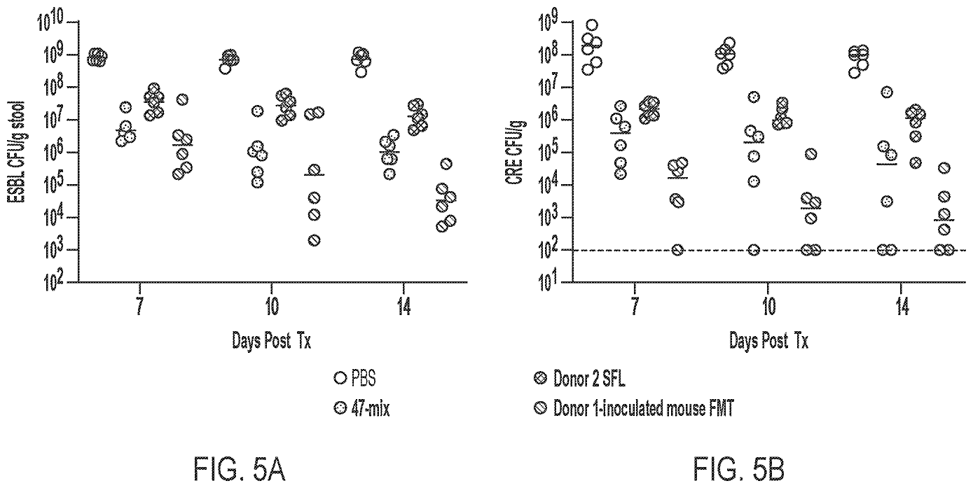

Fecal samples (pellets) were collected at various time points post-treatment to quantify colony forming units (CFU) as measure of CRE or ESBL colonization levels. Colonization levels of ESBL and CRE in mice are shown in FIGS. 5A and 5B, respectively.

Administration of the 47-mix bacterial composition resulted in a reduction in both ESBL and CRE colonization by at least 2-logs by day 7. Colonization of both ESBL and CRE were further reduced at both 10 days and 14 days post treatment.

### Example 4: Efficacy of Bacterial Compositions Comprising Bacterial Strains and Taurine to Decolonize ESBL or CRE

Mice are treated with antibiotics (e.g., 0.5 g/L ampicillin) for 7 days prior to challenge with carbapenem-resistant Klebsiella pneumoniae challenge (Klebsiella pneuomoniae ATCC BAA-2814, “CRE”) or ESBL-producing Enterobacteriaceae challenge (Escherichia coli ATCC BAA-2777, “ESBL”). On day-3, mice are challenged with CRE or ESBL. Mice are administered the following treatment conditions:
PBS (control),
PBS+taurine (taurine only),
live biotherapeutic product (any of the compositions described herein), and
live biotherapeutic product+taurine (any of the compositions described herein+taurine).

Taurine may be administered in the same composition with the live biotherapeutic product or in a separate composition, which may be administered prior to, at the same time, or after administration of the live biotherapeutic product.

Fecal samples (pellets) are collected at various time points post-treatment to quantify colony forming units (CFU) as measure of CRE or ESBL colonization levels.

What is claimed is:

1. A composition comprising two or more purified bacterial strains of species selected from the group consisting of a purified bacterial strain of Collinsella aerofaciens, Bifidobacterium longum, Bifidobacterium pseudocatenulatum, Bifidobacterium adolescentis, Bacteroides caccae, Bacteroides xylanisolvens, Bacteroides cellulosilyticus, Bacteroides uniformis, Bacteroides_B vulgatus, Bacteroides fragilis, Bacteroides thetaiotaomicron, Bacteroides faecis, Bacteroides ovatus, Odoribacter splanchnicus, Parabacteroides distasonis, Parabacteroides merdae, Alistipes putredinis, Alistipes shahii, Paraclostridium massiliensis, Paeniclostridium sordellii, Absiella innocuum, Absiella innocuum, Erysipelatoclostridium ramosum, Clostridium_M citroniae, Clostridium_M clostridioforme, Eubacterium_E hallii, Agathobacter rectale, Blautia producta, Blautia_A obeum, Dorea longicatena, Coprococcus_B comes, Anaerostipes caccae, Agathobaculum sp, Phascolarctobacterium faecium, Escherichia coli, Fusobacterium_A sp, Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri.

2. A composition comprising two or more purified bacterial strains, wherein the two or more purified bacterial strains comprise 16S rDNA sequences having at least 97% sequence identity with nucleic acid sequences selected from the group consisting of SEQ ID NOs: 1-47.

3. The composition of claim 1 or 2, wherein the composition comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, or at least 47 purified bacterial strains.

4. A composition comprising purified bacterial strains of species Bacteroides faecis, Bacteroides vulgatus, Phascolartcobacterium faecium, and Clostridium citroniae.

5. A composition comprising purified bacterial strains comprising 16S rDNA sequences having at least 97% sequence identity to nucleic acid sequences of SEQ ID NOs: 10, 29, 30, and 36.

6. The composition of any one of claims 1-5, wherein the composition comprises bacterial strains that originate from more than one human donor.

7. The composition of any one of claims 1-6, further comprising taurine.

8. A composition comprising
(1) one or more purified bacterial strains belonging to the phylum Firmicutes or Bacteroidetes;
(2) one or more purified bacterial strains selected from the group consisting of an Escherichia species and a Fusobacterium species; and
(3) taurine.

9. A composition comprising
(1) one or more purified bacterial strains belonging to the phylum Firmicutes;
(2) one or more purified bacterial strains of Escherichia species; and
(3) taurine.

10. A composition comprising
(1) one or more purified bacterial strains belonging to the phylum Firmicutes;
(2) one or more purified bacterial strains of Fusobacterium species; and
(3) taurine.

11. A composition comprising
(1) one or more purified bacterial strains belonging to the phylum Bacteroidetes;
(2) one or more purified bacterial strains of Escherichia species and
(3) taurine.

12. A composition comprising
(1) one or more purified bacterial strains belonging to the phylum Bacteroidetes;
(2) one or more purified bacterial strains of Fusobacterium species; and
(3) taurine.

13. A composition comprising a purified bacterial strain belonging to Escherichia species, a purified bacterial strain belonging to Fusobacterium species, and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

14. A composition comprising a purified bacterial strain belonging to Escherichia species, a purified bacterial strain belonging to Fusobacterium species, and one or more purified bacterial strains of species selected from the group consisting of Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, and Phascolarctobacterium faecium; and taurine.

15. A composition comprising a purified bacterial strain belonging to Escherichia species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

16. A composition comprising purified bacterial strain belonging to Fusobacterium species and one or more purified bacterial strains of species selected from the group consisting of Bifidobacterium pseudocatenulatum, Bifidobacterium adolescentis, Bifidobacterium longum, Clostridium citroniae, Clostridium clostridioforme, Clostridium bifermentans, Clostridium sordelli, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, and Phascolarctobacterium faecis; and taurine.

17. A composition comprising a purified bacterial strain belonging to Escherichia species, a purified bacterial strain belonging to Fusobacterium species, and one or more purified bacterial strains of species selected from the group consisting of Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

18. A composition comprising a purified bacterial strain belonging to Escherichia species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

19. A composition comprising a purified bacterial strain belonging to Fusobacterium species and one or more purified bacterial strains of species selected from the group consisting of Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides fragilis, Bacteroides uniformis, Bacteroides vulgatus, Odoribacter splanchnicus, Parabacteroides distasonis, Parabacteroides merdae, Alistipes putredinis, and Alistipes shahii; and taurine.

20. A composition comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species, a purified bacterial strain belonging to Fusobacterium species; and taurine.

21. A composition comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Escherichia species; and taurine.

22. A composition comprising Clostridium bolteae, Anaerotruncus colihominis, Sellimonas intestinalis, Clostridium symbiosum, Blautia producta, Dorea longicatena, Erysipelotrichaceae bacterium, Flavinofractor plautii, a purified bacterial strain belonging to Fusobacterium species; and taurine.

23. A composition comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species, a purified bacterial strain belonging to Fusobacterium species; and taurine.

24. A composition comprising Anaerotruncus colihominis, Sellimonas intestinalis, Clostridium symbiosum, Dorea longicatena, Erysipelotrichaceae bacterium, Flavinofractor plautii, a purified bacterial strain belonging to Escherichia species; and taurine.

25. A composition comprising Anaerotruncus colihominis, Sellimonas intestinalis, Clostridium symbiosum, Dorea longicatena, Erysipelotrichaceae bacterium, Flavinofractor plautii, a purified bacterial strain belonging to Fusobacterium species; and taurine.

26. A composition comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species, a purified bacterial strain belonging to Fusobacterium species; and taurine.

27. A composition comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Escherichia species; and taurine.

28. A composition comprising Clostridium saccharogumia (Clostridium ramosum JCM 1298), Flavonifractor plautii (Pseudoflavonifractor capillosus ATCC 29799), Clostridium hathewayi (Clostridium saccharolyticum WM1), Blautia coccoides (Lachnospiraceae bacterium 6_1_63FAA), Clostridium spp. (Clostridium bolteae ATCC BAA-613), cf. Clostridium sp. MLG055 (Erysipelotrichaceae bacterium 2_2_44A), Clostridium indolis (Anaerostipes caccae DSM 14662), Anaerotruncus colihominis (Anaerotruncus colihominis DSM 17241), Ruminococcus sp. ID8 (Lachnospiraceae bacterium 2_1_46FAA), Clostridium lavalense (Clostridium asparagiforme DSM 15981), Clostridium symbiosum (Clostridium symbiosum WAL-14163), Clostridium ramosum, Eubacterium contortum (Clostridium sp. D5), Clostridium scindens (Lachnospiraceae bacterium 5_1_57FAA), Lachnospiraceae bacterium A4 (Lachnospiraceae bacterium 3_1_57FAA_CT1), Clostridium sp. 316002/08 (Clostriales bacterium 1_7_47FAA), Lachnospiraceae bacterium A4 (Lachnospiraceae bacterium 3_1_57FAA_CT1), a purified bacterial strain belonging to Fusobacterium species; and taurine.

29. A composition comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species, a purified bacterial strain belonging to Fusobacterium species; and taurine.

30. A composition comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Escherichia species; and taurine.

31. A composition comprising Clostridium bolteae, Anaerotruncus colihominis, Sellimonas intestinalis, Clostridium symbiosum, Blautia producta, Erysipelotrichaceae bacterium, Flavinofractor plautii, a purified bacterial strain belonging to Fusobacterium species; and taurine.

32. A composition comprising Collinsella aerofaciens, Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Odoribacter splanchnicus, Parabacteroides distasonis, Parabacteroides merdae, Alistipes putredinis, Alistipes shahii, Bifidobacterium pseudocatenulatum, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, a purified bacterial strain belonging to Fusobacterium species; and taurine.

33. A composition comprising Collinsella aerofaciens, Bacteroides ovatus, Bacteroides thetaiotamicron, Bacteroides xyalinosolvens, Bacteroides cellulosilyticus, Bacteroides uniformis, Bacteroides vulgatus, Parabacteroides distasonis, Alistipes putredinis, Alistipes shahii, Bifidobacterium adolescentis, Bifidobacterium longum, Clostridium citroniae, Clostridium clostridioforme, Clostridium innocuum, Erysipelatoclostridium ramosum, Erysipelotrichaceae bacterium 6_1_45, Eubacterium rectale, Anaerostipes caccae, Blautia obeum, Blautia producta, Coprococcus comes, Dorea longicatena, Agathobaculum butyriciproducens, Phascolarctobacterium faecis, a purified bacterial strain belonging to Escherichia species, a purified bacterial strain belonging to Fusobacterium species; and taurine.

34. A composition comprising Bacteroides ovatus, Bacteroides thetaiotamicron, Bacteroides xyalinosolvens, Bacteroides caccae, Bacteroides cellulosilyticus, Bacteroides faecis, Bacteroides uniformis, Bacteroides vulgatus, Alistipes putredinis, Alistipes shahii, Clostridium citroniae, Clostridium clostridioforme, Clostridium innocuum, Erysipelatoclostridium ramosum, Erysipelotrichaceae bacterium 6_1_45, Eubacterium hallii, Eubacterium rectale, Blautia obeum, Blautia producta, Coprococcus comes, Dorea longicatena, a purified bacterial strain belonging to Escherichia species, a purified bacterial strain belonging to Fusobacterium species; and taurine.

35. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

36. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Clostridium clostridioforme, Blautia obeum, Clostridium innocuum, Agathobaculum butyriciproducens, Clostridium innocuum, Dorea longicatena, Collinsella aerofaciens, Eubacterium hallii, Bacteroides faecis, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

37. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Clostridium clostridioforme, Paraclostridium bifermentans, Clostridium citroniae, Anaerostipes caccae, Fusobacterium mortiferum, Blautia obeum, Clostridium innocuum, Agathobaculum butyriciproducens, Clostridium innocuum, Dorea longicatena, Collinsella aerofaciens, Eubacterium hallii, Bacteroides faecis, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

38. A composition comprising Blautia producta, Bifidobacterium longum, Bifidobacterium adolescentis, Paeniclostridium sordellii, Bifidobacterium pseudocatenulatum, Erysipelatoclostridium ramosum, Escherichia coli, Paraclostridium bifermentans, Clostridium citroniae, Anaerostipes caccae, Fusobacterium mortiferum, Blautia obeum, Clostridium innocuum, Agathobaculum butyriciproducens, Odoribacter sp., Bacteroides fragilis, Bacteroides ovatus, Clostridium bolteae, Anaerotruncus colihominis, Drancourtella massiliensis, Clostridium symbiosum, Blautia producta, Dorea longicatena, Erysipelotrichaceae bacterium, Flavonifractor plautii; and taurine.

39. A composition comprising Bacteroides cellulosilyticus, Bacteroides ovatus, Bacteroides thetaiotaomicron, Bacteroides uniformis, Bacteroides vulgatus, Bifidobacterium adolescentis, Bifidobacterium longum, Blautia producta, Clostridium citroniae, Clostridium clostridioforme, Clostridium innocuum, Clostridium innocuum, Coprococcus comes, Dorea longicatena, Erysipelatoclostridium ramosum, Eubacterium rectale, Parabacteroides distasonis, Bacteroides xylanisolvens, Blautia obeum, Alistipes putredinis, Collinsella aerofaciens, Eubacterium hallii, Anaerostipes caccae, Phascolarctobacterium faecium, Agathobaculum butyriciproducens, Fusobacterium mortiferum, Escherichia coli; and taurine.

40. A composition comprising Bacteroides caccae, Bacteroides cellulosilyticus, Bacteroides ovatus, Bacteroides thetaiotaomicron, Bacteroides uniformis, Bacteroides vulgatus, Bifidobacterium longum, Blautia producta, Clostridium clostridioforme, Clostridium innocuum, Coprococcus comes, Dorea longicatena, Erysipelatoclostridium ramosum, Clostridium citroniae, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Agathobaculum butyriciproducens, Bacteroides fragilis, Fusobacterium mortiferum, Escherichia coli; and taurine.

41. A composition comprising Bacteroides ovatus, Bacteroides vulgatus, Bifidobacterium adolescentis, Bifidobacterium longum, Bifidobacterium pseudocatenulatum, Blautia producta, Clostridium citroniae, Clostridium clostridioforme, Clostridium innocuum, Clostridium innocuum, Paeniclostridium sordellii, Coprococcus comes, Dorea longicatena, Erysipelatoclostridium ramosum, Eubacterium rectale, Odoribacter splanchnicus, Parabacteroides distasonis, Parabacteroides merdae, Bacteroides xylanisolvens, Blautia obeum, Alistipes putredinis, Collinsella aerofaciens, Eubacterium hallii, Alistipes shahii, Anaerostipes caccae, Phascolarctobacterium faecium, Agathobaculum butyriciproducens, Fusobacterium mortiferum, Paraclostridium bifermentans, Escherichia coli; and taurine.

42. A composition comprising Bacteroides ovatus, Bacteroides vulgatus, Bifidobacterium longum, Blautia producta, Clostridium citroniae, Clostridium clostridioforme, Clostridium innocuum, Coprococcus comes, Dorea longicatena, Erysipelatoclostridium ramosum, Odoribacter splanchnicus, Parabacteroides distasonis, Bacteroides xylanisolvens, Blautia obeum, Alistipes putredinis, Collinsella aerofaciens, Alistipes shahii, Anaerostipes caccae, Phascolarctobacterium faecium, Fusobacterium mortiferum, Escherichia coli; and taurine.

43. A composition comprising Bacteroides faecis, Bacteroides ovatus, Bacteroides vulgatus, Bifidobacterium adolescentis, Bifidobacterium longum, Blautia producta, Clostridium citroniae, Clostridium clostridioforme, Clostridium innocuum, Clostridium innocuum, Paeniclostridium sordellii, Dorea longicatena, Erysipelatoclostridium ramosum, Odoribacter splanchnicus, Blautia obeum, Alistipes putredinis, Collinsella aerofaciens, Eubacterium hallii, Anaerostipes caccae, Phascolarctobacterium faecium, Agathobaculum butyriciproducens, Bacteroides fragilis, Fusobacterium mortiferum, Paraclostridium bifermentans, Escherichia coli; and taurine.

44. A composition comprising Bacteroides faecis, Bacteroides ovatus, Bacteroides vulgatus, Bifidobacterium longum, Blautia producta, Clostridium citroniae, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Phascolarctobacterium faecium, Agathobaculum butyriciproducens, Fusobacterium mortiferum, Escherichia coli; and taurine.

45. A composition comprising bacterial strains of species Bacteroides vulgatus, Clostridium citroniae, and Phascolarctobacterium faecium, and one or more purified bacterial strains of species selected from the group consisting of Bacteroides faecis, Bacteroides ovatus, Bifidobacterium longum, Blautia producta, Clostridium clostridioforme, Clostridium innocuum, Dorea longicatena, Erysipelatoclostridium ramosum, Odoribacter splanchnicus, Alistipes putredinis, Collinsella aerofaciens, Blautia obeum, Anaerostipes caccae, Agathobaculum butyriciproducens, Fusobacterium mortiferum, Escherichia coli; and
taurine.

46. A composition comprising Collinsella aerofaciens, Bifidobacterium longum, Bacteroides ovatus, Bacteroides faecis, Bacteroides vulgatus, Odoribacter splanchnicus, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Dorea longicatena, Agathobaculum butyriciproducens, Phascolarctobacterium faecium, Escherichia coli, Fusobacterium mortiferum; and taurine.

47. A composition comprising Collinsella aerofaciens, Bifidobacterium longum, Bacteroides ovatus, Bacteroides vulgatus, Parabacteroides distasonis, Alistipes putredinis, Clostridium citroniae, Clostridium clostridioforme, Erysipelatoclostridium ramosum, Erysipelotrichaceae bacterium 6_1_45, Anaerostipes caccae, Blautia obeum, Blautia producta, Agathobaculum butyriciproducens, Phascolarctobacterium faecium, Escherichia coli, Fusobacterium mortiferum; and taurine.

48. A composition comprising Collinsella aerofaciens, Bifidobacterium longum, Bacteroides ovatus, Bacteroides vulgatus, Alistipes putredinis, Clostridium citroniae, Clostridium clostridioforme, Erysipelatoclostridium ramosum, Erysipelotrichaceae bacterium 6_1_45, Blautia obeum, Blautia producta, Dorea longicatena, Phascolarctobacterium faecium, Escherichia coli, Fusobacterium mortiferum; and taurine.

49. A composition comprising purified bacterial strains of species Bacteroides faecis, Bacteroides vulgatus, Phascolartcobacterium faecium, and Clostridium citroniae; and taurine.

50. The composition of any one of claims 8-49, further comprising one or more bacterial strain selected from the group consisting of Barnesiella intestinihominis, Blautia luti, Blautia faecis, Blautia wexlerae, Ruminococcus faecis, Clostridium bolteae, Butyricimonas synergistica, Bilophila wadsworthia, Akkermansia muciniphila, Parasuterella excrementinihominis, and Prevotella copri.

51. The composition of any one of claims 8-50, wherein the composition comprises between about 40 mg to about 3000 mg taurine.

52. The composition of claim 51, wherein the composition comprises between about 100 mg to about 2000 mg taurine.

53. The composition of claim 51, wherein the composition comprises between about 500 mg to about 1500 mg taurine.

54. The composition of any one of claims 1-53, wherein the composition is effective in suppressing the replication, survival, and/or colonization of one or more pathogenic organisms.

55. The composition of any one of claims 1-54, wherein the composition is effective in treating an infection by a pathogenic organism in a subject.

56. The composition of claim 54 or 55, wherein the pathogenic organism is susceptible to antibiotics.

57. The composition of claim 54 or 55, wherein the pathogenic organism is resistant to one or more antibiotics.

58. The composition of any one of claim 54, 55, or 57, wherein the pathogenic organism is a multi-drug resistant organism.

59. The composition of claim 58, wherein the multi-drug resistant organism is Carbapenem Resistant Enterobacteriaceae (CRE) or Extended spectrum beta-lactamase (ESBL)-producing Enterobacteriaceae.

60. The composition of claim 59, wherein the Carbapenem Resistant Enterobacteriaceae (CRE) is carbapenem-resistant Klebsiella pneumoniae.

61. The composition of claim 60, wherein the carbapenem-resistant Klebsiella pneumoniae is carbapenem-resistant Klebsiella pneumoniae ATCC 700721.

62. The composition of claim 59, wherein the ESBL-producing Enterobacteriaceae is ESBL-producing K. pneumoniae or EBSL-producing E. coli.

63. The composition of claim 62, wherein the ESBL-producing Enterobacteriaceae is ESBL-producing K. pneumoniae subsp. pneumoniae (ATCC 700721), ESBL-producing E. coli ATCC BAA 2777, adherent/invasive E. coli (AIEC), Shiga toxin-producing E. coli (STEC), Verocytotoxin-producing E. coli (VTEC), enterohemorrhagic E. coli (EHEC), enteropathogenic E. coli (EPEC), enteroaggregative E. coli (EAEC), enteroinvasive E. coli (EIEC), or diffusely adherent E. coli (DAEC).

64. The composition of any one of claims 1-63, wherein the bacterial strains are lyophilized.

65. The composition of any one of claims 1-63, wherein the bacterial strains are spray-dried.

66. The composition of any one of claims 1-65, wherein one or more of the bacterial strains are in spore form.

67. The composition of any one of claims 1-66, wherein each of the bacterial strains is in spore form.

68. The composition of any one of claims 1-66, wherein one or more of the bacterial strains are in vegetative form.

69. The composition of any one of claim 1-65 or 68, wherein each of the bacterial strains is in vegetative form.

70. The composition of any one of claims 1-69, wherein the composition further comprises one or more enteric polymers.

71. A pharmaceutical composition comprising the composition of any one of claims 1-70, further comprising a pharmaceutically acceptable excipient.

72. The pharmaceutical composition of claim 71, wherein the pharmaceutical composition is formulated for oral delivery.

73. The pharmaceutical composition of claim 71 or 72, wherein the pharmaceutical composition is formulated for rectal delivery.

74. The pharmaceutical composition of any one of claims 71-73, wherein the pharmaceutical composition is formulated for delivery to the intestine.

75. The pharmaceutical composition of any one of claims 71-74, wherein the pharmaceutical composition is formulated for delivery to the colon.

76. The pharmaceutical composition of any one of claims 71-75, wherein the pharmaceutical composition is administered as one dose.

77. The pharmaceutical composition of any one of claims 71-75, wherein the pharmaceutical composition is administered as multiple doses.

78. The pharmaceutical composition of claim 76 or 77, wherein each dose comprises the administration of multiple capsules.

79. A food product comprising the composition of any one of claims 1-78 and a nutrient.

80. A method of suppressing infection by a pathogenic organism in a subject, comprising administering to the subject a therapeutically effective amount of the composition of any one of claims 1-78 or the food product of claim 79.

81. A method of reducing or preventing colonization by a pathogenic organism in a subject, comprising administering to the subject a therapeutically effective amount of the composition of any one of claims 1-78 or the food product of claim 79.

82. A method of treating infection by a pathogenic organism in a subject, comprising administering to the subject a therapeutically effective amount of the composition of any one of claims 1-78 or the food product of claim 79.

83. The method of any one of claims 80-82, wherein the pathogenic organism is a multi-drug resistant organism.

84. The method of claim 80-83, wherein the pathogenic organism is Klebsiella pneumoniae.

85. The method of claim 84, wherein the Klebsiella pneumoniae is multi-drug resistant.

86. The method of claim 85, wherein the multi-drug resistant Klebsiella pneumoniae is carbapenem-resistant Klebsiella pneumoniae.

87. The method of claim 80-86, wherein the pathogenic organism is Extended spectrum beta-lactamase (ESBL) producing Enterobacteriaceae.

88. The method of claim 87, wherein the ESBL producing Enterobacteriaceae is Escherichia coli (E. coli).

89. The method of claim 88, wherein the E. coli is adherent/invasive E. coli (AIEC), Shiga toxin-producing E. coli (STEC), Verocytotoxin-producing E. coli (VTEC), enterohemorrhagic E. coli (EHEC), enteropathogenic E. coli (EPEC), enteroaggregative E. coli (EAEC), enteroinvasive E. coli (EIEC), or diffusely adherent E. coli (DAEC).

90. The method of any one of claims 80-89, wherein the subject is human.

91. The method of any one of claims 80-90, wherein the composition is administered to the subject more than once.

92. The method of any one of claims 80-91, wherein the composition is administered to the subject by oral administration.

93. The method of any one of claims 80-91, wherein the composition is administered to the subject by rectal administration.

94. The method of any one of claims 80-93, wherein the administering suppresses the replication, survival, and/or colonization of the pathogenic organism.

95. The method of any one of claims 80-94, wherein the administration of the composition is not preceded by administration of vancomycin.

96. The method of any one of claims 80-94, further comprising administering an antibiotic to the subject prior to administration of the composition.

97. The method of claim 96, wherein the antibiotic is vancomycin.

98. The method of any one of claims 80-97, further comprising administering a composition comprising taurine to the subject.

99. The method of claim 98, wherein the composition comprising taurine is administered to the subject prior to and/or after administration of the composition of any one of claims 1-78 or the food product of claim 79.

100. The method of any one of claims 80-97, wherein the subject has been administered a composition comprising taurine prior to the administration of the composition of any one of claims 1-78 or the food product of claim 79.

101. The method of any one of claims 98-100, wherein the subject is administered between about 40 mg to about 3000 mg taurine per day.

102. The method of any one of claims 98-101, wherein the subject receives between about 100 mg to about 2000 mg taurine per day.

103. The method of any one of claims 98-102, wherein the subject receives between about 500 mg to about 1500 mg taurine per day.
